(19) United States
(12) Patent Application Publication
(10) Pub. No.: US 2021/0139477 A1
(43) Pub. Date: May 13, 2021

(54) ALK5 INHIBITORS, CONJUGATES, AND USES THEREOF

(71) Applicant: Silverback Therapeutics, Inc., Seattle, WA (US)

(72) Inventors: Sean Wesley Smith, Seattle, WA (US); Craig Alan Coburn, Seattle, WA (US); Peter Robert Baum, Seattle, WA (US); Robert Finley Dubose, Seattle, WA (US)

(21) Appl. No.: 16/931,431

(22) Filed: Jul. 16, 2020

Related U.S. Application Data
(60) Provisional application No. 62/874,886, filed on Jul. 16, 2019.

(51) Int. Cl. C07D 471/04; C07D 401/14; C07D 405/14; A61K 47/68

(52) U.S. Cl. CPC C07D 471/04; A61K 47/6801; C07D 405/14; C07D 401/14

(57) ABSTRACT

ALK5 inhibitor compounds, conjugates, and pharmaceutical compositions for use in the treatment of disease, such as cancer, are disclosed herein. The disclosed compounds are useful, among other things, in the treating of cancer and fibrosis and modulating ALK5. Additionally, compounds incorporated into a conjugate with an antibody construct are described herein.

### STATEMENT REGARDING SEQUENCE LISTING

The sequence listing associated with this application is provided in text format in lieu of a paper copy, and is hereby incorporated by reference into the specification. The name of the text file containing the sequence listing is Sequence_Listing.txt. The text file is 296 KB, was created on Jul. 16, 2020, and is being submitted electronically via efs-web.

### BACKGROUND

One of the leading causes of death in the United States is cancer. The conventional methods of cancer treatment, like chemotherapy, surgery, or radiation therapy, tend to be either highly toxic or nonspecific to a cancer, or both, resulting in limited efficacy and harmful side effects. However, the immune system has the potential to be a powerful, specific tool in fighting cancers. In many cases tumors can specifically express genes whose products are required for inducing or maintaining the malignant state. These proteins may serve as antigen markers for the development and establishment of more specific anti-cancer immune response. The boosting of this specific immune response has the potential to be a powerful anti-cancer treatment that can be more effective than conventional methods of cancer treatment and can have fewer side effects.

Fibrosis is the formation of excess fibrous connective tissue or scar tissue in an organ or tissue in a reparative or reactive process. Fibrosis can occur in many tissues within the body, typically as a result of inflammation or damage, which include the lungs, liver, heart, and brain. Scar tissue blocks arteries, immobilizes joints and damages internal organs, wreaking havoc on the body's ability to maintain vital functions. Every year, millions of people are hospitalized due to the damaging effects of fibrosis. However, current therapeutics for treating fibrotic diseases are lacking or have drawbacks. Thus, there remains a considerable need for alternative or improved treatments for fibrotic diseases.

### BRIEF SUMMARY

The present disclosure provides, inter alia, compounds represented by the structure of Formula (I):

[chemical structure]

or a salt thereof, wherein the variable M¹, M², R¹, R², R⁶, Y, w, and n are as described herein.

In some embodiments, compounds disclosed herein are attached to a linker to form compound-linkers.

In some embodiments, compounds disclosed herein are covalently bound to an antibody construct or a targeting moiety, optionally via a linker.

Also disclosed herein are pharmaceutical compositions of the compounds or conjugates described herein.

In some aspects, the present disclosure provides a method for treating cancer, comprising administering a compound, a conjugate, or a pharmaceutical composition as described herein to a subject in need thereof.

In some aspects, the present disclosure provides a method for enhancing an immune response (e.g., an anti-cancer immune response) in a subject comprising administering a compound, a conjugate, or a pharmaceutical composition as described herein to a subject in need thereof.

In some aspects, the present disclosure provides a method for treating fibrosis, comprising administering a compound, a conjugate, or a pharmaceutical composition as described herein to a subject in need thereof. In some aspects, the fibrosis is cancer-associated. In some aspects, the fibrosis is not cancer-associated. In one aspect, the fibrosis is scleroderma. In another aspect, the fibrosis is systemic fibrosis. In one aspect, the fibrotic disease is steatohepatitis., e.g., non-alcoholic steatohepatitis (NASH).

### DETAILED DESCRIPTION

The instant disclosure provides ALK5 inhibitor compounds, linkers, conjugates, and products comprising the same, and to methods for their synthesis and use. While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

The present disclosure provides compounds, conjugates and pharmaceutical compositions for use in the treatment of disease. In certain embodiments, the compounds of the disclosure are activin receptor-like kinase 5 (ALK5) inhibitors. Activin receptor-like kinase 5 (ALK5), which is also commonly known as transforming growth factor beta receptor 1 (TGF-βR1), is a serine/threonine kinase transmembrane receptor. It is a part of the TGFβ signaling pathway and is involved in signal transduction from the cell surface to the cytoplasm. The TGFβ signaling pathway regulates gene expression of genes involved in cellular processes such as differentiation, apoptosis, wound healing, and cell growth. ALK5 and TGF-βR1 can be used interchangeably.

In the absence of TGFβ ligands, ALK5 remains a homodimeric cell surface receptor. However, ligand binding to type II TGFβ receptor (TGFβR2) induces the formation of the TGFβR1/TGFβR2 complex, which leads to phosphorylation of Mothers Against Decapentaplegic homolog 2 (Smad2) and Mothers Against Decapentaplegic homolog 3 (Smad3) and subsequent modulation of a number of downstream signaling targets involved in the regulation of gene expression. As such, inhibitors of ALK5 may be useful in altering or modulating the expression of genes involved in cancer, and thus, may be useful in treating and preventing cancer.

The compounds of the present disclosure may act as ALK5 inhibitors. The compounds, salts, and conjugates of the present disclosure may be useful for treatment and/or prevention, e.g., vaccination, of cancer, autoimmune diseases, inflammation, fibrosis, sepsis, allergy, asthma, graft rejection, graft-versus-host disease, immunodeficiencies, and infectious diseases.

In certain embodiments, the compounds, salts, and conjugates have utility in the treatment of cancer either as single agents or in combination therapy. In certain embodiments, the compounds, salts, and conjugates have utility as single agent immunomodulators, vaccine adjuvants and in combination with conventional cancer therapies. In certain embodiments, the compounds and salts are incorporated into a conjugate that can be utilized, for example, to enhance an immune response. In certain embodiments, the disclosure provides conjugates including a compound or salt described herein and an antibody construct.

### Definitions

Prior to setting forth this disclosure in more detail, it may be helpful to an understanding thereof to provide definitions of certain terms to be used herein. Additional definitions are set forth throughout this disclosure.

Unless defined otherwise, all technical and scientific terms used herein have the same meaning as is commonly understood by one of skill in the art to which this invention belongs.

As used in the specification and claims, the singular form “a,” “an,” and “the” includes plural references unless the context clearly dictates otherwise. It should be understood that the terms “a” and “an” as used herein refer to “one or more” of the enumerated components.

The use of the alternative (e.g., “or”) should be understood to mean either one, both, or any combination thereof of the alternatives. As used herein, the terms “include” and “comprise” are used synonymously.

The phrase “at least one of” when followed by a list of items or elements refers to an open ended set of one or more of the elements in the list, which may, but does not necessarily, include more than one of the elements.

The term “about” as used herein in the context of a number refers to a range centered on that number and spanning 15% less than that number and 15% more than that number. The term “about” used in the context of a range refers to an extended range spanning 15% less than that the lowest number listed in the range and 15% more than the greatest number listed in the range.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size, or thickness, are to be understood to include any integer within the recited range, unless otherwise indicated.

The phrase “targeting moiety” refers to a structure that has a selective affinity for or selectively binds to a target molecule relative to other non-target molecules. A targeting moiety may include, for example, an antibody, an antibody construct, a peptide, a polypeptide, a ligand, carbohydrate, a polynucleotide, an oligonucleotide, or a receptor or a binding portion thereof. The target biological molecule may be a biological receptor or other structure of a cell, such as a tumor antigen.

As used herein, the term “antibody” refers to an immunoglobulin molecule that specifically binds to, or is immunologically reactive toward, a specific antigen. The portion of the antibody that binds a specific antigen may be referred to as an “antigen binding domain.” An antibody can include, for example, polyclonal, monoclonal, and genetically engineered antibodies, and antigen binding fragments thereof. An antibody can be, for example, murine, chimeric, humanized, heteroconjugate, bispecific, diabody, triabody, or tetrabody. An antigen binding fragment includes an antigen binding domain and can be in the form of, for example, a Fab′, F(ab′)₂, Fab, Fv, rIgG, scFv, hcAbs (heavy chain antibodies), a single domain antibody, V_{HH}, V_{NAR}, sdAb, or nanobody.

As used herein, an “antigen binding domain” refers to a region of a molecule that specifically binds to an antigen. An antigen binding domain can be an antigen-binding portion of an antibody or an antibody fragment. An antigen binding domain can be one or more fragments of an antibody that can retain the ability to specifically bind to an antigen. An antigen binding domain can be an antigen binding fragment. In some embodiments, an antigen binding domain can recognize a single antigen. An antigen binding domain can recognize, for example, two or three antigens. As used herein, “recognize” with regard to antibody interactions refers to the association or binding between an antigen binding domain of an antibody or portion thereof and an antigen.

As used herein, “specifically binds” and the like refers to the specific association or specific binding between the antigen binding domain and the antigen, as compared with the interaction of the antigen binding domain with a different antigen (i.e., non-specific binding). In some embodiments, an antigen binding domain that recognizes or specifically binds to an antigen has a dissociation constant (KD) of <<100 nM, <10 nM, <1 nM, <0.1 nM, <0.01 nM, or <0.001 nM (e.g., 10⁻⁸ M or less; from about 10⁻⁸ M to about 10⁻¹³ M; from about 10⁻⁹ M to about 10⁻¹³ M). Specific binding does not require that the antigen binding domain not associate with or bind to any other antigen, but rather that it preferentially associates with or binds to the target antigen of interest, as compared to off-target association with or binding to an unrelated antigen.

As used herein, an “antibody construct” refers to a molecule, e.g., a protein, peptide, antibody or portion thereof, that contains an antigen binding domain and an Fc region (e.g., an Fc domain from within the Fc region).

As used herein, a “Fc domain” can be from within an Fc region of an antibody or from within a non-antibody molecule domain that can bind to an Fc receptor.

As used herein, a “tumor antigen” can be an antigenic substance associated with a tumor or cancer cell, and can trigger an immune response in a host.

As used herein, “identical” or “identity” refer to the similarity between a DNA, RNA, nucleotide, amino acid, or protein sequence to another DNA, RNA, nucleotide, amino acid, or protein sequence. Identity can be expressed in terms of a percentage of sequence identity of a first sequence to a second sequence. Percent (%) sequence identity with respect to a reference DNA sequence can be the percentage of DNA nucleotides in a candidate sequence that are identical with the DNA nucleotides in the reference DNA sequence after aligning the sequences. Percent (%) sequence identity with respect to a reference amino acid sequence can be the percentage of amino acid residues in a candidate sequence that are identical with the amino acid residues in the reference amino acid sequence after aligning the sequences and introducing gaps, if necessary, to achieve the maximum percent sequence identity, and not considering any conservative substitutions as part of the sequence identity. In certain embodiments, the percent sequence identity values are generated using the NCBI BLAST 2.0 software as defined by Altschul et al., Nucleic Acids Res. 25:3389-3402, 2007, with the parameters set to default values.

As used herein, a compound of the disclosure, e.g., a compound or salt of Formulas (IA), (IB), (IC), (ID), (IE), (IF), and Table 10, or pharmaceutically acceptable isomer, racemate, hydrate, solvate, isotope, or salt thereof, may be referred to herein as a “TGFβR1 inhibitor,” an “ALK5 inhibitor,” a drug, “D,” or a payload, “P,” particularly when referenced as part of a conjugate. In some embodiments, an ALK5 inhibitor inhibits the ALK5 activity by about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 100% as compared to ALK5 activity in the absence of the inhibitor.

In some aspects, an ALK5 inhibitor has an IC₅₀ value of between 0.1 nM and 1000 nM, between 0.1 nm and 100 nM, or between 0.1 nM and 80 nM in an ALK5 enzyme inhibition assay. An exemplary ALK5 enzyme inhibition assay is as set forth in the example section.

In some aspects, ALK5 inhibitor has an IC₅₀ value of between 0.1 nM and 1000 nM, between 0.1 nm and 100 nM, between 0.1 nM and 80 nM, or between 0.1 nM and 10 nM in a TGF-βR1 reporter assay. An exemplary TGF-βR1 reporter assay is as set forth in the example section.

In some aspects, an ALK5 inhibitor has an IC₅₀ value of between 0.1 nM and 1000 nM, between 0.1 nm and 100 nM, or between 0.1 nM and 80 nM in a ALK5 enzyme inhibition assay and an IC₅₀ value of between 0.1 nM and 1000 nM, between 0.1 nm and 100 nM, between 0.1 nM and 80 nM, or between 0.1 nM and 10 nM in a TGF-βR1 reporter assay.

“LP,” “linker-payload,” “L³-D,” or “linker-ALK5 inhibitor,” “linker-compound,” “linker-drug,” may be used herein to refer to a compound of Formula (I), (IA), (IB), (IC), (ID), (IE), (IF), and Table 16, or pharmaceutically acceptable isomer, racemate, hydrate, solvate, isotope, or salt thereof, bound to a linker.

As used herein, “conjugate” refers to an antibody, antibody construct, or targeting moiety that is attached (e.g., conjugated) either directly or through a linker group to a compound described herein, e.g., a compound of Formula (I), (IA), (IB), (IC), (ID), (IE), (IF), and Table 16, or pharmaceutically acceptable isomer, racemate, hydrate, solvate, isotope, or salt thereof.

As used herein, drug-to-antibody ratio (“DAR”) refers to a particular number of compounds (ALK5 inhibitors) of a conjugate that are covalently attached or linked, directly or indirectly (via a linker), to an antibody, antibody construct, or targeting moiety. For a conjugate having more than one compound covalently attached or linked, the linked compounds may be the same or different. In certain embodiments, a conjugate is represented by the following formula:

A [symbol] L-D)_{z},

wherein A is an antibody, an antibody construct, or a targeting moiety, Lisa linker, D is a compound of Formula (I), (IA), (IB), (IC), (ID), (IE), (IF), and Table 16, or pharmaceutically acceptable isomer, racemate, hydrate, solvate, isotope, or salt thereof, and z is from 1 to about 20. In some embodiments, z ranges from 1 to about 10, from 1 to about 9, from 1 to about 8, from 2 to about 8, from 1 to about 6, from 1 to about 3 or from about 3 to about 5. In certain embodiments, z is 2, about 3, about 4, about 5, about 6, about 7, or about 8. In further embodiments, conjugates are represented by the following formula:

A [symbol] D)_{z},

wherein A is an antibody, an antibody construct, or a targeting moiety, D is a compound of Formula (I), (IA), (IB), (IC), (ID), (IE), (IF), and Table 16, or pharmaceutically acceptable isomer, racemate, hydrate, solvate, isotope, or salt thereof, and z ranges from 1 to about 20. In some embodiments, z ranges from 1 to about 10, from 1 to about 9, from 1 to about 8, from 2 to about 8, from 1 to about 6, from 1 to about 3 or from about 3 to about 5. In certain embodiments, z is 2, about 3, about 4, about 5, about 6, about 7, or about 8. A population of conjugates found in, for example, a composition or formulation will have an average DAR. In some embodiments, the average DAR for the conjugates of a composition or formulation will range from 1 to about 10, from 1 to about 9, from 1 to about 8, from 1 to about 6, from 1 to about 3, from about 2 to about 8, from about 2 to about 6, from about 2.5 to about 5.5, from about 2.5 to about 4.5, from about 2 to about 4, from about 3.5 to about 5.5, from about 3 to about 5, from about 3.5 to about 4.5, or from about 3 to about 4. In certain embodiments, the average DAR for the conjugates of a composition or formulation will be about 1.5, about 2, about 2.5, about 3, about 3.5, about 4, about 4.5, about 5, about 5.5, about 6, about 6.5, about 7, about 7.5, or about 8.

As used herein, a compound of this disclosure, e.g., a compound of Formula (I), (IA), (IB), (IC), (ID), (IE), (IF), and Table 16, or pharmaceutically acceptable isomer, racemate, hydrate, solvate, isotope, or salt thereof, also may be referred to as a TGFβR1 inhibitor, an ALK5 inhibitor, a drug, D, or a payload, particularly when referenced as part of a conjugate. “LP”, “linker-payload”, “L³-D”, or “compound-linker” may be used interchangeably herein to refer to a compound of Formula (I), (IA), (IB), (IC), (ID), (IE), (IF), and Table 16, or pharmaceutically acceptable isomer, racemate, hydrate, solvate, isotope, or salt thereof, covalently bound to a linker, L.

As used herein, the abbreviations for the natural L-enantiomeric amino acids are conventional and can be as follows: alanine (A, Ala); arginine (R, Arg); asparagine (N, Asn); aspartic acid (D, Asp); cysteine (C, Cys); glutamic acid (E, Glu); glutamine (Q, Gin); glycine (G, Gly); histidine (H, His); isoleucine (I, He); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val).

As used herein, a “target binding domain” refers to a construct that contains an antigen binding domain from an antibody or from a non-antibody that can bind to the antigen.

The term “targeting moiety” refers to a structure that has a selective affinity for a target molecule relative to other non-target molecules. The targeting moiety binds to a target molecule. A targeting moiety may include, for example, an antibody, a peptide, a ligand, a receptor, or a binding portion thereof. The target molecule may be an antigen, such as a biological receptor or other structure of a cell such as a tumor antigen.

As used herein, a “tumor antigen” can be an antigenic substance associated with a tumor or cancer cell, and can trigger an immune response in a host.

The term “C_{x-y}” or “Cₓ-C_{y}” when used in conjunction with a chemical moiety, such as alkyl, alkenyl, or alkynyl is meant to include groups that contain from x to y carbons in the chain. For example, the term “C₁-C₆alkyl” refers to substituted or unsubstituted saturated hydrocarbon groups, including straight-chain alkyl and branched-chain alkyl groups that contain from 1 to 6 carbons. The term —C_{x-y}alkylene- refers to a substituted or unsubstituted alkylene chain with from x to y carbons in the alkyl ene chain. For example —C₁₋₆ alkylene- may be selected from methylene, ethylene, propylene, butylene, pentylene, and hexylene, any one of which is optionally substituted.

The terms “C_{x-y}alkenyl” and “C_{x-y}alkynyl” refer to substituted or unsubstituted unsaturated aliphatic groups analogous in length and possible substitution to the alkyls described above, but that contain at least one double or triple bond, respectively. The term —C_{x-y}alkenylene-refers to a substituted or unsubstituted alkenylene chain with from x to y carbons in the alkenylene chain. For example, —C₂₋₆alkenylene- may be selected from ethenylene, propenylene, butenylene, pentenylene, and hexenylene, any one of which is optionally substituted. An alkenylene chain may have one double bond or more than one double bond in the alkenylene chain. The term —C_{x-y}alkynylene- refers to a substituted or unsubstituted alkynylene chain with from x to y carbons in the alkynylene chain. For example, —C₂₋₆alkynylene- may be selected from ethynylene, propynylene, butynylene, pentynylene, and hexynylene, any one of which is optionally substituted. An alkynylene chain may have one triple bond or more than one triple bond in the alkynylene chain.

“Alkylene” refers to a straight divalent hydrocarbon chain linking the rest of the molecule to a radical group, consisting solely of carbon and hydrogen, containing no unsaturation, and preferably having from one to twelve carbon atoms, for example, methylene, ethylene, propylene, butylene, and the like. The alkylene chain is attached to the rest of the molecule through a single bond and to the radical group through a single bond. The points of attachment of the alkylene chain to the rest of the molecule and to the radical group are through the terminal carbons respectively. In other embodiments, an alkylene comprises one to five carbon atoms (i.e., C₁-C₅ alkylene). In other embodiments, an alkylene comprises one to four carbon atoms (i.e., C₁-C₄ alkylene). In other embodiments, an alkylene comprises one to three carbon atoms (i.e., C₁-C₃ alkylene). In other embodiments, an alkylene comprises one to two carbon atoms (i.e., C₁-C₂ alkylene). In other embodiments, an alkylene comprises one carbon atom (i.e., C₁ alkylene). In other embodiments, an alkylene comprises five to eight carbon atoms (i.e., C₅-C₈ alkylene). In other embodiments, an alkylene comprises two to five carbon atoms (i.e., C₂-C₅ alkylene). In other embodiments, an alkylene comprises three to five carbon atoms (i.e., C₃-C₅ alkylene). “Alkenylene” refers to a straight divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. The points of attachment of the alkenylene chain to the rest of the molecule and to the radical group are through the terminal carbons, respectively. In other embodiments, an alkenylene comprises two to five carbon atoms (i.e., C₂-C₅ alkenylene). In other embodiments, an alkenylene comprises two to four carbon atoms (i.e., C₂-C₄ alkenylene). In other embodiments, an alkenylene comprises two to three carbon atoms (i.e., C₂-C₃ alkenylene). In other embodiments, an alkenylene comprises two carbon atom (i.e., C₂ alkenylene). In other embodiments, an alkenylene comprises five to eight carbon atoms (i.e., C₅-C₈ alkenylene). In other embodiments, an alkenylene comprises three to five carbon atoms (i.e., C₃-C₅ alkenylene). “Alkynylene” refers to a straight divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. The points of attachment of the alkynylene chain to the rest of the molecule and to the radical group are through the terminal carbons respectively. In other embodiments, an alkynylene comprises two to five carbon atoms (i.e., C₂-C₅ alkynylene). In other embodiments, an alkynylene comprises two to four carbon atoms (i.e., C₂-C₄ alkynylene). In other embodiments, an alkynylene comprises two to three carbon atoms (i.e., C₂-C₃ alkynylene). In other embodiments, an alkynylene comprises two carbon atom (i.e., C₂ alkynylene). In other embodiments, an alkynylene comprises five to eight carbon atoms (i.e., C₅-C₈ alkynylene). In other embodiments, an alkynylene comprises three to five carbon atoms (i.e., C₃-C₅ alkynylene).

“Heteroalkylene” refers to a straight divalent hydrocarbon chain including at least one heteroatom in the chain, containing no unsaturation, and preferably having from one to twelve carbon atoms and from one to 6 heteroatoms, e.g., —O—, —NH—, —S—. The heteroalkylene chain is attached to the rest of the molecule through a single bond and to the radical group through a single bond. The points of attachment of the heteroalkylene chain to the rest of the molecule and to the radical group are through the terminal atoms of the chain. In other embodiments, a heteroalkylene comprises one to five carbon atoms and from one to three heteroatoms. In other embodiments, a heteroalkylene comprises one to four carbon atoms and from one to three heteroatoms. In other embodiments, a heteroalkylene comprises one to three carbon atoms and from one to two heteroatoms. In other embodiments, a heteroalkylene comprises one to two carbon atoms and from one to two heteroatoms. In other embodiments, a heteroalkylene comprises one carbon atom and from one to two heteroatoms. In other embodiments, a heteroalkylene comprises five to eight carbon atoms and from one to four heteroatoms. In other embodiments, a heteroalkylene comprises two to five carbon atoms and from one to three heteroatoms. In other embodiments, a heteroalkylene comprises three to five carbon atoms and from one to three heteroatoms.

The term “carbocycle” as used herein refers to a saturated, unsaturated or aromatic ring in which each atom of the ring is carbon. Carbocycle includes 3- to 10-membered monocyclic rings, 6- to 12-membered bicyclic rings, and 6- to 12-membered bridged rings. Each ring of a bicyclic carbocycle may be selected from saturated, unsaturated, and aromatic rings. In an exemplary embodiment, an aromatic ring, e.g., phenyl, may be fused to a saturated or unsaturated ring, e.g., cyclohexane, cyclopentane, or cyclohexene. A bicyclic carbocycle includes any combination of saturated, unsaturated and aromatic bicyclic rings, as valence permits. A bicyclic carbocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. Exemplary carbocycles include cyclopentyl, cyclohexyl, cyclohexenyl, adamantyl, phenyl, indanyl, and naphthyl.

The term “aryl” refers to an aromatic monocyclic or aromatic multicyclic hydrocarbon ring system. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene.

The term “cycloalkyl” refers to a saturated ring in which each atom of the ring is carbon. Cycloalkyl may include monocyclic and polycyclic rings such as 3- to 10-membered monocyclic rings, 6- to 12-membered bicyclic rings, and 6- to 12-membered bridged rings. In certain embodiments, a cycloalkyl comprises three to ten carbon atoms. In other embodiments, a cycloalkyl comprises five to seven carbon atoms. The cycloalkyl may be attached to the rest of the molecule by a single bond. Examples of monocyclic cycloalkyls include, e.g., cyclopropyl, cyclobutyl, cyclopentyl, cyclohexyl, cycloheptyl, and cyclooctyl. Polycyclic cycloalkyl radicals include, for example, adamantyl, norbomyl (i.e., bicyclo[2.2.1]heptanyl), decalinyl, 7,7 dimethyl bicyclo[2.2.1]heptanyl, and the like.

The term “halo” or, alternatively, “halogen” or “halide,” means fluoro, chloro, bromo or iodo. In some embodiments, halo is fluoro, chloro, or bromo.

The term “haloalkyl” refers to an alkyl radical, as defined above, that is substituted by one or more halo radicals, for example, trifluoromethyl, dichloromethyl, bromomethyl, 2,2,2-trifluoroethyl, l-chloromethyl-2-fluoroethyl, and the like.

The term “heterocycle” as used herein refers to a saturated, unsaturated or aromatic ring comprising one or more heteroatoms. Exemplary heteroatoms include N, O, Si, P, B, and S atoms. Heterocycles include 3- to 10-membered monocyclic rings, 6- to 12-membered bicyclic rings, and 6- to 12-membered bridged rings. A bicyclic heterocycle includes any combination of saturated, unsaturated and aromatic bicyclic rings, as valence permits. In an exemplary embodiment, an aromatic ring, e.g., pyridyl, may be fused to a saturated or unsaturated ring, e.g., cyclohexane, cyclopentane, morpholine, piperidine or cyclohexene. A bicyclic heterocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. Examples of unsaturated heterocycles include dihydropyrrole, dihydrofuran, oxazoline, pyrazoline, and dihydropyridine.

The term “heteroaryl” includes aromatic single ring structures, preferably 5- to 7-membered rings, more preferably 5- to 6-membered rings, whose ring structures include at least one heteroatom, preferably one to four heteroatoms, more preferably one or two heteroatoms. The term “heteroaryl” also includes polycyclic ring systems having two or more rings in which two or more atoms are common to two adjoining rings wherein at least one of the rings is heteroaromatic, e.g., the other rings can be aromatic or non-aromatic carbocyclic, or heterocyclic. Heteroaryl groups include, for example, pyrrole, furan, thiophene, imidazole, oxazole, thiazole, pyrazole, pyridine, pyrazine, pyridazine, and pyrimidine, and the like.

The term “heterocycloalkyl” refers to a saturated ring with carbon atoms and at least one heteroatom. Exemplary heteroatoms include N, O, Si, P, B, and S atoms. Heterocycloalkyl may include monocyclic and polycyclic rings such as 3- to 10-membered monocyclic rings, 6- to 12-membered bicyclic rings, and 6- to 12-membered bridged rings. The heteroatoms in the heterocycloalkyl radical are optionally oxidized. One or more nitrogen atoms, if present, are optionally quaternized. The heterocycloalkyl is attached to the rest of the molecule through any atom of the heterocycloalkyl, valence permitting, such as any carbon or nitrogen atoms of the heterocycloalkyl. Examples of heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl.

The term “substituted” refers to moieties having substituents replacing a hydrogen on one or more carbons or substitutable heteroatoms, e.g., an NH or NH₂ of a compound. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 0, 1, or 2), —R^{b}—S(O)ₜR^{a} (where t is 1 or 2), —R^{b}—S(O)ₜOR^{a} (where t is 0, 1, or 2), and —R^{b}—S(O)ₜN(R^{a})₂ (where t is 0, 1, or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl, any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 0, 1, or 2), —R^{b}—S(O)ₜR^{a} (where t is 0, 1, or 2), —R^{b}—S(O)ₜOR^{a} (where t is 0, 1, or 2) and —R^{b}—S(O)ₜN(R^{a})₂ (where t is 0, 1, or 2); wherein:
each R^{a} is, independently, hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, is independently substituted with one or more alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 0, 1, or 2), —R^{b}—S(O)ₜR^{a} (where t is 0, 1, or 2), —R^{b}—S(O)ₜOR^{a} (where t is 0, 1, or 2), or —R^{b}—S(O)ₜN(R^{a})₂ (where t is 0, 1, or 2);
each R^{b} is, independently, a direct bond or a straight or branched alkylene, alkenylene, or alkynylene chain, and
each R^{c} is, independently, a straight or branched alkylene, alkenylene or alkynylene chain.

“Protecting group” refers to a moiety, except alkyl groups, that when attached to a reactive group in a molecule masks, reduces or prevents that reactivity. Examples of protecting groups can be found in T. W. Greene and P. G. M. Wuts, Protective Groups in Organic Synthesis, 3.sup.rd edition, John Wiley & Sons, New York, 1999, and Harrison and Harrison et al., Compendium of Synthetic Organic Methods, Vols. 1-8 (John Wiley and Sons, 1971-1996), which are incorporated herein by reference in their entirety. Representative amino or amine protecting groups include, formyl, acyl groups (such as acetyl, trifluoroacetyl, and benzoyl), benzyl, alkoxycarbonyl (such as benzyloxycarbonyl (CBZ), and tert-butoxycarbonyl (Boc)), trimethyl silyl (TMS), 2-trimethylsilyl-ethanesulfonyl (SES), trityl and substituted trityl groups, allyloxycarbonyl, 9-fluorenylmethyloxycarbonyl (FMOC), nitro-veratryloxycarbonyl (NVOC), sulfonyl, and the like. Compounds described herein can include protecting groups (e.g., a hydrogen on a reactive nitrogen atom of a compound described herein can be replaced by an amino protecting group).

“Isomer” is used herein to encompass all chiral, diastereomeric or racemic forms of a structure, unless a particular stereochemistry or isomeric form is specifically indicated. Such compounds can be enriched or resolved optical isomers at any or all asymmetric atoms as are apparent from the depictions, at any degree of enrichment. Both racemic and diastereomeric mixtures, as well as the individual optical isomers can be synthesized so as to be substantially free of their enantiomeric or diastereomeric partners, and these are all within the scope of certain embodiments of the invention. The isomers resulting from the presence of a chiral center comprise a pair of nonsuperimposable-isomers that are called “enantiomers.” Single enantiomers of a pure compound are optically active (i.e., they are capable of rotating the plane of plane polarized light and designated R or S).

“Isolated optical isomer” means a compound which has been substantially purified from the corresponding optical isomer(s) of the same formula. For example, the isolated isomer may be at least about 80%, at least 80% or at least 85% pure by weight. In other embodiments, the isolated isomer is at least 90% pure or at least 98% pure, or at least 99% pure by weight.

“Substantially enantiomerically or diastereomerically” pure means a level of enantiomeric or diastereomeric enrichment of one enantiomer with respect to the other enantiomer or diastereomer of at least about 80%, and more specifically in excess of 80%, 85%, 90%, 95%, 98%, 99%, 99.5% or 99.9%.

The terms “racemate” and “racemic mixture” refer to an equal mixture of two enantiomers. A racemate is labeled “(±)” because it is not optically active (i.e., will not rotate plane-polarized light in either direction since its constituent enantiomers cancel each other out). All compounds with an asterisk (*) adjacent to a tertiary or quaternary carbon are optically active isomers, which may be purified from the respective racemate and/or synthesized by appropriate chiral synthesis.

A “hydrate” is a compound that exists in combination with water molecules. The combination can include water in stoichiometric quantities, such as a monohydrate or a dihydrate, or can include water in random amounts. As the term is used herein a “hydrate” refers to a solid form; that is, a compound in a water solution, while it may be hydrated, is not a hydrate as the term is used herein.

A “solvate” is similar to a hydrate except that a solvent other that water is present. For example, methanol or ethanol can form an “alcoholate”, which can again be stoichiometric or non-stoichiometric. As the term is used herein a “solvate” refers to a solid form; that is, a compound in a solvent solution, while it may be solvated, is not a solvate as the term is used herein.

“Isotope” refers to atoms with the same number of protons but a different number of neutrons, and an isotope of a compound of Formula (I) includes any such compound wherein one or more atoms are replaced by an isotope of that atom. For example, carbon 12, the most common form of carbon, has six protons and six neutrons, whereas carbon 13 has six protons and seven neutrons, and carbon 14 has six protons and eight neutrons. Hydrogen has two stable isotopes, deuterium (one proton and one neutron) and tritium (one proton and two neutrons). While fluorine has a number of isotopes, fluorine 19 is longest-lived. Thus, an isotope of a compound having the structure of Formula (I) includes, but not limited to, compounds of Formula (I) wherein one or more carbon 12 atoms are replaced by carbon-13 and/or carbon-14 atoms, wherein one or more hydrogen atoms are replaced with deuterium and/or tritium, and/or wherein one or more fluorine atoms are replaced by fluorine-19.

“Salt” generally refers to an organic compound, such as a carboxylic acid or an amine, in ionic form, in combination with a counter ion. For example, salts formed between acids in their anionic form and cations are referred to as “acid addition salts”. Conversely, salts formed between bases in the cationic form and anions are referred to as “base addition salts.”

### Target Genes and Proteins

The present disclosure provides conjugates for use in the treatment of disease. As used herein, “conjugate” refers to an antibody, antibody construct, or targeting moiety that is attached (i.e., conjugated) either directly or through a linker group to an ALK5 inhibitor compound described herein. Antibodies, antibody constructs, and targeting moieties in the present disclosure have a selective affinity for a target molecule relative to other non-target molecules. The antibody, antibody construct, and/or a targeting moiety binds to a target molecule. The target molecule may be an antigen, such as a biological receptor or other structure of a cell such as a tumor antigen. The target genes and proteins disclosed herein may serve as antigen markers for the development and establishment of more specific disease treatment, for example, more specific anti-cancer immune response.

CTLA4 gene encodes CTLA4 protein (cytotoxic T-lymphocyte-associated protein 4), also known as CD152 (cluster of differentiation 152), which is a protein receptor that acts as an immune checkpoint and downregulates immune responses. CTLA4 is constitutively expressed in Tregs but only upregulated in conventional T cells after activation. CTLA4 acts as an “off” switch when bound to CD80 or CD86 on the surface of antigen-presenting cells. The monoclonal antibody Ipilimamab has been developed to target CTLA4.

PDCD1 encodes programmed cell death protein 1, also known as PD-1 and CD279 (cluster of differentiation 279), which is a cell surface receptor that plays a cell surface receptor that plays an important role in down-regulating the immune system and promoting self-tolerance by suppressing T cell inflammatory activity. PD-1 is a cell surface receptor that belongs to the immunoglobulin superfamily and is expressed on T cells and pro-B cells. PD-1 is an immune checkpoint and guards against autoimmunity through a dual mechanism of promoting apoptosis (programmed cell death) in antigen specific T-cells in lymph nodes while simultaneously reducing apoptosis in regulatory T cells (anti-inflammatory, suppressive T cells). The human IgG4 anti-PD-1 monoclonal antibody Opdivo® (nivolumab) and humanized antibody Keytruda® (pembrolizumab) have been developed to target PD-1. The antibodies pidilizumab (CT-011, Cure Tech) and BMS-936559 are in clinical development.

CD274 encodes PD-L1 (programmed death-ligand 1), also known as CD274 (cluster of differentiation 274). PD-L1 is a 40 kDa type 1 transmembrane protein that has been speculated to play a major role in suppressing the immune system during particular events such as pregnancy, tissue allografts, autoimmune disease and other disease states such as hepatitis. The binding of PD-L1 to PD-1 or B7.1 transmits an inhibitory signal which reduces the proliferation of CD8+ T cells at the lymph nodes and supplementary to that PD-1 is also able to control the accumulation of foreign antigen specific T cells in the lymph nodes through apoptosis which is further mediated by a lower regulation of the gene Bcl-2. The monoclonal antibodies Atezolizumab, Durvalumab, avelumab, and MDX-1106 have been developed to target PD-L1.

TNFR2 (tumor necrosis factor receptor 2), also known as TNFRSF1B (tumor necrosis factor receptor super family IB) and CD120b, is a single-pass type I membrane protein and the member of TNFR superfamily containing 4 cysteine-rich domains (CRD) repeats. In addition to the full length membrane-anchored form, soluble TNFR2 can be generated via two distinct mechanisms: (1) shedding via proteolytic processing of the full membrane anchored from, and (2) translation from an alternatively spliced message encoding the extracellular domains of TNFR2. TNFR2 is the receptor with high affinity for TNF-alpha and approximately 5-fold lower affinity for homotrimeric lymphotoxin-alpha. The mouse monoclonal antibodies against TNFR2 described by SEQ ID NO: 56-SEQ ID NO: 82, and SEQ ID NO: 95-SEQ ID NO: 103, and anti-TNFR2 antibodies described by SEQ ID NO: 104 and SEQ ID NO: 105 have been developed to target TNFR2.

TNFRSF4 encodes OX40, also known as TNFRSF4 (tumor necrosis factor receptor superfamily, member 4), a member of the TNFR-superfamily of receptors which is not constitutively expressed on resting naïve T cells, unlike CD28. OX40 is a secondary co-stimulatory immune checkpoint molecule, expressed after 24 to 72 hours following activation; its ligand, OX40L, is also not expressed on resting antigen presenting cells, but is following their activation. Expression of OX40 is dependent on full activation of the T cell; without CD28, expression of OX40 is delayed and of fourfold lower levels. The monoclonal antibody Vonlerolizumab has been developed to target OX40.

CD27 is a member of the tumor necrosis factor receptor superfamily. The protein encoded by this gene is a member of the TNF-receptor superfamily. This receptor is required for generation and long-term maintenance of T cell immunity. It binds to ligand CD70, and plays a key role in regulating B-cell activation and immunoglobulin synthesis. This receptor transduces signals that lead to the activation of NF-κB and MAPK8/JNK. Adaptor proteins TRAF2 and TRAF5 have been shown to mediate the signaling process of this receptor. CD27-binding protein (SIVA), a proapoptotic protein, can bind to this receptor and is thought to play an important role in the apoptosis induced by this receptor. The monoclonal antibody Varlilumab has been developed to target CD27.

IL2RA encodes CD25, also known as IL2RA (interleukin-2 receptor alpha chain), which is a type I transmembrane protein present on activated T cells, activated B cells, some thymocytes, myeloid precursors, and oligodendrocytes. IL2RA is expressed in most B-cell neoplasms, some acute nonlymphocytic leukemias, neuroblastomas, mastocytosis and tumor infiltrating lymphocytes. It functions as the receptor for HTLV-1 and is consequently expressed on neoplastic cells in adult T cell lymphoma/leukemia. Its soluble form, called sIL-2R may be elevated in these diseases and is occasionally used to track disease progression. The humanized monoclonal antibody Zinbryta® (Daclizumab) has been developed to target CD25.

TNFRSF18 encodes GITR (glucocorticoid-induced TNFR-related protein), also known as TNFRSF18 (tumor necrosis factor receptor superfamily member 18) and AITR (activation-inducible TNFR family receptor), which is a protein that is a member of the tumor necrosis factor receptor (TNF-R) superfamily. GITR (glucocorticoid-induced tumor necrosis factor receptor) is a surface receptor molecule that has been shown to be involved in inhibiting the suppressive activity of T-regulatory cells and extending the survival of T-effector cells. The anti-GITR antibodies described by SEQ ID NO: 37-SEQ ID NO: 42 and SEQ ID NO: 187-SEQ ID NO: 188, and antibody TRX518 have been developed to target GITR.

LAG-3 (lymphocyte-activation gene 3) encodes a cell surface molecule with diverse biologic effects on T cell function. LAG-3 is an immune checkpoint receptor. The LAG3 protein, which belongs to immunoglobulin (Ig) superfamily, comprises a 503-amino acid type I transmembrane protein with four extracellular Ig-like domains, designated D1 to D4. LAG-3 is expressed on activated T cells, natural killer cells, B cells and plasmacytoid dendritic cells. The anti-LAG-3 antibodies described by SEQ ID NO: 43-SEQ ID NO: 48 and SEQ ID NO: 111-SEQ ID NO: 112 have been developed to target LAG-3.

GARP (glycoprotein A repetitions predominant) is a transmembrane protein containing leucine rich repeats, which is present on the surface of stimulated Treg clones but not on Th clones. The anti-GARP antibodies described by SEQ ID NO: 113-SEQ ID NO: 122 have been developed to target GARP.

4-1BB is a type 2 transmembrane glycoprotein belonging to the TNF superfamily, expressed on activated T Lymphocytes. 4-1BB can be expressed by activated T cells. 4-1BB expression can be found on dendritic cells, B cells, follicular dendritic cells, natural killer cells, granulocytes and cells of blood vessel walls at sites of inflammation. The anti-4-1BB antibodies described by SEQ ID NO: 50-SEQ ID NO: 55 and SEQ ID NO: 123-SEQ ID NO: 128 have been developed to target 4-1BB.

ICOS (Inducible T-cell COStimulator) encodes a CD28-superfamily costimulatory molecule that is expressed on activated T cells. The protein encoded by this gene belongs to the CD28 and CTLA-4 cell-surface receptor family. ICOS forms homodimers and plays an important role in cell-cell signaling, immune responses and regulation of cell proliferation. The anti-ICOS antibodies described by SEQ ID NO: 129-SEQ ID NO: 132 have been developed to target ICOS.

CD70 is expressed on highly activated lymphocytes, such as in T- and B-cell lymphomas. CD70 is a cytokine that belongs to the tumor necrosis factor (TNF) ligand family. This cytokine is a ligand for TNFRSF27/CD27. It is a surface antigen on activated, but not on resting, T and B lymphocytes. CD70 induces proliferation of co-stimulated T cells, enhances the generation of cytolytic T cells, and contributes to T cell activation. This cytokine is also reported to play a role in regulating B-cell activation, cytotoxic function of natural killer cells, and immunoglobulin synthesis. The monoclonal antibody Vorsetuzumab has been developed to target CD70.

PDGFRβ (beta-type platelet-derived growth factor receptor) encodes a typical receptor tyrosine kinase, which is a transmembrane protein consisting of an extracellular ligand binding domain, a transmembrane domain and an intracellular tyrosine kinase domain. The molecular mass of the mature, glycosylated PDGFRβ protein is approximately 180 kDA. The monoclonal antibody Rinucumab has been developed to target PDGFRβ.

CD73 (cluster of differentiation 73), known as ecto-5′-nucleotidase (ecto-5′-NT, EC 3.1.3.5) is a glycosyl-phosphatidylinositol (GPI)-linked 70-kDa cell surface enzyme found in most tissues. CD73 commonly serves to convert AMP to adenosine. Ecto-5-prime-nucleotidase (5-prime-ribonucleotide phosphohydrolase; EC 3.1.3.5) catalyzes the conversion at neutral pH of purine 5-prime mononucleotides to nucleosides, the preferred substrate being AMP. The enzyme consists of a dimer of 2 identical 70-kD subunits bound by a glycosyl phosphatidyl inositol linkage to the external face of the plasma membrane. The enzyme is used as a marker of lymphocyte differentiation. The monoclonal antibody Oleclumab and the anti-CD73 antibodies described in SEQ ID NO: 139-SEQ ID NO: 140 have been developed to target CD73.

CD38 (cluster of differentiation 38), also known as cyclic ADP ribose hydrolase, is a glycoprotein found on the surface of many immune cells (white blood cells), including CD4+, CD8+, B lymphocytes and natural killer cells. CD38 also functions in cell adhesion, signal transduction and calcium signaling. The loss of CD38 function is associated with impaired immune responses, metabolic disturbances, and behavioral modifications including social amnesia possibly related to autism. The CD38 protein is a marker of cell activation. It has been connected to HIV infection, leukemias, myelomas, solid tumors, type II diabetes mellitus and bone metabolism, as well as some genetically determined conditions. CD38 produces an enzyme which regulates the release of oxytocin within the central nervous system. The monoclonal antibody Daratumumab has been developed to target CD38.

Integrin αvβ3 is a type of integrin that is a receptor for vitronectin. Integrin αvβ3 consists of two components, integrin alpha V and integrin beta 3 (CD61), and is expressed by platelets. Integrin αvβ3 is a receptor for phagocytosis on macrophages or dendritic cells. The monoclonal antibodies Etaracizumab and Intetumumab have been developed to target Integrin αvβ3.

Integrin αvβ8, a VN receptor, is identified as a potential negative regulator of cell growth. The cytoplasmic domain of β8 is divergent in sequence, lacking all amino acid homology with the highly homologous cytoplasmic domains of the other αv-associating integrin β subunits (β1, β3, β5, and β6). The β8 cytoplasmic domain is divergent in function. αvβ8 has a restricted distribution and is most highly expressed in nonproliferating cell types. The anti-Integrin αvβ8 antibodies as described in SEQ ID NO: 147-SEQ ID NO: 148 have been developed to target Integrin αvβ8.

CD248 encodes endosialin. Endosialin is a member of the “Group XIV”, a novel family of C-type lectin transmembrane receptors which play a role not only in cell-cell adhesion processes but also in host defense. Endosialin has been associated with angiogenesis in the embryo, uterus and in tumor development and growth. Monoclonal antibody Ontuxizumab has been developed to target endosialin.

FAP (fibroblast activation protein alpha) is a 170 kDa melanoma membrane-bound gelatinase, protein that in humans is encoded by the FAP gene. The protein encoded by this gene is a homodimeric integral membrane gelatinase belonging to the serine protease family. It is selectively expressed in reactive stromal fibroblasts of epithelial cancers, granulation tissue of healing wounds, and malignant cells of bone and soft tissue sarcomas. This protein is thought to be involved in the control of fibroblast growth or epithelial-mesenchymal interactions during development, tissue repair, and epithelial carcinogenesis. The anti-FAP antibodies as described in SEQ ID NO: 151-SEQ ID NO: 168 have been developed to target FAP.

Integrin αv subunit associates with one of five integrin β subunits, β1, β3, β5, β6, or β8, to form five distinct αVβ heterodimers. The integrin αVβ heterodimers on the cell surface interact with cell adhesive proteins, such as collagen, fibrinogen, fibronectin, and vitronectin. These interactions play an important role in cell adhesion or migration, especially in tumor metastasis. Monoclonal antibody intetumumab and anti-Integrin αv antibodies as described in SEQ ID NO: 171-SEQ ID NO: 174 have been developed to target Integrin αv.

Integrinαvβ6 is an epithelial-specific integrin that is a receptor for the extracellular matrix (ECM) proteins fibronectin, vitronectin, tenascin and the latency associated peptide (LAP) of TGF-β. Integrin αvβ6 is not expressed in healthy adult epithelia but is upregulated during wound healing and in cancer. Integrin αvβ6 has been shown to modulate invasion, inhibit apoptosis, regulate the expression of matrix metalloproteases (MMPs) and activate TGF-β1. The anti-Integrin αvβ6 antibodies as described in SEQ ID NO: 175-SEQ ID NO: 182 have been developed to target Integrin αvβ6.

ASGR1, also known as asialoglycoprotein receptor 1, is a major subunit of asialoglycoprotein receptor. Asialoglycoprotein receptor is a hetero-oligomeric protein composed of major and minor subunits and is highly expressed on the surface of hepatocytes, several human carcinoma cell lines, and liver cancers. Asialoglycoprotein receptor mediates the endocytosis of plasma glycoproteins to which the terminal sialic acid residue on their complex carbohydrate moieties has been removed. The receptor recognizes terminal galactose and N-acetylgalactosamine units. After ligand binding to the receptor, the resulting complex is internalized and transported to a sorting organelle, where receptor and ligand are disassociated. The receptor then returns to the cell membrane surface. The asialoglycoprotein receptor may facilitate hepatic infection by multiple viruses including hepatitis B. ASGR1 includes mammalian ASGR1 proteins, e.g., mouse, rat, rabbit, guinea pig, pig, sheep, dog, non-human primate, and human. In some embodiments, ASGR1 refers to an alternatively spliced variant. In some embodiments, ASGR1 is a human ASGR1 having the amino acid sequence set forth in accession NP_001184145.1 or NP_001662.1.

In some embodiments, an anti-ASGR1 antibody or antigen binding fragment thereof comprises:
a) a heavy chain CDR1 (HCDR1) comprising the amino acid sequence of SEQ ID NO:241, an HCDR2 comprising the amino acid sequence selected from any one of SEQ ID NOS:242-244, an HCDR3 comprising the amino acid sequence of SEQ ID NO:245; and a light chain CDR1 (LCDR1) comprising the amino acid sequence of SEQ ID NO:246, a LCDR2 comprising the amino acid sequence selected from any one of SEQ ID NOS:247-249, and a LCDR3 comprising the amino acid sequence of SEQ ID NO:250;
b) a HCDR1 comprising the amino acid sequence of SEQ ID NO:251, an HCDR2 comprising the amino acid sequence of SEQ ID NO:252, an HCDR3 comprising the amino acid sequence of SEQ ID NO:253; and a LCDR1 comprising the amino acid sequence of SEQ ID NO:254, a LCDR2 comprising the amino acid sequence of SEQ ID NO:255 or SEQ ID NO:256, and a LCDR3 comprising the amino acid sequence of SEQ ID NO:257;
c) a HCDR1 comprising the amino acid sequence of SEQ ID NO:258, an HCDR2 comprising the amino acid sequence of SEQ ID NO:259, an HCDR3 comprising the amino acid sequence of SEQ ID NO:260; and a LCDR1 comprising the amino acid sequence of SEQ ID NO:261, a LCDR2 comprising the amino acid sequence of SEQ ID NO:262, and a LCDR3 comprising the amino acid sequence of SEQ ID NO:263;
d) a HCDR1 comprising the amino acid sequence of SEQ ID NO:264, an HCDR2 comprising the amino acid sequence of SEQ ID NO:265, an HCDR3 comprising the amino acid sequence of SEQ ID NO:266; and a LCDR1 comprising the amino acid sequence of SEQ ID NO:267, a LCDR2 comprising the amino acid sequence selected from any one of SEQ ID NOS:268-270, and a LCDR3 comprising the amino acid sequence of SEQ ID NO:271;
e) a HCDR1 comprising the amino acid sequence of SEQ ID NO:272, a HCDR2 comprising the amino acid sequence of SEQ ID NO:273, an HCDR3 comprising the amino acid sequence of SEQ ID NO:274; and a LCDR1 comprising the amino acid sequence of SEQ ID NO:275, a LCDR2 comprising the amino acid sequence of SEQ ID NO:276, and a LCDR3 comprising the amino acid sequence of SEQ ID NO:277; or
f) a HCDR1 comprising the amino acid sequence of SEQ ID NO:235, a HCDR2 comprising the amino acid sequence of SEQ ID NO:236, an HCDR3 comprising the amino acid sequence of SEQ ID NO:237; and a LCDR1 comprising the amino acid sequence of SEQ ID NO:238, a LCDR2 comprising the amino acid sequence of SEQ ID NO:239, and a LCDR3 comprising the amino acid sequence of SEQ ID NO:240.
   Antibodies. Antibody Constructs, and Targeting Moieties

Disclosed herein are antibodies, antibody constructs, and targeting moieties that may each be individually combined with an ALK5 inhibitor compound as disclosed herein. In certain embodiments, ALK5 inhibitor compounds of this disclosure are conjugated either directly or through a linker to an antibody, an antibody construct, or a targeting moiety to form a conjugate.

Terms understood by those in the art of antibody technology are each given the meaning acquired in the art, unless expressly defined differently herein. An intact antibody comprises at least two heavy (H) chains and two light (L) chains inter-connected by disulfide bonds, as well as an antigen binding fragment or portion (which includes an antigen binding domain) of an intact antibody that has or retains the capacity to bind a target molecule. A monoclonal antibody or antigen-binding portion thereof may be non-human, chimeric, humanized, or human, preferably humanized or human. Immunoglobulin structure and function are reviewed, for example, in Greenfield et al., Ed., Antibodies: A Laboratory Manual, Second Edition, Chapter 2 (Cold Spring Harbor Laboratory Press, Cold Spring Harbor, 2014).

For example, the terms “VL” and “VH” refer to the variable binding region from an antibody light and heavy chain, respectively. The variable binding regions are made up of discrete, well-defined sub-regions known as “complementarity determining regions” (CDRs) and “framework regions” (FRs). The term “CL” refers to an “immunoglobulin light chain constant region” or a “light chain constant region,” i.e., a constant region from an antibody light chain. The term “CH” refers to an “immunoglobulin heavy chain constant region” or a “heavy chain constant region,” which is further divisible, depending on the antibody isotype into CH1, CH2, and CH3 (IgA, IgD, IgG), or CH1, CH2, CH3, and CH4 domains (IgE, IgM). Exemplary heavy chain constant regions include human IgG1 heavy chain constant region (SEQ ID NO:278), human IgG1null heavy chain constant region (SEQ ID NO:279), mouse IgG2a heavy chain constant region (SEQ ID NO:281), and rat IgG2b heavy chain constant region (SEQ ID NO:283).

An antigen binding domain of an antibody may comprise one or more light chain (L) CDRs and one or more heavy chain (H) CDRs. For example, an antigen binding domain of an antibody may comprise one or more of the following: a light chain complementary determining region 1 (LCDR1), a light chain complementary determining region 2 (LCDR2), or a light chain complementary determining region 3 (LCDR3). Another exemplary antigen binding domain may comprise one or more of the following: a heavy chain complementary determining region 1 (HCDR1), a heavy chain complementary determining region 2 (HCDR2), or a heavy chain complementary determining region 3 (HCDR3). Another exemplary antigen binding domain of an antibody may comprise one or more of the following: LCDR1, LCDR2, LCDR3, HCDR1, HCDR2, and HCDR3. In some embodiments, an antigen binding domain of an antibody includes all six CDRs, (i.e., LCDR1, LCDR2, LCDR3, HCDR1, HCDR2, and HCDR3).

An antibody, an antibody construct, or a targeting moiety of this disclosure may comprise an antibody light chain variable region having an amino acid sequence with at least one, about two, about three, about four, about five, about six, about seven, about eight, about nine or about ten modifications (e.g., insertion, deletion, mutation), provided that the modifications are not within the light chain CDRs. In certain embodiments, the light chain variable region amino acid sequence does not have more than about 25, about 20, about 15, about 10, about 9, about 8, about 7, about 6, about 5, about 4, about 3, about 2, or 1 modifications relative to the natural or original light chain variable region amino acid sequence, provided that the modifications are not within the light chain CDRs. An antibody may comprise a heavy chain variable region of an amino acid sequence having at least one, two, about three, about four, about five, about six, about seven, about eight, about nine, or about ten modifications (e.g., insertion, deletion, mutation), provided that the modifications are not within the heavy chain CDRs. In certain embodiments, the heavy chain variable region amino acid sequence does not have more than about 25, about 20, about 15, about 10, about 9, about 8, about 7, about 6, about 5, about 4, about 3, 2, or 1 modifications relative to the natural or original heavy chain variable region amino acid sequence, provided that the modifications are not within the heavy chain CDRs.

As used herein, a “Fab” (fragment antigen binding) is a fragment or portion of an antibody that binds to antigens and includes the variable region and CH1 of the heavy chain linked to the light chain via an inter-chain disulfide bond. An antibody construct or targeting moiety may comprise an antigen binding fragment from an antibody. An antigen binding fragment from an antibody may include (i) an antigen binding fragment (Fab), which is a monovalent fragment comprising the V_{L}, V_{H}, CL and CH domains, optionally comprising all or a portion (including at least one Cys residue) of a hinge region (Fab′); (ii) a F(ab′)₂ fragment, a bivalent fragment comprising two Fab fragments linked by a disulfide bridge at the hinge region; (iii) a single-domain antibody (sdAb) or nanobody, which comprises a single monomeric variable antibody domain; or (iv) a Fv fragment comprising the V_{L} and V_{H} domains of a single arm of an antibody. Although the two domains of the Fv fragment, V_{L} and V_{H}, may be coded for by separate genes, they may be linked by a synthetic linker to be made as a single protein chain in which the V_{L} and V_{H} regions pair to form monovalent molecules referred to as as single chain variable fragments (scFv).

An antigen binding domain of an antibody, antibody construct, or targeting moiety may be selected from any domain that specifically binds the antigen including, but not limited to, from a monoclonal antibody, a polyclonal antibody, a recombinant antibody, or binding functional fragment thereof, for example, a heavy chain variable domain (V_{H}) and a light chain variable domain (V_{L}), or a DARPin, an affimer, an avimer, a knottin, a monobody, an affinity clamp, an ectodomain, a receptor ectodomain, a receptor, a cytokine, a ligand, an immunocytokine, a T cell receptor, or a recombinant T cell receptor. An antibody construct or targeting moiety may be in the form of a single chain antibody, an anticalin, a centyrin, an affibody, a knottin, a diabody, a DARPin, or a peptibody. In certain embodiments, an antibody construct or a targeting moiety is an antibody.

An antibody may be of any class, e.g., IgA, IgD, IgE, IgG, and IgM. Several of these classes may be further subdivided into isotypes, e.g., IgG1, IgG2, IgG3, IgG4, IgA1, and IgA2. The heavy-chain constant regions (Fc) that corresponds to the different classes of immunoglobulins may be α, δ, ε, γ, or μ. The light chains may be one of either kappa (κ) or lambda (λ), based on the amino acid sequences of the constant domains. A nonlimiting exemplary human kappa constant domain is shown in SEQ ID NO:280. Another exemplary light chain constant region is mouse kappa constant domain shown in SEQ ID NO:282. Another exemplary light chain constant domain is rat kappa constant domain shown in SEQ ID NO:284.

An antibody construct or targeting moiety may contain, for example, two, three, four, five, six, seven, eight, nine, ten, or more antigen binding domains. An antibody construct or targeting moiety may contain two antigen binding domains in which each antigen binding domain can recognize the same antigen. An antibody construct or targeting moiety may contain two antigen binding domains in which each antigen binding domain can recognizes a different antigen. In certain embodiments, an antibody construct or targeting moiety may comprise an Fc fusion protein. In further embodiments, an antibody construct or targeting moiety may comprise an antibody.

In any of the aforementioned embodiments, an antigen binding domain specifically binds to a tumor antigen, such as mesothelin (MSLN), HER2, CEA, TROP2, EPHA2, p-cadherin, UPK1B, FOLH1, LYPD3, and PVRL4 (Nectin-4). An antigen binding domain may specifically bind to a molecule on an antigen presenting cell (APC).

As used herein, “an Fc region constant domain portion” or “Fc region portion” refers to the heavy chain constant region segment of the Fc fragment (the “fragment crystallizable” region or Fc region) from an antibody, which can in include one or more constant domains, such as CH2, CH3, CH4, or any combination thereof. In certain embodiments, an Fc region portion includes the CH2 and CH3 domains of an IgG, IgA, or IgD antibody and any combination thereof, or the CH3 and CH4 domains of an IgM or IgE antibody and any combination thereof.

By way of background, the Fc region is responsible for the effector functions of an immunoglobulin, such as ADCC (antibody-dependent cell-mediated cytotoxicity), ADCP (antibody-dependent cellular phagocytosis), CDC (complement-dependent cytotoxicity) and complement fixation, binding to Fc receptors (e.g., CD16, CD32, FcRn), greater in vivo half-life relative to a polypeptide lacking an Fc region, protein A binding, and perhaps even placental transfer (see Capon et al., Nature 337:525, 1989).

An Fc region or domain may interact with different types of FcRs. The different types of FcRs may include, for example, FcγRI, FcγRIIA, FcγRIIB, FcγRIIIA, FcγRIIIB, FcαRI, FcμR, FcεRI, FcεRII, and FcRn. FcRs may be located on the membrane of certain immune cells including, for example, B lymphocytes, natural killer cells, macrophages, neutrophils, follicular dendritic cells, eosinophils, basophils, platelets, and mast cells. Once the FcR is engaged by the Fc region or domain, the FcR may initiate functions including, for example, clearance of an antigen-antibody complex via receptor-mediated endocytosis, antibody-dependent cell-mediated cytotoxicity (ADCC), antibody dependent cell-mediated phagocytosis (ADCP), and ligand-triggered transmission of signals across the plasma membrane that can result in alterations in secretion, exocytosis, and cellular metabolism. FcRs may deliver signals when FcRs are aggregated by antibodies and multivalent antigens at the cell surface. The aggregation of FcRs with immunoreceptor tyrosine-based activation motifs (ITAMs) may sequentially activate SRC family tyrosine kinases and SYK family tyrosine kinases. ITAM comprises a twice-repeated YxxL sequence flanking seven variable residues. The SRC and SYK kinases may connect the transduced signals with common activation pathways.

In some embodiments, an Fc region or domain can exhibit reduced binding affinity to one or more Fc receptors. In some embodiments, an Fc region or domain can exhibit reduced binding affinity to one or more Fcgamma receptors. In some embodiments, an Fc region or domain can exhibit reduced binding affinity to FcRn receptors. In some embodiments, an Fc region or domain can exhibit reduced binding affinity to Fcgamm and FcRn receptors. In some embodiments, an Fc region or domain is an Fc null region or domain. As used herein, an “Fc null” refers to a domain that exhibits weak to no binding to any of the Fey receptors. In some embodiments, an Fc null region or domain exhibits a reduction in binding affinity (e.g., increase in Kd) to Fc gamma receptors of at least about 1000-fold.

The Fc region or domain may have one or more, two or more, three or more, or four or more, or up to five amino acid substitutions that decrease binding of the Fc region or domain to an Fc receptor. In some embodiments, an Fc region or domain exhibits decreased binding to FcγRI (CD64), FcγRIIA (CD32), FcγRIIIA (CD16a), FcγRIIIB (CD16b), or any combination thereof. In order to decrease binding affinity of an Fc region or domain to an Fc receptor, the Fc region or domain may comprise one or more amino acid substitutions that has the effect of reducing the affinity of the Fc region or domain to an Fc receptor. In certain embodiments, the one or more substitutions comprise any one or more of IgG1 heavy chain mutations corresponding to E233P, L234V, L234A, L235A, L235E, ΔG236, G237A, E318A, K320A, K322A, A327G, A330S, or P331S according to the EU index of Kabat numbering.

In some embodiments, the Fc region or domain can comprise a sequence of the IgG1 isoform that has been modified from the wild-type IgG1 sequence. A modification can comprise a substitution at more than one amino acid residue, such as at 5 different amino acid residues including L235V/F243L/R292P/Y300L/P396L (IgG1VLPLL) according to the EU index of Kabat numbering. A modification can comprise a substitution at more than one amino acid residue such as at 2 different amino acid residues including S239D/I332E (IgG1DE) according to the EU index of Kabat numbering. A modification can comprise a substitution at more than one amino acid residue such as at 3 different amino acid residues including S298A/E333A/K334A (IgG1AAA) according to the EU index of Kabat numbering.

An antibody construct may consist of two identical light protein chains and two identical heavy protein chains, all held together covalently by disulfide linkages. The N-terminal regions of the light and heavy chains together may form the antigen recognition site of an antibody. Structurally, various functions of an antibody may be confined to discrete protein domains. The sites that can recognize and can bind antigen may consist of three complementarities determining regions (CDRs) that may lie within the variable heavy chain region and variable light chain region at the N-terminal end of the heavy chain and the light chain. The constant domains may provide the general framework of the antibody and may not be involved directly in binding the antibody to an antigen, but may be involved in various effector functions, such as participation of the antibody in antibody-dependent cellular cytotoxicity, and may bind Fc receptors. The constant domains may include an Fc region. The constant domains may include an Fc region or domain. The variable regions of natural light and heavy chains may have the same general structures, and each domain may comprise four framework regions, whose sequences can be somewhat conserved, connected by three hyper-variable regions or CDRs. The four framework regions (FR) may largely adopt a β-sheet conformation and the CDRs can form loops connecting, and in some aspects forming part of, the β-sheet structure. The CDRs in each chain may be held in close proximity by the framework regions and with the CDRs from the other chain, may contribute to the formation of the antigen binding site.

An antibody construct may comprise a light chain of an amino acid sequence having at least one, two, three, four, five, six, seven, eight, nine or ten modifications and in certain embodiments, not more than 40, 35, 30, 25, 20, 15 or 10 modifications of the amino acid sequence relative to the natural or original amino acid sequence. An antibody construct may comprise a heavy chain of an amino acid sequence having at least one, two, three, four, five, six, seven, eight, nine or ten modifications and in certain embodiments, not more than 40, 35, 30, 25, 20, 15 or 10 modifications of the amino acid sequence relative to the natural or original amino acid sequence.

An antibody construct may be an antibody. Antibodies may be selected from different classes of immunoglobins, e.g., IgA, IgD, IgE, IgG, and IgM. The several different classes may be further divided into isotypes, e.g., IgG1, IgG2, IgG3, IgG4, IgA1, and IgA2. An antibody may further comprise a light chain and a heavy chain, often more than one chain. The heavy-chain constant regions (Fc) that corresponds to the different classes of immunoglobulins may be α, δ, ε, γ, and μ, respectively. The light chains may be one of either kappa (κ) or lambda (λ), based on the amino acid sequences of the constant domains. The Fc region or domain may further comprise an Fc region. An Fc receptor may bind an Fc region or domain. Antibody constructs may also include any fragment or recombinant forms thereof, including but not limited to, single chain variable fragments (scFvs).

An antibody construct may comprise an antigen-binding antibody fragment. An antibody fragment may include (i) a Fab fragment, a monovalent fragment consisting of the V_{L}, V_{H}, CL and C_{H1} domains; (ii) a F(ab′)₂ fragment, a bivalent fragment comprising two Fab fragments linked by a disulfide bridge at the hinge region; and (iii) a Fv fragment consisting of the V_{L} and V_{H} domains of a single arm of an antibody. Although the two domains of the Fv fragment, V_{L} and V_{H}, may be coded for by separate genes, they may be linked by a synthetic linker to be made as a single protein chain in which the V_{L} and V_{H} regions pair to form monovalent molecules.

F(ab′)₂ and Fab′ moieties may be produced by genetic engineering or by treating immunoglobulin (e.g., monoclonal antibody) with a protease such as pepsin and papain, and may include an antibody fragment generated by digesting immunoglobulin near the disulfide bonds existing between the hinge regions in each of the two H chains. The Fab fragment may also contain the constant domain of the light chain and the first constant domain (C_{H1}) of the heavy chain. Fab′ fragments may differ from Fab fragments by the addition of a few residues at the carboxyl terminus of the heavy chain C_{H1} domain including one or more cysteine(s) from the antibody hinge region.

An Fv may be the minimum antibody fragment which contains a complete antigen-recognition and antigen-binding site. This region may consist of a dimer of one heavy chain and one light chain variable domain in tight, non-covalent association. In this configuration, the three CDRs of each variable domain may interact to define an antigen-binding site on the surface of the V_{H}-V_{L} dimer. A single variable domain (or half of an Fv comprising only three CDRs specific for an antigen) may recognize and bind to antigen, although the binding can be at a lower affinity than the affinity of the entire binding site.

An antibody construct may include an Fc region or domain comprising an Fc region or several Fc region or domains. The Fc region or domain of an antibody may interact with FcRs found on immune cells. The Fc region or domain may also mediate the interaction between effector molecules and cells, which may lead to activation of the immune system. In the IgG, IgA, and IgD antibody isotypes, the Fc region may comprise two identical protein fragments, which can be derived from the second and third constant domains of the antibody's heavy chains. In the IgM and IgE antibody isotypes, the Fc regions may comprise three heavy chain constant domains. In the IgG antibody isotype, the Fc regions may comprise a highly-conserved N-glycosylation site, which may be important for FcR-mediated downstream effects.

An antibody construct used herein may be “chimeric” or “humanized.” Chimeric and humanized forms of non-human (e.g., murine) antibodies can be chimeric immunoglobulins, immunoglobulin chains or fragments thereof (such as Fv, Fab, Fab′, F(ab′)₂ or other target-binding subdomains of antibodies), which may contain minimal sequences derived from non-human immunoglobulin. In general, the humanized antibody may comprise substantially all of at least one, and typically two, variable domains, in which all or substantially all of the CDRs correspond to those of a non-human immunoglobulin and all or substantially all of the framework regions are those of a human immunoglobulin sequence. The humanized antibody may also comprise at least a portion of an immunoglobulin constant region (Fc), typically that of a human immunoglobulin consensus sequence.

An antibody construct may be a human antibody. As used herein, “human antibodies” can include antibodies having, for example, the amino acid sequence of a human immunoglobulin and may include antibodies isolated from human immunoglobulin libraries or from animals transgenic for one or more human immunoglobulins that do not express endogenous immunoglobulins. Human antibodies may be produced using transgenic mice which are incapable of expressing functional endogenous immunoglobulins, but which may express human immunoglobulin genes. Completely human antibodies that recognize a selected epitope may be generated using guided selection. In this approach, a selected non-human monoclonal antibody, e.g., a mouse antibody, may be used to guide the selection of a completely human antibody recognizing the same epitope.

An antibody, antibody construct, or targeting moiety may be a derivatized antibody. For example, derivatized antibodies may be modified by glycosylation, acetylation, pegylation, phosphorylation, amidation, derivatization by known protecting/blocking groups, proteolytic cleavage, linkage to a cellular ligand or other protein.

In any of the aforementioned embodiments, the antibody, antibody construct, or targeting moiety can be used to generate a conjugate with an ALK5 inhibitor compound of this disclosure as described herein.

An antibody may have a sequence that has been modified to alter at least one constant region-mediated biological effector function relative to the corresponding wild type sequence. For example, in some embodiments, the antibody can be modified to reduce at least one constant region-mediated biological effector function relative to an unmodified antibody, e.g., reduced binding to the Fc receptor (FcR). FcR binding may be reduced by, for example, mutating the immunoglobulin constant region segment of the antibody at particular regions necessary for FcR interactions.

An antibody or Fc region or domain may be modified to acquire or improve at least one constant region-mediated biological effector function relative to an unmodified antibody or Fc region or domain, e.g., to enhance FcγR interactions. For example, an antibody with a constant region that binds to FcγRIIA, FcγRIIB and/or FcγRIIIA with greater affinity than the corresponding wild type constant region may be produced according to the methods described herein. An Fc region or domain that binds to FcγRIIA, FcγRIIB and/or FcγRIIIA with greater affinity than the corresponding wild type Fc region or domain may be produced according to the methods described herein or known to the skilled artisan.

In certain embodiments, an Fc region or domain found in an antibody, antibody construct, or targeting moiety of the present disclosure will be capable of mediating one or more of these effector functions, or will lack one or more or all of these activities or have one or more of the effector activities increased by way of, for example, one or more mutations as compared to the unmodified Fc region or domain.

In addition, antibodies have a hinge sequence that is typically situated between the Fab and Fc region (but a lower section of the hinge may include an amino-terminal portion of the Fc region). By way of background, an immunoglobulin hinge acts as a flexible spacer to allow the Fab portion to move freely in space. In contrast to the constant regions, hinges are structurally diverse, varying in both sequence and length between immunoglobulin classes and even among subclasses. For example, a human IgG1 hinge region is freely flexible, which allows the Fab fragments to rotate about their axes of symmetry and move within a sphere centered at the first of two inter-heavy chain disulfide bridges. By comparison, a human IgG2 hinge is relatively short and contains a rigid poly-proline double helix stabilized by four inter-heavy chain disulfide bridges, which restricts the flexibility. A human IgG3 hinge differs from the other subclasses by its unique extended hinge region (about four times as long as the IgG1 hinge), containing 62 amino acids (including 21 prolines and 11 cysteines), forming an inflexible poly-proline double helix and providing greater flexibility because the Fab fragments are relatively far away from the Fc fragment. A human IgG4 hinge is shorter than IgG1 but has the same length as IgG2, and its flexibility is intermediate between that of IgG1 and IgG2.

An antibody construct may contain, for example, two, three, four, five, six, seven, eight, nine, ten, or more antigen binding domains. An antibody construct may contain two antigen binding domains in which each antigen binding domain can recognize the same antigen. An antibody construct may contain two antigen binding domains in which each antigen binding domain can recognize different antigens. An antigen binding domain may be in a scaffold, in which a scaffold is a supporting framework for the antigen binding domain. An antigen binding domain may be in a non-antibody scaffold. An antigen binding domain may be in an antibody scaffold. An antibody construct may comprise an antigen binding domain in a scaffold. The antibody construct may comprise an Fc fusion protein. In some embodiments, the antibody construct is an Fc fusion protein. An antigen binding domain may specifically bind to a tumor antigen. An antigen binding domain may specifically bind to an antigen having at least 80%, at least 90%, at least 95%, at least 99%, or 100% sequence identity to a tumor antigen. An antigen binding domain may specifically bind to an antigen on an antigen presenting cell (APC). An antigen binding domain may specifically bind to an antigen having at least 80%, at least 90%, at least 95%, at least 99%, or 100% sequence identity to an antigen on an antigen presenting cell (APC).

The antigen binding domain of an antibody construct may be at least 80% identical to an antigen binding domain selected from, but not limited to, a monoclonal antibody, a polyclonal antibody, a recombinant antibody, or a functional fragment thereof, for example, a heavy chain variable domain (V_{H}) and a light chain variable domain (V_{L}), or a DARPin, an affimer, an avimer, a knottin, a monobody, an affinity clamp, an ectodomain, a receptor ectodomain, a receptor, a cytokine, a ligand, an immunocytokine, a T cell receptor, or a recombinant T cell receptor.

In certain embodiments, an antibody construct of the disclosure comprises an Fc region or domain that may comprise an Fc region, in which the Fc region or domain may be the part of the Fc region that interacts with Fc receptors. The Fc region or domain of an antibody construct may interact with Fc-receptors (FcRs) found on immune cells. The Fc region or domain may also mediate the interaction between effector molecules and cells, which can lead to activation of the immune system. The Fc region may be derived from IgG, IgA, or IgD antibody isotypes, and may comprise two identical protein fragments, which are derived from the second and third constant domains of the antibody's heavy chains. In an Fc region or domain derived from an IgG antibody isotype, the Fc region or domain may comprise a highly-conserved N-glycosylation site, which may be essential for FcR-mediated downstream effects. The Fc region or domain may be derived from IgM or IgE antibody isotypes, in which the Fc region or domain may comprise three heavy chain constant domains.

In certain embodiments, an antibody construct comprises an Fc region or domain that may comprise an Fc region, in which the Fc region or domain may be the part of the Fc region that interacts with Fc receptors. The Fc region or domain of an antibody construct may interact with Fc-receptors (FcRs) found on immune cells. The Fc region or domain may also mediate the interaction between effector molecules and cells, which can lead to activation of the immune system. The Fc region may be derived from IgG, IgA, or IgD antibody isotypes, and may comprise two identical protein fragments, which are derived from the second and third constant domains of the antibody's heavy chains. In an Fc region or domain derived from an IgG antibody isotype, the Fc region or domain may comprise a highly-conserved N-glycosylation site, which may be essential for FcR-mediated downstream effects. The Fc region or domain may be derived from IgM or IgE antibody isotypes, in which the Fc region or domain may comprise three heavy chain constant domains.

An Fc region or domain may interact with different types of FcRs. The different types of FcRs may include, for example, FcγRI, FcγRIIA, FcγRIIB, FcγRIIIA, FcγRIIIB, FcαRI, FcμR, FcεRI, FcεRII, and FcRn. FcRs may be located on the membrane of certain immune cells including, for example, B lymphocytes, natural killer cells, macrophages, neutrophils, follicular dendritic cells, eosinophils, basophils, platelets, and mast cells. Once the FcR is engaged by the Fc region or domain, the FcR may initiate functions including, for example, clearance of an antigen-antibody complex via receptor-mediated endocytosis, antibody-dependent cell-mediated cytotoxicity (ADCC), antibody dependent cell-mediated phagocytosis (ADCP), and ligand-triggered transmission of signals across the plasma membrane that can result in alterations in secretion, exocytosis, and cellular metabolism. FcRs may deliver signals when FcRs are aggregated by antibodies and multivalent antigens at the cell surface. The aggregation of FcRs with immunoreceptor tyrosine-based activation motifs (ITAMs) may sequentially activate SRC family tyrosine kinases and SYK family tyrosine kinases. ITAM comprises a twice-repeated YxxL sequence flanking seven variable residues. The SRC and SYK kinases may connect the transduced signals with common activation pathways.

In some embodiments, an Fc region or domain of the antibody construct portion of a conjugate can exhibit increased binding affinity to one or more Fc receptors. In some embodiments, an Fc region or domain can exhibit increased binding affinity to one or more Fcgamma receptors. In some embodiments, an Fc region or domain can exhibit increased binding affinity to FcRn receptors. In some embodiments, an Fc region or domain can exhibit increased binding affinity to Fcgamma and FcRn receptors.

In some embodiments, an Fc region or domain of the antibody construct portion of a conjugate can exhibit reduced binding affinity to one or more Fc receptors. In some embodiments, an Fc region or domain can exhibit reduced binding affinity to one or more Fcgamma receptors. In some embodiments, an Fc region or domain can exhibit reduced binding affinity to FcRn receptors. In some embodiments, an Fc region or domain can exhibit reduced binding affinity to Fcgamma and FcRn receptors. In some embodiments, an Fc region or domain is an Fc null region or domain. In some embodiments, an Fc region or domain can exhibit reduced binding affinity to FcRn receptors, but have the same or increased binding affinity to one or more Fcgamma receptors as compared to a wildtype IgG. In some embodiments, an Fc region or domain can exhibit increased binding affinity to FcRn receptors, but have the same or decreased binding affinity to one or more Fcgamma receptors.

The Fc region or domain may have one or more, two or more, three or more, or four or more amino acid substitutions that decrease binding of the Fc region or domain to an Fc receptor. In certain embodiments, an Fc region or domain has decreased binding affinity for one or more of FcγRI (CD64), FcγRIIA (CD32), FcγRIIIA (CD16a), FcγRIIIB (CD16b), or any combination thereof. In order to decrease binding affinity of an Fc region or domain to an Fc receptor, the Fc region or domain may comprise one or more amino acid substitutions that reduces the binding affinity of the Fc region or domain to an Fc receptor.

In certain embodiments, the one or more substitutions comprise any one or more of IgG1 heavy chain mutations corresponding to E233P, L234V, L234A, L235A, L235E, ΔG236, G237A, E318A, K320A, K322A, A327G, A330S, or P331S according to the EU index of Rabat numbering.

In some embodiments, the Fc region or domain can comprise a sequence of an IgG isoform that has been modified from the wild-type IgG sequence. In some embodiments, the Fc region or domain can comprise a sequence of the IgG1 isoform that has been modified from the wild-type IgG1 sequence. In some embodiments, the modification comprises substitution of one or more amino acids that reduce binding affinity of an IgG Fc region or domain to all Fey receptors. A modification can be substitution of E233, L234 and L235, such as E233P/L234V/L235A or E233P/L234V/L235A/ΔG236, according to the EU index of Rabat. A modification can be a substitution of P238, such as P238A, according to the EU index of Rabat. A modification can be a substitution of D265, such as D265A, according to the EU index of Rabat. A modification can be a substitution of N297, such as N297A, according to the EU index of Rabat. A modification can be a substitution of A327, such as A327Q, according to the EU index of Rabat. A modification can be a substitution of P329, such as P239A, according to the EU index of Rabat.

In some embodiments, an IgG Fc region or domain comprises at least one amino acid substitution that reduces its binding affinity to FcγRI, as compared to a wild-type or reference IgG Fc region or domain. A modification can comprise a substitution at F241, such as F241A, according to the EU index of Kabat. A modification can comprise a substitution at F243, such as F243A, according to the EU index of Kabat. A modification can comprise a substitution at V264, such as V264A, according to the EU index of Kabat. A modification can comprise a substitution at D265, such as D265A according to the EU index of Kabat.

In some embodiments, an IgG Fc region or domain comprises at least one amino acid substitution that increases its binding affinity to FcγR1, as compared to a wild-type or reference IgG Fc region or domain. A modification can comprise a substitution at A327 and P329, such as A327Q/P329A, according to the EU index of Kabat.

In some embodiments, the modification comprises substitution of one or more amino acids that reduce binding affinity of an IgG Fc region or domain to FcγRII and FcγRIIIA receptors. A modification can be a substitution of D270, such as D270A, according to the EU index of Kabat. A modification can be a substitution of Q295, such as Q295A, according to the EU index of Kabat. A modification can be a substitution of A327, such as A237S, according to the EU index of Kabat.

In some embodiments, the modification comprises substitution of one or more amino acids that increases binding affinity of an IgG Fc region or domain to FcγRII and FcγRIIIA receptors. A modification can be a substitution of T256, such as T256A, according to the EU index of Kabat. A modification can be a substitution of K290, such as K290A, according to the EU index of Kabat.

In some embodiments, the modification comprises substitution of one or more amino acids that increases binding affinity of an IgG Fc region or domain to FcγRII receptor. A modification can be a substitution of R255, such as R255A, according to the EU index of Kabat. A modification can be a substitution of E258, such as E258A, according to the EU index of Kabat. A modification can be a substitution of S267, such as S267A, according to the EU index of Kabat. A modification can be a substitution of E272, such as E272A, according to the EU index of Kabat. A modification can be a substitution of N276, such as N276A, according to the EU index of Kabat. A modification can be a substitution of D280, such as D280A, according to the EU index of Kabat. A modification can be a substitution of H285, such as H285A, according to the EU index of Kabat. A modification can be a substitution of N286, such as N286A, according to the EU index of Kabat. A modification can be a substitution of T307, such as T307A, according to the EU index of Kabat. A modification can be a substitution of L309, such as L309A, according to the EU index of Kabat. A modification can be a substitution of N315, such as N315A, according to the EU index of Kabat. A modification can be a substitution of K326, such as K326A, according to the EU index of Kabat. A modification can be a substitution of P331, such as P331A, according to the EU index of Kabat. A modification can be a substitution of S337, such as S337A, according to the EU index of Kabat. A modification can be a substitution of A378, such as A378A, according to the EU index of Kabat. A modification can be a substitution of E430, such as E430, according to the EU index of Kabat.

In some embodiments, the modification comprises substitution of one or more amino acids that increases binding affinity of an IgG Fc region or domain to FcγRII receptor and reduces the binding affinity to FcγRIIIA receptor. A modification can be a substitution of H268, such as H268A, according to the EU index of Kabat. A modification can be a substitution of R301, such as R301A, according to the EU index of Kabat. A modification can be a substitution of K322, such as K322A, according to the EU index of Kabat.

In some embodiments, the modification comprises substitution of one or more amino acids that decreases binding affinity of an IgG Fc region or domain to FcγRII receptor but does not affect the binding affinity to FcγRIIIA receptor. A modification can be a substitution of R292, such as R292A, according to the EU index of Kabat. A modification can be a substitution of K414, such as K414A, according to the EU index of Kabat.

In some embodiments, the modification comprises substitution of one or more amino acids that decreases binding affinity of an IgG Fc region or domain to FcγRII receptor and increases the binding affinity to FcγRIIIA receptor. A modification can be a substitution of S298, such as S298A, according to the EU index of Kabat. A modification can be substitution of S239, 1332 and A330, such as S239D/I332E/A330L. A modification can be substitution of S239 and 1332, such as S239D/I332E.

In some embodiments, the modification comprises substitution of one or more amino acids that decreases binding affinity of an IgG Fc region or domain to FcγRIIIA receptor. A modification can be substitution of F241 and F243, such as F241S/F243S or F241I/F243I, according to the EU index of Kabat.

In some embodiments, the modification comprises substitution of one or more amino acids that decreases binding affinity of an IgG Fc region or domain to FcγRIIIA receptor and does not affect the binding affinity to FcγRII receptor. A modification can be a substitution of S239, such as S239A, according to the EU index of Kabat. A modification can be a substitution of E269, such as E269A, according to the EU index of Kabat. A modification can be a substitution of E293, such as E293A, according to the EU index of Kabat. A modification can be a substitution of Y296, such as Y296F, according to the EU index of Kabat. A modification can be a substitution of V303, such as V303A, according to the EU index of Kabat. A modification can be a substitution of A327, such as A327G, according to the EU index of Kabat. A modification can be a substitution of K338, such as K338A, according to the EU index of Kabat. A modification can be a substitution of D376, such as D376A, according to the EU index of Kabat.

In some embodiments, the modification comprises substitution of one or more amino acids that increases binding affinity of an IgG Fc region or domain to FcγRIIIA receptor and does not affect the binding affinity to FcγRII receptor. A modification can be a substitution of E333, such as E333A, according to the EU index of Kabat. A modification can be a substitution of K334, such as K334A, according to the EU index of Kabat. A modification can be a substitution of A339, such as A339T, according to the EU index of Kabat. A modification can be substitution of S239 and 1332, such as S239D/I332E.

In some embodiments, the modification comprises substitution of one or more amino acids that increases binding affinity of an IgG Fc region or domain to FcγRIIIA receptor. A modification can be substitution of L235, F243, R292, Y300 and P396, such as L235V/F243L/R292P/Y300L/P396L (IgG1VLPLL) according to the EU index of Kabat. A modification can be substitution of S298, E333 and K334, such as S298A/E333A/K334A, according to the EU index of Kabat. A modification can be substitution of K246, such as K246F, according to the EU index of Kabat.

Other substitutions in an IgG Fc region or domain that affect its interaction with one or more Fey receptors are disclosed in U.S. Pat. Nos. 7,317,091 and 8,969,526 (the disclosures of which are incorporated by reference herein).

In some embodiments, an IgG Fc region or domain comprises at least one amino acid substitution that reduces the binding affinity to FcRn, as compared to a wild-type or reference IgG Fc region or domain. A modification can comprise a substitution at H435, such as H435A according to the EU index of Kabat. A modification can comprise a substitution at 1253, such as 1253A according to the EU index of Kabat. A modification can comprise a substitution at H310, such as H310A according to the EU index of Kabat. A modification can comprise substitutions at 1253, H310 and H435, such as I253A/H310A/H435A according to the EU index of Kabat.

A modification can comprise a substitution of one amino acid residue that increases the binding affinity of an IgG Fc region or domain for FcRn, relative to a wildtype or reference IgG Fc region or domain. A modification can comprise a substitution at V308, such as V308P according to the EU index of Kabat. A modification can comprise a substitution at M428, such as M428L according to the EU index of Kabat. A modification can comprise a substitution at N434, such as N434A according to the EU index of Kabat or N434H according to the EU index of Kabat. A modification can comprise substitutions at T250 and M428, such as T250Q and M428L according to the EU index of Kabat. A modification can comprise substitutions at M428 and N434, such as M428L and N434S, N434A or N434H according to the EU index of Kabat. A modification can comprise substitutions at M252, S254 and T256, such as M252Y/S254T/T256E according to the EU index of Kabat. A modification can be a substitution of one or more amino acids selected from P257L, P257N, P257I, V279E, V279Q, V279Y, A281S, E283F, V284E, L306Y, T307V, V308F, Q31IV, D376V, and N434H. Other substitutions in an IgG Fc region or domain that affect its interaction with FcRn are disclosed in U.S. Pat. No. 9,803,023 (the disclosure of which is incorporated by reference herein).

In certain embodiments, the antibody construct comprises an antigen binding domain and an Fc region or domain.

In certain embodiments, the antigen binding domain specifically binds to an antigen that is at least 80% identical to an antigen on a T cell, a B cell, a stellate cell, an endothelial cell, a tumor cell, an APC, a fibroblast cell, a fibrocyte cell, or a cell associated with the pathogenesis of fibrosis. In certain embodiments, the antigen binding domain specifically binds to an antigen that is at least 80% identical to an antigen on a T cell, an APC, and/or a B cell. In certain embodiments, the antigen binding domain specifically binds to an antigen that is at least 80% identical to an antigen on a hepatocyte. In certain embodiments, the antigen binding domain may specifically bind to an antigen that is at least 80% identical to an antigen selected from the group consisting of CLTA4, PD-1, OX40, LAG-3, GITR, GARP, CD25, CD27, PD-L1, TNFR2, ICOS, 41BB, CD70, CD73, CD38, or VTCN1. In certain embodiments, the antigen binding domain may specifically bind to an antigen that is at least 80% identical to an antigen selected from the group consisting of ASGR1 and ASGR2 (asialoglycoprotein receptor 1 and 2). In certain embodiments, the antigen binding domain specifically binds to an antigen that is at least 80% identical to an antigen on a stellate cell, an endothelial cell, a fibroblast cell, a fibrocyte cell, or a cell associated with the pathogenesis of fibrosis or cancer. In certain embodiments, the antigen binding domain may specifically bind to an antigen that is at least 80% identical to an antigen selected from the group consisting of PDGFRβ, integrin αvβ1, integrin αvβ3, integrin αvβ6, integrin αvβ8, Endosialin, FAP, ADAM12, LRRC15, MMP14, PDPN, CDH11 and F2RL2, In certain embodiments, the antigen binding domain may specifically bind to an antigen that is at least 80% identical to an antigen selected from the group consisting of FAP, ADAM12, LRRC15, MMP14, PDPN, CDH11 and F2RL2, In certain embodiments, the antigen binding domain specifically binds to an antigen that is at least 80% identical to an antigen on a tumor cell, a tumor antigen. In certain embodiments, the antigen binding domain specifically binds to an antigen that is at least 80% identical to an antigen selected from the group consisting of MUC16, UPK1B, VTCN1, TMPRSS3, TMEM238, Clorf186, TMPRSS4, CLDN6, CLDN8, STRA6, MSLN or CD73.

In certain embodiments, the antigen binding domain specifically binds to an antigen on a T cell, a B cell, a stellate cell, an endothelial cell, a tumor cell, an APC, a fibroblast cell, a fibrocyte cell, or a cell associated with the pathogenesis of fibrosis. In certain embodiments, the antigen binding domain specifically binds to an antigen on a T cell, an APC, and/or a B cell. In certain embodiments, the antigen binding domain specifically binds to an antigen on a hepatocyte. In certain embodiments, the antigen binding domain may specifically bind to an antigen selected from the group consisting of CLTA4, PD-1, OX40, LAG-3, GITR, GARP, CD25, CD27, PD-L1, TNFR2, ICOS, 41BB, CD70, CD73, CD38 or VTCN1. In certain embodiments, the antigen binding domain may specifically bind to an antigen selected from the group consisting of ASGR1 and ASGR2. In certain embodiments, the antigen binding domain specifically binds to an antigen on a stellate cell, an endothelial cell, a fibroblast cell, a fibrocyte cell, or a cell associated with the pathogenesis of fibrosis or cancer. In certain embodiments, the antigen binding domain may specifically bind to an antigen selected from the group consisting of, PDGFRβ, integrin αvβ1, integrin αvβ3, integrin αvβ6, integrin αvβ8, Endosialin, FAP, ADAM12, LRRC15, MMP14, PDPN, CDH11 and F2RL2. In certain embodiments, the antigen binding domain may specifically bind to an antigen selected from the group consisting of FAP, ADAM12, LRRC15, MMP14, PDPN, CDH11 and F2RL2. In certain embodiments, the antigen is LRRC15. In certain embodiments, the antigen binding domain specifically binds to an antigen on a tumor cell, a tumor antigen. In certain embodiments, the antigen binding domain specifically binds to an antigen selected from the group consisting of MUC16, UPK1B, VTCN1, TMPRSS3, TMEM238, Clorf186, TMPRSS4, CLDN6, CLDN8, STRA6, MSLN or CD73.

An antibody construct may comprise an antibody with modifications of at least one amino acid residue. Modifications may be substitutions, additions, mutations, deletions, or the like. An antibody modification can be an insertion of an unnatural amino acid.

An antigen binding domain may comprise at least 80% sequence identity to any sequence in Table 1. An antigen binding domain may comprise a set of CDRs set forth in Table 1. An antibody construct may comprise an antigen binding domain that binds an antigen, wherein the antigen binding domain comprises at least at least 80%, at least 90%, at least 95%, at least 97%, at least 98%, at least 99%, or at least 100% sequence identity to:
a) HCDR1 comprising an amino acid sequence of SEQ ID NO: 1, HCDR2 comprising an amino acid sequence of SEQ ID NO: 2, HCDR3 comprising an amino acid sequence of SEQ ID NO: 3, LCDR1 comprising an amino acid sequence of SEQ ID NO: 4, LCDR2 comprising an amino acid sequence of SEQ ID NO: 5, or LCDR3 comprising an amino acid sequence of SEQ ID NO: 6;
b) HCDR1 comprising an amino acid sequence of SEQ ID NO: 7, HCDR2 comprising an amino acid sequence of SEQ ID NO: 8, HCDR3 comprising an amino acid sequence of SEQ ID NO: 9, LCDR1 comprising an amino acid sequence of SEQ ID NO: 10, LCDR2 comprising an amino acid sequence of SEQ ID NO: 11, or LCDR3 comprising an amino acid sequence of SEQ ID NO: 12;
c) HCDR1 comprising an amino acid sequence of SEQ ID NO: 13, HCDR2 comprising an amino acid sequence of SEQ ID NO: 14, HCDR3 comprising an amino acid sequence of SEQ ID NO: 15, LCDR1 comprising an amino acid sequence of SEQ ID NO: 16, LCDR2 comprising an amino acid sequence of SEQ ID NO: 17, or LCDR3 comprising an amino acid sequence of SEQ ID NO: 18;
d) HCDR1 comprising an amino acid sequence of SEQ ID NO: 19, HCDR2 comprising an amino acid sequence of SEQ ID NO: 20, HCDR3 comprising an amino acid sequence of SEQ ID NO: 21, LCDR1 comprising an amino acid sequence of SEQ ID NO: 22, LCDR2 comprising an amino acid sequence of SEQ ID NO: 23, or LCDR3 comprising an amino acid sequence of SEQ ID NO: 24;
e) HCDR1 comprising an amino acid sequence of SEQ ID NO: 25, HCDR2 comprising an amino acid sequence of SEQ ID NO: 26, HCDR3 comprising an amino acid sequence of SEQ ID NO: 27, LCDR1 comprising an amino acid sequence of SEQ ID NO: 28, LCDR2 comprising an amino acid sequence of SEQ ID NO: 29, or LCDR3 comprising an amino acid sequence of SEQ ID NO: 30;
f) HCDR1 comprising an amino acid sequence of SEQ ID NO: 31, HCDR2 comprising an amino acid sequence of SEQ ID NO: 32, HCDR3 comprising an amino acid sequence of SEQ ID NO: 33, LCDR1 comprising an amino acid sequence of SEQ ID NO: 34, LCDR2 comprising an amino acid sequence of SEQ ID NO: 35, or LCDR3 comprising an amino acid sequence of SEQ ID NO: 36;
g) HCDR1 comprising an amino acid sequence of SEQ ID NO: 37, HCDR2 comprising an amino acid sequence of SEQ ID NO: 38, HCDR3 comprising an amino acid sequence of SEQ ID NO: 39, LCDR1 comprising an amino acid sequence of SEQ ID NO: 40, LCDR2 comprising an amino acid sequence of SEQ ID NO: 41, or LCDR3 comprising an amino acid sequence of SEQ ID NO: 42;
h) HCDR1 comprising an amino acid sequence of SEQ ID NO: 43, HCDR2 comprising an amino acid sequence of SEQ ID NO: 44, HCDR3 comprising an amino acid sequence of SEQ ID NO: 45, LCDR1 comprising an amino acid sequence of SEQ ID NO: 46, LCDR2 comprising an amino acid sequence of SEQ ID NO: 47, or LCDR3 comprising an amino acid sequence of SEQ ID NO: 48;
i) HCDR1 comprising an amino acid sequence of SEQ ID NO: 50, HCDR2 comprising an amino acid sequence of SEQ ID NO: 51, HCDR3 comprising an amino acid sequence of SEQ ID NO: 52, LCDR1 comprising an amino acid sequence of SEQ ID NO: 53, LCDR2 comprising an amino acid sequence of SEQ ID NO: 54, or LCDR3 comprising an amino acid sequence of SEQ ID NO: 55;
j) HCDR1 comprising an amino acid sequence of SEQ ID NO: 56, HCDR2 comprising an amino acid sequence of SEQ ID NO: 57, HCDR3 comprising an amino acid sequence of SEQ ID NO: 58, LCDR1 comprising an amino acid sequence of SEQ ID NO: 59, LCDR2 comprising an amino acid sequence of SEQ ID NO: 60, or LCDR3 comprising an amino acid sequence of SEQ ID NO: 61;
k) HCDR1 comprising an amino acid sequence of SEQ ID NO: 62, HCDR2 comprising an amino acid sequence of SEQ ID NO: 63, HCDR3 comprising an amino acid sequence of SEQ ID NO: 64, LCDR1 comprising an amino acid sequence of SEQ ID NO: 65, LCDR2 comprising an amino acid sequence of SEQ ID NO: 66, or LCDR3 comprising an amino acid sequence of SEQ ID NO: 67;
l) HCDR1 comprising an amino acid sequence of SEQ ID NO: 68, HCDR2 comprising an amino acid sequence of SEQ ID NO: 69, HCDR3 comprising an amino acid sequence of SEQ ID NO: 70, LCDR1 comprising an amino acid sequence of SEQ ID NO: 71, LCDR2 comprising an amino acid sequence of SEQ ID NO: 72, or LCDR3 comprising an amino acid sequence of SEQ ID NO: 73;
m) HCDR1 comprising an amino acid sequence of SEQ ID NO: 74, HCDR2 comprising an amino acid sequence of SEQ ID NO: 75, HCDR3 comprising an amino acid sequence of SEQ ID NO: 76, LCDR1 comprising an amino acid sequence of SEQ ID NO: 77, LCDR2 comprising an amino acid sequence of SEQ ID NO: 78, or LCDR3 comprising an amino acid sequence of SEQ ID NO: 79;
n) HCDR1 comprising an amino acid sequence of SEQ ID NO: 74, HCDR2 comprising an amino acid sequence of SEQ ID NO: 75, HCDR3 comprising an amino acid sequence of SEQ ID NO: 76, LCDR1 comprising an amino acid sequence of SEQ ID NO: 80, LCDR2 comprising an amino acid sequence of SEQ ID NO: 81, or LCDR3 comprising an amino acid sequence of SEQ ID NO: 82;
o) HCDR1 comprising an amino acid sequence of SEQ ID NO: 199, HCDR2 comprising an amino acid sequence of SEQ ID NO: 200, HCDR3 comprising an amino acid sequence of SEQ ID NO: 201, LCDR1 comprising an amino acid sequence of SEQ ID NO: 202, LCDR2 comprising an amino acid sequence of SEQ ID NO: 203, or LCDR3 comprising an amino acid sequence of SEQ ID NO: 204;
p) HCDR1 comprising an amino acid sequence of SEQ ID NO: 205, HCDR2 comprising an amino acid sequence of SEQ ID NO: 206, HCDR3 comprising an amino acid sequence of SEQ ID NO: 207, LCDR1 comprising an amino acid sequence of SEQ ID NO: 208, LCDR2 comprising an amino acid sequence of SEQ ID NO: 209, or LCDR3 comprising an amino acid sequence of SEQ ID NO: 210;
q) HCDR1 comprising an amino acid sequence of SEQ ID NO: 211, HCDR2 comprising an amino acid sequence of SEQ ID NO: 212, HCDR3 comprising an amino acid sequence of SEQ ID NO: 213, LCDR1 comprising an amino acid sequence of SEQ ID NO: 214, LCDR2 comprising an amino acid sequence of SEQ ID NO: 215, or LCDR3 comprising an amino acid sequence of SEQ ID NO: 216;
r) HCDR1 comprising an amino acid sequence of SEQ ID NO: 217, HCDR2 comprising an amino acid sequence of SEQ ID NO: 218, HCDR3 comprising an amino acid sequence of SEQ ID NO: 219, LCDR1 comprising an amino acid sequence of SEQ ID NO: 220, LCDR2 comprising an amino acid sequence of SEQ ID NO: 221, or LCDR3 comprising an amino acid sequence of SEQ ID NO: 222;
s) HCDR1 comprising an amino acid sequence of SEQ ID NO: 223, HCDR2 comprising an amino acid sequence of SEQ ID NO: 224, HCDR3 comprising an amino acid sequence of SEQ ID NO: 225, LCDR1 comprising an amino acid sequence of SEQ ID NO: 226, LCDR2 comprising an amino acid sequence of SEQ ID NO: 227, or LCDR3 comprising an amino acid sequence of SEQ ID NO: 228;
t) HCDR1 comprising an amino acid sequence of SEQ ID NO: 229, HCDR2 comprising an amino acid sequence of SEQ ID NO: 230, HCDR3 comprising an amino acid sequence of SEQ ID NO: 231, LCDR1 comprising an amino acid sequence of SEQ ID NO: 232, LCDR2 comprising an amino acid sequence of SEQ ID NO: 233, or LCDR3 comprising an amino acid sequence of SEQ ID NO: 234;
u) HCDR1 comprising an amino acid sequence of SEQ ID NO: 235, HCDR2 comprising an amino acid sequence of SEQ ID NO: 236, HCDR3 comprising an amino acid sequence of SEQ ID NO: 237 LCDR1 comprising an amino acid sequence of SEQ ID NO: 238, LCDR2 comprising an amino acid sequence of SEQ ID NO: 239, or LCDR3 comprising an amino acid sequence of SEQ ID NO: 240;
v) HCDR1 comprising an amino acid sequence of SEQ ID NO: 241, HCDR2 comprising an amino acid sequence of any one of SEQ ID NOS: 242-244, HCDR3 comprising an amino acid sequence of SEQ ID NO: 245, LCDR1 comprising an amino acid sequence of SEQ ID NO: 246, LCDR2 comprising an amino acid sequence of any one of SEQ ID NOS: 247-249, or LCDR3 comprising an amino acid sequence of SEQ ID NO: 250;
w) HCDR1 comprising an amino acid sequence of SEQ ID NO: 251, HCDR2 comprising an amino acid sequence of SEQ ID NO: 252, HCDR3 comprising an amino acid sequence of SEQ ID NO: 253, LCDR1 comprising an amino acid sequence of SEQ ID NO: 254, LCDR2 comprising an amino acid sequence of SEQ ID NO: 255 or 256, or LCDR3 comprising an amino acid sequence of SEQ ID NO: 257;
x) HCDR1 comprising an amino acid sequence of SEQ ID NO: 258, HCDR2 comprising an amino acid sequence of SEQ ID NO: 259, HCDR3 comprising an amino acid sequence of SEQ ID NO: 260, LCDR1 comprising an amino acid sequence of SEQ ID NO: 261, LCDR2 comprising an amino acid sequence of SEQ ID NO: 262, or LCDR3 comprising an amino acid sequence of SEQ ID NO: 263;
y) HCDR1 comprising an amino acid sequence of SEQ ID NO: 264, HCDR2 comprising an amino acid sequence of SEQ ID NO: 265, HCDR3 comprising an amino acid sequence of SEQ ID NO: 266, LCDR1 comprising an amino acid sequence of SEQ ID NO: 267, LCDR2 comprising an amino acid sequence of any one of SEQ ID NOS: 268-270, or LCDR3 comprising an amino acid sequence of SEQ ID NO: 271; or
z) HCDR1 comprising an amino acid sequence of SEQ ID NO: 272, HCDR2 comprising an amino acid sequence of SEQ ID NO: 273, HCDR3 comprising an amino acid sequence of SEQ ID NO: 274, LCDR1 comprising an amino acid sequence of SEQ ID NO: 275, LCDR2 comprising an amino acid sequence of SEQ ID NO: 276, or LCDR3 comprising an amino acid sequence of SEQ ID NO: 277.

An antibody construct may comprise an antigen binding domain comprising one or more variable domains. An antibody construct may comprise an antigen binding domain comprising a light chain variable domain (V_{L} domain). A binding domain may comprise at least 80%, at least 90%, at least 95%, at least 97%, at least 98%, at least 99%, or at least 100% sequence identity to any V_{L} sequence in Table 2. An antibody construct may comprise an antigen binding domain comprising a heavy chain variable domain (V_{H} domain). An antigen binding domain may comprise at least 80%, at least 90%, at least 95%, at least 97%, at least 98%, at least 99%, or at least 100% sequence identity to any V_{H} sequence in Table 2. An antigen binding domain can comprise a pair of V_{H} and V_{L} sequences in Table 2. An antigen binding domain can comprise at least 80% sequence identity to any sequence in Table 2.

An antibody construct may comprise an antigen binding domain that specifically binds an antigen, wherein the antigen binding domain comprises:
a) a V_{H} sequence having at least 80%, at least 90%, at least 95%, at least 97%, at least 98%, at least 99%, or at least 100% sequence identity to an amino acid sequence of SEQ ID NO: 83, and a V_{L} sequence having at least 80%, at least 90%, at least 95%, at least 97%, at least 98%, at least 99%, or at least 100% sequence identity to an amino acid sequence of SEQ ID NO: 84;
b) a V_{H} sequence having at least 80%, at least 90%, at least 95%, at least 97%, at least 98%, at least 99%, or at least 100% sequence identity to an amino acid sequence of SEQ ID NO: 85, and a V_{L} sequence having at least 80%, at least 90%, at least 95%, at least 97%, at least 98%, at least 99%, or at least 100% sequence identity to an amino acid sequence of SEQ ID NO: 86;
c) a V_{H} sequence having at least 80%, at least 90%, at least 95%, at least 97%, at least 98%, at least 99%, or at least 100% sequence identity to an amino acid sequence of SEQ ID NO: 87, and a V_{L} sequence having at least 80%, at least 90%, at least 95%, at least 97%, at least 98%, at least 99%, or at least 100% sequence identity to an amino acid sequence of SEQ ID NO: 88;
d) a V_{H} sequence having at least 80%, at least 90%, at least 95%, at least 97%, at least 98%, at least 99%, or at least 100% sequence identity to an amino acid sequence of SEQ ID NO: 89, and a V_{L} sequence having at least 80%, at least 90%, at least 95%, at least 97%, at least 98%, at least 99%, or at least 100% sequence identity to an amino acid sequence of SEQ ID NO: 90;
e) a V_{H} sequence having at least 80%, at least 90%, at least 95%, at least 97%, at least 98%, at least 99%, or at least 100% sequence identity to an amino acid sequence of SEQ ID NO: 91, and a V_{L} sequence having at least 80%, at least 90%, at least 95%, at least 97%, at least 98%, at least 99%, or at least 100% sequence identity to an amino acid sequence of SEQ ID NO: 92;
f) a V_{H} sequence having at least 80%, at least 90%, at least 95%, at least 97%, at least 98%, at least 99%, or at least 100% sequence identity to an amino acid sequence of SEQ ID NO: 93, and a V_{L} sequence having at least 80%, at least 90%, at least 95%, at least 97%, at least 98%, at least 99%, or at least 100% sequence identity to an amino acid sequence of SEQ ID NO: 94;
g) a V_{H} sequence having at least 80%, at least 90%, at least 95%, at least 97%, at least 98%, at least 99%, or at least 100% sequence identity to an amino acid sequence of SEQ ID NO: 95, and a V_{L} sequence having at least 80%, at least 90%, at least 95%, at least 97%, at least 98%, at least 99%, or at least 100% sequence identity to an amino acid sequence of SEQ ID NO: 96;
h) a V_{H} sequence having at least 80%, at least 90%, at least 95%, at least 97%, at least 98%, at least 99%, or at least 100% sequence identity to an amino acid sequence of SEQ ID NO: 97, and a V_{L} sequence having at least 80%, at least 90%, at least 95%, at least 97%, at least 98%, at least 99%, or at least 100% sequence identity to an amino acid sequence of SEQ ID NO: 98;
i) a V_{H} sequence having at least 80%, at least 90%, at least 95%, at least 97%, at least 98%, at least 99%, or at least 100% sequence identity to an amino acid sequence of SEQ ID NO: 99, and a V_{L} sequence having at least 80%, at least 90%, at least 95%, at least 97%, at least 98%, at least 99%, or at least 100% sequence identity to an amino acid sequence of SEQ ID NO: 100;
j) a V_{H} sequence having at least 80%, at least 90%, at least 95%, at least 97%, at least 98%, at least 99%, or at least 100% sequence identity to an amino acid sequence of SEQ ID NO: 101, and a V_{L} sequence having at least 80%, at least 90%, at least 95%, at least 97%, at least 98%, at least 99%, or at least 100% sequence identity to an amino acid sequence of SEQ ID NO: 102;
k) a V_{H} sequence having at least 80%, at least 90%, at least 95%, at least 97%, at least 98%, at least 99%, or at least 100% sequence identity to an amino acid sequence of SEQ ID NO: 101, and a V_{L} sequence having at least 80%, at least 90%, at least 95%, at least 97%, at least 98%, at least 99%, or at least 100% sequence identity to an amino acid sequence of SEQ ID NO: 103;
l) a V_{H} sequence having at least 80%, at least 90%, at least 95%, at least 97%, at least 98%, at least 99%, or at least 100% sequence identity to an amino acid sequence of SEQ ID NO: 104, and a V_{L} sequence having at least 80%, at least 90%, at least 95%, at least 97%, at least 98%, at least 99%, or at least 100% sequence identity to an amino acid sequence of SEQ ID NO: 105;
m) a V_{H} sequence having at least 80%, at least 90%, at least 95%, at least 97%, at least 98%, at least 99%, or at least 100% sequence identity to an amino acid sequence of SEQ ID NO: 106, and a V_{L} sequence having at least 80%, at least 90%, at least 95%, at least 97%, at least 98%, at least 99%, or at least 100% sequence identity to an amino acid sequence of SEQ ID NO: 107;
n) a V_{H} sequence having at least 80%, at least 90%, at least 95%, at least 97%, at least 98%, at least 99%, or at least 100% sequence identity to an amino acid sequence of SEQ ID NO: 109, and a V_{L} sequence having at least 80%, at least 90%, at least 95%, at least 97%, at least 98%, at least 99%, or at least 100% sequence identity to an amino acid sequence of SEQ ID NO: 108;
o) a V_{H} sequence having at least 80%, at least 90%, at least 95%, at least 97%, at least 98%, at least 99%, or at least 100% sequence identity to an amino acid sequence of SEQ ID NO: 110, and a V_{L} sequence having at least 80%, at least 90%, at least 95%, at least 97%, at least 98%, at least 99%, or at least 100% sequence identity to an amino acid sequence of SEQ ID NO: 108;
p) a V_{H} sequence having at least 80%, at least 90%, at least 95%, at least 97%, at least 98%, at least 99%, or at least 100% sequence identity to an amino acid sequence of SEQ ID NO: 111, and a V_{L} sequence having at least 80%, at least 90%, at least 95%, at least 97%, at least 98%, at least 99%, or at least 100% sequence identity to an amino acid sequence of SEQ ID NO: 112;
q) a V_{H} sequence having at least 80%, at least 90%, at least 95%, at least 97%, at least 98%, at least 99%, or at least 100% sequence identity to an amino acid sequence of SEQ ID NO: 113, and a V_{L} sequence having at least 80%, at least 90%, at least 95%, at least 97%, at least 98%, at least 99%, or at least 100% sequence identity to an amino acid sequence of SEQ ID NO: 114;
r) a V_{H} sequence having at least 80%, at least 90%, at least 95%, at least 97%, at least 98%, at least 99%, or at least 100% sequence identity to an amino acid sequence of SEQ ID NO: 115, and a V_{L} sequence having at least 80%, at least 90%, at least 95%, at least 97%, at least 98%, at least 99%, or at least 100% sequence identity to an amino acid sequence of SEQ ID NO: 116;
s) a V_{H} sequence having at least 80%, at least 90%, at least 95%, at least 97%, at least 98%, at least 99%, or at least 100% sequence identity to an amino acid sequence of SEQ ID NO: 117, and a V_{L} sequence having at least 80%, at least 90%, at least 95%, at least 97%, at least 98%, at least 99%, or at least 100% sequence identity to an amino acid sequence of SEQ ID NO: 118;
t) a V_{H} sequence having at least 80%, at least 90%, at least 95%, at least 97%, at least 98%, at least 99%, or at least 100% sequence identity to an amino acid sequence of SEQ ID NO: 117, and a V_{L} sequence having at least 80%, at least 90%, at least 95%, at least 97%, at least 98%, at least 99%, or at least 100% sequence identity to an amino acid sequence of SEQ ID NO: 119;
u) a V_{H} sequence having at least 80%, at least 90%, at least 95%, at least 97%, at least 98%, at least 99%, or at least 100% sequence identity to an amino acid sequence of SEQ ID NO: 117, and a V_{L} sequence having at least 80%, at least 90%, at least 95%, at least 97%, at least 98%, at least 99%, or at least 100% sequence identity to an amino acid sequence of SEQ ID NO: 120;
v) a V_{H} sequence having at least 80%, at least 90%, at least 95%, at least 97%, at least 98%, at least 99%, or at least 100% sequence identity to an amino acid sequence of SEQ ID NO: 117, and a V_{L} sequence having at least 80%, at least 90%, at least 95%, at least 97%, at least 98%, at least 99%, or at least 100% sequence identity to an amino acid sequence of SEQ ID NO: 121;
w) a V_{H} sequence having at least 80%, at least 90%, at least 95%, at least 97%, at least 98%, at least 99%, or at least 100% sequence identity to an amino acid sequence of SEQ ID NO: 117, and a V_{L} sequence having at least 80%, at least 90%, at least 95%, at least 97%, at least 98%, at least 99%, or at least 100% sequence identity to an amino acid sequence of SEQ ID NO: 122;
x) a V_{H} sequence having at least 80%, at least 90%, at least 95%, at least 97%, at least 98%, at least 99%, or at least 100% sequence identity to an amino acid sequence of SEQ ID NO: 123, and a V_{L} sequence having at least 80%, at least 90%, at least 95%, at least 97%, at least 98%, at least 99%, or at least 100% sequence identity to an amino acid sequence of SEQ ID NO: 124;
y) a V_{H} sequence having at least 80%, at least 90%, at least 95%, at least 97%, at least 98%, at least 99%, or at least 100% sequence identity to an amino acid sequence of SEQ ID NO: 125, and a V_{L} sequence having at least 80%, at least 90%, at least 95%, at least 97%, at least 98%, at least 99%, or at least 100% sequence identity to an amino acid sequence of SEQ ID NO: 126;
z) a V_{H} sequence having at least 80%, at least 90%, at least 95%, at least 97%, at least 98%, at least 99%, or at least 100% sequence identity to an amino acid sequence of SEQ ID NO: 127, and a V_{L} sequence having at least 80%, at least 90%, at least 95%, at least 97%, at least 98%, at least 99%, or at least 100% sequence identity to an amino acid sequence of SEQ ID NO: 128;
aa) a V_{H} sequence having at least 80%, at least 90%, at least 95%, at least 97%, at least 98%, at least 99%, or at least 100% sequence identity to an amino acid sequence of SEQ ID NO: 130, and a V_{L} sequence having at least 80%, at least 90%, at least 95%, at least 97%, at least 98%, at least 99%, or at least 100% sequence identity to an amino acid sequence of SEQ ID NO: 129;
bb) a V_{H} sequence having at least 80%, at least 90%, at least 95%, at least 97%, at least 98%, at least 99%, or at least 100% sequence identity to an amino acid sequence of SEQ ID NO: 131, and a V_{L} sequence having at least 80%, at least 90%, at least 95%, at least 97%, at least 98%, at least 99%, or at least 100% sequence identity to an amino acid sequence of SEQ ID NO: 132;
cc) a V_{H} sequence having at least 80%, at least 90%, at least 95%, at least 97%, at least 98%, at least 99%, or at least 100% sequence identity to an amino acid sequence of SEQ ID NO: 133, and a V_{L} sequence having at least 80%, at least 90%, at least 95%, at least 97%, at least 98%, at least 99%, or at least 100% sequence identity to an amino acid sequence of SEQ ID NO: 134;
dd) a V_{H} sequence having at least 80%, at least 90%, at least 95%, at least 97%, at least 98%, at least 99%, or at least 100% sequence identity to an amino acid sequence of SEQ ID NO: 135, and a V_{L} sequence having at least 80%, at least 90%, at least 95%, at least 97%, at least 98%, at least 99%, or at least 100% sequence identity to an amino acid sequence of SEQ ID NO: 136;
ee) a V_{H} sequence having at least 80%, at least 90%, at least 95%, at least 97%, at least 98%, at least 99%, or at least 100% sequence identity to an amino acid sequence of SEQ ID NO: 137, and a V_{L} sequence having at least 80%, at least 90%, at least 95%, at least 97%, at least 98%, at least 99%, or at least 100% sequence identity to an amino acid sequence of SEQ ID NO: 138;
ff) a V_{H} sequence having at least 80%, at least 90%, at least 95%, at least 97%, at least 98%, at least 99%, or at least 100% sequence identity to an amino acid sequence of SEQ ID NO: 140, and a V_{L} sequence having at least 80%, at least 90%, at least 95%, at least 97%, at least 98%, at least 99%, or at least 100% sequence identity to an amino acid sequence of SEQ ID NO: 139;
gg) a V_{H} sequence having at least 80%, at least 90%, at least 95%, at least 97%, at least 98%, at least 99%, or at least 100% sequence identity to an amino acid sequence of SEQ ID NO: 141, and a V_{L} sequence having at least 80%, at least 90%, at least 95%, at least 97%, at least 98%, at least 99%, or at least 100% sequence identity to an amino acid sequence of SEQ ID NO: 142;
hh) a V_{H} sequence having at least 80%, at least 90%, at least 95%, at least 97%, at least 98%, at least 99%, or at least 100% sequence identity to an amino acid sequence of SEQ ID NO: 143, and a V_{L} sequence having at least 80%, at least 90%, at least 95%, at least 97%, at least 98%, at least 99%, or at least 100% sequence identity to an amino acid sequence of SEQ ID NO: 144;
ii) ii) a V_{H} sequence having at least 80%, at least 90%, at least 95%, at least 97%, at least 98%, at least 99%, or at least 100% sequence identity to an amino acid sequence of SEQ ID NO: 145, and a V_{L} sequence having at least 80%, at least 90%, at least 95%, at least 97%, at least 98%, at least 99%, or at least 100% sequence identity to an amino acid sequence of SEQ ID NO: 146;
jj) a V_{H} sequence having at least 80%, at least 90%, at least 95%, at least 97%, at least 98%, at least 99%, or at least 100% sequence identity to an amino acid sequence of SEQ ID NO: 147, and a V_{L} sequence having at least 80%, at least 90%, at least 95%, at least 97%, at least 98%, at least 99%, or at least 100% sequence identity to an amino acid sequence of SEQ ID NO: 148;
kk) a V_{H} sequence having at least 80%, at least 90%, at least 95%, at least 97%, at least 98%, at least 99%, or at least 100% sequence identity to an amino acid sequence of SEQ ID NO: 149, and a V_{L} sequence having at least 80%, at least 90%, at least 95%, at least 97%, at least 98%, at least 99%, or at least 100% sequence identity to an amino acid sequence of SEQ ID NO: 150;
ll) a V_{H} sequence having at least 80%, at least 90%, at least 95%, at least 97%, at least 98%, at least 99%, or at least 100% sequence identity to an amino acid sequence of SEQ ID NO: 151, and a V_{L} sequence having at least 80%, at least 90%, at least 95%, at least 97%, at least 98%, at least 99%, or at least 100% sequence identity to an amino acid sequence of SEQ ID NO: 153;
mm) a V_{H} sequence having at least 80%, at least 90%, at least 95%, at least 97%, at least 98%, at least 99%, or at least 100% sequence identity to an amino acid sequence of SEQ ID NO: 152, and a V_{L} sequence having at least 80%, at least 90%, at least 95%, at least 97%, at least 98%, at least 99%, or at least 100% sequence identity to an amino acid sequence of SEQ ID NO: 153;
nn) a V_{H} sequence having at least 80%, at least 90%, at least 95%, at least 97%, at least 98%, at least 99%, or at least 100% sequence identity to an amino acid sequence of SEQ ID NO: 154, and a V_{L} sequence having at least 80%, at least 90%, at least 95%, at least 97%, at least 98%, at least 99%, or at least 100% sequence identity to an amino acid sequence of SEQ ID NO: 155;
oo) a V_{H} sequence having at least 80%, at least 90%, at least 95%, at least 97%, at least 98%, at least 99%, or at least 100% sequence identity to an amino acid sequence of SEQ ID NO: 156, and a V_{L} sequence having at least 80%, at least 90%, at least 95%, at least 97%, at least 98%, at least 99%, or at least 100% sequence identity to an amino acid sequence of SEQ ID NO: 157;
pp) a V_{H} sequence having at least 80%, at least 90%, at least 95%, at least 97%, at least 98%, at least 99%, or at least 100% sequence identity to an amino acid sequence of SEQ ID NO: 158, and a V_{L} sequence having at least 80%, at least 90%, at least 95%, at least 97%, at least 98%, at least 99%, or at least 100% sequence identity to an amino acid sequence of SEQ ID NO: 159;
qq) a V_{H} sequence having at least 80%, at least 90%, at least 95%, at least 97%, at least 98%, at least 99%, or at least 100% sequence identity to an amino acid sequence of SEQ ID NO: 160, and a V_{L} sequence having at least 80%, at least 90%, at least 95%, at least 97%, at least 98%, at least 99%, or at least 100% sequence identity to an amino acid sequence of SEQ ID NO: 161;
rr) a V_{H} sequence having at least 80%, at least 90%, at least 95%, at least 97%, at least 98%, at least 99%, or at least 100% sequence identity to an amino acid sequence of SEQ ID NO: 162, and a V_{L} sequence having at least 80%, at least 90%, at least 95%, at least 97%, at least 98%, at least 99%, or at least 100% sequence identity to an amino acid sequence of SEQ ID NO: 163;
ss) a V_{H} sequence having at least 80%, at least 90%, at least 95%, at least 97%, at least 98%, at least 99%, or at least 100% sequence identity to an amino acid sequence of SEQ ID NO: 164, and a V_{L} sequence having at least 80%, at least 90%, at least 95%, at least 97%, at least 98%, at least 99%, or at least 100% sequence identity to an amino acid sequence of SEQ ID NO: 167;
tt) a V_{H} sequence having at least 80%, at least 90%, at least 95%, at least 97%, at least 98%, at least 99%, or at least 100% sequence identity to an amino acid sequence of SEQ ID NO: 164, and a V_{L} sequence having at least 80%, at least 90%, at least 95%, at least 97%, at least 98%, at least 99%, or at least 100% sequence identity to an amino acid sequence of SEQ ID NO: 168;
uu) a V_{H} sequence having at least 80%, at least 90%, at least 95%, at least 97%, at least 98%, at least 99%, or at least 100% sequence identity to an amino acid sequence of SEQ ID NO: 165, and a V_{L} sequence having at least 80%, at least 90%, at least 95%, at least 97%, at least 98%, at least 99%, or at least 100% sequence identity to an amino acid sequence of SEQ ID NO: 167;
vv) a V_{H} sequence having at least 80%, at least 90%, at least 95%, at least 97%, at least 98%, at least 99%, or at least 100% sequence identity to an amino acid sequence of SEQ ID NO: 165, and a V_{L} sequence having at least 80%, at least 90%, at least 95%, at least 97%, at least 98%, at least 99%, or at least 100% sequence identity to an amino acid sequence of SEQ ID NO: 168;
ww) a V_{H} sequence having at least 80%, at least 90%, at least 95%, at least 97%, at least 98%, at least 99%, or at least 100% sequence identity to an amino acid sequence of SEQ ID NO: 166, and a V_{L} sequence having at least 80%, at least 90%, at least 95%, at least 97%, at least 98%, at least 99%, or at least 100% sequence identity to an amino acid sequence of SEQ ID NO: 167;
xx) a V_{H} sequence having at least 80%, at least 90%, at least 95%, at least 97%, at least 98%, at least 99%, or at least 100% sequence identity to an amino acid sequence of SEQ ID NO: 166, and a V_{L} sequence having at least 80%, at least 90%, at least 95%, at least 97%, at least 98%, at least 99%, or at least 100% sequence identity to an amino acid sequence of SEQ ID NO: 168;
yy) a V_{H} sequence having at least 80%, at least 90%, at least 95%, at least 97%, at least 98%, at least 99%, or at least 100% sequence identity to an amino acid sequence of SEQ ID NO: 171, and a V_{L} sequence having at least 80%, at least 90%, at least 95%, at least 97%, at least 98%, at least 99%, or at least 100% sequence identity to an amino acid sequence of SEQ ID NO: 172;
zz) a V_{H} sequence having at least 80%, at least 90%, at least 95%, at least 97%, at least 98%, at least 99%, or at least 100% sequence identity to an amino acid sequence of SEQ ID NO: 174, and a V_{L} sequence having at least 80%, at least 90%, at least 95%, at least 97%, at least 98%, at least 99%, or at least 100% sequence identity to an amino acid sequence of SEQ ID NO: 173;
aaa) a V_{H} sequence having at least 80%, at least 90%, at least 95%, at least 97%, at least 98%, at least 99%, or at least 100% sequence identity to an amino acid sequence of SEQ ID NO: 175, and a V_{L} sequence having at least 80%, at least 90%, at least 95%, at least 97%, at least 98%, at least 99%, or at least 100% sequence identity to an amino acid sequence of SEQ ID NO: 176;
bbb) a V_{H} sequence having at least 80%, at least 90%, at least 95%, at least 97%, at least 98%, at least 99%, or at least 100% sequence identity to an amino acid sequence of SEQ ID NO: 177, and a V_{L} sequence having at least 80%, at least 90%, at least 95%, at least 97%, at least 98%, at least 99%, or at least 100% sequence identity to an amino acid sequence of SEQ ID NO: 178;
ccc) a V_{H} sequence having at least 80%, at least 90%, at least 95%, at least 97%, at least 98%, at least 99%, or at least 100% sequence identity to an amino acid sequence of SEQ ID NO: 179, and a V_{L} sequence having at least 80%, at least 90%, at least 95%, at least 97%, at least 98%, at least 99%, or at least 100% sequence identity to an amino acid sequence of SEQ ID NO: 180;
ddd) a V_{H} sequence having at least 80%, at least 90%, at least 95%, at least 97%, at least 98%, at least 99%, or at least 100% sequence identity to an amino acid sequence of SEQ ID NO: 181, and a V_{L} sequence having at least 80%, at least 90%, at least 95%, at least 97%, at least 98%, at least 99%, or at least 100% sequence identity to an amino acid sequence of SEQ ID NO: 182;
eee) a V_{H} sequence having at least 80%, at least 90%, at least 95%, at least 97%, at least 98%, at least 99%, or at least 100% sequence identity to an amino acid sequence of SEQ ID NO: 183, and a V_{L} sequence having at least 80%, at least 90%, at least 95%, at least 97%, at least 98%, at least 99%, or at least 100% sequence identity to an amino acid sequence of SEQ ID NO: 184;
fff) a V_{H} sequence having at least 80%, at least 90%, at least 95%, at least 97%, at least 98%, at least 99%, or at least 100% sequence identity to an amino acid sequence of SEQ ID NO: 185, and a V_{L} sequence having at least 80%, at least 90%, at least 95%, at least 97%, at least 98%, at least 99%, or at least 100% sequence identity to an amino acid sequence of SEQ ID NO: 186;
ggg) a V_{H} sequence having at least 80%, at least 90%, at least 95%, at least 97%, at least 98%, at least 99%, or at least 100% sequence identity to an amino acid sequence of SEQ ID NO: 187, and a V_{L} sequence having at least 80%, at least 90%, at least 95%, at least 97%, at least 98%, at least 99%, or at least 100% sequence identity to an amino acid sequence of SEQ ID NO: 188;
hhh) a V_{H} sequence having at least 80%, at least 90%, at least 95%, at least 97%, at least 98%, at least 99%, or at least 100% sequence identity to an amino acid sequence of SEQ ID NO: 189, and a V_{L} sequence having at least 80%, at least 90%, at least 95%, at least 97%, at least 98%, at least 99%, or at least 100% sequence identity to an amino acid sequence of SEQ ID NO: 190;
iii) a V_{H} sequence having at least 80%, at least 90%, at least 95%, at least 97%, at least 98%, at least 99%, or at least 100% sequence identity to an amino acid sequence of SEQ ID NO: 191, and a V_{L} sequence having at least 80%, at least 90%, at least 95%, at least 97%, at least 98%, at least 99%, or at least 100% sequence identity to an amino acid sequence of SEQ ID NO: 192;
jjj) a V_{H} sequence having at least 80%, at least 90%, at least 95%, at least 97%, at least 98%, at least 99%, or at least 100% sequence identity to an amino acid sequence of SEQ ID NO: 193, and a V_{L} sequence having at least 80%, at least 90%, at least 95%, at least 97%, at least 98%, at least 99%, or at least 100% sequence identity to an amino acid sequence of SEQ ID NO: 194;
kkk) a V_{H} sequence having at least 80%, at least 90%, at least 95%, at least 97%, at least 98%, at least 99%, or at least 100% sequence identity to an amino acid sequence of SEQ ID NO: 195, and a V_{L} sequence having at least 80%, at least 90%, at least 95%, at least 97%, at least 98%, at least 99%, or at least 100% sequence identity to an amino acid sequence of SEQ ID NO: 196;
lll) a V_{H} sequence having at least 80%, at least 90%, at least 95%, at least 97%, at least 98%, at least 99%, or at least 100% sequence identity to an amino acid sequence of SEQ ID NO: 197, and a V_{L} sequence having at least 80%, at least 90%, at least 95%, at least 97%, at least 98%, at least 99%, or at least 100% sequence identity to an amino acid sequence of SEQ ID NO: 198; or
mmm) a V_{H} sequence having at least 80%, at least 90%, at least 95%, at least 97%, at least 98%, at least 99%, or at least 100% sequence identity to an amino acid sequence of SEQ ID NO: 285, and a V_{L} sequence having at least 80%, at least 90%, at least 95%, at least 97%, at least 98%, at least 99%, or at least 100% sequence identity to an amino acid sequence of SEQ ID NO: 286.

An antibody construct may comprise a sequence from Table 1 and/or Table 2. An antibody construct may comprise a set of CDR sequences from Table 1 and/or a pair of V_{H} and V_{L} sequences from Table 2.

TABLE 1
ANTIBODY CDRS

| | | SEQ | |
|---|---|---|---|
| | | ID | |
| ANTIBODY | REGION | NO | SEQUENCE |
| Ipilumumab | HCDR1 | 1 | GFTFSSYT |
| | HCDR2 | 2 | ISYDGNNK |
| | HCDR3 | 3 | ARTGWLGPFDY |
| | LCDR1 | 4 | QSVGSSY |
| | LCDR2 | 5 | SSY |
| | LCDR3 | 6 | QQYGSSPWT |
| Opdivo® | HCDR1 | 7 | GITFSNSG |
| (nivolumab) | HCDR2 | 8 | IWYDGSKR |
| | HCDR3 | 9 | ATNDDY |
| | LCDR1 | 10 | QSVSSYL |
| | LCDR2 | 11 | DAS |
| | LCDR3 | 12 | QQSSNWPRT |
| Keytruda® | HCDR1 | 13 | GYTFTNYY |
| (pembrolizumab) | HCDR2 | 14 | INPSNGGT |
| | HCDR3 | 15 | ARRDYRFDMGFDY |
| | LCDR1 | 16 | KGVSTSGYSY |
| | LCDR2 | 17 | LAS |
| | LCDR3 | 18 | QHSRDLPLT |
| Vonlerolizumab | HCDR1 | 19 | GYTFTDSY |
| | HCDR2 | 20 | MYPDNGDS |
| | HCDR3 | 21 | VLAPRWYFSV |
| | LCDR1 | 22 | QDISNY |
| | LCDR2 | 23 | YTS |
| | LCDR3 | 24 | QQGHTLPPT |
| Varlilumab | HCDR1 | 25 | GFTFSSYD |
| | HCDR2 | 26 | IWYDGSNK |
| | HCDR3 | 27 | ARGSGNWGFFDY |
| | LCDR1 | 28 | QGISRW |
| | LCDR2 | 29 | AAS |
| | LCDR3 | 30 | QQYNTYPRT |
| Zinbryta® | HCDR1 | 31 | GYTFTSYR |
| (Daclizumab) | HCDR2 | 32 | INPSTGYT |
| | HCDR3 | 33 | ARGGGVFDY |
| | LCDR1 | 34 | SSSISY |
| | LCDR2 | 35 | TTS |
| | LCDR3 | 36 | HQRSTYPLT |
| Antibody | HCDR1 | 37 | SYGMH |
| to GITR | HCDR2 | 38 | VIWYEGSNKYYADSVKG |
| | HCDR3 | 39 | GGSMVRGDYYYGMDV |
| | LCDR1 | 40 | RASQGISSALA |
| | LCDR2 | 41 | DASSLES |
| | LCDR3 | 42 | QQFNSYPYT |
| Antibody | HCDR1 | 43 | DYYWN |
| to LAG-3 | HCDR2 | 44 | EINHRGSTNSNPSLKS |
| | HCDR3 | 45 | GYSDYEYNWFDP |
| | LCDR1 | 46 | RASQSISSYLA |
| | LCDR2 | 47 | DASNRAT |
| | LCDR3 | 48 | QQRSNWPLT |
| Utomilumab | HCDR1 | 50 | GYSFSTYW |
| | HCDR2 | 51 | 1YPGDSYT |
| | HCDR3 | 52 | ARGYGIFDY |
| | LCDR1 | 53 | N1GDQY |
| | LCDR2 | 54 | QDK |
| | LCDR3 | 55 | ATYTGFGSLAV |
| Antibody | HCDR1 | 56 | GYTFTDYN |
| to TNFR2 | HCDR2 | 57 | INPNYEST |
| variant 1 | HCDR3 | 58 | RDKGWYFDV |
| | LCDR1 | 59 | SSVKN |
| | LCDR2 | 60 | YTS |
| | LCDR3 | 61 | QQFTSSPYT |
| Anlibody | HCDR1 | 62 | GFSLSTSGMG |
| to TNFR2 | HCDR2 | 63 | IWWDDDK |
| variant 2 | HCDR3 | 64 | ARLTGTRYFDY |
| | LCDR1 | 65 | QDINKF |
| | LCDR2 | 66 | YTS |
| | LCDR3 | 67 | LQYGNLWT |
| Antibody | HCDR1 | 68 | GYTFTDYS |
| to TNFR2 | HCDR2 | 69 | INTETGEP |
| variant 3 | HCDR3 | 70 | ATYYGSSYVPDY |
| | LCDR1 | 71 | QNVGTA |
| | LCDR2 | 72 | WTS |
| | LCDR3 | 73 | QYSDYPYT |
| Antibody | HCDR1 | 74 | GYTFTDY |
| to TNFR2 | HCDR2 | 75 | WVDPEYGS |
| variant 4 | HCDR3 | 76 | ARDDGSYSPFDY |
| | LCDR1 | 77 | QNINKY |
| | (major) | | |
| | LCDR2 | 78 | YTS |
| | (major) | | |
| | LCDR3 | 79 | LQYVNLLT |
| | (major) | | |
| | LCDR1 | 80 | ENVVTY |
| | (minor) | | |
| | LCDR2 | 81 | GAS |
| | (minor) | | |
| | LCDR3 | 82 | QGYSYPYT |
| | (minor) | | |
| Antibody | HCDR1 | 199 | DYYIH |
| huAD208.4.1 | HCDR2 | 200 | LVYPYIGGTNYNQKFKG |
| to LRRC15 | HCDR3 | 201 | GDNKYDAMDY |
| | LCDR1 | 202 | RASQSVSTSSYSYMH |
| | LCDR2 | 203 | YASSLES |
| | LCDR3 | 204 | EQSWEIRT |
| Antibody | HCDR1 | 205 | NYWMH |
| huAD208.12.1 | HCDR2 | 206 | MIHPNSGSTKHNEKFRG |
| to LRRC15 | HCDR3 | 207 | SDFGNYRWYFDV |
| | LCDR1 | 208 | RASQSSSNNLH |
| | LCDR2 | 209 | YVSQSIS |
| | LCDR3 | 210 | QQSNSWPFT |
| Antibody | HCDR1 | 211 | DYYIH |
| huAD208.14.1 | HCDR2 | 212 | LVYPYIGGSSYNQQFKG |
| to LRRC15 | HCDR3 | 213 | GDNNYDAMDY |
| | LCDR1 | 214 | RASQSVSTSTYNYMH |
| | LCDR2 | 215 | YASNLES |
| | LCDR3 | 216 | HHTWEIRT |
| Antibody | HCDR1 | 217 | SYGVH |
| Hu139.10 to | HCDR2 | 218 | VIWAGGSTNYNSALMS |
| LRRC15 | HCDR3 | 219 | HMITEDYYGMDY |
| | LCDR1 | 220 | KSSQSLLNSRTRKNYLA |
| | LCDR2 | 221 | WASTRES |
| | LCDR3 | 222 | KQSYNLPT |
| Antibody | HCDR1 | 223 | NYWLG |
| muAD210.40.9 | HCDR2 | 224 | DIYPGGGNTYYNEKLKG |
| to LRRC15 | HCDR3 | 225 | WGDKKGNYFAY |
| | LCDR1 | 226 | TASSSVYSSYLH |
| | LCDR2 | 227 | STSNLAS |
| | LCDR3 | 228 | HQYHRSPT |
| Antibody | HCDR1 | 229 | NFGMN |
| muAD209.9.1 | HCDR2 | 230 | WINLYTGEPTFADDFKG |
| to LRRC15 | HCDR3 | 231 | KGETYYRYDGFAY |
| | LCDR1 | 232 | RSSKSLLHSNGNTHLY |
| | LCDR2 | 233 | RMSNLAS |
| | LCDR3 | 234 | MQLLEYPYT |
| Antibody | HCDR1 | 235 | SYAMS |
| nrAb-C | HCDR2 | 236 | AISGSGGSTYYADSVKG |
| to | HCDR3 | 237 | DFSSRRWYLEY |
| ASGR1 | LCDR1 | 238 | QGDSLRSYYAS |
| | LCDR2 | 239 | GKNNRPS |
| | LCDR3 | 240 | NSLERIGYLSYV |
| Antibody | HCDR1 | 241 | GYYMH |
| G2D to | HCDR2 | 242 | RINPNNGATNYNQNFKD |
| ASGR1 | HCDR2 | 243 | RINPNQGATNYNQNFKD |
| | HCDR2 | 244 | RINPNNAATNYNQNFKD |
| | HCDR3 | 245 | VNFYY |
| | LCDR1 | 246 | KASQVINSYLS |
| | LCDR2 | 247 | RANTLVD |
| | LCDR2 | 248 | RANTLVS |
| | LCDR2 | 249 | RANTLVE |
| | LCDR3 | 250 | LQYAEFPYT |
| Antibody | HCDR1 | 251 | SYTMH |
| K2E to | HCDR2 | 252 | YISPSSGYTEYNQKFKJD |
| ASGR1 | HCDR3 | 253 | KFDY |
| | LCDR1 | 254 | KASQDINSYLS |
| | LCDR2 | 255 | RANRLVD |
| | LCDR2 | 256 | RANRLVE |
| | LCDR3 | 257 | LQYDEFPFT |
| Antibody | HCDR1 | 258 | SYWIN |
| J4F to | HCDR2 | 259 | RIVPGSGSTYYNEMFKD |
| ASGR1 | HCDR3 | 260 | KPNFDV |
| | LCDR1 | 261 | KASQNVGTNVA |
| | LCDR2 | 262 | SASYRFS |
| | LCDR3 | 263 | QQYNSYPLT |
| Antibody | HCDR1 | 264 | SDYAWN |
| L4L to | HCDR2 | 265 | YISYSGSTRYNPSLKS |
| ASGR1 | HCDR3 | 266 | RYRYDEGYGMDY |
| | LCDR1 | 267 | RASENIYSNLA |
| | LCDR2 | 268 | AATNLAD |
| | LCDR2 | 269 | AATNLAS |
| | LCDR2 | 270 | AATNLAE |
| | LCDR3 | 271 | QHFWGTPPWT |
| Antibody | HCDR1 | 272 | SDYAWN |
| H8K to | HCDR2 | 273 | YRSYRGSTSYNPSLKS |
| ASGR1 | HCDR3 | 274 | RGYYGSSSHWYFDV |
| | LCDR1 | 275 | RASENIYSYLA |
| | LCDR2 | 276 | NAKTLAE |
| | LCDR3 | 277 | QHHYGTPLT |

TABLE 2
ANTIBODY V_{H} SEQUENCE AND V_{L} SEQUENCES

| | | SEQ | |
|---|---|---|---|
| | | ID | |
| ANTIBODY | REGION | NO: | SEQUENCE: |
| Ipilumumab | VH | 83 | qvqlvesgggvvqpgrslrlscaasgftfssytmhwvrqapgkglewvtfisy |
| | | | dgnnkyyadsvkgrfusrdnskntlylqmnslraedtaiyycartgwlgpfdy |
| | | | wgqgtlvtvss |
| | VL | 84 | eivltqspgtlslspgeratlscrasqsvgssylawyqqkpgqaprllivgaf |
| | | | sratgipdifsgsgsgtdftltisrlepedfavyycqqygsspwtfgqgtkve |
| | | | ik |
| Opdivo® | VH | 85 | qvqlvesgggvvqpgrslrldckasgitfsnsgmhwvrqapgkglewvaviw |
| (nivolumab) | | | ydgskiyyadsvkgrftisrdnskntlflqnmslraedtavyycatnddywg |
| | | | qgtlvtvss |
| | VL | 86 | Eivltqspatlslspgeratlscrasqsvssylawyqqkpgqaprllivdasn |
| | | | Ratgiparfsgsgsgtdftltisslepedfavyycqqssnwprtfgqgtkvei |
| | | | k |
| Keytruda® | VH | 87 | qvqlvqsgvevkkpgasvkvsckasgytftnyymyvvvrqapgqglewmgg |
| (pembroliznmab) | | | inpsnggtnfnekfknrvtlttdsstttaymelkslqfddtavyycarrdy |
| | | | rfdmgfdywgqgttvtvss |
| | VL | 88 | Eivltqspatlslspgeratlscraskgvstsgysylhwyqqkpgqaprlli |
| | | | Ylasylesgvparfsgsgsgtdftltisslepedfavyycqhsrdlpltfgg |
| | | | gtkveik |
| Atezolizumab | VH | 89 | evqlvesggglvqpggslrlscaasgftfsdswilnwrqapgkglewvawisp |
| | | | yggstyyadsvkgrftisadtskntaylqmnslraedtavyycarrhwpggfd |
| | | | ywgqgtlvtvss |
| | VL | 90 | diqmtqspsslsasvgdrvUtcrasqdvstavawyqqkpgkapklliysasfl |
| | | | ysgvpsrfsgsgsgtdftltisslqpedfatyycqqylyhpatfgqgtkveik |
| Durvalumab | VH | 91 | evqlvesggglvqpggslrlscaasgftfsywmsYvvrqapgkglewvamkqd |
| | | | gsekyyvdsvkgrftisrdnaknslylqmnslraedtavyycareggwfgela |
| | | | fdywgqgtlvtvss |
| | VL | 92 | Eivltqspgtlslspgeratlscrasqrvsssylawyqqkpgqaprlliydas |
| | | | sratgipdrfsgsgsgtdftitisrlepedfavyycqqygslpwtfgqgtkve |
| | | | ik |
| MDX-1106 | VH | 93 | qvqlvqsgaevkkpgssvkvscktsgdtfstyaiswvrqapgqglewmggiip |
| | | | ifgkahyaqkfqgrvtitadeststaymelsslisedtavyfcarkfhfVsgs |
| | | | pfgmdvwgqgttvtvss |
| | VL | 94 | Eivltqspatlslspgeratlscrasqsvssylawyqqkpgqaprlliydasn |
| | | | ratgiparfsgsgsgtdftltisslepedfavvycqqrsnwptfgqgtkveik |
| Antibody | VH | 95 | evqlqqsgaelvkpgasvkisckasgytftdynmdwvkqshgkslewigdin |
| to TNFR2 | | | pnycstsynqkfkgkatltvdkssstavmcvrsltscdtavfycardkgwyf |
| variant 1 | | | dvwgagttvtvss |
| | VL | 96 | envltqspaimsaslgekvtmscrasssvknmywyqqksdaspklwiyytsnl |
| | | | apgvparfsgsgsgnsysltissmegedaatyycqqftsspytfgggtklelk |
| Antibody | VH | 97 | Qvtlkesgpgilqpsqtlsltcsfsgfslstsgmgvgwiiqpsgkglewlahi |
| to TNFR2 | | | Wwdddkfynpslksqltiskdtsmqvflkltswtadtatyycarltgtryfdy |
| variant 2 | | | wgqgttltvss |
| | VL | 98 | Dvqmtqspsslsaslggkvtitckasqdinkfiawyqhkpgkgprllihvlstl |
| | | | qpgipskfsgsgsgrdysfsisnlepediatyyclqygnlwtfgggtkleit |
| Antibody | VH | 99 | Qiqlvqsgpelkkpgetvkisckasgytfidysmhwvkqapgkglkwmgwint |
| to TNFR2 | | | Etgeptyaddfkgrfafssetststaylqinnlknddtttyfcatyygssyvp |
| variant 3 | | | dywgqgtsltvss |
| | VL | 100 | divmtqshkfmstsvgdrvsitckasqnvgtavawyqhkpgqspklliyvvtss |
| | | | rhtgvpdrftgsgsgteftltisnvqsedladyfchqysdypytfgggtkleik |
| Antibody | VH | 101 | evqlqqsgpevgrpgssvkisckasgytftdyimhwvkqspgqglewigwvd |
| to TNFR2 | | | peygstdyaekfkkkatltadtssntayiqlssltsedtatyfcarddgsyspfdyw |
| variant 4 | | | gqgvmvtvss |
| | VL | 102 | diqmtqsppslsaslgdkvtitcqasqninkyiawyqqkpgkaprllirytstles |
| | (major) | | gtpsrfsgsgsgrdysfsisnvesediasyyclqyvnlltfgagtkleik |
| | VL | 103 | nivmtqspksmsmsvgervtltckasenvvtyvswyqqkpeqspklliygas |
| | (minor) | | nrytgvpdrftgsgsatdftltissvqaedladyhcgqgysypytfgggtklcik |
| Antibody | VH | 104 | evqlvesggglvqpggslrlscaasgftfsdyamswvrqapgkglewvavise |
| to TNFR2 | | | ngsdtyyadsvkgrftisrddskntlvlqmnslraedtavyycardrggavsyfd |
| variant 5 | | | vwgqgtlvtvss |
| | VL | 105 | diqmtqspsslsasvgdrvtitcrasqdvssylawyqqkpgkapklliyaassles |
| | | | gvpsrfsgsgsgtdftltisslqpedfatyycqqynslpytfgqgtkveikrT |
| Vonlerolizumab | VH | 106 | evqlvqsgaevkkpgasvkvsckasgytftdsymswviqapgqglewigdm |
| | | | ypdngdssyiKjkfrervtitrdtststaylelsslrsedtavyycvlaprwyfsv |
| | | | wgqgtlvtvss |
| | VL | 107 | diqmtqspsslsasvgdrvtilcrasqdisnylmvyqqkpgkapklliyytsrlrs |
| | | | gvpsrfsgsgsgtdftltisslqpedfatyycqqghtlpptfgqgtkveik |
| TRX518 | VL | 108 | eivmtqspatlsvspgeratlsckasqnvgtnvawyqqkpgqaprlliysasyry |
| | | | sgiparfsgsgsgtefiltisslqsedfavyycqqyntdpltfgggtkveik |
| | VH | 109 | qvtlresgpalvkptqtltltctfsgfslstsgmgvgwirqppgkalewlahiwwd |
| | | | ddkyynpslksrltiskdtsknqvvllmtnmdpvdtatyycartrryfpfaywg |
| | | | qgtlvtvss |
| | VH | 110 | qvtlresgpalvkptqtltltctfsgfslstsgmgvgwirqppgkalewlahiwwd |
| | | | ddkyyqpslksrltiskdtsknqvvltmtnmdpvdtiityycartnyfpfaywg |
| | | | qgtlvtvss |
| Antibody | VH | 111 | qvqlqqwgagllkpsetlsltcavyggsfsdyvwnwirqppgkglewigeinh |
| to LAG-3 | | | rgstnsnpslksrvtlsldtsknqfslklrsvtaadtavyycafgysdyeymvfdp |
| | | | wgqgtlvtvss |
| | VL | 112 | eivltqspatlslspgeratlscrasqsissylawyqqkpgqaprlliydasnrat |
| | | | giparfsgsgsgtdftltisslepedfavyycqqrsmvpltfgqgtnleik |
| Antibody | VH | 113 | mavlallfclvtfpscilsqvqlkesgpglvapsqslsitctvsgfsltgyginwv |
| to GARP | | | rqppgkglewlgmiwsdgstdynsvltsrlriskdnsnsqvnkmnsiqvddtary |
| variant 1 | | | ycardmyydydgamdywgqgtsvtvss |
| | VL | 114 | qvqlkesgpglvapsqslsitctvsgfsltgyginwvrqppgkglewlgmiwsd |
| | | | gstdynsvltsrlriskdnsnsqvflkmnslqvddtaryycardmyydydgam |
| | | | dywgqgtsvtvss |
| Antibody | VH | 115 | mkfpsqlllfllfritgiicdiqvtqsssylsvslgdrvtitckasdhiknwlawyqq |
| to GARP | | | kpgiaprlhsgatsleagvpsrfsgsgsgknftlsitslqtedvatyycqqywstp |
| variant 2 | | | wtfgggttleir |
| | VL | 116 | diqvtqsssylsvslgdntitckasdhiknwlawyqqkpgiaprllvsgatslea |
| | | | gvpsrfsgsgsgknfilsitslqtedvatyycqqywstpwtfgggttleir |
| Antibody | VH | 117 | evqlvqpgaelrnsgasvkvsckasgyrfisyyidwvrqapgqglewmgridp |
| to GARP | | | cdggtkyaqkfqgntftadtststayvelsslrscdtavyycamcwetvvvgdl |
| variant 3 | | | myeyeywgqgtqvtvss |
| | VL | 118 | diqmtqsptslsaslgdrvtitcqasqsissylawyqqkpgqapklliygasrlqtg |
| | | | vpsrfsgsgsgtsftltisgleaedagtyycqqydslpvtfgqgtkvelk |
| | VL | 119 | diqmtqspsslsaslgdrvtitcqasqsivsylawyqqkpgqapklliygasrlqt |
| | | | gvpsrfsgsgsgtsftltisgleaedagtyycqqyasapvtfgqgtgvelk |
| | VL | 120 | diqmtqspsslsaslgdrvtitcqasqsissylawyqqkpgqapklliygtsrlktg |
| | | | vpsrfsgsgsgtsftltisgleaedagtyycqqyysapvtfgqgtkvelk |
| | VL | 121 | diqmtqspsslspslgdrvtitcqasqtissflawyhqkpgqppklliyrasipqtg |
| | | | vpsrfsgsgsgtsftltiggleaedagtyycqqyvsapptfgqgtkvelk |
| | VL | 122 | diqmtqspsslsaslgdrvtitcqasqsissylawyqqkpgqapniliygasrlktg |
| | | | vpsrfsgsgsgtsftltisgleaedagtyycqqyasvpvtfgqgtkvelk |
| Antibody | VH | 123 | qvqlqqwgagllkpsetlsltcavyggsfsgvywswirqspekglewigeinhg |
| to 4-1BB | | | gyvtynpslesrvtisvdtsknqfslklssvtaadtavyycardygpgnydwyfd |
| variant 1 | | | lwgrgtlvtvss |
| | VL | 124 | eivltqspatlslspgeratlscrasqsvssylawyqqkpgqaprlliydasnratgi |
| | | | parfsgsgsgtdftltisslepedfavyycqqrsnwppaltfgggtkveik |
| Antibody | VH | 125 | evqlvesggglvqpggslrlscaasgftfsdywmswvrqapgkglewvadikn |
| to 4-1BB | | | dgsytnyapsltnrftisrdnaknslylqmnslraedtavyycareltgtwgqgtm |
| variant 2 | | | vtvss |
| | VL | 126 | divmtqspdslavslgcratinckssqsllssgnqknylawyqqkpgqppklliy |
| | | | yastrqsgvpdrfsgsgsgtdftltisslqaedvavyyclqydiypftfgqgtkleik |
| Utomilumab | VH | 127 | evqlvqsgaevkkpgeslrisckgsgysfstywiswvrqmpgkglewmgkiy |
| | | | pgdsytnyspsfqgqvtisadksistaylqwsslkasdtamyycargygifdyw |
| | | | gqgtlvtvss |
| | VL | 128 | syeltqppsvsvspgqtasitcsgdnigdqyahwyqqkpgqspvlviyqdknr |
| | | | psgiperfsgsnsgntatltisgtqamdeadyycatytgfgslavfgggtkltvl |
| Antibody | VL | 129 | diqmtqspssvsasvgdrvtitcrasqgisrllawyqqkpgkapklliyvasslqs |
| to ICOS | | | gvpsrfsgsgsgtdftltisslqpedfatyycqqansfpwtfgqgtkveik |
| variant 1 | | | |
| | VH | 130 | qvqlvqsgaevkkpgasvkvsckasgytftgyymhwvrqapgqglewmg |
| | | | winphsggtnyaqkfqgrvtmtrdtsistaymelsrlrsddtavyycartyyyds |
| | | | sgyyhdafdiwgqgtmvtvss |
| Antibody | VH | 131 | evqlvesggglvqpggslrlscaasgftfsdywmdwvnqapgkglvwvsnid |
| to ICOS | | | edgsiteyspfVkgrftisrdnakntlylqmnslraedtavyyctrwgrfgfdswg |
| variant 2 | | | qgtlvtvss |
| | VL | 132 | divmtqspdslavslgeratinckssqsllsgsfnyltwvqqkpgqppkllifyast |
| | | | rhtgvpdrfsgsgsgtdftltisslqaedvavyychhhynapptfgpgtkvdik |
| Vorsetuzumab | VH | 133 | qvqlvqsgaevkkpgasvkvsckasgytftnygmnwvrqapgqglkwmg |
| | | | wintytgeptyadafkgrvtmtrdtsistaymelsrlrsddtavyycardygdyg |
| | | | mdywgqgttvtvss |
| | VL | 134 | divmtqspdslavslgeratincrasksvstsgysfmhwyqqkpgqppklliyla |
| | | | snlesgvpdrfsgsgsgtdftltisslqaedvavyycqhsrevpwtfgqgtkveik |
| Rinucumab | VH | 135 | qlqlqesgpglvkpsetlsltctvsggsitsssyywgwirqppgkglewigsiyyr |
| | | | gstnynpslksrvtisvdssknqfylkvssvtavdtatyycarqngaarpswfdp |
| | | | wgqgtlvtvss |
| | VL | 136 | eivltqspdtislspgeratlscrasqsissiylawyqqkpgqaprlliygassrvtgi |
| | | | pdrfsvsgsgtdftltisrlepedfavyycqhygispftfgpgtkvdir |
| Oleclumab | VH | 137 | evqllesggglvqpggslrlscaasgftfssyayswvrqapgkglewvsaisgsg |
| | | | grtyyadsvkgiftisrdnskntlylqmnslraedtavyycarlgygrvdewgrgt |
| | | | lvtvss |
| | VL | 138 | qsvltqppsasgtpgqivtiscsgslsmgmpvnwyqqlpgtapklliyldnlrls |
| | | | gvpdrfegsksgtsaslaisglqsedeadyycatwddshpgwtfgggtkltvl |
| Antibody to CD73 | VL | 139 | diqmtqspsslsasvgdrvtitcrasqgisswlawyqqkpekapksliyaasslq |
| | | | sgvpsrfsgsgsgtdftltisslqpedfatyycqqynsypltfgggtkveik |
| | VH | 140 | qvqlvesgggwqpgrslrlscaasgftfsnygmhwvrqapgkglewvavily |
| | | | dgsnkyypdsvkgrftisrdnskntlylqmnslraedtavyycarggsswypds |
| | | | fdiwgqgtmvtvss |
| Daratumumab | VH | 141 | evqllesggglvqpggslrlscavsgftfnsfamswvrqapgkglewvsaisgs |
| | | | gggtyyadsvkgrftisrdnskntlylqmnslraedtavyfcakdkilwfgepvf |
| | | | dywgqgtlvtvss |
| | VL | 142 | eivltqspatlslspgeratlscrasqsvssylawyqqkpgqaprllivdasnratgi |
| | | | parfsgsgsgtdftltisslepedfavyycqqrsnwpptfgqgtkveik |
| Etaracizumab | VH | 143 | qvqlvesgggwqpgrslrlscaasgfifssydmswvrqapgkglewvakvss |
| | | | gggstyyldtvqgrftisrdnskntlvlqmnslraedtavyycarhlhgsfaswgq |
| | | | gttvtvss |
| | VL | 144 | eivltqspatlslspgeratlscqasqsisnflhwyqqrpgqaprllirvrsqsis |
| | | | giparfsgsgsgtdftltisslepedfavyycqqsgswpltfgggtkveik |
| Intetumumab | VH | 145 | qvqlvesgggwqpgrsrrlscaasgftfsrytmhwvrqapgkglewvavisfd |
| | | | gsnkyyvdsvkgrftisrdnsentlylqvnilraedtavyycareargsyafdiwg |
| | | | qgtmvtvss |
| | VL | 146 | eivltqspatlslspgeratlscrasqsvssylawyqqkpgqaprlliydasnratgi |
| | | | parfsgsgsgtdftltisslepedfavyvcqqrsnwppftfgpgtkvdik |
| Antibody to | VH | 147 | evqlvesggglvqpggslrlscavsgfvfsrywmswvrqapgkglewigeinp |
| Integrin | | | dsstinytsslkdrftisrdnaknslylqmnslraedtavyycaslittedywgqgtt |
| αvβ8 | | | vtvss |
| | VL | 148 | eivltqspsslslspgervtitckasqdinsylswyqqkpgkapklliyyanrlvdg |
| | | | vparfsgsgsgqdytltisslepedfavyyclqydefjpytfgggtkleikr |
| Ontuxizumab | VH | 149 | qvqlqesgpglvrpsqtlsltctasgytftdyvihwvkqppgrglewigyinpyd |
| | | | ddttynqkfkgntmlvdtssntaylrlssvtaedtavyycarrgnsydgyfdys |
| | | | mdywgsgtpvtvss |
| | VL | 150 | diqmtqspsslsasvgdrvtitcrasqnvgtavawlqqtpgkapklliysasnryt |
| | | | gvpsrfsgsgsgtdytftisslqpediatyycqqytnypmytfgqgtkvqik |
| Antibody | VH | 151 | qvqlqesgpglvkpsqtlsltcaisgdsvssnsvtwnwirqspsrglewlgrtyyr |
| to FAP | | | skvyndyavsvkgritinpdtsknqfylqlksvtpedaavyycardssilygdy |
| variant 1 | | | wgqgtlvtvss |
| | VH | 152 | qvqlqqsgpglvkpsqtlsltcaisgdsvssnsvtwnwirqspsrglewlgrtyyr |
| | | | skwyndyavsvkgritinpdtsknqfylqlksvtpedaavyycardssilygdy |
| | | | wgqgtlvtvs |
| | VL | 153 | qavltqpsslsaspgasasltctlpsginvgtyrifwfqqkpgsppqyllsyksdsd |
| | | | nhqgsgvpsrfsgskdasanagillisglqsedeadyycmiwhssawvfgggt |
| | | | kltvl |
| Antibody | VH | 154 | qvqlvqsgaevkkpgasvkvscktsgytftdyyihwvrqapgqglewmgwi |
| to FAP | | | npnrggtnyaqkfqgivtmtrdtsiataymelsrlrsddtavyycataslkiaavg |
| variant 2 | | | tfdcwgqgtlvtvss |
| | VL | 155 | syeltqppsvsvspgqtaritcsgdalskqyafwfqqkpgqapilviyqdtkrps |
| | | | gipgifsgsssgttvtltisgaqaddeadvycqsadssgtyvfgtgtkvtvl |
| Antibody | VH | 156 | evqlvetgggwqpgrslrlscaasgfsfsthgmywvrqppgkglewvavisy |
| to FAP | | | dgsdkkyadsvkgrftisrdnskntvylemssvraedtalyycfcrrdafdlwgq |
| variant 3 | | | gtmvtvss |
| | VL | 157 | syvltqppsvsvspgqtaritcsgdalpkkyaywyqqksgqapvlviyedtkrp |
| | | | sgiperfsgsssgtmatltisgaqvedeadyycystdssgnywvfgggtevtvl |
| Antibody | VH | 158 | evqlvesggglvepggslrlscaasgftfsdawmnwvrqapgkglewvgrikt |
| to FAP | | | ksdggttdyaapvrgrfsisrddskntlllemnslktedtaiyycfitvivvssespl |
| variant 4 | | | dhwgqgtlvtvss |
| | VL | 159 | syeltqppsvsvspgqtaritcsgdelpkqyaywyqqkpgqapvlviykdrqrp |
| | | | sgiperfsgsssgttvtltisgvqaedeadyycqsaysintyvifgggtkltvl |
| Antibody | VH | 160 | evqlvesggglvkpggslrlscaasgftfsdyymswirqapgkglewisyissgs |
| to FAP | | | sytnyadsvkgrftisrdnakksvylevngltvedtavyycarvrygdrematig |
| variant 5 | | | gfdfwgqgtlvtvss |
| | VL | 161 | syeltqppsvsvspgqtaritcsgdalpkqyaywyqqspgqapvlviykdserp |
| | | | sgiperfsgsssgttvtltisgvqaedeadyycqsadsggtsrifgggtkltvl |
| Antibody | VH | 162 | qvqlqesgpglvrstetlsltclvsgdsinshywswlrqspgrglewigyiyytgp |
| to FAP | | | tnynpslksrvsislgtskdqfslklssvtaadtaiyycamtodwrgsdfyyymd |
| variant 6 | | | vwgkgttvtvss |
| | VL | 163 | eivltqspgtlslslgeratlscrasqslannylawyqqkpgqaprllmydastrat |
| | | | gipdrfsgsgsgtdftltisrlcpcdfavyycqqfvtshhmyifgqgtkveik |
| Antibody | VH | 164 | Hvqlqesgpglvkpsetlsltctvsggsissnnyywgwirqtpgkglewigsiy |
| to FAP | | | ysgstnynpslksrvtisvdtsknqfslklssvtaadtavyycargarwqarpatri |
| variant 7 | | | dgvafdiwgqgtmvtvss |
| | VH | 165 | Qvqlqesgpglvkpsetlsltctvsggsissnnyywgwirqtpgkglewigsiy |
| | | | ysgstnynpslksrvtisvdtsknqfslklssvtaadtavyycargarwqarpatri |
| | | | dgvafdiwgqgtmvtvss |
| | VH | 166 | Evqlvqsgaevkkpgasvkvsckasgytfitsygiswvrqapgqglewmgwi |
| | | | sayngntnyaqklqgrvtmttdtststaymelrslrsddtavyycardwsrsgyyl |
| | | | pdywgqgtlvtvss |
| | VL | 167 | Ettltqspgtlslspgeratlscrasqtvtmylawyqqkpgqaprllmygasnraa |
| | | | gvpdrfsgsgsgtdftltisrlepedfavyycqqfgspytfgqgtkveik |
| | VL | 168 | Dvvmtqsplslpvtlgqpasiscrssqsllhsngynyldwylqrpgqsphlliflg |
| | | | snrasgvpdrfsgsgsgtdftlkisrveaedvgiyycmqalqtpptfgqgtkveik |
| Antibody to | VH | 171 | qvqlvesgggwqpgrsrrlscaasgftfsrytmhwvrqapgkglewvavisfd |
| Integrin | | | gsnkyyvdsvkgrftisrdnsentlylqvnilraedtavyycareargsyafdiwg |
| αv6 variant 1 | | | qgtmvtvss |
| | VL | 172 | eivltqspatlslspgeratlscrasqsvssylawyqqkpgqaprlliydasnratgi |
| | | | parfsgsgsgtdftltisslepedfavyycqqrsnwppftfgpgtkvdik |
| Antibody to | VL | 173 | diqmtqspsslsasvgdntitcrasqdisnylawyqqkpgkapklliyytskihs |
| Integrin | | | gvpsrfsgsgsgtdytftisslqpediatyycqqgntfpytfgqgtkveik |
| αv6 variant 2 | | | |
| | VH | 174 | qvqlqqsggelakpgasvkvsckasgytfssfwmhwvrqapgqglewigyin |
| | | | prsgyteyneifrdkatmttdtststaymelsslrsedtavyycasflgrgamdyw |
| | | | gqgttvtvss |
| Antibody to | VH | 175 | qvqlqesgpglvkpsqtlsltctvsggsissgvyywtwirqhpgnglewigyiy |
| Integrin | | | ysgstsynpslksrvtisvdtskkqfslnltsvtaadtavyycaregplrgdyyygl |
| αvβ6 variant 1 | | | dvwgqgttvtvss |
| | VL | 176 | eivltqspgtlslspgeratlscragqtissrylawyqqkpgqaprpliygassratg |
| | | | ipdrfsgsgsgtdftltisrlepedfavyycqqygssprtfgqgtkveik |
| Antibody to | VH | 177 | qvqlqesgpglvkpsqtlsltctvsggsissggyywswirqhpgkglewigyiy |
| Integrin | | | ysgstyynpslksntisvdtsknqfslklssvtaadtamyycaryTgpaagrgdf |
| αvβ6 variant 2 | | | yyfgmdvwgqgttvtvss |
| | VL | 178 | divmtqtplslsvtpgqpasifckssqsllnsdgktylcwylqkpgqppqlliyev |
| | | | smfsgvpdrfsgsgsgtdftlkisneaedvgvyycmqgiqlpwaffgqgtkveik |
| Antibody to | VH | 179 | qvqlvesgggvvqpgrslrlscaasgftfssygmhwvrqapgkglewvaviw |
| Integrin | | | yggsnkyyadsvkgrftisrdnskntlylqmnslraedtavyycardlaarrgdy |
| αvβ6 variant 3 | | | yyygmdvwgqgttvtvss |
| | VL | 180 | sseltqdpwsvalgqtvritcqgdslrsyylswyqqkpgqapvlviygknnrps |
| | | | gipdrfsgsnsgntasltitgaqaedeadyycnsrdssgnhlfgggtkltvl |
| Antibody to | VH | 181 | qvqlqesgpglvkpsqtlsltctvsggsissggvywswirqhpgkglewigyiy |
| Integrin | | | ysgrtynnpslksrvtisvdtsknqfslklssvtaadtavyycarvatgradyhfya |
| αvβ6 variant 4 | | | mdvwgqgttvtvss |
| | VL | 182 | syeltqpssvsvspgqtaritcsgdvlakksarwfhqkpgqapvlviykdserps |
| | | | giperfsgsssgttvtltisgaqvedeaayycysaadnnlvfgggtkltvl |
| Varlilumab | VH | 183 | qvqlvesgggwqpgrslrlscaasgftfssydmhwvrqapgkglewvaviw |
| | | | ydgsnkyyadsvkgrftisrdnskntlylqmnslraedtavyycargsgnwgff |
| | | | dywgqgtlvtvss |
| | VL | 184 | Diqmtqspsslsasvgdrvtitcrasqgisrwlawyqqkpckapkslivaasslq |
| | | | sgvpsrfsgsgsgtdftltisslqpedfatyycqqyntyprtfgqgtkveik |
| Zinbryta® | VH | 185 | Qvqlvqsgaevkkpgssvkvsckasgytftsyrmhwvrqapgqglewigyin |
| (Daclizumab) | | | pstgytevnqkfkdkatitadestntaymelsslrsedtavyycargggvfdywg |
| | | | qgtlvtvss |
| | VL | 186 | Diqmtqspstlsasvgdrvtitcsasssisymhwyqqkpgkapklliyttsnlas |
| | | | gvparfsgsgsgteftltisslqpddfatyychqrstypltfgqgtkvevk |
| Antibody | VH | 187 | qvqlvesgggwqpgrslrlscaasgftfssygmhwvrqapgkglewvaviw |
| to GITR | | | yegsnkyyadsvkgrftisrdnskntlylqmnslraedtavyycarggsmvrgd |
| | | | yyygmdvwgqgttvtvss |
| | VL | 188 | aiqltqspsslsasvgdrvtitcrasqgissalawyqqkpgkapklliydasslesg |
| | | | vpsrfsgsgsgtdftltisslqpedfatyycqqfnsypytfgqgtkleik |
| Antibody | VH | 189 | EVQLVQSGAEVKKPGASVKVSCKASGYKFSSYWIE |
| huM25 to | | | WVKQAPGQGLEWIGEILPGSDTTNYNEKFKDRATFT |
| LRRC15 | | | SDTSINTAYMELSRLRSDDTAVYYCARDRGNYRAW |
| | | | FGYWGQGTLVTVSS |
| | VL | 190 | DIQMTQSPSSLSASVGDRVTITCRASQDISNYLNWYQ |
| | | | QKPGGAVKFLIYYTSRLHSGVPSRFSGSGSGTDYTLTI |
| | | | SSLQPEDFATYFCQQGEALPWTFGGGTKVEIK |
| Antibody | VH | 191 | EVQLVQSGAEVKKPGSSVKVSCKASGFTFTDYYIHW |
| huAD208.4.1 | | | VKQAPGQGLEWIGLVYPYIGGTNYNQKFKGKATLT |
| to LRRC15 | | | VDTSTTTAYMEMSSLRSEDTAVYYCARGDNKYDAM |
| | | | DYWGQGTTVTVSS |
| | VL | 192 | DIVLTQSPDSLAVSLGERATINCRASQSVSTSSYSYM |
| | | | HWYQQKPGQPPKLLIKYASSLESGVPDRFSGSGSGTD |
| | | | FTLTISSLQAEDVAVYYCEQSWEIRTFGGGTKVEIK |
| Antibody | VH | 193 | EVQLVQSGAEVKKPGSSVKVSCKASGYTFTNYWMH |
| huAD208.12.1 | | | WVKQAPGQGLEWIGMIHPNSGSTKHNEKFRGKATL |
| to | | | TVDESTTTAYMELSSLRSEDTAVYYCARSDFGNYRW |
| LRRC15 | | | YFDVWGQGTTVTVSS |
| | VL | 194 | EIVLTQSPATLSLSPGERATLSCRASQSSSNNLHWYQ |
| | | | QKPGQAPRVLIKYVSQSISGIPARFSGSGSGTDFTLTIS |
| | | | SLEPEDFAVYFCQQSNSWPFTFGQGTKLEIK |
| Antibody | VH | 195 | EVQLVQSGAEVKKPGSSVKVSCKASGFTFTDYYIHW |
| huAD208.14.1 to | | | VKQAPGQGLEWIGLVYPYIGGSSYNQQFKGKATLTV |
| LRRC15 | | | DTSTSTAYMELSSLRSEDTAVYYCARGDNNYDAMD |
| | | | YWGQGTTVTVSS |
| | VL | 196 | DIVLTQSPDSLAVSLGERATISCRASQSVSTSTYNYM |
| | | | HWYQQKPGQPPKLLVKYASNLESGVPDRFSGSGSGT |
| | | | DFTLTISSL |
| | | | QAEDVAVYYCHHTWEIRTFGGGTKVEIK |
| Antibody | VH | 197 | EVQLVESGGGLVQPGGSLRLSCAVSGFSLTSYGVHW |
| hu139.10 to | | | VRQATGKGLEWLGVIWAGGSTNYNSALMSRLTISKE |
| LRRC15 | | | NAKSSVYLQMNSLRAGDTAMYYCATHMITEDYYG |
| | | | MDYWGQGTTVTVSS |
| | VL | 198 | DIVMTQSPDSLAVSLGERATINCKSSQSLLNSRTRKN |
| | | | YLAWYQQKPGQSPKLLIYWASTRESGVPDRFSGSGS |
| | | | GTDFTLTISS |
| | | | LQAEDVAVYYCKQSYNLPTFGGGTKVEIK |
| Antibody | VH | 285 | evqllesggglvqpggslrlscaasgftfssyamswvrqapgkglewvsaisgs |
| mAb-C to | | | ggstyyadsvkgrftisrdnskntlylqmnslraedtavyycakdfssrrwyley |
| ASGR1 | | | wgqgtlvtvss |
| | VL | 286 | sseltqdpavsvalgqtvritcqgdslrsyyaswyqqkpgqapvlviygknnrps |
| | | | gipdrfsgsssgntasltitgaqaedeadyycnslerigylsyvfgggtkltvl |
| Antibody | VH | 287 | EVKLVESGGGLVKPGGSLKLSCAASGFTFSSYAMSW |
| to mzASGR1 | | | VRQTPEKRLEWVSAISGSGGSTYYADSVKGRFTISRD |
| | | | NAKNTLYLQMSSLRSEDTAMYYCAKDFSSRRWYLE |
| | | | YWGQGTTLTVSS |
| | VL | 288 | QAVLTQEPALSVSLGQTVTLTCQGDSLRSYYASWYQ |
| | | | QKPGQAPVLVIYGKNNRPSGVPARFSGSLIGDKAALT |
| | | | ITGAQTEDEATYYCNSLERIGYLSYVFGGGTKVTVL |
| Antibody | VH | 289 | EVQLQQSGPELVKPGASVKISCKASGYSFTGYYMHW |
| to ASGR1 | | | VKQSHVKSLEWIGRINPNNGATNYNQNFKDKASLTV |
| G2D | | | DKSSSTAYMEFHSLTSEDSAVYYCTSVNFYYWGQGT |
| | | | TLTVSS |
| | VL | 290 | DIKMTQSPSSMYASLGERVTITCKASQVINSYLSWFQ |
| | | | QKPGKSPETLIYRANTLVDGVPSRFSGSGSGQDYSLTI |
| | | | SSLEYEDLGIYYCLQYAEFPYTFGGGTKLEIK |
| Antibody | VH | 291 | QVQLQQSAAELARPGASVKMSCKASGYTFTSYTMH |
| to ASGR1 | | | WVKQRPGQGLEWIGYISPSSGYTEYNQKFKDKTTLT |
| K2E | | | ADKSSSTAYMQLSSLTSEDSAVYYCARKFDYWGQG |
| | | | TTLTVSS |
| | VL | 292 | DIKMTQSPSSMYASLGERVTITCKASQDINSYLSWFQ |
| | | | QKPGKSPKTLIYRANRLVDGVPSRFSGSGSGQDFSLTI |
| | | | SSLEYEDMGIYYCLQYDEFPFTFGSGTKLEIK |
| Antibody | VH | 293 | DVQLQESGPGLVKPSQSLSLTCTVTGYSITSDYAWN |
| to ASGR1 | | | WIRQFPGNKLEWMGYISYSGSTRYNPSLKSRISITRDT |
| L4L | | | SKNQFFLQLNSVTTEDTATYYCARRYRYDEGYGMD |
| | | | YWGQGTSVTVSS |
| | VL | 294 | DIQMTQSPASLSVFVGEIVTITCRASENIYSNLAWYQ |
| | | | QKQGKSPQLLVYAATNLADGVPSRFTGSGSGTQFSL |
| | | | KINSLQSEDFGNYYCQHFWGTPPWTFGGGTKLEIK |
| Antibody | VH | 295 | DVQLQESGPGLVKPSQSLSLTCTVTGFSITSDYAWN |
| to ASGR1 | | | WIRQFPGNQLEWMGYRSYRGSTSYNPSLKSRISITRD |
| H8K | | | TSKNQFFLQLNSVTTEDTATYYCARRGYYGSSSHWY |
| | | | FDVWGAGTTVTVSS |
| | VL | 296 | DIQMTQSPASLSASVGETVTITCRASENIYSYLAWYQ |
| | | | QKQGKSPQLLVYNAKTLAEGVPSRFSGSGSGTQFSL |
| | | | KINSLQPEDFGSYYCQHHYGTPLTFGSGTKLELK |
| Antibody | VH | 297 | QVQLQQSGDDLVKPGASVKLSCKASGYTFTSYWIN |
| to ASGR1 | | | WIKQRPGQGLEWIGRIVPGSGSTYYNEMFKDKATLT |
| J4F | | | VDTSSSTAYIQLSSLSSEDSAVYFCARKPNFDVWGAG |
| | | | TTVTVSS |
| | VL | 298 | DIVMTQSQKFMSTSVGDRVSVTCKASQNVGTNVAW |
| | | | YQQKPGQSPKAVIYSASYRFSGVPDRLTGSGSGTDFT |
| | | | LTITNVQSEDLAEYFCQQYNSYPLTFGAGTKLELK |
| Antibody | VH | 299 | QVQLVQSGAEVKKPGASVKVSCKASGYTFTGYYMH |
| to ASGR1 | | | WVRQAPGQRLEWMGRINPNNGATNYNQNFKDRVTI |
| hzG2D | | | TRDTSASTAYMELSSLRSEDTAVYYCARVNFYYWG |
| | | | QGTTLTVSS |
| | VL | 300 | DIQMTQSPSSLSASVGDRVTITCKASQVINSYLSWFQ |
| | | | QKPGKAPKSLIYRANTLVDGVPSRFSGSGSGTDFTLTI |
| | | | SSLQPEDFATYYCLQYAEFPYTFGGGTKVEIK |
| Antibody | VH | 301 | QVQLVQSGAEVKKPGASVKVSCKASGYSFTGYYMH |
| to ASGR1 | | | WVRQAPGQRLEWMGRINPNNGATNYNQNFKDRVTI |
| hzG2.1D T28S | | | TRDTSASTAYMELSSLRSEDTAVYYCARVNFYYWG |
| | | | QGTTLTVSS |
| Antibody | VL | 302 | DIQMTQSPSSLSASVGDRVTITCKASQVINSYLSWFQ |
| to ASGR1 | | | QKPGKAPKSLIYRANTLVSGVPSRFSGSGSGTDFTLTI |
| hzG2.1D D56S | | | SSLQPEDFATYYCLQYAEFPYTFGGGTKVEIK |
| Antibody | VH | 303 | QVQLVQSGAEVKKPGASVKVSCKASGYSFTGYYMH |
| to ASGR1 | | | WVRQAPGQRLEWMGRINPNNGATNYNQNFKDKATI |
| hzG2.2D T28S, | | | TRDTSASTAYMELSSLRSEDTAVYYCARVNFYYWG |
| R66K, | | | QGTTLTVSS |
| V67A | | | |
| Antibody to | VL | 304 | DIQMTQSPSSLSASVGDRVTITCKASQVINSYLSWFQ |
| ASGR1 | | | QKPGKAPKSLIYRANTLVEGVPSRFSGSGSGTDFTLTI |
| hzG2.2D D56E | | | SSLQPEDFATYYCLQYAEFPYTFGGGTKVEIK |
| Antibody | VH | 305 | QVQLVQSGAEVKKPGASVKVSCKASGYSFTGYYMH |
| to ASGR1 | | | WVRQAPGQRLEWMGRINPNNGATNYNQNFKDKATI |
| hzG2.3D T28S, | | | TRDTSASTAYMELSSLRSEDTAVYYCTSVNFYYWGQ |
| R66K, V67A, | | | GTTLTVSS |
| A93T, R94S | | | |
| Antibody | VH | 306 | QVQLVQSGAEVKKPGASVKVSCKASGYSFTGYYMH |
| to ASGR1 | | | WVRQAPGQRLEWMGRINPNNGATNYNQNFKDKAS |
| hzG2.4D T28S, | | | LTVDTSASTAYMELSSLRSEDTAVYYCARVNFYYW |
| R66K, V67A, | | | GQGTTLTVSS |
| T68S, I69L, | | | |
| R71V | | | |
| Antibody | VH | 307 | QVQLVQSGAEVKKPGASVKVSCKASGYSFTGYYMH |
| to ASGR1 | | | WVRQAPGQRLEWMGRINPNNGATNYNQNFKDKAS |
| hzG2.5D T28S, | | | LTVDTSASTAYMELSSLRSEDTAVYYCTSVNFYYWG |
| R66K, V67A, | | | QGTTLTVSS |
| T68S, I69L, | | | |
| R71V, A93T, | | | |
| R94S | | | |
| Antibody | VH | 308 | QVQLVQSGAEVKKPGASVKVSCKASGYSFTGYYMH |
| to ASGR1 | | | WVRQAPGQRLEWMGRINPNNGATNYNQNFKDRVS |
| hzG2.6D T28S, | | | LTVDTSASTAYMELSSLRSEDTAVYYCARVNFYYW |
| T68S, I69L, | | | GQGTTLTVSS |
| R71V | | | |
| Antibody to ASGR1 | VH | 309 | QVQLVQSGAEVKKPGASVKVSCKASGYSFTGYYMH |
| hzG2.7D T28S, T68S, | | | WVRQAPGQRLEWMGRINPNNGATNYNQNFKDRVS |
| I69L, R71V, A93T, | | | LTVDTSASTAYMELSSLRSEDTAVYYCTSVNFYYWG |
| R94S | | | QGTTLTVSS |
| Antibody to ASGR1 | VH | 310 | QVQLVQSGAEVKKPGASVKVSCKASGYSFTGYYMH |
| hzG2.8D T28S, A93T, | | | WVRQAPGQRLEWMGRINPNNGATNYNQNFKDRVTI |
| R94S | | | TRDTSASTAYMELSSLRSEDTAVYYCTSVNFYYWGQ |
| | | | GTTLTVSS |
| Antibody to ASGR1 | VH | 311 | QVQLVQSGAEVKKPGASVKVSCKASGYSFTGYYMH |
| hzG2.9D T28S, N54Q | | | WVRQAPGQRLEWMGRINPNQGATNYNQNFKDRVTI |
| | | | TRDTSASTAYMELSSLRSEDTAVYYCARVNFYYWG |
| | | | QGTTLTVSS |
| Antibody to ASGR1 | VH | 312 | QVQLVQSGAEVKKPGASVKVSCKASGYSFTGYYMH |
| hzG2.10D T28S, N54Q, | | | WVRQAPGQRLEWMGRINPNQGATNYNQNFKDKATI |
| R66K, V67A | | | TRDTSASTAYMELSSLRSEDTAVYYCARVNFYYWG |
| | | | QGTTLTVSS |
| Antibody to ASGR1 | VH | 313 | QVQLVQSGAEVKKPGASVKVSCKASGYSFTGYYMH |
| hzG2.11D T28S, N54Q, | | | WVRQAPGQRLEWMGRINPNQGATNYNQNFKDKATI |
| R66K, V67A, | | | TRDTSASTAYMELSSLRSEDTAVYYCTSVNFYYWGQ |
| A93T, R94S | | | GTTLTVSS |
| Antibody to ASGR1 | VH | 314 | QVQLVQSGAEVKKPGASVKVSCKASGYSFTGYYMH |
| hzG2.12D T28S, N54Q, | | | WVRQAPGQRLEWMGRINPNQGATNYNQNFKDKAS |
| R66K, V67A, | | | LTVDTSASTAYMELSSLRSEDTAVYYCARVNFYYW |
| T68S, I69L, R71V | | | GQGTTLTVSS |
| Antibody to ASGR1 | VH | 315 | QVQLVQSGAEVKKPGASVKVSCKASGYSFTGYYMH |
| hzG2.13D T28S, N54Q, | | | WVRQAPGQRLEWMGRINPNQGATNYNQNFKDKAS |
| R66K, V67A, | | | LTVDTSASTAYMELSSLRSEDTAVYYCTSVNFYYWG |
| T68S, I69L, R71V, | | | QGTTLTVSS |
| A93T, R94S | | | |
| Antibody to ASGR1 | VH | 316 | QVQLVQSGAEVKKPGASVKVSCKASGYSFTGYYMH |
| hzG2.14D T28S, N54Q, | | | WVRQAPGQRLEWMGRINPNQGATNYNQNFKDRVS |
| T68S, I69L, | | | LTVDTSASTAYMELSSLRSEDTAVYYCARVNFYYW |
| R71V | | | GQGTTLTVSS |
| Antibody to ASGR1 | VH | 317 | QVQLVQSGAEVKKPGASVKVSCKASGYSFTGYYMH |
| hzG2.15D T28S, N54Q, | | | WVRQAPGQRLEWMGRINPNQGATNYNQNFKDRVS |
| T68S, I69L, | | | LTVDTSASTAYMELSSLRSEDTAVYYCTSVNFYYWG |
| R71V, A93T, R94S | | | QGTTLTVSS |
| Antibody to ASGR1 | VH | 318 | QVQLVQSGAEVKKPGASVKVSCKASGYSFTGYYMH |
| hzG2.16D T28S, | | | WVRQAPGQRLEWMGRINPNQGATNYNQNFKDRVTI |
| N54Q, A93T, R94S | | | TRDTSASTAYMELSSLRSEDTAVYYCTSVNFYYWGQ |
| | | | GTTLTVSS |
| Antibody to ASGR1 | VH | 319 | QVQLVQSGAEVKKPGASVKVSCKASGYSFTGYYMH |
| hzG2.17D T28S, G55A | | | WVRQAPGQRLEWMGRINPNNAATNYNQNFKDRVTI |
| | | | TRDTSASTAYMELSSLRSEDTAVYYCARVNFYYWG |
| | | | QGTTLTVSS |
| Antibody to ASGR1 | VH | 320 | QVQLVQSGAEVKKPGASVKVSCKASGYSFTGYYMH |
| hzG2.18D T28S, | | | WVRQAPGQRLEWMGRINPNNAATNYNQNFKDKATI |
| G55A, R66K, V67A | | | TRDTSASTAYMELSSLRSEDTAVYYCARVNFYYWG |
| | | | QGTTLTVSS |
| Antibody to ASGR1 | VH | 321 | QVQLVQSGAEVKKPGASVKVSCKASGYSFTGYYMH |
| hzG2.19D T28S, | | | WVRQAPGQRLEWMGRINPNNAATNYNQNFKDKATI |
| G55A, R66K, V67A, | | | TRDTSASTAYMELSSLRSEDTAVYYCTSVNFYYWGQ |
| A93T, R94S | | | GTTLTVSS |
| Antibody to ASGR1 | VH | 322 | QVQLVQSGAEVKKPGASVKVSCKASGYSFTGYYMH |
| hzG2.20D T28S, | | | WVRQAPGQRLEWMGRINPNNAATNYNQNFKDKAS |
| G55A, R66K, V67A, | | | LTVDTSASTAYMELSSLRSEDTAVYYCARVNFYYW |
| T68S, I69L, R71V | | | GQGTTLTVSS |
| Antibody to ASGR1 | VH | 323 | QVQLVQSGAEVKKPGASVKVSCKASGYSFTGYYMH |
| hzG2.21D T28S, | | | WVRQAPGQRLEWMGRINPNNAATNYNQNFKDKAS |
| G55A, R66K, V67A, | | | LTVDTSASTAYMELSSLRSEDTAVYYCTSVNFYYWG |
| T68S, I69L, R71V, | | | QGTTLTVSS |
| A93T, R94S | | | |
| Antibody to ASGR1 | VH | 324 | QVQLVQSGAEVKKPGASVKVSCKASGYSFTGYYMH |
| hzG2.22D T28S, | | | WVRQAPGQRLEWMGRINPNNAATNYNQNFKDRVS |
| G55A, T68S, I69L, | | | LTVDTSASTAYMELSSLRSEDTAVYYCARVNFYYW |
| R71V | | | GQGTTLTVSS |
| Antibody to ASGR1 | VH | 325 | QVQLVQSGAEVKKPGASVKVSCKASGYSFTGYYMH |
| hzG2.23D T28S, | | | WVRQAPGQRLEWMGRINPNNAATNYNQNFKDRVS |
| G55A, T68S, I69L, | | | LTVDTSASTAYMELSSLRSEDTAVYYCTSVNFYYWG |
| R71V, A93T, R94S | | | QGTTLTVSS |
| Antibody to ASGR1 | VH | 326 | QVQLVQSGAEVKKPGASVKVSCKASGYSFTGYYMH |
| hzG2.24D T28S, | | | WVRQAPGQRLEWMGRINPNNAATNYNQNFKDRVTI |
| G55A, A93T, R94S | | | TRDTSASTAYMELSSLRSEDTAVYYCTSVNFYYWGQ |
| | | | GTTLTVSS |
| Antibody to ASGR1 | VH | 327 | QVQLVQSGAEVKKPGASVKVSCKASGYTFTGYYMH |
| hzG2.25D N54Q, | | | WVRQAPGQRLEWMGRINPNQGATNYNQNFKDKATI |
| R66K, V67A | | | TRDTSASTAYMELSSLRSEDTAVYYCARVNFYYWG |
| | | | QGTTLTVSS |
| Antibody to ASGR1 | VH | 328 | QVQLVQSGAEVKKPGASVKVSCKASGYTFTGYYMH |
| hzG2.26D N54Q, | | | WVRQAPGQRLEWMGRINPNQGATNYNQNFKDKATI |
| R66K, V67A, A93T, | | | TRDTSASTAYMELSSLRSEDTAVYYCTSVNFYYWGQ |
| R94S | | | GTTLTVSS |
| Antibody to ASGR1 | VH | 329 | QVQLVQSGAEVKKPGASVKVSCKASGYTFTGYYMH |
| hzG2.27D N54Q, | | | WVRQAPGQRLEWMGRINPNQGATNYNQNFKDKAS |
| R66K, V67A, T68S, | | | LTVDTSASTAYMELSSLRSEDTAVYYCARVNFYYW |
| I69L, R71V | | | GQGTTLTVSS |
| Antibody to ASGR1 | VH | 330 | QVQLVQSGAEVKKPGASVKVSCKASGYTFTGYYMH |
| hzG2.28D N54Q, | | | WVRQAPGQRLEWMGRINPNQGATNYNQNFKDKAS |
| R66K, V67A, T68S, | | | LTVDTSASTAYMELSSLRSEDTAVYYCTSVNFYYWG |
| I69L, R71V, A93T, | | | QGTTLTVSS |
| R94S | | | |
| Antibody to ASGR1 | VH | 331 | QVQLVQSGAEVKKPGASVKVSCKASGYTFTGYYMH |
| hzG2.29D N54Q, | | | WVRQAPGQRLEWMGRINPNQGATNYNQNFKDRVS |
| T68S, I69L, R71V | | | LTVDTSASTAYMELSSLRSEDTAVYYCARVNFYYW |
| | | | GQGTTLTVSS |
| Antibody to ASGR1 | VH | 332 | QVQLVQSGAEVKKPGASVKVSCKASGYTFTGYYMH |
| hzG2.30D N54Q, | | | WVRQAPGQRLEWMGRINPNQGATNYNQNFKDRVS |
| T68S, I69L, R71V, | | | LTVDTSASTAYMELSSLRSEDTAVYYCTSVNFYYWG |
| A93T, R94S | | | QGTTLTVSS |
| Antibody to ASGR1 | VH | 333 | QVQLVQSGAEVKKPGASVKVSCKASGYTFTGYYMH |
| hzG2.31D N54Q, | | | WVRQAPGQRLEWMGRINPNQGATNYNQNFKDRVTI |
| A93T, R94S | | | TRDTSASTAYMELSSLRSEDTAVYYCTSVNFYYWGQ |
| | | | GTTLTVSS |
| Antibody to ASGR1 | VH | 334 | QVQLVQSGAEVKKPGASVKVSCKASGYTFTGYYMH |
| hzG2.32D G55A, | | | WVRQAPGQRLEWMGRINPNNAATNYNQNFKDKATI |
| R66K, V67A | | | TRDTSASTAYMELSSLRSEDTAVYYCARVNFYYWG |
| | | | QGTTLTVSS |
| Antibody to ASGR1 | VH | 335 | QVQLVQSGAEVKKPGASVKVSCKASGYTFTGYYMH |
| hzG2.33D G55A, | | | WVRQAPGQRLEWMGRINPNNAATNYNQNFKDKATI |
| R66K, V67A, A93T, | | | TRDTSASTAYMELSSLRSEDTAVYYCTSVNFYYWGQ |
| R94S | | | GTTLTVSS |
| Antibody to ASGR1 | VH | 336 | QVQLVQSGAEVKKPGASVKVSCKASGYTFTGYYMH |
| hzG2.34D G55A, | | | WVRQAPGQRLEWMGRINPNNAATNYNQNFKDKAS |
| R66K, V67A, T68S, | | | LTVDTSASTAYMELSSLRSEDTAVYYCARVNFYYW |
| I69L, R71V | | | GQGTTLTVSS |
| Antibody to ASGR1 | VH | 337 | QVQLVQSGAEVKKPGASVKVSCKASGYTFTGYYMH |
| hzG2.35D G55A, | | | WVRQAPGQRLEWMGRINPNNAATNYNQNFKDKAS |
| R66K. V67A, T68S, | | | LTVDTSASTAYMELSSLRSEDTAVYYCTSVNFYYWG |
| I69L, R71V, A93T, | | | QGTTLTVSS |
| R94S | | | |
| Antibody to ASGR1 | VH | 338 | QVQLVQSGAEVKKPGASVKVSCKASGYTFTGYYMH |
| hzG2.36D G55A, | | | WVRQAPGQRLEWMGRINPNNAATNYNQNFKDRVS |
| T68S, I69L, R71V | | | LTVDTSASTAYMELSSLRSEDTAVYYCARVNFYYW |
| | | | GQGTTLTVSS |
| Antibody to ASGR1 | VH | 339 | QVQLVQSGAEVKKPGASVKVSCKASGYTFTGYYMH |
| hzG2.37D G55A, | | | WVRQAPGQRLEWMGRINPNNAATNYNQNFKDRVS |
| T68S, I69L, R71V, | | | LTVDTSASTAYMELSSLRSEDTAVYYCTSVNFYYWG |
| A93T. R94S | | | QGTTLTVSS |
| Antibody to ASGR1 | VH | 340 | QVQLVQSGAEVKKPGASVKVSCKASGYTFTGYYMH |
| hzG2.38D G55A, | | | WVRQAPGQRLEWMGRINPNNAATNYNQNFKDRVTI |
| A93T, R94S | | | TRDTSASTAYMELSSLRSEDTAVYYCTSVNFYYWGQ |
| | | | GTTLTVSS |
| Antibody to ASGR1 | VH | 341 | QVQLVQSGAEVKKPGASVKVSCKASGYTFTGYYMH |
| hzG2.39D R66K, | | | WVRQAPGQRLEWMGRINPNNGATNYNQNFKDKATI |
| V67A | | | TRDTSASTAYMELSSLRSEDTAVYYCARVNFYYWG |
| | | | QGTTLTVSS |
| Antibody to ASGR1 | VH | 342 | QVQLVQSGAEVKKPGASVKVSCKASGYTFTGYYMH |
| hzG2.40D R66K, | | | WVRQAPGQRLEWMGRINPNNGATNYNQNFKDKATI |
| V67A, A93T, R94S | | | TRDTSASTAYMELSSLRSEDTAVYYCTSVNFYYWGQ |
| | | | GTTLTVSS |
| Antibody to ASGR1 | VH | 343 | QVQLVQSGAEVKKPGASVKVSCKASGYTFTGYYMH |
| hzG2.41D R66K, | | | WVRQAPGQRLEWMGRINPNNGATNYNQNFKDKAS |
| V67A, T68S, I69L, | | | LTVDTSASTAYMELSSLRSEDTAVYYCARVNFYYW |
| R71V | | | GQGTTLTVSS |
| Antibody to ASGR1 | VH | 344 | QVQLVQSGAEVKKPGASVKVSCKASGYTFTGYYMH |
| hzG2.42D R66K, | | | WVRQAPGQRLEWMGRINPNNGATNYNQNFKDKAS |
| V67A, T68S, I69L, | | | LTVDTSASTAYMELSSLRSEDTAVYYCTSVNFYYWG |
| R71V, A93T, R94S | | | QGTTLTVSS |
| Antibody to ASGR1 | VH | 345 | QVQLVQSGAEVKKPGASVKVSCKASGYTFTGYYMH |
| hzG2.43D T68S, I69L, | | | WVRQAPGQRLEWMGRINPNNGATNYNQNFKDRVS |
| R71V | | | LTVDTSASTAYMELSSLRSEDTAVYYCARVNFYYW |
| | | | GQGTTLTVSS |
| Antibody to ASGR1 | VH | 346 | QVQLVQSGAEVKKPGASVKVSCKASGYTFTGYYMH |
| hzG2.44D T68S, I69L, | | | WVRQAPGQRLEWMGRINPNNGATNYNQNFKDRVS |
| R71V, A93T, R94S | | | LTVDTSASTAYMELSSLRSEDTAVYYCTSVNFYYWG |
| | | | QGTTLTVSS |
| Antibody to ASGR1 | VH | 347 | QVQLVQSGAEVKKPGASVKVSCKASGYTFTGYYMH |
| hzG2.45D A93T, R94S | | | WVRQAPGQRLEWMGRINPNNGATNYNQNFKDRVTI |
| | | | TRDTSASTAYMELSSLRSEDTAVYYCTSVNFYYWGQ |
| | | | GTTLTVSS |
| Antibody to ASGR1 | VH | 348 | QVQLVQSGAEVKKPGASVKVSCKASGYTFTSYTMH |
| hzK2E | | | WVRQAPGQGLEWMGYISPSSGYTEYNQKFKDRVTM |
| | | | TRDTSTSTVYMELSSLRSEDTAVYYCARKFDYWGQ |
| | | | GTTVTVSS |
| Antibody to ASGR1 | VL | 349 | DIQMTQSPSSLSASVGDRVTITCKASQDINSYLSWFQ |
| hzK2E | | | QKPGKAPKSLIYRANRLVDGVPSRFSGSGSGTDFTLT |
| | | | ISSLQPEDFATYYCLQYDEFPFTFGQGTKLEIK |
| Antibody to ASGR1 | VH | 350 | QVQLVQSGAEVKKPGASVKVSCKASGYTFTSYTMH |
| hzK2.1E R66K, V67T | | | WVRQAPGQGLEWMGYISPSSGYTEYNQKFKDKTTM |
| | | | TRDTSTSTVYMELSSLRSEDTAVYYCARKFDYWGQ |
| | | | GTTVTVSS |
| Antibody to ASGR1 | VL | 351 | DIQMTQSPSSLSASVGDRVTITCKASQDINSYLSWFQ |
| hzK2.1E S46T | | | QKPGKAPKTLIYRANRLVDGVPSRFSGSGSGTDFTLT |
| | | | ISSLQPEDFATYYCLQYDEFPFTFGQGTKLEIK |
| Antibody to ASGR1 | VH | 352 | QVQLVQSGAEVKKPGASVKVSCKASGYTFTSYTMH |
| hzK2.2E R66K, V67T, | | | WVRQAPGQGLEWMGYISPSSGYTEYNQKFKDKTTL |
| M69L, R71A | | | TADTSTSTVYMELSSLRSEDTAVYYCARKFDYWGQ |
| | | | GTTVTVSS |
| Antibody to ASGR1 | VL | 353 | DTQMTQSPSSLSASVGDRVTITCKASQDINSYLSWFQ |
| hzK2.2E D56E | | | QKPGKAPKSLIYRANRLVEGVPSRFSGSGSGTDFTLTI |
| | | | SSLQPEDFATYYCLQYDEFPFTFGQGTKLEIK |
| Antibody to ASGR1 | VH | 354 | QVQLVQSGAEVKKPGASVKVSCKASGYTFTSYTMH |
| hzK2.3E R66K, V67T, | | | WVRQAPGQGLEWMGYISPSSGYTEYNQKFKDKTTL |
| M69L, R71A, T73K | | | TADKSTSTVYMELSSLRSEDTAVYYCARKFDYWGQ |
| | | | GTTVTVSS |
| Antibody to ASGR1 | VL | 355 | DIQMTQSPSSLSASVGDRVTITCKASQDINSYLSWFQ |
| hzK2.3E S46T D56E | | | QKPGKAPKTLIYRANRLVEGVPSRFSGSGSGTDFTLTI |
| | | | SSLQPEDFATYYCLQYDEFPFTFGQGTKLEIK |
| Antibody to ASGR1 | VH | 356 | QVQLVQSGAEVKKPGASVKVSCKASGYTFTSYTMH |
| hzK2.4E M69L, R71A | | | WVRQAPGQGLEWMGYISPSSGYTEYNQKFKDRVTL |
| | | | TADTSTSTVYMELSSLRSEDTAVYYCARKFDYWGQ |
| | | | GTTVTVSS |
| Antibody to ASGR1 | VH | 357 | QVQLQESGPGLVKPSQTLSLTCTVSGGSISSDYAWN |
| hzL4L | | | WIRQHPGKGLEWIGYISYSGSTRYNPSLKSRVTISVD |
| | | | TSKNQFSLKLSSVTAADTAVYYCARRYRYDEGYGM |
| | | | DYWGQGTTVTVSS |
| Antibody to ASGR1 | VL | 358 | DIQMTQSPSSLSASVGDRVTITCRASENIYSNLAWYQ |
| hzL4L | | | QKPGKAPKLLIYAATNLADGVPSRFSGSGSGTDFTLT |
| | | | ISSLQPEDFATYYCQHFWGTPPWTFGGGTKVEIK |
| Antibody to ASGR1 | VH | 359 | QVQLQESGPGLVKPSQTLSLTCTVSGGSITSDYAWN |
| hzL4.1L S30T, V67I, | | | WIRQHPGKGLEWIGYISYSGSTRYNPSLKSRISITRDT |
| T68S, S70T, V71R | | | SKNQFSLKLSSVTAADTAVYYCARRYRYDEGYGMD |
| | | | YWGQGTTVTVSS |
| Antibody to ASGR1 | VL | 360 | DIQMTQSPSSLSASVGDRVTITCRASENIYSNLAWYQ |
| hzL4.1LD56S | | | QKPGKAPKLLIYAATNLASGVPSRFSGSGSGTDFTLTI |
| | | | SSLQPEDFATYYCQHFWGTPPWTFGGGTKVEIK |
| Antibody to ASGR1 | VH | 361 | QVQLQESGPGLVKPSQTLSLTCTVSGYSITSDYAWN |
| hzL4.2L G27Y, S30T, | | | WIRQHPGKGLEWIGYISYSGSTRYNPSLKSRISITRDT |
| V67I, T68S, S70T, | | | SKNQFSLKLSSVTAADTAVYYCARRYRYDEGYGMD |
| V71R | | | YWGQGTTVTVSS |
| Antibody to ASGR1 | VL | 362 | DIQMTQSPSSLSASVGDRVTITCRASENIYSNLAWYQ |
| hzL4.2L D56E | | | QKPGKAPKLLIYAATNLAEGVPSRFSGSGSGTDFTLTI |
| | | | SSLQPEDFATYYCQHFWGTPPWTFGGGTKVEIK |
| Antibody to ASGR1 | VH | 363 | QVQLQESGPGLVKPSQTLSLTCTVSGYSITSDYAWN |
| hzL4.3L G27Y, S30T | | | WIRQHPGKGLEWIGYISYSGSTRYNPSLKSRVTISVD |
| | | | TSKNQFSLKLSSVTAADTAVYYCARRYRYDEGYGM |
| | | | DYWGQGTTVTVSS |
| Antibody to ASGR1 | VL | 364 | DIQMTQSPSSLSASVGDRVTITCRASENIYSNLAWYQ |
| hzL4.3L G57A | | | QKPGKAPKLLIYAATNLADAVPSRFSGSGSGTDFTLT |
| | | | ISSLQPEDFATYYCQHFWGTPPWTFGGGTKVEIK |
| Antibody to ASGR1 | VH | 365 | QVQLQESGPGLVKPSQTLSLTCTVSGYSISSDYAWN |
| hzL4.4L G27Y, V67I, | | | WIRQHPGKGLEWIGYISYSGSTRYNPSLKSRISITRDT |
| T68S, S70T, V71R | | | SKNQFSLKLSSVTAADTAVYYCARRYRYDEGYGMD |
| | | | YWGQGTTVTVSS |
| Antibody to ASGR1 | VH | 366 | QVQLQESGPGLVKPSQTLSLTCTVSGYSITSDYAWN |
| hzL4.5L G27Y, S30T, | | | WIRQHPGKGLEWIGYISYSGSTRYNPSLKSRVTITRD |
| S70T, V71R | | | TSKNQFSLKLSSVTAADTAVYYCARRYRYDEGYGM |
| | | | DYWGQGTTVTVSS |
| Antibody to ASGR1 | VH | 367 | QVQLQESGPGLVKPSQTLSLTCTVSGYSISSDYAWN |
| hzL4.6L G27Y, S70T, | | | WIRQHPGKGLEWIGYISYSGSTRYNPSLKSRVTITRD |
| V71R | | | TSKNQFSLKLSSVTAADTAVYYCARRYRYDEGYGM |
| | | | DYWGQGTTVTVSS |
| Antibody to ASGR1 | VH | 368 | QVQLQESGPGLVKPSQTLSLTCTVSGYSITSDYAWN |
| hzL4.7L G27Y, S30T, | | | WIRQHPGKGLEWIGYISYSGSTRYNPSLKSRISISVDT |
| V67I, T68S | | | SKNQFSLKLSSVTAADTAVYYCARRYRYDEGYGMD |
| | | | YWGQGTTVTVSS |
| Antibody to ASGR1 | VH | 369 | QVQLQESGPGLVKPSQTLSLTCTVSGYSISSDYAWN |
| hzL4.8L G27Y, V67I, | | | WIRQHPGKGLEWIGYISYSGSTRYNPSLKSRISISVDT |
| T68S | | | SKNQFSLKLSSVTAADTAVYYCARRYRYDEGYGMD |
| | | | YWGQGTTVTVSS |
| Antibody to ASGR1 | VH | 370 | QVQLQESGPGLVKPSQTLSLTCTVSGGSISSDYAWN |
| hzH8K | | | WIRQHPGKGLEWIGYRSYRGSTSYNPSLKSRVTISVD |
| | | | TSKNQFSLKLSSVTAADTAVYYCARRGYYGSSSHW |
| | | | YFDVWGQGTTVTVSS |
| | VL | 371 | DIQMTQSPSSLSASVGDRVTITCRASENIYSYLAWYQ |
| | | | QKPGKAPKLLIYNAKTLAEGVPSRFSGSGSGTDFTLTI |
| | | | SSLQPEDFATYYCQHHYGTPLTFGGGTKVEIK |
| Antibody to ASGR1 | VH | 372 | QVQLQESGPGLVKPSQTLSLTCTVSGGSITSDYAWN |
| hzH8.1K S30T | | | WIRQHPGKGLEWIGYRSYRGSTSYNPSLKSRVTISVD |
| | | | TSKNQFSLKLSSVTAADTAVYYCARRGYYGSSSHW |
| | | | YFDVWGQGTTVTVSS |
| Antibody to ASGR1 | VH | 373 | QVQLQESGPGLVKPSQTLSLTCTVSGFSITSDYAWN |
| hzH8.2K G27F, S30T | | | WIRQHPGKGLEWIGYRSYRGSTSYNPSLKSRVTISVD |
| | | | TSKNQFSLKLSSVTAADTAVYYCARRGYYGSSSHW |
| | | | YFDVWGQGTTVTVSS |
| Antibody to ASGR1 | VH | 374 | QVQLQESGPGLVKPSQTLSLTCTVSGGSISSDYAWN |
| hzH8.3K V67I, T68S, | | | WIRQHPGKGLEWIGYRSYRGSTSYNPSLKSRISITRDT |
| S70T, V71R | | | SKNQFSLKLSSVTAADTAVYYCARRGYYGSSSHWYF |
| | | | DVWGQGTTVTVSS |
| Antibody to ASGR1 | VH | 375 | QVQLQESGPGLVKPSQTLSLTCTVSGGSITSDYAWN |
| hzH8.4K S30T, V67I, | | | WIRQHPGKGLEWIGYRSYRGSTSYNPSLKSRISITRDT |
| T68S, S70T, V71R | | | SKNQFSLKLSSVTAADTAVYYCARRGYYGSSSHWYF |
| | | | DVWGQGTTVTVSS |
| Antibody to ASGR1 | VH | 376 | QVQLQESGPGLVKPSQTLSLTCTVSGFSITSDYAWN |
| hzH8.5K G27F, S30T, | | | WIRQHPGKGLEWIGYRSYRGSTSYNPSLKSRISITRDT |
| V67I, T68S, S70T, | | | SKNQFSLKLSSVTAADTAVYYCARRGYYGSSSHWYF |
| V71R | | | DVWGQGTTVTVSS |
| Antibody to ASGR1 | VH | 377 | QVQLQESGPGLVKPSQTLSLTCTVSGGSITSDYAWN |
| hzH8.6K S30T, V67I, | | | WIRQHPGKGLEWIGYRSYRGSTSYNPSLKSRISISVDT |
| T68S | | | SKNQFSLKLSSVTAADTAVYYCARRGYYGSSSHWYF |
| | | | DVWGQGTTVTVSS |
| Antibody to ASGR1 | VH | 378 | QVQLQESGPGLVKPSQTLSLTCTVSGGSITSDYAWN |
| hzH8.7K S30T, S70T, | | | WIRQHPGKGLEWIGYRSYRGSTSYNPSLKSRVTITRD |
| V71R | | | TSKNQFSLKLSSVTAADTAVYYCARRGYYGSSSHW |
| | | | YFDVWGQGTTVTVSS |
| Antibody to ASGR1 | VH | 379 | QVQLQESGPGLVKPSQTLSLTCTVSGGSISSDYAWN |
| hzH8.8K V67I, T68S | | | WIRQHPGKGLEWIGYRSYRGSTSYNPSLKSRISISVDT |
| | | | SKNQFSLKLSSVTAADTAVYYCARRGYYGSSSHWYF |
| | | | DVWGQGTTVTVSS |
| Antibody to ASGR1 | VH | 380 | QVQLQESGPGLVKPSQTLSLTCTVSGGSISSDYAWN |
| hzH8.9K S70T, V71R | | | WIRQHPGKGLEWIGYRSYRGSTSYNPSLKSRVTITRD |
| | | | TSKNQFSLKLSSVTAADTAVYYCARRGYYGSSSHW |
| | | | YFDVWGQGTTVTVSS |
| Antibody to ASGR1 | VH | 381 | QVQLVQSGAEVKKPGASVKVSCKASGYTFTSYWIN |
| hzJ4F | | | WVRQAPGQGLEWMGRIVPGSGSTYYNEMFKDRVT |
| | | | MTRDTSTSTVYMELSSLRSEDTAVYYCARKPNFDV |
| | | | WGQGTTVTVSS |
| | VL | 382 | DIQMTQSPSSLSASVGDRVTITCKASQNVGTNVAWY |
| | | | QQKPGKAPKLLIYSASYRFSGVPSRFSGSGSGTDFTLT |
| | | | ISSLQPEDFATYYCQQYNSYPLTFGGGTKVEIK |
| Antibody to ASGR1 | VH | 383 | QVQLVQSGAEVKKPGASVKVSCKASGYTFTSYWIN |
| hzJ4.1F V37I | | | WIRQAPGQGLEWMGRIVPGSGSTYYNEMFKDRVTM |
| | | | TRDTSTSTVYMELSSLRSEDTAVYYCARKPNFDVWG |
| | | | QGTTVTVSS |
| Antibody to ASGR1 | VL | 384 | DIVMTQSPSSLSASVGDRVTITCKASQNVGTNVAWY |
| hzJ4.1F Q3V | | | QQKPGKAPKLLIYSASYRFSGVPSRFSGSGSGTDFTLT |
| | | | ISSLQPEDFATYYCQQYNSYPLTFGGGTKVEIK |
| Antibody to ASGR1 | VH | 385 | QVQLVQSGAEVKKPGASVKVSCKASGYTFTSYWIN |
| hzJ4.2F V37I, R38K, | | | WIKQAPGQGLEWIGRIVPGSGSTYYNEMFKDRVTMT |
| M48I | | | RDTSTSTVYMELSSLRSEDTAVYYCARKPNFDVWGQ |
| | | | GTTVTVSS |
| Antibody to ASGR1 | VL | 386 | DIVMTQSPSSLSASVGDRVTITCKASQNVGTNVAWY |
| hzJ4.2F Q3V L46A | | | QQKPGKAPKALIYSASYRFSGVPSRFSGSGSGTDFTL |
| | | | TISSLQPEDFATYYCQQYNSYPLTFGGGTKVEIK |
| Antibody to ASGR1 | VH | 387 | QVQLVQSGAEVKKPGASVKVSCKASGYTFTSYWIN |
| hzJ4.3F V37I, R66K, | | | WIRQAPGQGLEWMGRIVPGSGSTYYNEMFKDKATM |
| V67A | | | TRDTSTSTVYMELSSLRSEDTAVYYCARKPNFDVWG |
| | | | QGTTVTVSS |
| Antibody to ASGR1 | VL | 388 | DIQMTQSPSSLSASVGDRVTITCKASQNVGTNVAWY |
| hzJ4.3F L46A | | | QQKPGKAPKALIYSASYRFSGVPSRFSGSGSGTDFTL |
| | | | TISSLQPEDFATYYCQQYNSYPLTFGGGTKVEIK |
| Antibody to ASGR1 | VH | 389 | QVQLVQSGAEVKKPGASVKVSCKASGYTFTSYWIN |
| hzJ4.4F V37I, R38K, | | | WIKQAPGQGLEWIGRIVPGSGSTYYNEMFKDKATMT |
| M48I, R66K, V67A | | | RDTSTSTVYMELSSLRSEDTAVYYCARKPNFDVWGQ |
| | | | GTTVTVSS |
| Antibody to ASGR1 | VL | 390 | DIQMTQSPSSLSASVGDRVTITCKASQNVGTNVAWY |
| hzJ4.4F 146A, L47V | | | QQKPGKAPKAVIYSASYRFSGVPSRFSGSGSGTDFTL |
| | | | TISSLQPEDFATYYCQQYNSYPLTFGGGTKVEIK |
| Antibody to ASGR1 | VH | 391 | QVQLVQSGAEVKKPGASVKVSCKASGYTFTSYWIN |
| hzJ4.5F R66K, V67A | | | WVRQAPGQGLEWMGRIVPGSGSTYYNEMFKDKAT |
| | | | MTRDTSTSTVYMELSSLRSEDTAVYYCARKPNFDV |
| | | | WGQGTTVTVSS |
| Antibody to ASGR1 | VL | 392 | DIQMTQSPSSLSASVGDRVTITCKASQNVGTNVAWY |
| hzJ4.5F L46A, L47V, | | | QQKPGKAPKAVIYSASYRFSGVPSRLSGSGSGTDFTL |
| F62L | | | TISSLQPEDFATYYCQQYNSYPLTFGGGTKVEIK |
| Antibody to ASGR1 | VH | 393 | QVQLVQSGAEVKKPGASVKVSCKASGYTFTSYWIN |
| hzJ4.6F R66K, V67A, | | | WVRQAPGQGLEWMGRIVPGSGSTYYNEMFKDKAT |
| V78A | | | MTRDTSTSTAYMELSSLRSEDTAVYYCARKPNFDV |
| | | | WGQGTTVTVSS |
| Antibody to ASGR1 | VL | 394 | DIQMTQSPSSLSASVGDRVTITCKASQNVGTNVAWY |
| hzJ4.6F L46A, L47V, | | | QQKPGKAPKAVIYSASYRFSGVPDRLTGSGSGTDFTL |
| S60D, F62L, S63T | | | TISSLQPEDFATYYCQQYNSYPLTFGGGTKVEIK |
| Antibody to ASGR1 | VH | 395 | QVQLVQSGAEVKKPGASVKVSCKASGYTFTSYWIN |
| hzJ4.7F R66K, V67A, | | | WVRQAPGQGLEWMGRIVPGSGSTYYNEMFKDKAT |
| V78A, M80I, E81Q | | | MTRDTSTSTAYIQLSSLRSEDTAVYYCARKPNFDVW |
| | | | GQGTTVTVSS |
| Antibody to ASGR1 | VH | 396 | QVQLVQSGAEVKKPGASVKVSCKASGYTFTSYWIN |
| hzJ4.8F R66K, V67A, | | | WVRQAPGQGLEWMGRIVPGSGSTYYNEMFKDKATL |
| M69L, R71V | | | TVDTSTSTVYMELSSLRSEDTAVYYCARKPNFDVWG |
| | | | QGTTVTVSS |
| Antibody to ASGR1 | VH | 397 | QVQLVQSGAEVKKPGASVKVSCKASGYTFTSYWIN |
| hzJ4.9F M69L, R71V | | | WVRQAPGQGLEWMGRIVPGSGSTYYNEMFKDRVTL |
| | | | TVDTSTSTVYMELSSLRSEDTAVYYCARKPNFDVWG |
| | | | QGTTVTVSS |
| Antibody to ASGR1 | VH | 398 | QVQLVQSGAEVKKPGASVKVSCKASGYTFTSYWIN |
| hzJ4.10F R71V | | | WVRQAPGQGLEWMGRIVPGSGSTYYNEMFKDRVT |
| | | | MTVDTSTSTVYMELSSLRSEDTAVYYCARKPNFDV |
| | | | WGQGTTVTVSS |
| Antibody to ASGR1 | VH | 169 | QVQLVQSGAEVKKPGASVKVSCKASGYTFTS |
| hzJ4.11F V78A | | | YWINWVRQAPGQGLEWMGRIVPGSGSTYYNE |
| | | | MFKDRVTMTRDTSTSTAYMELSSLRSEDTAV |
| | | | YYCARKPNFDVWGQGTTVTVSS |
| Antibody to ASGR1 | VH | 170 | QVQLVQSGAEVKKPGASVKVSCKASGYTFTS |
| hzJ4.12F V78A, M80I, | | | YWINWVRQAPGQGLEWMGRIVPGSGSTYYNE |
| E81Q | | | MFKDRVTMTRDTSTSTAYIQLSSLRSEDTAV |
| | | | YYCARKPNFDVWGQGTTVTVSS |

### Target Binding Domain

An antibody construct may further comprise a target binding domain. A target binding domain may comprise a domain that binds to a target. A target may be an antigen. A target binding domain may comprise an antigen binding domain. A target binding domain may be a domain that can specifically bind to an antigen. A target binding domain may be an antigen-binding portion of an antibody or an antibody fragment. A target binding domain may be one or more fragments of an antibody that can retain the ability to specifically bind to an antigen. A target binding domain may be any antigen binding fragment. A target binding domain may be in a scaffold, in which a scaffold is a supporting framework for the antigen binding domain. A target binding domain may comprise an antigen binding domain in a scaffold.

A target binding domain may comprise an antigen binding domain which refers to a portion of an antibody comprising the antigen recognition portion, i.e., an antigenic determining variable region of an antibody sufficient to confer recognition and binding of the antigen recognition portion to a target, such as an antigen, i.e., the epitope. A target binding domain may comprise an antigen binding domain of an antibody.

An Fv can be the minimum antibody fragment which contains a complete antigen-recognition and antigen-binding site. This region may consist of a dimer of one heavy chain and one light chain variable domain in tight, non-covalent association. In this configuration, the three CDRs of each variable domain may interact to define an antigen-binding site on the surface of the V_{H}-V_{L} dimer. A single variable domain (or half of an Fv comprising only three CDRs specific for an antigen) can recognize and bind antigen, although at a lower affinity than the entire binding site.

A target binding domain may be at least 80% identical to an antigen binding domain selected from, but not limited to, a monoclonal antibody, a polyclonal antibody, a recombinant antibody, or a functional fragment thereof, for example, a heavy chain variable domain (V_{H}) and a light chain variable domain (V_{L}), a single chain variable fragment (scFv), or a DARPin, an affimer, an avimer, a knottin, a monobody, an affinity clamp, an ectodomain, a receptor ectodomain, a receptor, a cytokine, a ligand, an immunocytokine, a T cell receptor, or a recombinant T cell receptor.

A target binding domain may be attached to an antibody construct. For example, an antibody construct may be fused with a target binding domain to create an antibody construct target binding domain fusion. The antibody construct-target binding domain fusion may be the result of the nucleic acid sequence of the target binding domain being expressed in frame with the nucleic acid sequence of the antibody construct. The antibody construct-target binding domain fusion may be the result of an in-frame genetic nucleotide sequence or a contiguous peptide sequence encoding the antibody construct with the target binding domain. As another example, a target binding domain may be linked to an antibody construct. A target binding domain may be linked to an antibody construct by a chemical conjugation. A target binding domain may be attached to a terminus of an Fc region. A target binding domain may be attached to a terminus of an Fc region or domain. A target binding domain may be attached to a terminus of an antibody construct. A target binding domain may be attached to a terminus of an antibody. A target binding domain may be attached to a light chain of an antibody. A target binding domain may be attached to a terminus of a light chain of an antibody. A target binding domain may be attached to a heavy chain of an antibody. A target binding domain may be attached to a terminus of a heavy chain of an antibody. The terminus may be a C-terminus. An antibody construct may be attached to 1, 2, 3, and/or 4 target binding domains. The target binding domain may direct the antibody construct to, for example, a particular cell or cell type. A target binding domain of an antibody construct may be selected in order to recognize an antigen, e.g., an antigen expressed on an immune cell. An antigen can be a peptide or fragment thereof. An antigen may be expressed on an antigen-presenting cell. An antigen may be expressed on a dendritic cell, a macrophage, or a B cell. As another example, an antigen may be a tumor antigen. The tumor antigen may be any tumor antigen described herein. When multiple target binding domains are attached to an antibody construct, the target binding domains may bind to the same antigen. When multiple target binding domains are attached to an antibody construct, the target binding domains may bind different antigens.

In certain embodiments, an antibody construct specifically binds a second antigen. In certain embodiments, the target binding domain is linked to said antibody construct at a C-terminal end of said Fc region or domain.

In certain embodiments, the target binding domain specifically binds to an antigen that is at least 80% identical to an antigen on a T cell, a B cell, a stellate cell, an endothelial cell, a tumor cell, an APC, a fibroblast cell, a fibrocyte cell, or a cell associated with the pathogenesis of fibrosis. In certain embodiments, the target binding domain specifically binds to an antigen that is at least 80% identical to an antigen on a T cell, an APC, and/or a B cell. In certain embodiments, the target binding domain may specifically bind to an antigen that is at least 80% identical to an antigen selected from the group consisting of CLTA4, PD-1, OX40, LAG-3, GITR, GARP, CD25, CD27, PD-L1, TNFR2, ICOS, 41BB, CD70, CD73, CD38, or VTCN1. In certain embodiments, the target binding domain specifically binds to an antigen that is an antigen on a T cell, a B cell, a stellate cell, an endothelial cell, a tumor cell, an APC, a fibroblast cell, a fibrocyte cell, or a cell associated with the pathogenesis of fibrosis. In certain embodiments, the target binding domain specifically binds to an antigen that is an antigen on a T cell, an APC, and/or a B cell. In certain embodiments, the target binding domain may specifically bind to an antigen that is at least 80% identical to an antigen selected from the group consisting of CLTA4, PD-1, OX40, LAG-3, GITR, GARP, CD25, CD27, PD-L1, TNFR2, ICOS, 41BB, CD70, CD73, CD38, or VTCN1.

### Attachment of Linkers to Antibody Construct

The conjugates described herein may comprise a linker, e.g., a peptide linker. Linkers of the conjugates and methods may not affect the binding of active portions of a conjugate (e.g, active portions include antigen binding domains, Fc region or domains, target binding domains, antibodies, compounds, inhibitors or the like) to a target, which can be a cognate binding partner such as an antigen. A linker can form a linkage between different parts of a conjugate, e.g., between an antibody construct or targeting moiety and a compound of the disclosure. In certain embodiments, a conjugate comprises multiple linkers. In certain embodiments, wherein a conjugate comprises multiple linkers, the linkers may be the same linkers or different linkers.

A linker may be bound to an antibody construct or targeting moiety by a bond between the antibody construct targeting moiety and the linker. A linker may be bound to an anti-tumor antigen antibody construct by a bond between the anti-tumor antigen antibody construct and the linker. A linker may be bound to a terminus of an amino acid sequence of an antibody construct, or could be bound to a side chain modification to the antibody construct, such as the side chain of a lysine, serine, threonine, cysteine, tyrosine, aspartic acid, glutamine, a non-natural amino acid residue, or glutamic acid residue. A linker may be bound to a terminus of an amino acid sequence of an Fc region of an antibody construct, or may be bound to a side chain modification of an Fc region of an antibody construct, such as the side chain of a lysine, serine, threonine, cysteine, tyrosine, aspartic acid, glutamine, a non-natural amino acid residue, or glutamic acid residue. A linker may be bound to a terminus of an amino acid sequence of an Fc region or domain of an antibody construct, or may be bound to a side chain modification of an Fc region or domain of an antibody construct, such as the side chain of a lysine, serine, threonine, cysteine, tyrosine, aspartic acid, glutamine, a non-natural amino acid residue, or glutamic acid residue.

A linker may be bound to an antibody construct at a hinge cysteine. A linker may be bound to an antibody construct at a light chain constant domain lysine. A linker may be bound to an antibody construct at an engineered cysteine in the light chain. A linker may be bound to an antibody construct at an Fc region lysine. A linker may be bound to an antibody construct at an Fc region or domain lysine. A linker may be bound to an antibody construct at an Fc region cysteine. A linker may be bound to an antibody construct at an Fc region or domain cysteine. A linker may be bound to an antibody construct at a light chain glutamine, such as an engineered glutamine. A linker may be bound to an antibody construct at an unnatural amino acid engineered into the light chain. A linker may be bound to an antibody construct at an unnatural amino acid engineered into the heavy chain. Amino acids can be engineered into an amino acid sequence of an antibody construct, for example, a linker of a conjugate. Engineered amino acids may be added to a sequence of existing amino acids. Engineered amino acids may be substituted for one or more existing amino acids of a sequence of amino acids.

A linker may be conjugated to an antibody construct via a sulfhydryl group on the antibody construct. A linker may be conjugated to an antibody construct via a primary amine on the antibody construct. A linker may be conjugated to an antibody construct via residue of an unnatural amino acid on an antibody construct, e.g., a ketone moiety.

In certain embodiments, when one or more linkers are bound, e.g., covalently, to an antibody construct at sites on the construct, an Fc region or domain of the antibody construct can bind to Fc receptors. In certain embodiments, an antibody construct bound to a linker or an antibody construct bound to a linker bound to a compound of the present invention, retains the ability of the Fc region or domain of the antibody to bind to Fc receptors. In certain embodiments, when a linker is connected to an antibody construct, the antigen binding domain of an antibody construct bound to a linker or an antibody construct bound to a linker bound to a compound of the present invention can bind its antigen. In certain embodiments, when a linker is connected to an antibody construct at the sites described herein, a target binding domain of an antibody construct bound to a linker or an antibody construct bound to a linker bound to a compound of the present invention can bind its antigen.

In certain embodiments, a linker or linker bound to a compound of the present invention may be attached to an amino acid residue of an IgG Fc region or domain selected from: 221, 222, 224, 227, 228, 230, 231, 223, 233, 234, 235, 236, 237, 238, 239, 240, 241, 243, 244, 245, 246, 247, 249, 250, 258, 262, 263, 264, 265, 266, 267, 268, 269, 270, 271, 272, 273, 274, 275, 276, 278, 280, 281, 283, 285, 286, 288, 290, 291, 292, 293, 294, 295, 296, 297, 298, 299, 300, 302, 305, 313, 317, 318, 320, 322, 323, 324, 325, 326, 327, 328, 329, 330, 331, 332, 333, 334, 335, 336, 396, 428, or any subset thereof, wherein numbering of amino acid residues in the Fc region or domain is according to the EU index as in Kabat.

In certain embodiments, a linker or linker bound to a compound of the present invention is not attached to an amino acid residue of an IgG Fc region or domain selected from: 221, 222, 224, 227, 228, 230, 231, 223, 233, 234, 235, 236, 237, 238, 239, 240, 241, 243, 244, 245, 246, 247, 249, 250, 258, 262, 263, 264, 265, 266, 267, 268, 269, 270, 271, 272, 273, 274, 275, 276, 278, 280, 281, 283, 285, 286, 288, 290, 291, 292, 293, 294, 295, 296, 297, 298, 299, 300, 302, 305, 313, 317, 318, 320, 322, 323, 324, 325, 326, 327, 328, 329, 330, 331, 332, 333, 334, 335, 336, 396, 428, or any subset thereof, wherein numbering of amino acid residues in the Fc region or domain is according to the EU index as in Kabat.

### Lysine-Based Bioconjugation

An antibody construct can be conjugated to a linker via lysine-based bioconjugation. An antibody construct can be exchanged into an appropriate buffer, for example, phosphate, borate, PBS, histidine, Tris-Acetate at a concentration of about 2 mg/mL to about 10 mg/mL. An appropriate number of equivalents of a construct of a compound of the present invention, and a linker, linker-payload, as described herein, can be added as a solution with stirring. Dependent on the physical properties of the linker-payload, a co-solvent can be introduced prior to the addition of the linker-payload to facilitate solubility. The reaction can be stirred at room temperature for 2 hours to about 12 hours depending on the observed reactivity. The progression of the reaction can be monitored by LC-MS. Once the reaction is deemed complete, the remaining linker-payloads can be removed by applicable methods and the antibody conjugate can be exchanged into the desired formulation buffer. Lysine-linked conjugates can be synthesized starting with ab antibody (mAb) and linker-payload, e.g., 10 equivalents, following Scheme A below (Conjugate=antibody conjugate). Monomer content and drug-antibody construct ratios (molar ratios) can be determined by methods described herein.

[chemical structure]

### Cysteine-Based Bioconjugation

An antibody construct can be conjugated to a linker via cysteine-based bioconjugation. An antibody construct can be exchanged into an appropriate buffer, for example, phosphate, borate, PBS, histidine, Tris-Acetate at a concentration of about 2 mg/mL to about 10 mg/mL with an appropriate number of equivalents of a reducing agent, for example, dithiothreitol or tris(2-carboxyethyl)phosphine. The resultant solution can be stirred for an appropriate amount of time and temperature to effect the desired reduction. A construct of a compound of the present invention and a linker can be added as a solution with stirring. Dependent on the physical properties of the linker-payload, a co-solvent can be introduced prior to the addition of the linker-payload to facilitate solubility. The reaction can be stirred at room temperature for about 1 hour to about 12 hours depending on the observed reactivity. The progression of the reaction can be monitored by liquid chromatography-mass spectrometry (LC-MS). Once the reaction is deemed complete, the remaining free linker-payload can be removed by applicable methods and the antibody conjugate can be exchanged into the desired formulation buffer. Such cysteine-based conjugates can be synthesized starting with an antibody (mAb) and linker-payload, e.g., 7 equivalents, using the conditions described in Scheme B below (Conjugate=antibody conjugate). Monomer content and drug-antibody ratios can be determined by methods described herein.

[chemical structure]

### Compounds

The following is a discussion of compounds and salts thereof that may be used in the methods of the disclosure. The compounds and salts thereof described in Formulas (IA), (IB), (IC), (ID), (IE), (IF), and Table 16 may be covalently bound, to linkers, L3, which may further be covalently bound to antibody constructs or targeting moieties.

In a first aspect, disclosed herein is a compound represented by Formula (I):

[chemical structure]

or a pharmaceutically acceptable salt thereof, wherein:

one of M¹ and M² is

[chemical structure]

and

the other of M¹ and M² is selected from:

[chemical structure]

R¹ and R² are, at each occurrence, independently selected from hydrogen, halogen, —OR¹¹, —SR¹¹, —N(R¹¹)₂, —NO₂, —CN, phenyl, and —C₁-C₆ alkyl, wherein said —C₁-C₆ alkyl is optionally substituted with one or more substituents independently selected from halogen, —OR¹¹, —SR¹¹, —S(O)R¹⁰, —S(O)₂R¹¹, —S(O)₂N(R¹¹)₂—N(R¹¹)₂, —C(O)R¹⁰, —C(O)N(R¹¹)₂, —N(R¹¹)C(O)R¹⁰, —C(O)OR¹¹, —OC(O)R¹⁰, —NO₂, and —CN;

R³ is, at each occurrence, independently selected from halogen, —C₁-C₃ alkyl, —C₁-C₃ haloalkyl —OH, —NO₂, —CN, —O—C₁-C₃ alkyl, and —O—C₁-C₃ haloalkyl;

each R⁴ is, at each occurrence independently selected from hydrogen and C₁-C₃ alkyl or two R⁴ join together with atoms to which they are attached to form a 5- or 6-membered heterocycle optionally substituted with one or more substituents independently selected from halogen, —C₁-C₃ alkyl, —OH, —O—C₁-C₃ alkyl, and —O—C₁-C₃ haloalkyl;

R⁵ is hydrogen, halogen, —OR⁶¹, —SR⁶¹, —N(R⁶¹)₂, —NO₂, —CN, and C₁-C₆ alkyl, wherein said —C₁-C₆ alkyl is optionally substituted with one or more substituents independently selected from halogen, —OR⁶¹, —SR⁶¹, —N(R⁶¹)₂, —NO₂, and —CN;

R⁶ is, at each occurrence, independently selected from:
halogen, —OR²¹, —SR²¹, —N(R²¹)₂, —C(O)R²⁰, —C(O)N(R²¹)₂, —N(R²¹)C(O)R²⁰, —C(O)OR²¹, —OC(O)R²¹, —S(O)R²⁰, —S(O)₂R²¹, —S(O)₂N(R²¹)₂, —OC(O)OR²¹, —OC(O)N(R²¹)₂, —NR²¹C(═O)OR²¹, —N(R²¹)C(O)N(R²¹)₂, —NO₂ and —CN;
C₁-C₁₀ alkyl, C₂-C₁₀ alkenyl, and C₂-C₁₀ alkynyl, each of which is optionally substituted with one or more substituents independently selected from halogen, —OR²¹, —SR²¹, —N(R²¹)₂, —C(O)R²⁰, —C(O)N(R²¹)₂, —N(R²¹)C(O)R²⁰, —C(O)OR²¹, —OC(O)R²¹, —S(O)R²⁰, —S(O)₂R²¹, —S(O)₂N(R²¹)₂, —OC(O)OR²¹, —OC(O)N(R²¹)₂, —NR²¹C(═O)OR²¹, —N(R²¹)C(O)N(R²¹)_{2J}—NO₂, ═O, ═S, ═N(R²¹), —CN, a C₃-C₁₀ carbocycle, and a 3- to 10-membered heterocycle wherein said C₃-C₁₀ carbocycle and said 3- to 10-membered heterocycle are optionally substituted with one or more substituents independently selected from R^{X}; and
a C₃-C₁₀ carbocycle and a 3- to 10-membered heterocycle, each of which is optionally substituted with one or more substituents independently selected from halogen, —OR²⁰, —OH, —SR²⁰, —SH, —N(R²¹)₂, —C(O)R²⁰, —C(O)N(R²¹)₂, —N(R²¹)C(O)R²⁰, —C(O)OR²¹, —OC(O)R²¹, —S(O)R²⁰, —S(O)₂R²¹, —S(O)₂N(R²¹)₂, —OC(O)OR²¹, —OC(O)N(R²¹)₂, —NR²¹C(═O)OR²¹, —N(R²¹)C(O)N(R²¹)₂, —NO₂, ═O, ═S, ═N(R²¹), —CN, —C₂-C₆ alkenyl, —C₂-C₆ alkynyl and C₁-C₆ alkyl wherein said C₁-C₆ alkyl is optionally substituted with one or more substituents independently selected from R^{Y};

R⁷ and R⁸ are independently selected from hydrogen, halogen, C₁-C₃ alkyl, —OH, —O—C₁-C₃ alkyl, and —O—C₁-C₃ haloalkyl, or R⁷ and R⁸ join together with the atoms to which they are attached to form a C₅-C₆ carbocycle or 5- or 6-membered heterocycle each of which is optionally substituted with one or more substituents independently selected from halogen, —OR³¹, —SR³¹, —N(R³¹)₂, —NO₂, —CN and C₁-C₆ alkyl wherein said C₁-C₆ alkyl is optionally substituted with one or more substituents independently selected from halogen, —OR³¹, —SR³¹, —N(R³¹)₂, —NO₂, and —CN;

Y is selected from —O— and —N(R⁹)— and R⁹ is, at each occurrence, independently selected from:
hydrogen; and —C₁-C₆ alkyl optionally substituted with one or more substituents independently selected from halogen, —OR⁴¹, —SR⁴¹, —S(O)R⁴⁰, —S(O)₂R⁴¹, —S(O)₂N(R⁴¹)₂—N(R⁴¹)₂, —C(O)R⁴⁰, —C(O)N(R⁴¹)₂, —N(R⁴¹)C(O)R⁴⁰, —C(O)OR⁴¹, —OC(O)R⁴⁰, —NO₂, and —CN;

each R¹⁰, R²⁰, and R⁴⁰ is independently selected at each occurrence from:
—C₁-C₁₀ alkyl, —C₂-C₁₀ alkenyl, and —C₂-C₁₀ alkynyl, each of which is optionally substituted with one or more substituents independently selected from R^{Y}; and
a C₃-C₁₂ carbocycle and a 3- to 12-membered heterocycle, each of which is optionally substituted with one or more substituents independently selected from R^{X};

each R¹¹, R²¹, R³¹, R⁴¹, and R⁶¹ is independently selected at each occurrence from:
hydrogen;
—C₁-C₁₀ alkyl, —C₂-C₁₀ alkenyl, and —C₂-C₁₀ alkynyl, each of which is optionally substituted with one or more substituents independently selected from R^{Y}; and
a C₃-C₁₂ carbocycle and a 3- to 12-membered heterocycle, each of which is optionally substituted with one or more substituents independently selected from R^{X},
or two R¹¹, R²¹, R³¹, R⁴¹, or R⁶¹ on the same N atom are taken together with the N atom to which they are attached to form a N-Containing heterocycle optionally substituted with R^{X};

each R^{X} is independently selected at each occurrence from:
halogen, —OR⁵¹, —SR⁵¹, N(R⁵¹)₂, —C(O)R⁵⁰, —C(O)N(R⁵¹)₂, —N(R⁵¹)C(O)R⁵⁰, —C(O)OR⁵¹, —OC(O)R⁵¹, —S(O)R⁵⁰, —S(O)₂R⁵¹, —S(O)₂N(R⁵¹)₂, —OC(O)OR⁵¹, —OC(O)N(R⁵¹)₂, —NR⁵¹C(═O)OR⁵¹, —N(R⁵¹)C(O)N(R⁵¹)₂, —NO_{2J} ═O, ═S, ═N(R⁵¹), —CN, —C₂-C₆ alkenyl, —C₂-C₆ alkynyl and C₁-C₆ alkyl wherein said C₁-C₆ alkyl is optionally substituted with one or more substituents independently selected from —OR⁵¹, —SR⁵¹, —N(R⁵¹)₂, —C(O)R⁵⁰, —C(O)N(R⁵¹)₂, —N(R⁵¹)C(O)R⁵⁰, —C(O)OR⁵¹, —OC(O)R⁵¹, —S(O)R⁵⁰, —S(O)₂R⁵¹, —S(O)₂N(R⁵¹)₂, —OC(O)OR⁵¹, —OC(O)N(R⁵¹)₂, —NR⁵¹C(═O)OR⁵¹, —N(R⁵¹)C(O)N(R⁵¹)₂, and ═O;

each R^{Y} is independently selected at each occurrence from:
halogen, —OR⁵¹, —SR⁵¹, N(R⁵¹)₂, —C(O)R⁵⁰, —C(O)N(R⁵¹)₂, —N(R⁵¹)C(O)R⁵⁰, —C(O)OR⁵¹, —OC(O)R⁵¹, —S(O)R⁵⁰, —S(O)₂R⁵¹, —S(O)₂N(R⁵¹)₂, —OC(O)OR⁵¹, —OC(O)N(R⁵¹)₂, —NR⁵¹C(═O)OR⁵¹, —N(R⁵¹)C(O)N(R⁵¹)₂, —NO₂, ═O, ═S, ═N(R⁵¹), and —CN;

each R⁵⁰ is independently selected at each occurrence from:
—C₁-C₁₀ alkyl, —C₂-C₁₀ alkenyl, and —C₂-C₁₀ alkynyl, each of which is optionally substituted with one or more substituents independently selected from halogen, —OH, —CN, —NO₂, —NH₂, ═O, ═S, —O—C₁-C₁₀ alkyl, C₃-C₁₂ carbocycle, and a 3- to 12-membered heterocycle; and
a C₃-C₁₂ carbocycle and a 3- to 12-membered heterocycle, each of which is optionally substituted with one or more substituents independently selected from halogen, —OH, —CN, —NO₂, —NH₂, ═O, ═S, —C₁-C₁₀ alkyl, —O—C₁-C₁₀ alkyl, and —C₁-C₁₀haloalkyl;

each R⁵¹ is independently selected at each occurrence from:
hydrogen;
—C₁-C₁₀ alkyl, —C₂-C₁₀ alkenyl, and —C₂-C₁₀ alkynyl, each of which is optionally substituted with one or more substituents independently selected from halogen, —OH, —CN, —NO₂, —NH₂, ═O, ═S, —O—C₁-C₁₀ alkyl, C₃-C₁₂ carbocycle, and a 3- to 12-membered heterocycle; and
a C₃-C₁₂ carbocycle and a 3- to 12-membered heterocycle, each of which is optionally substituted with one or more substituents independently selected from halogen, —OH, —CN, —NO₂, —NH₂, ═O, ═S, —C₁-C₁₀ alkyl, —O—C₁-C₁₀ alkyl, and —C₁-C₁₀haloalkyl;

Z¹, Z², Z³, and Z⁴ are independently selected from N or C(H);

n is selected from 1, 2, and 3;

m is 0, 1, or 2;

s is selected from 0 and 1; and

w is selected from 0, 1, 2, 3, 4, and 5.

In certain embodiments, a compound represented by Formula (I) or a salt thereof, wherein one of M¹ and M² is

[chemical structure]

and the other of M¹ and M² is

[chemical structure]

and the remaining variables (e.g., R¹-R⁸, R¹⁰, R²⁰, R⁴⁰, R¹¹, R²¹, R³¹, R⁴¹, R⁵⁰, R⁵¹, R⁶¹, Y, R^{X}, R^{Y}, Z¹, Z², Z³, Z⁴, n, m, s, and w) are as set forth in the first aspect.

In a third aspect, disclosed herein is a compound represented by Formula (I) or a salt thereof, wherein one of M¹ and M² is

[chemical structure]

and the other of M¹ and M² is

[chemical structure]

and the remaining variables are as set forth in the first aspect.

In a fourth aspect, disclosed herein is a compound represented by Formula (I) wherein one of one of M¹ and M² is

[chemical structure]

and the other of M¹ and M² is

[chemical structure]

and the remaining variables are as set forth in the first aspect.

In a fifth aspect, disclosed herein is a compound represented by Formula (I) or a salt thereof, wherein one of one of M¹ and M² is

[chemical structure]

and the other of M¹ and M² is

[chemical structure]

and the remaining variables are as set forth in the first aspect.

In a sixth aspect, disclosed herein is a compound represented by Formula (I) or a salt thereof, wherein M¹ and M² is as set forth in the fifth aspect and R⁷ and R⁸ are independently selected from hydrogen, halogen, —C₁-C₃ alkyl, —OH, —O—C₁-C₃ alkyl, and —O—C₁-C₃ haloalkyl, or R⁷ and R⁸ join together with the atoms to which they are attached to form a C₅-C₆ carbocycle or 5- or 6-membered heterocycle each of which is optionally substituted with one or more substituents independently selected from halogen, —OR³¹, —SR³¹, —N(R³¹)₂, and —C₁-C₆ alkyl wherein said C₁-C₆ alkyl is optionally substituted with one or more substituents independently selected from halogen, —OR³¹, —SR³¹, and —N(R³¹)₂; and the remaining variables are as set forth in the first aspect.

In a seventh aspect, disclosed herein is a compound represented by Formula (I) or a salt thereof, wherein M¹ and M² is as set forth in the fifth aspect and R⁷ and R⁸ are independently selected from hydrogen, halogen, —C₁-C₃ alkyl, —OH, —O—C₁-C₃ alkyl, and —O—C₁-C₃ haloalkyl, or R⁷ and R⁸ join together with the atoms to which they are attached to form an unsubstituted C₅-C₆ carbocycle or an unsubstituted 5- or 6-membered heterocycle; and the remaining variables are as set forth in the first aspect.

In an eighth aspect, disclosed herein is a compound represented by Formula (I) or a salt thereof, wherein M¹ and M² is as set forth in any one of aspects 5-7 wherein the 5- or 6-membered heterocycle of R⁷ and R⁸ is a 5- or 6-membered heterocycle contains one ring heteroatom selected from nitrogen contains one ring heteroatom selected from nitrogen; and the remaining variables are as set forth in the first aspect. In a ninth aspect, disclosed herein is a compound represented by Formula (I) or a salt thereof, wherein M¹ and M² is as set forth in any one of aspects 5-7 wherein R⁷ and R⁸ join together with the atoms to which they are attached to form a phenyl ring optionally substituted with one or more substituents independently selected from halogen, —OR³¹, —SR³¹, —N(R³¹)₂, and C₁-C₆ alkyl wherein said C₁-C₆ alkyl is optionally substituted with one or more substituents independently selected from halogen, —OR³¹, —SR³¹, and —N(R³¹)₂; and the remaining variables are as set forth in the first aspect.

In a tenth aspect, disclosed herein is a compound represented by Formula (I) or a salt thereof, wherein M¹ and M² is as set forth in any one of aspects 5-7 wherein R⁷ and R⁸ join together with the atoms to which they are attached to form an unsubstituted phenyl ring; and the remaining variables are as set forth in the first aspect.

In an eleventh aspect, disclosed herein is a compound represented by Formula (I) or a salt thereof, wherein M¹ and M² is as set forth in any one of aspects 5-7 wherein R⁷ and R⁸ are each hydrogen; and the remaining variables are as set forth in the first aspect.

In a twelfth aspect, disclosed herein is a compound represented by Formula (I) or a salt thereof, wherein M¹, M², R⁷ and R⁸ are as set forth in any one of aspects 1-11 and wherein m is 1 or 2 and R³ is, at each occurrence, independently selected from halogen, —C₁-C₃ alkyl, —C₁-C₃ haloalkyl, —OH, —O—C₁-C₃ alkyl, and —OC₁-C₃ haloalkyl; and the remaining variables are as set forth in the first aspect.

In a thirteenth aspect, disclosed herein is a compound represented by Formula (I) or a salt thereof, wherein M¹, M², R⁷ and R⁸ are as set forth in any one of aspects 1-11 and wherein m is 1 and R³ is, at each occurrence, independently selected from halogen, —C₁-C₃ alkyl, —C₁-C₃ haloalkyl —OH, and —O—C₁-C₃ alkyl; and the remaining variables are as set forth in the first aspect.

In a fourteenth aspect, disclosed herein is a compound represented by Formula (I) or a salt thereof, wherein M¹, M², R⁷ and R⁸ are as set forth in any one of aspects 1-11 and wherein m is zero; and the remaining variables are as set forth in the first aspect.

In a fifteenth aspect, disclosed herein is a compound represented by Formula (I) or a salt thereof, wherein one of one of M¹ and M² is

[chemical structure]

and the other of M¹ and M² is

[chemical structure]

and the remaining variables are as set forth in the first aspect.

In a sixteenth aspect, disclosed herein is a compound represented by Formula (I) or a salt thereof, wherein M¹ and M² is as set forth in aspect 15 wherein Z¹, Z², Z³, and Z⁴ are —C(H); and the remaining variables are as set forth in the first aspect.

In a seventeenth aspect, disclosed herein is a compound represented by Formula (I) or a salt thereof, wherein M¹ and M² is as set forth in aspect 15 wherein Z² is N and Z¹, Z³, and Z⁴ are —C(H); and the remaining variables are as set forth in the first aspect.

In a eighteenth aspect, disclosed herein is a compound represented by Formula (I) or a salt thereof, wherein M¹ and M² are as set forth in aspect 15 wherein Z¹ is N and Z², Z³, and Z⁴ are —C(H); and the remaining variables are as set forth in the first aspect.

In a nineteenth aspect, disclosed herein is a compound represented by Formula (I) or a salt thereof, wherein M², R⁷, R⁸, m, R³, Z¹, Z², Z³, and Z⁴ are as set forth in any one of aspects 1-18 wherein M¹ is

[chemical structure]

and the remaining variables are as set forth in the first aspect.

In a twentieth aspect, disclosed herein is a compound represented by Formula (I) or a salt thereof, wherein M¹, R⁷, R⁸, m, R³, Z¹, Z², Z³, and Z⁴ are as set forth in any one of aspects 1-18 wherein M² is

[chemical structure]

and the remaining variables are as set forth in the first aspect.

In a twenty-first aspect, disclosed herein is a compound represented by Formula (I) or a salt thereof, wherein M¹, M², R⁷, R⁸, m, R³, Z¹, Z², Z³, and Z⁴ are as set forth in any one of aspects 1-20 wherein R⁵ is hydrogen, halogen, or C₁-C₃ alkyl optionally substituted with halogen; and the remaining variables are as set forth in the first aspect.

In a twenty-second aspect, disclosed herein is a compound represented by Formula (I) or a salt thereof, wherein M¹, M², R⁷, R⁸, m, R³, Z¹, Z², Z³, and Z⁴ are as set forth in any one of aspects 1-20 wherein R⁵ is hydrogen or C₁-C₃ alkyl; and the remaining variables are as set forth in the first aspect.

In a twenty-third aspect, disclosed herein is a compound represented by Formula (I) or a salt thereof, wherein M¹, M², R⁷, R⁸, m, R³, Z¹, Z², Z³, and Z⁴ are as set forth in any one of aspects 1-20 and wherein R⁵ is methyl; and the remaining variables are as set forth in the first aspect.

In a twenty-fourth aspect, disclosed herein is a compound represented by Formula (I) or a salt thereof, wherein one of M¹ and M² is

[chemical structure]

and the other of M¹ and M² is selected from:

[chemical structure]

and the remaining variables are as set forth in the first aspect.

In a twenty-fifth aspect, disclosed herein is a compound represented by Formula (I) or a salt thereof, wherein M¹ is as set forth in aspect 24 and wherein M² is

[chemical structure]

and the remaining variables are as set forth in the first aspect.

In a twenty-sixth aspect, disclosed herein is a compound represented by Formula (I) or a salt thereof, wherein M¹, M², R⁷, R⁸, m, R³, R⁵, Z¹, Z², Z³, and Z⁴ are as set forth in any one of aspects 1-25 and wherein R¹ and R² are, at each occurrence, independently selected from hydrogen, halogen, —OR¹¹, —SR¹¹, —N(R¹¹)₂, phenyl, and —C₁-C₆ alkyl wherein said C₁-C₆ alkyl is optionally substituted with one or more substituents independently selected from halogen, —OR¹¹, —SR¹¹, —S(O)R¹⁰, —S(O)₂R¹¹, —S(O)₂N(R¹¹)2—N(R¹¹)₂, —C(O)R¹⁰, —C(O)N(R¹¹)₂, —N(R¹¹)C(O)R¹⁰, —C(O)OR¹¹, and —OC(O)R¹⁰; and the remaining variables are as set forth in the first aspect.

In a twenty-seventh aspect, disclosed herein is a compound represented by Formula (I) or a salt thereof, wherein M¹, M², R⁷, R⁸, m, R³, R⁵, Z¹, Z², Z³, and Z⁴ are as set forth in any one of aspects 1-25 and wherein R¹ and R² are independently selected at each occurrence from hydrogen, phenyl, and —C₁-C₃ alkyl wherein said —C₁-C₃ alkyl is optionally substituted with one or more substituents independently selected from halogen, —OR¹¹, and —C(O)OR¹¹; and the remaining variables are as set forth in the first aspect.

In a twenty-eighth aspect, disclosed herein is a compound represented by Formula (I) or a salt thereof, wherein M¹, M², R⁷, R⁸, m, R³, R⁵, Z¹, Z², Z³, and Z⁴ are as set forth in any one of aspects 1-25 and wherein R¹ and R² are independently selected at each occurrence from hydrogen, —CH₃, —CH₂OH, CH₂CO₂CH₃, and phenyl; and the remaining variables are as set forth in the first aspect.

In a twenty-ninth aspect, disclosed herein is a compound represented by Formula (I) or a salt thereof, wherein M¹, M², R⁷, R⁸, m, R³, R⁵, Z¹, Z², Z³, and Z⁴ are as set forth in any one of aspects 1-25 and wherein R¹ and R² are each hydrogen; and the remaining variables are as set forth in the first aspect.

In a thirtieth aspect, disclosed herein is a compound represented by Formula (I) or a salt thereof, wherein M¹, M², R⁷, R⁸, m, R¹, R², R³, R⁵, Z¹, Z², Z³, and Z⁴ are as set forth in any one of aspects 1-29 and wherein s is zero; and the remaining variables are as set forth in the first aspect.

In a thirty-first aspect, disclosed herein is a compound represented by Formula (I) or a salt thereof, wherein M¹, M², R⁷, R⁸, m, R¹, R², R³, R⁵, Z¹, Z², Z³, and Z⁴ are as set forth in any one of aspects 1-29 and wherein s is one; and the remaining variables are as set forth in the first aspect.

In a thirty-second aspect, disclosed herein is a compound represented by Formula (I) or a salt thereof, wherein M¹, M², R⁷, R⁸, m, R¹, R², R³, R⁵, Z¹, Z², Z³, and Z⁴ are as set forth in any one of aspects 1-29 and wherein s is one and n is two or three; and the remaining variables are as set forth in the first aspect.

In a thirty-third aspect, disclosed herein is a compound represented by Formula (I) or a salt thereof, wherein M¹, M², R⁷, R⁸, m, R¹, R², R³, R⁵, Z¹, Z², Z³, and Z⁴ are as set forth in any one of aspects 1-29 and wherein s is zero or one and n is one; and the remaining variables are as set forth in the first aspect.

In a thirty-fourth aspect, disclosed herein is a compound represented by Formula (I) or a salt thereof, wherein M¹, M², R⁷, R⁸, m, R¹, R², R³, R⁵, Z¹, Z², Z³, and Z⁴ are as set forth in any one of aspects 1-29 and wherein s is zero or one and n is two; and the remaining variables are as set forth in the first aspect.

In a thirty-fifth aspect, disclosed herein is a compound represented by Formula (I) or a salt thereof, wherein M¹, M², R⁷, R⁸, m, R¹, R², R³, R⁵, Z¹, Z², Z³, and Z⁴ are as set forth in any one of aspects 1-29 and wherein s is zero or one and n is three; and the remaining variables are as set forth in the first aspect.

In a thirty-sixth aspect, disclosed herein is a compound represented by Formula (I) or a salt thereof, wherein M¹, M², R⁷, R⁸, m, s, n R¹, R², R³, R⁵, Z¹, Z², Z³, and Z⁴ are as set forth in any one of aspects 1-35 and wherein Y is selected from —O— and —N(R⁹)— and R⁹ is, at each occurrence, independently selected from: hydrogen; and —C₁-C₆ alkyl optionally substituted with one or more substituents independently selected from halogen, —OR⁴¹, —SR⁴¹, —S(O)R⁴⁰, —S(O)₂R⁴¹, —S(O)₂N(R⁴¹)₂—N(R⁴¹)₂, —C(O)R⁴⁰, —C(O)N(R⁴¹)₂, —N(R⁴¹)C(O)R⁴⁰, —C(O)OR⁴¹, and —OC(O)R⁴⁰; and the remaining variables are as set forth in the first aspect.

In a thirty-seventh aspect, disclosed herein is a compound represented by Formula (I) or a salt thereof, wherein M¹, M², R⁷, R⁸, m, s, n R¹, R², R³, R⁵, Z¹, Z², Z³, and Z⁴ are as set forth in any one of aspects 1-35 and wherein Y is selected from —O— and —N(R⁹)— and R⁹ is, at each occurrence, independently selected from: hydrogen; and unsubstituted-C₁-C₆ alkyl; and the remaining variables are as set forth in the first aspect.

In a thirty-eight aspect, disclosed herein is a compound represented by Formula (I) or a salt thereof, wherein M¹, M², R⁷, R⁸, m, s, n R¹, R², R³, R⁵, Z¹, Z², Z³, and Z⁴ are as set forth in any one of aspects 1-35 and wherein Y is selected from O, N(H), and N(Me); and unsubstituted-C₁-C₆ alkyl; and the remaining variables are as set forth in the first aspect.

In a thirty-ninth aspect, disclosed herein is a compound represented by Formula (I) or a salt thereof, wherein M¹, M², R⁷, R⁸, m, s, n R¹, R², R³, R⁵, R⁹, Y, Z¹, Z², Z³, and Z⁴ are as set forth in any one of aspects 1-38 and wherein w is zero; and the remaining variables are as set forth in the first aspect.

In a fortieth aspect, disclosed herein is a compound represented by Formula (I) or a salt thereof, wherein M¹, M², R⁷, R⁸, m, s, n R¹, R², R³, R⁵, R⁹, Y, Z¹, Z², Z³, and Z⁴ are as set forth in any one of aspects 1-38 and wherein w is 1, 2, 3, 4, or 5; and the remaining variables are as set forth in the first aspect.

In a forty-first aspect, disclosed herein is a compound represented by Formula (I) or a salt thereof, wherein M¹, M², R⁷, R⁸, m, s, n R¹, R², R³, R⁵, R⁹, Y, Z¹, Z², Z³, and Z⁴ are as set forth in any one of aspects 1-38 and wherein w is 1, 2, or 3; and the remaining variables are as set forth in the first aspect.

In a forty-second aspect, disclosed herein is a compound represented by Formula (I) or a salt thereof, wherein M¹, M², R⁷, R⁸, m, s, n R¹, R², R³, R⁵, R⁹, Y, Z¹, Z², Z³, and Z⁴ are as set forth in any one of aspects 1-38 and wherein w is 1 or 2; and the remaining variables are as set forth in the first aspect.

In a forty-third aspect, disclosed herein is a compound represented by Formula (I) or a salt thereof, wherein M¹, M², R⁷, R⁸, m, s, n R¹, R², R³, R⁵, R⁹, Y, Z¹, Z², Z³, and Z⁴ are as set forth in any one of aspects 1-38 having Formula (I), (IA), (IB), (IC), (ID) or (IE):

[chemical structure]

or a pharmaceutically acceptable salt of any one of Formula (I), (IA), (IB), (IC), (ID) or (IE); and the remaining variables are as set forth in the first aspect.

In a forty-fourth aspect, disclosed herein is a compound represented by Formula (I) wherein M¹, M², R⁷, R⁸, m, s, n R¹, R², R³, R⁵, R⁹, Y, Z¹, Z², Z³, and Z⁴ are as set forth in any one of aspects 1-38 having formula (IC) or (ID)

[chemical structure]

or a pharmaceutically acceptable salt of any one of formula (IC), or (ID); and the remaining variables are as set forth in the first aspect.

In a forty-fifth aspect, disclosed herein is a compound represented by Formula (I) wherein M¹, M², R⁷, R⁸, m, s, n R¹, R², R³, R⁵, R⁹, Y, Z¹, Z², Z³, and Z⁴ are as set forth in any one of aspects 1-38 having formula (IF)

[chemical structure]

wherein w is 1, 2, 3, or 4; or a pharmaceutically acceptable salt thereof); and the remaining variables are as set forth in the first aspect.

In a forty-sixth aspect, disclosed herein is a compound represented by Formula (IF) or a salt thereof, wherein M¹, M², R⁷, R⁸, m, s, n, R¹, R², R³, R⁵, R⁹, Y, Z¹, Z², Z³, and Z⁴ are as set forth in any one of aspects 1-38 and w is 2 or 3; and the remaining variables are as set forth in the first aspect.

In a forty-seventh aspect, disclosed herein is a compound represented by Formula (IF) or a salt thereof, wherein M¹, M², R⁷, R⁸, m, s, n, R¹, R², R³, R⁵, R⁹, Y, Z¹, Z², Z³, and Z⁴ are as set forth in any one of aspects 1-38 and w is 2; and the remaining variables are as set forth in the first aspect.

In a forty-eighth aspect, disclosed herein is a compound represented by Formula (I), (IA), (IB), (IC), (ID), (IE) or (IF) or a salt thereof, wherein M¹, M², R⁷, R⁸, m, s, n, w, R¹, R², R³, R⁵, R⁹, Y, Z¹, Z², Z³, and Z⁴ are as set forth in any one of aspects 1-47 and R⁶ is independently selected at each occurrence from:
halogen, —OR²¹, —N(R²¹)₂ and —CN; and
C₁-C₆ alkyl optionally substituted with one or more substituents independently selected from halogen, —OR²¹, —SR²¹, —N(R²¹)₂, —C(O)R²⁰, —C(O)N(R²¹)₂, —N(R²¹)C(O)R²⁰, —C(O)OR²¹, —OC(O)R²¹, —S(O)R²⁰, —S(O)₂R²¹, —S(O)₂N(R²¹)₂, —OC(O)OR²¹, —OC(O)N(R²¹)₂, —NR²¹C(═O)OR²¹, —N(R²¹)C(O)N(R²¹)₂, —NO₂, ═O, ═S, ═N(R²¹), —CN, a C₃-C₁₀ carbocycle, and a 3- to 10-membered heterocycle wherein said C₃-C₁₀ carbocycle and said 3- to 10-membered heterocycle are optionally substituted with one or more substituents independently selected from R^{X}; and
a C₃-C₁₀ carbocycle and a 3- to 10-membered heterocycle, each of which is optionally substituted with one or more substituents independently selected from halogen, —OR²⁰, —OH, —SR²⁰, —SH, —N(R²¹)₂, —C(O)R²⁰, —C(O)N(R²¹)₂, —N(R²¹)C(O)R²⁰, —C(O)OR²¹, —OC(O)R²¹, —S(O)R²⁰, —S(O)₂R²¹, —S(O)₂N(R²¹)₂, —OC(O)OR²¹, —OC(O)N(R²¹)₂, —NR²¹C(═O)OR²¹, —N(R²¹)C(O)N(R²¹)₂, —NO₂, ═O, ═S, ═N(R²¹), —CN, —C₂-C₆ alkenyl, —C₂-C₆ alkynyl and C₁-C₆ alkyl wherein said C₁-C₆ alkyl is optionally substituted with one or more substituents independently selected from R^{Y}; and the remaining variables are as set forth in the first aspect.

In a forty-ninth aspect, disclosed herein is a compound represented by Formula (I), (IA), (IB), (IC), (ID), (IE) or (IF) or a salt thereof, wherein M¹, M², R⁷, R⁸, m, s, n, w, R¹, R², R³, R⁵, R⁹, Y, Z¹, Z², Z³, and Z⁴ are as set forth in any one of aspects 1-47 and R⁶ is independently selected at each occurrence from:
halogen, —OR²¹, and —N(R²¹)₂; and
C₁-C₆ alkyl optionally substituted with one or more substituents independently selected from halogen, —OR²¹, —SR²¹, —N(R²¹)₂, —C(O)R²⁰, —C(O)N(R²¹)₂, —N(R²¹)C(O)R²⁰, —C(O)OR²¹, —OC(O)R²¹, —S(O)R²⁰, —S(O)₂R²¹, —S(O)₂N(R²¹)₂, —OC(O)OR²¹, —OC(O)N(R²¹)₂, —NR²¹C(═O)OR²¹, —N(R²¹)C(O)N(R²¹)₂, —NO₂, ═O, ═S, ═N(R²¹), —CN, a C₃-C₁₀ carbocycle, and a 3- to 10-membered heterocycle wherein said C₃-C₁₀ carbocycle and said 3- to 10-membered heterocycle are optionally substituted with one or more substituents independently selected from R^{X}; and
phenyl and a 6-membered heterocycle comprising 1, 2, or 3 ring heteroatoms selected from nitrogen, each of which is optionally substituted with one or more substituents independently selected from halogen, —OR²⁰, —OH, —SR²⁰, —SH, —N(R²¹)₂, —C(O)R²⁰, —C(O)N(R²¹)₂, —N(R²¹)C(O)R²⁰, —C(O)OR²¹, —OC(O)R²¹, —S(O)R²⁰, —S(O)₂R²¹, —S(O)₂N(R²¹)₂, —OC(O)OR²¹, —OC(O)N(R²¹)₂, —NR²¹C(═O)OR²¹, —N(R²¹)C(O)N(R²¹)₂, —NO₂, ═O, ═S, ═N(R²¹), —CN, —C₂-C₆ alkenyl, —C₂-C₆ alkynyl and C₁-C₆ alkyl wherein said C₁-C₆ alkyl is optionally substituted with one or more substituents independently selected from R^{Y}; and the remaining variables are as set forth in the first aspect. In some such aspects, at least one R⁶ is phenyl optionally substituted with one or more substituents independently selected from halogen, —OR²⁰, —OH, —SR²⁰, —SH, —N(R²¹)₂, —C(O)R²⁰, —C(O)N(R²¹)₂, —N(R²¹)C(O)R²⁰, —C(O)OR²¹, —OC(O)R²¹, —S(O)R²⁰, —S(O)₂R²¹, —S(O)₂N(R²¹)₂, —OC(O)OR²¹, —OC(O)N(R²¹)₂, —NR²¹C(═O)OR²¹, —N(R²¹)C(O)N(R²¹)₂, —NO₂, ═O, ═S, ═N(R²¹), —CN, —C₂-C₆ alkenyl, —C₂-C₆ alkynyl and C₁-C₆ alkyl wherein said C₁-C₆ alkyl is optionally substituted with one or more substituents independently selected from R^{Y}.

In a fiftieth aspect, disclosed herein is a compound represented by Formula (I), (IA), (IB), (IC), (ID), (IE) or (IF) or a salt thereof, wherein M¹, M², R⁷, R⁸, m, s, n, w, R¹, R², R³, R⁵, R⁹, Y, Z¹, Z², Z³, and Z⁴ are as set forth in any one of aspects 1-47 and R⁶ is independently selected at each occurrence from:
halogen, —OR²¹, and —N(R²¹)₂; and
C₁-C₆ alkyl optionally substituted with halogen; and
a C₃-C₁₀ carbocycle and a 3- to 10-membered heterocycle, each of which is optionally substituted with C₁-C₆ alkyl wherein said C₁-C₆ alkyl is optionally substituted with halogen; and the remaining variables are as set forth in the first aspect.

In a fifty-first aspect, disclosed herein is a compound represented by Formula (I), (IA), (IB), (IC), (ID), (IE) or (IF) or a salt thereof, wherein M¹, M², R⁷, R⁸, m, s, n, w, R¹, R², R³, R⁵, R⁹, Y, Z¹, Z², Z³, and Z⁴ are as set forth in any one of aspects 1-47 and R⁶ is independently selected at each occurrence from:
halogen, and —OR²¹; and
C₁-C₆ alkyl optionally substituted with halogen; and
phenyl and a 6-membered heterocycle comprising 1, 2, or 3 ring heteroatoms selected from nitrogen, each of which is optionally substituted with C₁-C₆ alkyl wherein said C₁-C₆ alkyl is optionally substituted with one or more substituents independently selected from R^{Y}; and the remaining variables are as set forth in the first aspect.

In a fifty-second aspect, disclosed herein is a compound represented by Formula (I), (IA), (IB), (IC), (ID), (IE) or (IF) or a salt thereof, wherein M¹, M², R⁶, R⁷, R⁸, m, s, n, w, R¹, R², R³, R⁵, R⁹, Y, Z¹, Z², Z³, and Z⁴ are as set forth in any one of aspects 1-51 and R²¹ is C₁-C₃alkyl, phenyl or a 6-membered heterocycle comprising 1, 2, or 3 ring heteroatoms selected from nitrogen; and the remaining variables are as set forth in the first aspect.

In a fifty-third aspect, disclosed herein is a compound represented by Formula (I), (IA), (IB), (IC), (ID), (IE) or (IF) or a salt thereof, wherein M¹, M², R⁶, R⁷, R⁸, m, s, n, w, R¹, R², R³, R⁵, R⁹, R²¹, Y, Z¹, Z², Z³, and Z⁴ are as set forth in any one of aspects 1-52 and R^{Y} on the phenyl or heterocycle of R⁶ is selected from halogen; and the remaining variables are as set forth in the first aspect.

In a fifty-fourth aspect, disclosed herein is a compound represented by Formula (I), (IA), (IB), (IC), (ID), (IE) or (IF) or a salt thereof, wherein M¹, M², R⁶, R⁷, R⁸, m, s, n, w, R¹, R², R³, R⁵, R⁹, R²¹, Y, Z¹, Z², Z³, and Z⁴ are as set forth in any one of aspects 1-52 and R^{Y} on the phenyl or heterocycle of R⁶ is selected from fluorine or chlorine; and the remaining variables are as set forth in the first aspect.

In a fifty-fifth aspect, disclosed herein is a compound represented by Formula (I), (IA), (IB), (IC), (ID), (IE) or (IF) or a salt thereof, wherein M¹, M², R⁷, R⁸, m, s, n, w, R¹, R², R³, R⁵, R⁹, R²¹, Y, Z¹, Z², Z³, and Z⁴ are as set forth in any one of aspects 1-54 and R⁶ is independently selected at each occurrence from: F, Cl, —OCH₃, —CF₃, —CN, —CH₃, —CH₂CH₃, —CH(CH₃)₂, —OCF₃, —CH₂CF₃, —CH(OH)(CF₃), N(CH₃)₂, pyridyl, cyclohexyl, cyclopentyl, —O-phenyl, —O-pyridyl, and phenyl optionally substituted with one or more substituents independently selected from F, and —CH₂NH₂; and the remaining variables are as set forth in the first aspect. In some such aspects, R⁶ does not comprise cyano.

In a fifty-sixth aspect, disclosed herein is a compound represented by Formula (I) or a salt thereof, wherein M¹, M², R⁷, R⁸, m, s, n, R¹, R², R³, R⁵, R⁹, R²¹, Y, Z¹, Z², Z³, and Z⁴ are as set forth in any one of aspects 1-55 and

[chemical structure]

is:

[chemical structure]

[chemical structure]

[chemical structure]

[chemical structure]

wherein [symbol] represents the point of attachment to

[chemical structure]

and the remaining variables are as set forth in the first aspect.

In a fifty-seventh aspect, disclosed herein is a compound represented by Formula (I) or a salt thereof, wherein M¹, M², R⁷, R⁸, m, R³, R⁵, R⁹, R²¹, Z¹, Z², Z³, and Z⁴ are as set forth in any one of aspects 1-55 and wherein

[chemical structure]

is:

[chemical structure]

[chemical structure]

[chemical structure]

[chemical structure]

[chemical structure]

[chemical structure]

wherein [symbol] represents the point of attachment to

[chemical structure]

and the remaining variables are as set forth in the first aspect.

Exemplary compounds of the present invention include those set forth in Table 16 and salts thereof (including pharmaceutically acceptable salts thereof).

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, compounds described herein are intended to include all Z-, E- and tautomeric forms as well.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

[chemical structure]

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, compounds described herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, and ¹²⁵I are all contemplated. All isotopic variations of the compounds of the present invention, whether radioactive or not, are encompassed within the scope of the present invention.

In certain embodiments, the compounds disclosed herein have some or all of the ⁴H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Compounds of the present invention also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

Included in the present disclosure are salts, particularly pharmaceutically acceptable salts, of the compounds described herein. The compounds of the present disclosure that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride, particularly bromide.

The compounds described herein may in some cases exist as diastereomers, enantiomers, or other stereoisomeric forms. The compounds presented herein include all diastereomeric, enantiomeric, and epimeric forms as well as the appropriate mixtures thereof. Separation of stereoisomers may be performed by chromatography or by forming diastereomers and separating by recrystallization, or chromatography, or any combination thereof. (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis.

The methods and compositions described herein include the use of amorphous forms as well as crystalline forms (also known as polymorphs). The compounds described herein may be in the form of pharmaceutically acceptable salts. As well, in some embodiments, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

In certain embodiments, compounds or salts of the compounds may be prodrugs, e.g., wherein a hydroxyl in the parent compound is presented as an ester or a carbonate, or carboxylic acid present in the parent compound is presented as an ester. The term “prodrug” is intended to encompass compounds which, under physiologic conditions, are converted into pharmaceutical agents of the present disclosure. One method for making a prodrug is to include one or more selected moieties which are hydrolyzed under physiologic conditions to reveal the desired molecule. In other embodiments, the prodrug is converted by an enzymatic activity of the host animal such as specific target cells in the host animal. For example, esters or carbonates (e.g., esters or carbonates of alcohols or carboxylic acids and esters of phosphonic acids) are preferred prodrugs of the present disclosure.

Prodrug forms of the herein described compounds, wherein the prodrug is metabolized in vivo to produce a compound as set forth herein are included within the scope of the claims. In some cases, some of the herein-described compounds may be a prodrug for another derivative or active compound.

Prodrugs are often useful because, in some situations, they may be easier to administer than the parent drug. They may, for instance, be bioavailable by oral administration whereas the parent is not. Prodrugs may help enhance the cell permeability of a compound relative to the parent drug. The prodrug may also have improved solubility in pharmaceutical compositions over the parent drug. Prodrugs may be designed as reversible drug derivatives, for use as modifiers to enhance drug transport to site-specific tissues or to increase drug residence inside of a cell.

In certain embodiments, the prodrug may be converted, e.g., enzymatically or chemically, to the parent compound under the conditions within a cell. In certain embodiments, the parent compound comprises an acidic moiety, e.g., resulting from the hydrolysis of the prodrug, which may be charged under the conditions within the cell. In particular embodiments, the prodrug is converted to the parent compound once it has passed through the cell membrane into a cell. In certain embodiments, the parent compound has diminished cell membrane permeability properties relative to the prodrug, such as decreased lipophilicity and increased hydrophilicity.

In particular embodiments, the parent compound with the acidic moiety is retained within a cell for a longer duration than the same compound without the acidic moiety.

The parent compound, with an acidic moiety, may be retained within the cell, i.e., drug residence, for 10% or longer, such as 15% or longer, such as 20% or longer, such as 25% or longer, such as 30% or longer, such as 35% or longer, such as 40% or longer, such as 45% or longer, such as 50% or longer, such as 55% or longer, such as 60% or longer, such as 65% or longer, such as 70% or longer, such as 75% or longer, such as 80% or longer, such as 85% or longer, or even 90% or longer relative to the same compound without an acidic moiety.

In some embodiments, the design of a prodrug increases the lipophilicity of the pharmaceutical agent. In some embodiments, the design of a prodrug increases the effective water solubility. See, e.g., Fedorak et al., Am. J. Physiol., 269:G210-218 (1995); McLoed et al., Gastroenterol, 106:405-413 (1994); Hochhaus et al., Biomed Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein for such disclosure). According to another embodiment, the present disclosure provides methods of producing the above-defined compounds. The compounds may be synthesized using conventional techniques. Advantageously, these compounds are conveniently synthesized from readily available starting materials.

Synthetic chemistry transformations and methodologies useful in synthesizing the compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

### Linkers and Linker-Payloads

The compounds and salts described herein may be bound to a linker, e.g., a peptide linker. In certain embodiments, the linker is also bound to an antibody, an antibody construct, or a targeting moiety, and may be referred to as an antibody conjugate, an antibody construct conjugate, or a targeting moiety conjugate, respectively, or may be referred to simply as a conjugate. Linkers of the conjugates may not affect the binding of active portions of a conjugate, e.g., the antigen binding domains, Fc region or domains, target binding domain, antibody, targeting moiety, or the like, to a target, which can be a cognate binding partner, such as an antigen. A conjugate can comprise multiple linkers, each having one or more compounds attached. The multiple linkers can be the same linker or different linkers contained on a single conjugate or on separate conjugates.

As will be appreciated by skilled artisans, a linker connects one or more ALK5 inhibitors to an antibody or antigen-binding fragment thereof by forming a covalent linkage to the compound at one location and a covalent linkage to the antibody or antigen-binding fragment thereof at another location. The covalent linkages can be formed by reaction between functional groups on the linker and functional groups on the ALK5 inhibitor and on the antibody or antigen-binding fragment thereof. As used herein, the expression “linker” can include (i) unattached forms of the linker that can include a functional group capable of covalently attaching the linker to an ALK5 inhibitor and a functional group capable of covalently attached the linker to an antibody or antigen-binding fragment thereof; (ii) partially attached forms of the linker that can include a functional group capable of covalently attaching the linker to an antibody or antigen-binding fragment thereof and that can be covalently attached to an ALK5 inhibitor compound, or vice versa; and (iii) fully attached forms of the linker that can be covalently attached to both an ALK5 inhibitor compound and to an antibody or antigen-binding fragment thereof. In some specific embodiments, the functional groups on a linker and covalent linkages formed between the linker and an antibody or antigen-binding fragment thereof can be specifically illustrated as Rx and Rx′, respectively.

A linker can be short, flexible, rigid, cleavable, non-cleavable, hydrophilic, or hydrophobic. A linker can contain segments that have different characteristics, such as segments of flexibility or segments of rigidity. The linker can be chemically stable to extracellular environments, for example, chemically stable in the blood stream, or may include linkages that are not stable or selectively stable. The linker can include linkages that are designed to cleave and/or immolate or otherwise breakdown specifically or non-specifically inside cells. A cleavable linker can be sensitive to enzymes. A cleavable linker can be cleaved by enzymes such as proteases.

A cleavable linker can include a valine-citrulline (Val-Cit) peptide, a valine-alanine (Val-Ala) peptide, a phenylalanine-lysine (Phe-Lys) or other peptide, such as a peptide that forms a protease recognition and cleavage site. Such a peptide-containing linker can contain a pentafluorophenyl group. A peptide-containing linker can include a succimide or a maleimide group. A peptide-containing linker can include a para aminobenzoic acid (PABA) group. A peptide-containing linker can include an aminobenzyloxycarbonyl (PABC) group. A peptide-containing linker can include a PABA or PABC group and a pentafluorophenyl group. A peptide-containing linker can include a PABA or PABC group and a succinimide group. A peptide-containing linker can include a PABA or PABC group and a maleimide group.

A non-cleavable linker is generally protease-insensitive and insensitive to intracellular processes. A non-cleavable linker can include a maleimide group. A non-cleavable linker can include a succinimide group. A non-cleavable linker can be maleimido-alkyl-C(O)— linker. A non-cleavable linker can be maleimidocaproyl linker. A maleimidocaproyl linker can be N-maleimidomethylcyclohexane-1-carboxylate. A maleimidocaproyl linker can include a succinimide group. A maleimidocaproyl linker can include pentafluorophenyl group.

A linker can be a combination of a maleimidocaproyl group and one or more polyethylene glycol molecules. A linker can be a maleimide-PEG4 linker. A linker can be a combination of a maleimidocaproyl linker containing a succinimide group and one or more polyethylene glycol molecules. A linker can be a combination of a maleimidocaproyl linker containing a pentafluorophenyl group and one or more polyethylene glycol molecules. A linker can contain a maleimide(s) linked to polyethylene glycol molecules in which the polyethylene glycol can allow for more linker flexibility or can be used lengthen the linker.

A linker can be a (maleimidocaproyl)-(valine-alanine)-(para-aminobenzyloxycarbonyl) linker. A linker can be a (maleimidocaproyl)-(valine-citrulline)-(para-aminobenzyloxycarbonyl) linker. A linker can be a (maleimidocaproyl)-(phenylalanine-lysine)-(para-aminobenzyloxycarbonyl) linker. A linker can be a linker suitable for attachment to an engineered cysteine (THIOMAB). A THIOMAB linker can be a (maleimidocaproyl)-(valine-citrulline)-(para-aminobenzyloxycarbonyl)-linker.

A linker can also contain segments of alkylene, alkenylene, alkynylene, polyether, polyester, polyamide, polyamino acids, peptides, polypeptides, cleavable peptides, and/or aminobenzyl-carbamates. A linker can contain a maleimide at one end and an N-hydroxysuccinimidyl ester at the other end. A linker can contain a lysine with an N-terminal amine acetylated, and a valine-citrulline, valine-alanine or phenylalanine-lysine cleavage site. A linker can be a link created by a microbial transglutaminase, wherein the link can be created between an amine-containing moiety and a moiety engineered to contain glutamine as a result of the enzyme catalyzing a bond formation between the acyl group of a glutamine side chain and the primary amine of a lysine chain. A linker can contain a reactive primary amine. A linker can be a Sortase A linker. A Sortase A linker can be created by a Sortase A enzyme fusing an LPXTG recognition motif (SEQ ID NO:49) to an N-terminal GGG motif to regenerate a native amide bond. The linker created can therefore link to a moiety attached to the LPXTG recognition motif (SEQ ID NO:49) with a moiety attached to the N-terminal GGG motif. A linker can be a link created between an unnatural amino acid on one moiety reacting with oxime bond that was formed by modifying a ketone group with an alkoxyamine on another moiety. A moiety can be part of a conjugate. A moiety can be part of an antibody. A moiety can be part of an immune-stimulatory compound, such as ALK5 inhibitor. A moiety can be part of a binding domain. A linker can be unsubstituted or substituted, for example, with a substituent. A substituent can include, for example, hydroxyl groups, amino groups, nitro groups, cyano groups, azido groups, carboxyl groups, carboxaldehyde groups, imine groups, alkyl groups, alkenyl groups, alkynyl groups, alkoxy groups, acyl groups, acyloxy groups, amide groups, and ester groups.

In the conjugates, a compound or salt of any one of Formulas (IA), (IB), (IC), (ID), (IE), (IF), and Table 16 is linked to the antibody by way of a linker(s), also referred to herein as L³. L³, as used herein, may be selected from any of the linker moieties discussed herein. The linker linking the compound or salt to the antibody construct of a conjugate may be short, long, hydrophobic, hydrophilic, flexible or rigid, or may be composed of segments that each independently have one or more of the above-mentioned properties such that the linker may include segments having different properties. The linkers may be polyvalent such that they covalently link more than one compound or salt to a single site on the antibody construct, or monovalent such that covalently they link a single compound or salt to a single site on the antibody construct.

A linker can be polyvalent such that it covalently links more than one ALK5 compound to a single site on the antibody or antigen-binding fragment thereof, or monovalent such that it covalently links a single ALK5 compound to a single site on the antibody or antigen-binding fragment thereof.

In certain embodiments for a compound of Formulas (IA), (IB), (IC), (ID), (IE), (IF), and Table 16, or pharmaceutically acceptable isomer, tautomer, racemate, hydrate, solvate, isotope, or salt thereof, the compound may further comprise a linker (L), which results a linker-payload. The linker may be covalently bound to any position, valence permitting, on a compound of Formulas (IA), (IB), (IC), (ID), (IE), (IF), and Table 16, or a pharmaceutically acceptable isomer, tautomer, racemate, hydrate, solvate, isotope, or salt thereof. For example, the linker may be bound to R⁷ or R⁸. In some embodiments, a linker is bound to R⁷. In further embodiments, a linker is bound to a nitrogen atom, e.g., an amine, or oxygen atom, e.g., a hydroxyl, of a compound of Formulas (IA), (IB), (IC), (ID), (IE), (IF), and Table 16, or pharmaceutically acceptable isomer, tautomer, racemate, hydrate, solvate, isotope, or salt thereof. The linker may comprise a reactive moiety, e.g., an electrophile that can react to form a covalent bond with a reactive moiety of an antibody, an antibody construct, or a targeting moiety, e.g., a lysine, serine, threonine, cysteine, tyrosine, aspartic acid, glutamine, a non-natural amino acid residue, or glutamic acid residue. In some embodiments, a compound of Formulas (IA), (IB), (IC), (ID), (IE), (IF), and Table 16, or pharmaceutically acceptable isomer, tautomer, racemate, hydrate, solvate, isotope, or salt thereof, may be covalently bound through the linker to an antibody, an antibody construct, or a targeting moiety.

In the conjugates, a compound of Formulas (IA), (IB), (IC), (ID), (IE), (IF), and Table 16, or pharmaceutically acceptable isomer, tautomer, racemate, hydrate, solvate, isotope, or salt thereof, is linked to an antibody, an antibody construct, or a targeting moiety by way of a linker(s), also referred to herein as L or L³. L, as used herein, may be selected from any of the linker moieties discussed herein. The linker linking the compound or salt to an antibody, an antibody construct, or a targeting moiety of a conjugate may be short, long, hydrophobic, hydrophilic, flexible or rigid, or may be composed of segments that each independently have one or more of the above-mentioned properties, such that the linker may include segments having different properties. The linkers may be polyvalent such that they covalently link more than one compound or salt to a single site on an antibody, an antibody construct, or a targeting moiety, or monovalent, such that covalently they link a single compound or salt to a single site on an antibody, an antibody construct, or a targeting moiety.

Linkers of the disclosure (L³) may have from about 10 to about 500 atoms in a linker, such as from about 10 to about 400 atoms, such as about 10 to about 300 atoms in a linker. In certain embodiments, linkers of the disclosure have from about 30 to about 400 atoms, such as from about 30 to about 300 atoms in the linker.

As will be appreciated by skilled artisans, the linkers may link a compound of Formula (I), (IA), (IB), (IC), (ID), (IE), (IF), or Table 16, or pharmaceutically acceptable isomer, tautomer, racemate, hydrate, solvate, isotope, or salt thereof, of any one of Formulas (IA), (IB), (IC), (ID), (IE), (IF), and Table 16 to an antibody, an the antibody construct, or a targeting moiety by a covalent linkages between the linker and the antibody, the antibody construct, or the targeting moiety, and the compound, to form a conjugate. As used herein, the expression “linker” is intended to include (i) unconjugated forms of the linker that include a functional group capable of covalently linking the linker to a compound of Formula (I), (IA), (IB), (IC), (ID), (IE), (IF), or Table 16, or pharmaceutically acceptable isomer, tautomer, racemate, hydrate, solvate, isotope, or salt thereof, the present invention and a functional group capable of covalently linking the linker to an antibody, an antibody construct, or a targeting moiety; (ii) partially conjugated forms of the linker that include a functional group capable of covalently linking the linker to the an antibody, the antibody construct, or the targeting moiety, and that is covalently linked to at least one compound of Formula (I), (IA), (IB), (IC), (ID), (IE), (IF), or Table 16, or pharmaceutically acceptable isomer, tautomer, racemate, hydrate, solvate, isotope,(s) or salt thereof(s) of any one of Formulas (IA), (IB), (IC), (ID), (IE), (IF), and Table 16, or vice versa; and (iii) fully conjugated forms of the linker that is covalently linked to both a compound of Formula (I), (IA), (IB), (IC), (ID), (IE), (IF), or Table 16, or pharmaceutically acceptable isomer, tautomer, racemate, hydrate, solvate, isotope, or salt thereof, and an antibody, an antibody construct, or the targeting moiety. Some embodiments pertain. One embodiment pertains to a conjugate formed by contacting an antibody, an antibody construct, or a targeting moiety that binds a cell surface receptor or tumor-associated antigen expressed on a tumor cell with a linker-compound described herein under conditions in which the linker-compound covalently links to the antibody, the antibody construct, or the targeting moiety. Further embodiments pertain construct. One embodiment pertains to a method of making a conjugate formed by contacting a linker-compound under conditions in which the linker-compound covalently links to an antibody, an the antibody construct, or a targeting moiety.

In certain embodiments, a compound of Formula (I), (IA), (IB), (IC), (ID), (IE), (IF), or Table 16, or pharmaceutically acceptable isomer, tautomer, racemate, hydrate, solvate, isotope, or salt thereof, Salts described in the section entitled “Compounds” is covalently bound to a linker (L) to form a linker-payload ((L-P)-L³). The linker may be covalently bound to any position of the compound, valence permitting. The linker may comprise a reactive moiety, e.g., an electrophile that can react to form a covalent bond with a moiety of an antibody, an antibody construct, or a targeting moiety, such as, for example, a lysine, serine, threonine, cysteine, tyrosine, aspartic acid, glutamine, a non-natural amino acid residue, or glutamic acid residue. In some embodiments, a linker-payload, comprising a compound or salt of a compound in the section entitled “Compounds” herein and a linker, L, is covalently bound through L the linker to an antibody, an antibody construct, or a targeting moiety. In certain embodiments, any one of the compounds or salts described in the section entitled “Compounds” is covalently bound to a linker (L³). The linker may be covalently bound to any position, valence permitting. The linker may comprise a reactive moiety, e.g., an electrophile that can react to form a covalent bond with a moiety of an antibody construct such as, for example, a lysine, serine, threonine, cysteine, tyrosine, aspartic acid, glutamine, a non-natural amino acid residue, or glutamic acid residue. In some embodiments, a compound or salt of a compound in the section entitled “Compounds” herein is covalently bound through the linker to an antibody construct.

In certain embodiments, a linker-payload, comprising an ALK5 inhibitor compound or salt thereof of this disclosure and a linker, L, is covalently bound through L to an antibody. In further embodiments, a linker-payload, comprising an ALK5 inhibitor compound or salt thereof of this disclosure and a linker, L, is covalently bound through L to an antibody construct. In still further embodiments, a linker-payload, comprising an ALK5 inhibitor compound or salt thereof of this disclosure and a linker, L, is covalently bound through L to a targeting moiety. In any of the aforementioned embodiments, for a linker-payload comprising a compound of Formula (I), (IA), (IB), (IC), (ID), (IE), (IF), or Table 16, or pharmaceutically acceptable isomer, tautomer, racemate, hydrate, solvate, isotope, or salt thereof, Lisa noncleavable linker. Alternatively, in any of the aforementioned embodiments, for a linker-payload comprising a compound of Formula (I), (IA), (IB), (IC), (ID), (IE), (IF), or Table 16, or pharmaceutically acceptable isomer, tautomer, racemate, hydrate, solvate, isotope, or salt thereof, Lisa cleavable linker, such as a linker cleavable by a lysosomal enzyme. In any of the aforementioned embodiments, the antibody, the antibody construct, or the targeting moiety may specifically bind to a tumor antigen. In any of the aforementioned embodiments, the antibody, antibody construct, or targeting moiety may further comprise a second antigen or target binding domain.

In some embodiments, an ALK5 inhibitor compound of this disclosure is covalently attached to an antibody, an antibody construct, or a targeting moiety. In particular embodiments, an ALK5 inhibitor compound of this disclosure is covalently attached to an antibody. In certain embodiments, an ALK5 inhibitor compound of this disclosure is covalently attached to an antibody construct. In certain other embodiments, the compound is covalently attached to a targeting moiety. In any of the aforementioned embodiments, the antibody, the antibody construct, or the targeting moiety may specifically bind to a tumor antigen. In any of the aforementioned embodiments, the antibody, antibody construct, or targeting moiety may further comprise a second antigen or target binding domain.

Exemplary polyvalent linkers that may be used to link compounds of the invention to an antibody construct are described. For example, Fleximer® linker technology has the potential to enable high-DAR conjugates with good physicochemical properties. As shown below, the Fleximer® linker technology is based on incorporating drug molecules into a solubilizing poly-acetal backbone via a sequence of ester bonds:

[chemical structure]

The methodology renders highly-loaded conjugates (DAR up to 20) whilst maintaining good physicochemical properties. This methodology can be utilized with an ALK5 compound as shown in the scheme below, where Drug′ refers to the ALK5 compound.

To utilize the Fleximer® linker technology depicted in the scheme above, an aliphatic alcohol can be present or introduced into the ALK5 compound. The alcohol moiety is then attached to an alanine moiety, which is then synthetically incorporated into the Fleximer® linker. Liposomal processing of the conjugate in vitro releases the parent alcohol-containing drug.

In some embodiments, a moiety, construct, or conjugate described herein includes the symbol [symbol] , which indicates the point of attachment, e.g., the point of attachment of a chemical or functional moiety to the compound, the point of attachment of a linker to a compound of the disclosure, or the point of attachment of a linker to an antibody, an antibody construct, or a targeting moiety.

By way of example and not limitation, some cleavable and noncleavable linkers that may be included in the conjugates are described below, in addition to any other described herein.

Sulfamide linkers may be used to link many compounds of the present invention to an antibody construct. Sulfamide linkers are as described herein and e.g., U.S. Patent Publication Number 2019/0038765, the linkers of which are incorporated by reference herein

Cleavable linkers can be cleavable in vitro, in vivo, or both. Cleavable linkers can include chemically or enzymatically unstable or degradable linkages. Cleavable linkers can rely on processes inside the cell to liberate a compound of Formula (I), such as reduction in the cytoplasm, exposure to acidic conditions in the lysosome, or cleavage by specific proteases or other enzymes within the cell. Cleavable linkers can incorporate one or more chemical bonds that are either chemically or enzymatically cleavable while the remainder of the linker can be non-cleavable.

In some embodiments, Lisa linker comprising a reactive moiety. In some embodiments, for a linker-payload comprising a compound of Formula (I), (IA), (IB), (IC), (ID), (IE), (IF), or Table 16, or pharmaceutically acceptable isomer, tautomer, racemate, hydrate, solvate, isotope, or salt thereof, -L is represented by the formula:

[chemical structure]

In some embodiments, -L is represented by the formula:

[chemical structure]

wherein each R³⁰ is independently selected from optionally substituted C₁-C₆ alkyl and optionally substituted phenyl, and RX is the reactive moiety. RX may comprise a leaving group. RX may be a maleimide. L may be further covalently bound to an antibody construct. In some embodiments, L is represented by the formula:

[chemical structure]

wherein RX* is a bond, a succinimide moiety, or a hydrolyzed succinimide moiety bound to a residue of an antibody construct, wherein

[chemical structure]

on RX* represents the point of attachment to a residue of the antibody construct; and each R³⁰ is independently selected from optionally substituted C₁-C₆ alkyl and optionally substituted phenyl.

In some embodiments, for a linker-payload comprising a compound of Formula (I), (IA), (IB), (IC), (ID), (IE), (IF), or Table 16, or pharmaceutically acceptable isomer, tautomer, racemate, hydrate, solvate, isotope, or salt thereof, and linker L; L comprises a methylene carbamate unit.

In some embodiments, for a linker-payload (L-P) comprising a compound of Formula (I), (IA), (IB), (IC), (ID), (IE), (IF), or Table 16, or pharmaceutically acceptable isomer, tautomer, racemate, hydrate, solvate, isotope, or salt thereof, and linker L-RX*; the L-P is part of a conjugate and RX* comprises a hydrolyzed succinamide moiety and is bound to a cysteine residue of an antibody, an antibody construct, or a targeting moiety. In any of the aforementioned embodiments, the antibody, antibody construct, or targeting moiety comprises an antigen binding domain that specifically binds to an antigen selected from mesothelin (MSLN), HER2, CEA, TROP2, EPHA2, p-cadherin, UPK1B, FOLH1, LYPD3, PVRL4 (Nectin-4), and ASGR1. The antibody, antibody construct, or targeting moiety may specifically bind to MSLN, HER2, TROP2, EPHA2, or ASGR1.

By way of example and not limitation, some cleavable and noncleavable linkers that may be included in the conjugates are described below, in addition to any others described herein.

A linker can contain a chemically labile group such as hydrazone and/or disulfide groups. Linkers comprising chemically labile groups can exploit differential properties between the plasma and some cytoplasmic compartments. The intracellular conditions that can facilitate release of a compound Formula (I), (IA), (IB), (IC), (ID), (IE), (IF), or Table 16, or pharmaceutically acceptable isomer, tautomer, racemate, hydrate, solvate, isotope, or salt thereof for hydrazone containing linkers can be the acidic environment of endosomes and lysosomes, while the disulfide containing linkers can be reduced in the cytosol, which can contain high thiol concentrations, e.g., glutathione. The plasma stability of a linker containing a chemically labile group can be increased by introducing steric hindrance using substituents near the chemically labile group.

Acid-labile groups, such as hydrazone, can remain intact during systemic circulation in the blood's neutral pH environment (pH 7.3-7.5) and can undergo hydrolysis and can release a compound of the present invention once the antibody conjugate is internalized into mildly acidic endosomal (pH 5.0-6.5) and lysosomal (pH 4.5-5.0) compartments of the cell. This pH dependent release mechanism can be associated with nonspecific release of the drug. To increase the stability of the hydrazone group of the linker, the linker can be varied by chemical modification, e.g., substitution, allowing tuning to achieve more efficient release in the lysosome with a minimized loss in circulation.

In some embodiments, for a linker-payload comprising a compound of Formula (I), (IA), (IB), (IC), (ID), (IE), (IF), or Table 16, or pharmaceutically acceptable isomer, tautomer, racemate, hydrate, solvate, isotope, or salt thereof; and a linker L, -L comprises a hydrazone moiety. For example, L may be selected from:

[chemical structure]

wherein M is selected from C₁-C₆ alkyl, aryl, and —O—C₁-C₆ alkyl.

Hydrazone-containing linkers can contain additional cleavage sites, such as additional acid-labile cleavage sites and/or enzymatically labile cleavage sites. Conjugates including exemplary hydrazone-containing linkers can include, for example, the following structures:

[chemical structure]

wherein D is a compound or salt of any one of Formulas (IA), (IB), (IC), (ID), (IE), (IF), and Table 16 and Ab is an antibody construct, respectively, and n represents the number of compound-bound linkers (LP) bound to the antibody construct. In certain linkers, such as linker (Ia), the linker can comprise two cleavable groups, a disulfide and a hydrazone moiety. For such linkers, effective release of the unmodified free compound can require acidic pH or disulfide reduction and acidic pH. Linkers such as (Ib) and (Ic) can be effective with a single hydrazone cleavage site.

Other acid-labile groups that can be included in linkers include cis-aconityl-containing linkers. cis-Aconityl chemistry can use a carboxylic acid juxtaposed to an amide bond to accelerate amide hydrolysis under acidic conditions.

Cleavable linkers can also include a disulfide group. Disulfides can be thermodynamically stable at physiological pH and can be designed to release a compound of Formula (I), (IA), (IB), (IC), (ID), (IE), (IF), or Table 16, or pharmaceutically acceptable isomer, tautomer, racemate, hydrate, solvate, isotope, or salt thereof; upon internalization inside cells, wherein the cytosol can provide a significantly more reducing environment compared to the extracellular environment. Scission of disulfide bonds can require the presence of a cytoplasmic thiol cofactor, such as (reduced) glutathione (GSH), such that disulfide-containing linkers can be reasonably stable in circulation, selectively releasing a compound of Formula (I), (IA), (IB), (IC), (ID), (IE), (IF), or Table 16, or pharmaceutically acceptable isomer, tautomer, racemate, hydrate, solvate, isotope, or salt thereof; in the cytosol. The intracellular enzyme protein disulfide isomerase, or similar enzymes capable of cleaving disulfide bonds, can also contribute to the preferential cleavage of disulfide bonds inside cells. GSH can be present in cells in the concentration range of 0.5-10 mM compared with a significantly lower concentration of GSH or cysteine, the most abundant low-molecular weight thiol, in circulation at approximately 5 μM. Tumor cells, where irregular blood flow can lead to a hypoxic state, can result in enhanced activity of reductive enzymes and therefore even higher glutathione concentrations. The in vivo stability of a disulfide-containing linker can be enhanced by chemical modification of the linker, e.g., use of steric hindrance adjacent to the disulfide bond.

Conjugates comprising a compound of Formula (I), (IA), (IB), (IC), (ID), (IE), (IF), or Table 16, or pharmaceutically acceptable isomer, tautomer, racemate, hydrate, solvate, isotope, or salt thereof, and including exemplary disulfide-containing linkers can include the following structures:

[chemical structure]

wherein D is a compound of Formula (I), (IA), (IB), (IC), (ID), (IE), (IF), or Table 16, or pharmaceutically acceptable isomer, tautomer, racemate, hydrate, solvate, isotope, or salt thereof, and Ab is an antibody, an antibody construct, or a targeting moiety, n represents the number of compounds bound to linkers (L) bound to the antibody, antibody construct, or targeting moiety and R is independently selected at each occurrence from, for example, hydrogen or alkyl. Increasing steric hindrance adjacent to the disulfide bond can increase the stability of the linker. Structures such as (CIIa) and (CIIc) can show increased in vivo stability when one or more R groups is selected from a lower alkyl, such as methyl.

Another type of linker that can be used is a linker that is specifically cleaved by an enzyme. For example, the linker can be cleaved by a lysosomal enzyme. Such linkers can be peptide-based or can include peptidic regions that can act as substrates for enzymes. Peptide based linkers can be more stable in plasma and extracellular milieu than chemically labile linkers.

Peptide bonds can have good serum stability, as lysosomal proteolytic enzymes can have very low activity in blood due to endogenous inhibitors and the unfavorably high pH value of blood compared to lysosomes. Release of a compound of Formula (I), (IA), (IB), (IC), (ID), (IE), (IF), or Table 16, or pharmaceutically acceptable isomer, tautomer, racemate, hydrate, solvate, isotope, or salt thereof, from an antibody, an antibody construct, or a targeting moiety conjugate can occur due to the action of lysosomal proteases, e.g., cathepsin and plasmin. These proteases can be present at elevated levels in certain tumor tissues. The linker can be cleavable by a lysosomal enzyme. The lysosomal enzyme can be, for example, cathepsin B, β-glucuronidase, or β-galactosidase.

The cleavable peptide can be selected from tetrapeptides such as Gly-Phe-Leu-Gly (SEQ ID NO: 235), Ala-Leu-Ala-Leu (SEQ ID NO: 236) or dipeptides such as Val-Cit, Val-Ala, and Phe-Lys. Dipeptides can have lower hydrophobicity compared to longer peptides.

A variety of dipeptide-based cleavable linkers can be used with an antibody, an antibody construct, or a targeting moiety construct to form conjugates of a compound of Formula (I), (IA), (IB), (IC), (ID), (IE), (IF), or Table 16, or pharmaceutically acceptable isomer, tautomer, racemate, hydrate, solvate, isotope, or salt thereof, described herein.

Enzymatically cleavable linkers can include a self-immolative spacer to spatially separate a compound of Formula (I), (IA), (IB), (IC), (ID), (IE), (IF), or Table 16, or pharmaceutically acceptable isomer, tautomer, racemate, hydrate, solvate, isotope, or salt thereof, from the site of enzymatic cleavage. The direct attachment of a compound of Formula (I), (IA), (IB), (IC), (ID), (IE), (IF), or Table 16, or pharmaceutically acceptable isomer, tautomer, racemate, hydrate, solvate, isotope, or salt thereof, to a peptide linker can result in proteolytic release of a compound of Formula (I), (IA), (IB), (IC), (ID), (IE), (IF), or Table 16, or pharmaceutically acceptable isomer, tautomer, racemate, hydrate, solvate, isotope, or salt thereof, or of an amino acid adduct of a compound of Formula (I), (IA), (IB), (IC), (ID), (IE), (IF), or Table 16, or pharmaceutically acceptable isomer, tautomer, racemate, hydrate, solvate, isotope, or salt thereof, thereby impairing its activity. The use of a self-immolative spacer can allow for the elimination of the fully active, chemically unmodified compound of Formula (I), (IA), (IB), (IC), (ID), (IE), (IF), or Table 16, or pharmaceutically acceptable isomer, tautomer, racemate, hydrate, solvate, isotope, or salt thereof, upon amide bond hydrolysis.

One self-immolative spacer can be a bifunctional para-aminobenzyl alcohol (PABA) group, which can link to a peptide through an amino group, forming an amide bond, while an amine containing compound of Formula (I), (IA), (IB), (IC), (ID), (IE), (IF), or Table 16, or pharmaceutically acceptable isomer, tautomer, racemate, hydrate, solvate, isotope, or salt thereof, can be attached through carbamate functionalities to the benzylic hydroxyl group of the linker (to give a p-amidobenzylcarbarnate, PABC). The resulting pro-compound can be activated upon protease-mediated cleavage, leading to a 1,6-elimination reaction releasing the unmodified compound of Formula (I), (IA), (IB), (IC), (ID), (IE), (IF), or Table 16, or pharmaceutically acceptable isomer, tautomer, racemate, hydrate, solvate, isotope, or salt thereof, carbon dioxide, and remnants of the linker group.

The following scheme depicts the fragmentation of p-amidobenzyl carbamate and release of a compound of Formula (I), (IA), (IB), (IC), (ID), (IE), (IF), or Table 16, or pharmaceutically acceptable isomer, tautomer, racemate, hydrate, solvate, isotope, or salt thereof:

[chemical structure]

wherein D represents the unmodified drug or payload having the structure of a compound of Formula (I), (IA), (IB), (IC), (ID), (IE), (IF), or Table 16, or pharmaceutically acceptable isomer, tautomer, racemate, hydrate, solvate, isotope, or salt thereof.

In some embodiments, for a linker-payload comprising a compound of Formula (I), (IA), (IB), (IC), (ID), (IE), (IF), or Table 16, or pharmaceutically acceptable isomer, tautomer, racemate, hydrate, solvate, isotope, or salt thereof, L is represented by the formula:

[chemical structure]

wherein peptide comprises from one to ten amino acids, and [symbol] represents the point of attachment to the compound (payload).

In some embodiments, for a linker-payload comprising a compound of Formula (I), (IA), (IB), (IC), (ID), (IE), (IF), or Table 16, or pharmaceutically acceptable isomer, tautomer, racemate, hydrate, solvate, isotope, or salt thereof, L is represented by the formula:

[chemical structure]

wherein peptide comprises from one to ten amino acids and RX is a reactive moiety, and [symbol] represents the point of attachment to the compound (payload).

In some embodiments, for a linker-payload comprising a compound of Formula (I), (IA), (IB), (IC), (ID), (IE), (IF), or Table 16, or pharmaceutically acceptable isomer, tautomer, racemate, hydrate, solvate, isotope, or salt thereof, L is represented by the formula:

[chemical structure]

wherein peptide comprises from one to ten amino acids, L⁴ is the C-terminus of the peptide and L⁵ is selected from a bond, alkylene and heteroalkylene, wherein L⁵ is optionally substituted with one or more groups independently selected from R³², RX is a reactive moiety; and R³² is independently selected at each occurrence from halogen, —OH, —CN, —O-alkyl, —SH, ═O, ═S, —NH₂, —NO₂; and C₁₋₁₀ alkyl, C₂₋₁₀ alkenyl, and C₂₋₁₀ alkynyl, each of which is independently optionally substituted at each occurrence with one or more substituents selected from halogen, —OH, —CN, —O-alkyl, —SH, ═O, ═S, —NH₂, and —NO₂. The reactive moiety may be selected from an electrophile, e.g., an αβ-unsaturated carbonyl, such as a maleimide, and a leaving group. In some embodiments, RX comprises a leaving group. In certain embodiments, RX is a maleimide.

In some embodiments, for a linker-payload comprising a compound of Formula (I), (IA), (IB), (IC), (ID), (IE), (IF), or Table 16, or pharmaceutically acceptable isomer, tautomer, racemate, hydrate, solvate, isotope, or salt thereof, the L-P is part of a conjugate and L is represented by the formula:

[chemical structure]

wherein Antibody is an antibody, an RX* antibody construct, or a targeting moiety peptide comprises from one to 10 amino acids, RX* is a reactive moiety that has reacted with a moiety on the antibody, antibody construct, or targeting moiety to form a conjugate, and [symbol] represents the point of attachment to the compound (payload).

In further embodiments, L-P is part of a conjugate and -L- is represented by the formula:

[chemical structure]

wherein peptide comprises from one to ten amino acids, L⁴ is the C-terminus of the peptide and L⁵ is selected from a bond, an alkylene and a heteroalkylene, each of which is optionally substituted with one or more groups independently selected from R¹²; [symbol] on the left represents the point of attachment to the compound (payload), RX* is a bond, a succinimide moiety, or a hydrolyzed succinimide moiety attached at the [symbol] on the right to a residue of an antibody, an antibody construct, or a targeting moiety.

In some embodiments, L-P is part of a conjugate and -L- is represented by the formula:

[chemical structure]

wherein peptide comprises from one to ten amino acids, L⁴ represents the C-terminus of the peptide and L⁵ is selected from a bond, alkylene and heteroalkylene, wherein L⁵ is optionally substituted with one or more groups independently selected from R³²; RX* is a bond, a succinimide moiety, or a hydrolyzed succinimide moiety bound to a residue of an antibody, an antibody construct, or a targeting moiety, wherein

[chemical structure]

on RX* represents the point of attachment to the residue of the antibody, antibody construct, or targeting moiety; and R³² is independently selected at each occurrence from halogen, —OH, —CN, —O-alkyl, —SH, ═O, ═S, —NH₂, —NO₂; and C₁₋₁₀ alkyl, C₂₋₁₀ alkenyl, and C₂₋₁₀ alkynyl, each of which is independently optionally substituted at each occurrence with one or more substituents selected from halogen, —OH, —CN, —O-alkyl, —SH, ═O, ═S, —NH₂, and —NO₂. In some embodiments, the peptide of L comprises Val-Cit or Val-Ala.

In any of the aforementioned embodiments, L is:

[chemical structure]

Heterocyclic variants of this self-immolative group may also be used.

The enzymatically cleavable linker can be a β-glucuronic acid-based linker. Facile release of a compound of Formula (I), (IA), (IB), (IC), (ID), (IE), (IF), or Table 16, or pharmaceutically acceptable isomer, tautomer, racemate, hydrate, solvate, isotope, or salt thereof, can be realized through cleavage of the β-glucuronide glycosidic bond by the lysosomal enzyme β-glucuronidase. This enzyme can be abundantly present within lysosomes and can be overexpressed in some tumor types, while the enzyme activity outside cells can be low. β-Glucuronic acid-based linkers can be used to circumvent the tendency of an antibody construct conjugate of a compound of Formula (I), (IA), (IB), (IC), (ID), (IE), (IF), or Table 16, or pharmaceutically acceptable isomer, tautomer, racemate, hydrate, solvate, isotope, or salt thereof, to undergo aggregation due to the hydrophilic nature of β-glucuronides. In some embodiments, β-glucuronic acid-based linkers can link an antibody, an antibody construct, or a targeting moiety to a hydrophobic compound of Formula (I), (IA), (IB), (IC), (ID), (IE), (IF), or Table 16, or pharmaceutically acceptable isomer, tautomer, racemate, hydrate, solvate, isotope, or salt thereof.

The following scheme depicts the release of a compound of Formula (I), (IA), (IB), (IC), (ID), (IE), (IF), or Table 16, or pharmaceutically acceptable isomer, tautomer, racemate, hydrate, solvate, isotope, or salt thereof, (D) from a conjugate of a compound of Formula (I), (IA), (IB), (IC), (ID), (IE), (IF), or Table 16, or pharmaceutically acceptable isomer, tautomer, racemate, hydrate, solvate, isotope, or salt thereof, containing a β-glucuronic acid-based linker:

[chemical structure]

wherein Ab indicates an antibody, an antibody construct, or a targeting moiety.

A variety of cleavable β-glucuronic acid-based linkers useful for linking drugs such as auristatins, camptothecin analogues, doxorubicin analogues, CBI minor-groove binders, and psymberin to antibodies have been described. These β-glucuronic acid-based linkers may be used in the conjugates. In some embodiments, an enzymatically cleavable linker is a β-galactoside-based linker. β-Galactoside is present abundantly within lysosomes, while the enzyme activity outside cells is low.

Additionally, a compound of Formula (I), (IA), (IB), (IC), (ID), (IE), (IF), or Table 16, or pharmaceutically acceptable isomer, tautomer, racemate, hydrate, solvate, isotope, or salt thereof, containing a phenol group can be covalently bonded to a linker through the phenolic oxygen. One such linker relies on a methodology in which a diamino-ethane “Space Link” is used in conjunction with traditional “PABO”-based self-immolative groups to deliver phenols.

Cleavable linkers can include non-cleavable portions or segments, and/or cleavable segments or portions can be included in an otherwise non-cleavable linker to render it cleavable. By way of example only, polyethylene glycol (PEG) and related polymers can include cleavable groups in the polymer backbone. For example, a polyethylene glycol or polymer linker can include one or more cleavable groups such as a disulfide, a hydrazone or a dipeptide.

Other degradable linkages that can be included in linkers can include ester linkages formed by the reaction of PEG carboxylic acids or activated PEG carboxylic acids with alcohol groups on a compound of Formula (I), (IA), (IB), (IC), (ID), (IE), (IF), or Table 16, or pharmaceutically acceptable isomer, tautomer, racemate, hydrate, solvate, isotope, or salt thereof, wherein such ester groups can hydrolyze under physiological conditions to release a compound of Formula (I), (IA), (IB), (IC), (ID), (IE), (IF), or Table 16, or pharmaceutically acceptable isomer, tautomer, racemate, hydrate, solvate, isotope, or salt thereof. Hydrolytically degradable linkages can include carbonate linkages; imine linkages resulting from reaction of an amine and an aldehyde; phosphate ester linkages formed by reacting an alcohol with a phosphate group; acetal linkages that are the reaction product of an aldehyde and an alcohol; orthoester linkages that are the reaction product of a formate and an alcohol; and oligonucleotide linkages formed by a phosphoramidite group, including at the end of a polymer, and a 5′ hydroxyl group of an oligonucleotide.

A linker can contain an enzymatically cleavable peptide, for example, a linker comprising structural formula (CIIIa), (CIIIb), (CIIIc), or (CIIId):

[chemical structure]

or a salt thereof, wherein: “peptide” represents a peptide (illustrated in N→C orientation, wherein peptide includes the amino and carboxy “termini”) that is cleavable by a lysosomal enzyme; T represents a polymer comprising one or more ethylene glycol units or an alkylene chain, or combinations thereof; R^{a} is selected from hydrogen, alkyl, sulfonate and methyl sulfonate; R^{Y} is hydrogen or C₁₋₄ alkyl-(O)ᵣ—(C₁₋₄ alkylene)ₛ-G¹ or C₁₋₄ alkyl-(N)—[(C₁₋₄ alkylene)-G¹]₂; R^{z} is C₁₋₄ alkyl-(O)ᵣ—(C₁₋₄ alkylene)ₛ-G²; G¹ is —SO₃H, —CO₂H, PEG 4-32, or a sugar moiety; G² is —SO₃H, —CO₂H, or a PEG 4-32 moiety; r is 0 or 1; s is 0 or 1; p is an integer ranging from 0 to 5; q is 0 or 1; x is 0 or 1; y is 0 or 1; [symbol] represents the point of attachment of the linker to a compound of Formula (I), (IA), (IB), (IC), (ID), (IE), (IF), or Table 16, or pharmaceutically acceptable isomer, tautomer, racemate, hydrate, solvate, isotope, or salt thereof; and * represents the point of attachment to the remainder of the linker.

In certain embodiments, a peptide can be selected to contain natural amino acids, unnatural amino acids, or any combination thereof. In some embodiments, a peptide can be a tripeptide or a dipeptide. In particular embodiments, a dipeptide comprises L-amino acids, such as Val-Cit; Cit-Val; Ala-Ala; Ala-Cit; Cit-Ala; Asn-Cit; Cit-Asn; Cit-Cit; Val-Glu; Glu-Val; Ser-Cit; Cit-Ser; Lys-Cit; Cit-Lys; Asp-Cit; Cit-Asp; Ala-Val; Val-Ala; Phe-Lys; Lys-Phe; Val-Lys; Lys-Val; Ala-Lys; Lys-Ala; Phe-Cit; Cit-Phe; Leu-Cit; Cit-Leu; Ile-Cit; Cit-Ile; Phe-Arg; Arg-Phe; Cit-Trp; and Trp-Cit, or salts thereof.

Exemplary embodiments of linkers according to structural formula (CIIIa) are illustrated below (as illustrated, the linkers include a reactive group suitable for covalently linking the linker to an antibody, an antibody construct, or a targeting moiety):

[chemical structure]

[chemical structure]

wherein [symbol] indicates an attachment site of a linker (L) to a compound of Formula (I), (IA), (IB), (IC), (ID), (IE), (IF), or Table 16, or pharmaceutically acceptable isomer, tautomer, racemate, hydrate, solvate, isotope, or salt thereof.

Exemplary embodiments of linkers according to structural formula (CIIIb), (CIIIc), or (CIIId) that can be included in the conjugates can include the linkers illustrated below (as illustrated, the linkers include a reactive group suitable for covalently linking the linker to an antibody construct):

[chemical structure]

[chemical structure]

[chemical structure]

[chemical structure]

[chemical structure]

[chemical structure]

[chemical structure]

[chemical structure]

[chemical structure]

wherein [symbol] indicates an attachment site to a compound of Formula (I), (IA), (IB), (IC), (ID), (IE), (IF), or Table 16, or pharmaceutically acceptable isomer, tautomer, racemate, hydrate, solvate, isotope, or salt thereof.

The linker can contain an enzymatically cleavable sugar moiety, for example, a linker comprising structural formula (CIVa), (CIVb), (CIVc), (CIVd), or (CIVe):

[chemical structure]

or a salt thereof, wherein: q is 0 or 1; r is 0 or 1; X¹ is CH₂, O or NH; [symbol] represents the point of attachment of the linker (L) to a compound of Formula (I), (IA), (IB), (IC), (ID), (IE), (IF), or Table 16, or pharmaceutically acceptable isomer, tautomer, racemate, hydrate, solvate, isotope, or salt thereof; and * represents the point of attachment to the remainder of the linker.

Exemplary embodiments of linkers according to structural formula (CIVa) that may be included in the antibody construct conjugates of a compound of Formula (I), (IA), (IB), (IC), (ID), (IE), (IF), or Table 16, or pharmaceutically acceptable isomer, tautomer, racemate, hydrate, solvate, isotope, or salt thereof, described herein can include the linkers illustrated below (as illustrated, the linkers include a group suitable for covalently linking the linker to an antibody construct):

[chemical structure]

[chemical structure]

[chemical structure]

wherein [symbol] represents the point of attachment of the linker (L) to a compound of Formula (I), (IA), (IB), (IC), (ID), (IE), (IF), or Table 16, or pharmaceutically acceptable isomer, tautomer, racemate, hydrate, solvate, isotope, or salt thereof.

Exemplary embodiments of linkers according to structural formula (CIVb) that may be included in the conjugates include the linkers illustrated below (as illustrated, the linkers include a group suitable for covalently linking the linker to an antibody construct):

[chemical structure]

[chemical structure]

[chemical structure]

wherein [symbol] represents the point of attachment of the linker (L) to a compound of Formula (I), (IA), (IB), (IC), (ID), (IE), (IF), or Table 16, or pharmaceutically acceptable isomer, tautomer, racemate, hydrate, solvate, isotope, or salt thereof.

Exemplary embodiments of linkers according to structural formula (CIVc) that may be included in the conjugates include the linkers illustrated below (as illustrated, the linkers include a group suitable for covalently linking the linker to an antibody construct):

[chemical structure]

[chemical structure]

[chemical structure]

[chemical structure]

wherein [symbol] represents the point of attachment of the linker (L) to a compound of Formula (I), (IA), (IB), (IC), (ID), (IE), (IF), or Table 16, or pharmaceutically acceptable isomer, tautomer, racemate, hydrate, solvate, isotope, or salt thereof.

Exemplary embodiments of linkers according to structural formula (CIVd) that may be included in the conjugates include the linkers illustrated below (as illustrated, the linkers include a group suitable for covalently linking the linker to an antibody construct):

[chemical structure]

[chemical structure]

wherein [symbol] represents the point of attachment of the linker (L) to a compound of Formula (I), (IA), (IB), (IC), (ID), (IE), (IF), or Table 16, or pharmaceutically acceptable isomer, tautomer, racemate, hydrate, solvate, isotope, or salt thereof.

Exemplary embodiments of linkers according to structural formula (CIVe) that may be included in the conjugates include the linkers illustrated below (as illustrated, the linkers include a group suitable for covalently linking the linker to an antibody construct):

[chemical structure]

wherein [symbol] represents the point of attachment of the linker (L) to a compound of Formula (I), (IA), (IB), (IC), (ID), (IE), (IF), or Table 16, or pharmaceutically acceptable isomer, tautomer, racemate, hydrate, solvate, isotope, or salt thereof.

Although cleavable linkers can provide certain advantages, the linkers comprising the conjugate need not be cleavable. For non-cleavable linkers, the payload compound release may not depend on the differential properties between the plasma and some cytoplasmic compartments. The release of the payload compound can occur after internalization of the conjugate via antigen-mediated endocytosis and delivery to lysosomal compartment, where the antibody, antibody construct, or targeting moiety can be degraded to the level of amino acids through intracellular proteolytic degradation. This process can release a payload compound derivative (a metabolite of the conjugate containing a non-cleavable linker-heterocyclic compound), which is formed by the payload compound, the linker, and the amino acid residue or residues to which the linker was covalently attached. The payload compound derivative from conjugates with non-cleavable linkers can be more hydrophilic and less membrane permeable, which can lead to less bystander effects and less nonspecific toxicities compared to conjugates with a cleavable linker. Conjugates with non-cleavable linkers can have greater stability in circulation than conjugates with cleavable linkers. Non-cleavable linkers can include alkylene chains, or can be polymeric, such as, for example, based upon polyalkylene glycol polymers, amide polymers, or can include segments of alkylene chains, polyalkylene glycols and/or amide polymers. The linker can contain a polyethylene glycol segment having from 1 to 6 ethylene glycol units.

The linker can be non-cleavable in vivo, for example, a linker-payload comprising a compound of Formula (I), (IA), (IB), (IC), (ID), (IE), (IF), or Table 16, or pharmaceutically acceptable isomer, tautomer, racemate, hydrate, solvate, isotope, or salt thereof, and a linker L; -L is represented by the formulas below:

[chemical structure]

or salts thereof, wherein: R^{a} is selected from hydrogen, alkyl, sulfonate and methyl sulfonate; R^{X} is a reactive moiety including a functional group capable of covalently linking the linker to an antibody construct; and [symbol] represents the point of attachment of the linker (L) to a compound of Formula (I), (IA), (IB), (IC), (ID), (IE), (IF), or Table 16, or pharmaceutically acceptable isomer, tautomer, racemate, hydrate, solvate, isotope, or salt thereof.

In some embodiments, for a linker-payload comprising a compound of Formula (I), (IA), (IB), (IC), (ID), (IE), (IF), or Table 16, or pharmaceutically acceptable isomer, tautomer, racemate, hydrate, solvate, isotope, or salt thereof, and a linker L; -L is represented by the formula:

[chemical structure]

wherein n=0-9 and [symbol] represents the point of attachment to the compound (payload).

In some embodiments, for a linker-payload comprising a compound of Formula (I), (IA), (IB), (IC), (ID), (IE), (IF), or Table 16, or pharmaceutically acceptable isomer, tautomer, racemate, hydrate, solvate, isotope, or salt thereof, and a linker L; -L is represented by the formula:

[chemical structure]

wherein RX comprises a reactive moiety, e.g., a maleimide or a leaving group, n=0-9, and [symbol] represents the point of attachment to the compound (payload).

In some embodiments, for a conjugate comprising a compound of Formula (I), (IA), (IB), (IC), (ID), (IE), (IF), or Table 16, or pharmaceutically acceptable isomer, tautomer, racemate, hydrate, solvate, isotope, or salt thereof, a linker L, and an antibody, an antibody construct, or a targeting moiety; -L- is represented by the formula:

[chemical structure]

RX⁺ is a bond, a succinimide moiety, or a hydrolyzed succinimide moiety attached at the [symbol] on the right to a residue of the antibody, antibody construct, or targeting moiety, [symbol] on the left represents the point of attachment to the compound (payload), and n=0-9.

Exemplary embodiments of linkers according to structural formula (CVa)-(Ve) that may be included in the conjugates include the linkers illustrated below (as illustrated, the linkers include a group suitable for covalently linking the linker to an antibody construct, and [symbol] represents the point of attachment of the linker (L) to a compound of Formula (I), (IA), (IB), (IC), (ID), (IE), (IF), or Table 16, or pharmaceutically acceptable isomer, tautomer, racemate, hydrate, solvate, isotope, or salt thereof:

[chemical structure]

Attachment groups that are used to attach the linkers to an antibody, an antibody construct, or a targeting moiety can be electrophilic in nature and include, for example, maleimide groups, activated disulfides, active esters such as NHS esters and HOBt esters, haloformates, acid halides, alkyl, and benzyl halides such as haloacetamides. There are also emerging technologies related to “self-stabilizing” maleimides and “bridging disulfides” that can be used with ALK5 inhibitor compounds of this disclosure. Examples of cysteine based linkers are provided in PCT Patent Application Publication Number WO 2020/092385, the linkers of which are incorporated by reference herein.

Maleimide groups are frequently used in the preparation of conjugates because of their specificity for reacting with thiol groups of, for example, cysteine groups of an antibody, an antibody construct or a targeting moiety. The reaction between a thiol group of an antibody, an antibody construct or a targeting moiety and a drug with a linker (linker-aoyload) including a maleimide group proceeds according to the following scheme:

[chemical structure]

The reverse reaction leading to maleimide elimination from a thio-substituted succinimide may also take place. This reverse reaction is undesirable as the maleimide group may subsequently react with another available thiol group such as other proteins in the body having available cysteines. Accordingly, the reverse reaction can undermine the specificity of a conjugate. One method of preventing the reverse reaction is to incorporate a basic group into the linking group shown in the scheme above. Without wishing to be bound by theory, the presence of the basic group may increase the nucleophilicity of nearby water molecules to promote ring-opening hydrolysis of the succinimide group. The hydrolyzed form of the attachment group is resistant to deconjugation in the presence of plasma proteins. So-called “self-stabilizing” linkers provide conjugates with improved stability. A representative schematic is slum n below

[chemical structure]

The hydrolysis reaction schematically represented above may occur at either carbonyl group of the succinimide group. Accordingly, two possible isomers may result, as shown below:

[chemical structure]

The identity of the base as well as the distance between the base and the maleimide group can be modified to tune the rate of hydrolysis of the thio-substituted succinimide group and optimize the delivery of a conjugate to a target by, for example, improving the specificity and stability of the conjugate.

Bases suitable for inclusion in a linker, e.g., any L with a maleimide group prior to conjugation to an antibody, an antibody construct, or a targeting moiety may facilitate hydrolysis of a nearby succinimide group formed after conjugation of the antibody, antibody construct, or targeting moiety to the linker. Bases may include, for example, amines (e.g., —N(R²⁶)(R²⁷), where R²⁶ and R²⁷ are independently selected from H and C₁₋₆ alkyl), nitrogen-containing heterocycles (e.g., a 3- to 12-membered heterocycle including one or more nitrogen atoms and optionally one or more double bonds), amidines, guanidines, and carbocycles or heterocycles substituted with one or more amine groups (e.g., a 3- to 12-membered aromatic or non-aromatic cycle optionally including a heteroatom such as a nitrogen atom and substituted with one or more amines of the type —N(R²⁶)(R²⁷), where R²⁶ and R²⁷ are independently selected from H or C₁₋₆ alkyl). A basic unit may be separated from a maleimide group by, for example, an alkylene chain of the form —(CH₂)ₘ—, where m is an integer from 0 to 10. An alkylene chain may be optionally substituted with other functional groups as described herein.

A linker (L) with a maleimide group may include an electron withdrawing groups, such as —C(O)R, ═O, —CN, —NO₂, —CX₃, —X, —C(O)OR, —C(O)NR₂, —C(O)R, —C(O)X, —SO₂R, —SO₂OR, —SO₂NHR, —SO₂NR₂, —PO₃R₂, —P(O)(CH₃)NHR, —NO, —NR₃⁺, —CR═CR₂, and —C≡CR, where each R is independently selected from H and C₁₋₆ alkyl and each X is independently selected from F, Br, Cl, and I. Self-stabilizing linkers may also include aryl, e.g., phenyl, or heteroaryl, e.g., pyridine, groups optionally substituted with electron withdrawing groups, such as those described herein.

Examples of self-stabilizing linkers are provided in, e.g., U.S. Patent Application Publication Number US 2013/0309256, the linkers of which are incorporated by reference herein. It will be understood that a self-stabilizing linker useful in conjunction with the compounds of the present invention may be equivalently described as unsubstituted maleimide-including linkers, thio-substituted succinimide-including linkers, or hydrolyzed, ring-opened thio-substituted succinimide-including linkers.

In some embodiments, for a linker-payload comprising a compound of Formula (I), (IA), (IB), (IC), (ID), (IE), (IF), or Table 16, or pharmaceutically acceptable isomer, tautomer, racemate, hydrate, solvate, isotope, or salt thereof, and a linker L; -L comprises a self-stabilizing moiety. For example, L may be selected from:

[chemical structure]

In the scheme provided above, the bottom structure may be referred to as (maleimido)-DPR-Val-Cit-PAB, where DPR refers to diaminopropinoic acid, Val refers to valine, Cit refers to citrulline, and PAB refers to para-aminobenzylcarbonyl. [symbol] represent the point of attachment to a compound of Formula (I), (IA), (IB), (IC), (ID), (IE), (IF), or Table 16, or pharmaceutically acceptable isomer, tautomer, racemate, hydrate, solvate, isotope, or salt thereof.

A method for bridging a pair of sulfhydryl groups derived from reduction of a native hinge disulfide bond has been disclosed and is depicted in the schematic below. An advantage of this methodology is the ability to synthesize homogenous conjugates by full reduction of IgGs (to give 4 pairs of sulfhydryls from interchain disulfides, wherein the DAR can range from 1 to 8) followed by reaction with 4 equivalents of the alkylating agent. Conjugates containing “bridged disulfides” are also claimed to have increased stability.

[chemical structure]

Similarly, as depicted below, a maleimide derivative that is capable of bridging a pair of sulfhydryl groups has been developed.

[chemical structure]

A linker of the disclosure, L, can contain the following structural formulas (CVIa), (CVIb), or (CVIc):

[chemical structure]

or salts thereof, wherein: R^{q} is H or —O—(CH₂CH₂O)₁₁—CH₃; x is 0 or 1; y is 0 or 1; G² is —CH₂CH₂CH₂SO₃H or —CH₂CH₂O—(CH₂CH₂O)₁₁—CH₃; R^{w} is —O—CH₂CH₂SO₃H or —NH(CO)—CH₂CH₂O—(CH₂CH₂O)₁₂—CH₃; and * represents the point of attachment to the remainder of the linker.

Exemplary embodiments of linkers according to structural formula (CVIa) and (CVIb), which can be included in linker-payload and conjugate structures of this disclosure, include the linkers illustrated below (as illustrated, the linkers include a group suitable for covalently linking the linker to an antibody construct):

[chemical structure]

[chemical structure]

[chemical structure]

[chemical structure]

[chemical structure]

wherein [symbol] represents the point of attachment of the linker (L) to a compound of Formula (I), (IA), (IB), (IC), (ID), (IE), (IF), or Table 16, or pharmaceutically acceptable isomer, tautomer, racemate, hydrate, solvate, isotope, or salt thereof.

Exemplary embodiments of linkers according to structural formula (CVIc), which can be included in linker-payload and conjugate structure of this disclosure, include the linkers illustrated below (as illustrated, the linkers include a group suitable for covalently linking the linker to an antibody construct):

[chemical structure]

wherein [symbol] represents the point of attachment of the linker (L) to a compound of Formula (I), (IA), (IB), (IC), (ID), (IE), (IF), or Table 16, or pharmaceutically acceptable isomer, tautomer, racemate, hydrate, solvate, isotope, or salt thereof.

Some exemplary linkers (L) are described in the following paragraphs. In some embodiments for a compound of Formula (I), (IA), (IB), (IC), (ID), (IE), (IF), or Table 16, or pharmaceutically acceptable isomer, tautomer, racemate, hydrate, solvate, isotope, or salt thereof, wherein attachment of the linker is to a nitrogen of the compound and conjugation is to a cysteine residue of an antibody or targeting moiety, -L is represented by the formulas set forth in Table 3 below:

TABLE 3

| Cmpd Name | Structural Formula |
|---|---|
| L1 | [chemical structure] |
| L2 | [chemical structure] |
| L3 | [chemical structure] |
| L4 | [chemical structure] |
| L5 | [chemical structure] |
| L6 | [chemical structure] |
| L7 | [chemical structure] |
| L8 | [chemical structure] |
| L9 | [chemical structure] |
| L10 | [chemical structure] |
| L11 | [chemical structure] |

wherein

[chemical structure]

represents attachment to a nitrogen of a compound or salt of any one of Formula (I), (IA), (IB), (IC), (ID), (IE), or Table 16; L⁴ represents the C-terminus of the peptide and L⁵ is selected from a bond, alkylene and heteroalkylene, wherein L⁵ is optionally substituted with one or more groups independently selected from R³⁰, and R³⁰ is independently selected at each occurrence from halogen, —OH, —CN, —O-alkyl, —SH, ═O, ═S, —NH₂, —NO₂; and C₁-C₁₀alkyl, C₂-C₁₀alkenyl, and C₂-C₁₀alkynyl, each of which is independently optionally substituted at each occurrence with one or more substituents selected from halogen, —OH, —CN, —O-alkyl, —SH, ═O, ═S, —NH₂, and —NO₂; and RX represents a reactive moiety. The reactive moiety may be selected, for example, from an electrophile, e.g., an α,β-un saturated carbonyl, such as a maleimide, and a leaving group. For example, -L can be represented by the formulas set forth in Table 4 below:

TABLE 4

| Cmpd | |
|---|---|
| Name | Structural Formula |
| L12 | [chemical structure] |
| L13 | [chemical structure] |
| L14 | [chemical structure] |
| L15 | [chemical structure] |
| L16 | [chemical structure] |
| L17 | [chemical structure] |
| L18 | [chemical structure] |
| L19 | [chemical structure] |
| L20 | [chemical structure] |
| L21 | [chemical structure] |
| L22 | [chemical structure] |
| L23 | [chemical structure] |
| L24 | [chemical structure] |
| L25 | [chemical structure] |
| L26 | [chemical structure] |

wherein

[chemical structure]

represents attachment to a nitrogen of a compound or salt of any one of Formula (I), (IA), (IB), (IC), (ID), (IE), or Table 16 and L⁴ represents the C-terminus of the peptide and L⁵ is selected from a bond, alkylene and heteroalkylene, wherein L⁵ is optionally substituted with one or more groups independently selected from R³⁰, and R³⁰ is independently selected at each occurrence from halogen, —OH, —CN, —O-alkyl, —SH, ═O, ═S, —NH₂, —NO₂; and C₁-C₁₀alkyl, C₂-C₁₀alkenyl, and C₂-C₁₀alkynyl, each of which is independently optionally substituted at each occurrence with one or more substituents selected from halogen, —OH, —CN, —O-alkyl, —SH, ═O, ═S, —NH₂, and —NO₂.

When conjugated to the cysteine residue of the antibody or targeting moiety, such linkers can be, for example, represented by the Formulas set forth in Table 5 below:

TABLE 5

| Cmpd Name | Structural Formula |
|---|---|
| L27 | [chemical structure] |
| L28 | [chemical structure] |
| L29 | [chemical structure] |
| L30 | [chemical structure] |
| L31 | [chemical structure] |
| L32 | [chemical structure] |
| L33 | [chemical structure] |
| L34 | [chemical structure] |
| L35 | [chemical structure] |
| L36 | [chemical structure] |
| L37 | [chemical structure] |

wherein RX* is a bond, a succinimide moiety, or a hydrolyzed succinimide moiety bound to a cysteine residue of the antibody construct, wherein

[chemical structure]

on RX* represents the point of attachment to such residue; L⁴ when present represents the C-terminus of the peptide and L⁵ is selected from a bond, alkylene and heteroalkylene, wherein L⁵ is optionally substituted with one or more groups independently selected from R³⁰; and R³⁰ when present is independently selected at each occurrence from halogen, —OH, —CN, —O-alkyl, —SH, ═O, ═S, —NH₂, —NO₂; and C₁-C₁₀alkyl, C₂-C₁₀alkenyl, and C₂-C₁₀alkynyl, each of which is independently optionally substituted at each occurrence with one or more substituents selected from halogen, —OH, —CN, —O-alkyl, —SH, ═O, ═S, —NH₂, and —NO₂. A particularly preferred peptide is val-ala or val-cit.

In some embodiments for a compound of Formula (I), (IA), (IB), (IC), (ID), (IE), (IF), or Table 16, or pharmaceutically acceptable isomer, tautomer, racemate, hydrate, solvate, isotope, or salt thereof, wherein attachment of the linker is to a nitrogen of a compound of Formula (I), (IA), (IB), (IC), (ID), (IE), (IF), or Table 16, or pharmaceutically acceptable isomer, tautomer, racemate, hydrate, solvate, isotope, or salt thereof, and conjugation is to a lysine residue of an antibody or other targeting moiety, -L is represented by the formulas set forth in Table 6 below:

TABLE 6

| Cmpd | |
|---|---|
| Name | Structural Formula |
| L38 | [chemical structure] |
| L39 | [chemical structure] |

wherein

[chemical structure]

represents attachment to a nitrogen of a compound of Formula (I), (IA), (IB), (IC), (ID), (IE), (IF), or Table 16, or pharmaceutically acceptable isomer, tautomer, racemate, hydrate, solvate, isotope, or salt thereof, and RX represents a reactive moiety. The reactive moiety may be selected from, for example, a leaving group. For example, -L can be represented by the formulas set forth in Table 7 below:

TABLE 7

| Cmpd Name | Structural Formula |
|---|---|
| L40 | [chemical structure] |
| L41 | [chemical structure] |

When conjugated to the lysine residue of an antibody or other targeting moiety, such linkers, can, for example, be represented by the Formulas set forth in Table 8 below wherein RX* is a bond to a nitrogen of the lysine residue of the antibody construct or targeting moiety, wherein

[chemical structure]

on RX* represents the point of attachment to such residue:

TABLE 8

| Cmpd | |
|---|---|
| Name | Structural Formula |
| L42 | [chemical structure] |
| L43 | [chemical structure] |

As noted,

[chemical structure]

represents attachment to a nitrogen of a compound of Formula (I), (IA), (IB), (IC), (ID), (IE), (IF), or Table 16, or pharmaceutically acceptable isomer, tautomer, racemate, hydrate, solvate, isotope, or salt thereof.

As is known by skilled artisans, the linker selected for a particular conjugate may be influenced by a variety of factors, including the site of attachment to the antibody, antibody construct, or targeting moiety (e.g., lysine, cysteine, or other amino acid residues), structural constraints of the drug pharmacophore, and the lipophilicity of the drug. The specific linker selected for a conjugate should seek to balance these different factors for the specific antibody, antibody construct, or targeting moiety/drug combination.

For example, cytotoxic conjugates have been observed to effect killing of bystander antigen-negative cells present in the vicinity of the antigen-positive tumor cells. The mechanism of the bystander effect by cytotoxic conjugates has indicated that metabolic products formed during intracellular processing of the conjugates may play a role. Neutral cytotoxic metabolites generated by metabolism of the conjugates in antigen-positive cells appear to play a role in bystander cell killing while charged metabolites may be prevented from diffusing across the membrane into the medium, or from the medium across the membrane and, therefore, cannot effect cell killing via the bystander effect. In some embodiments, a linker is selected to attenuate the bystander effect caused by cellular metabolites of the conjugate. In further embodiments, a linker is selected to increase the bystander effect.

The properties of the linker, or linker-payload, may also impact aggregation of a conjugate under conditions of use and/or storage. Conjugates reported in the literature contain about 3-4 drug molecules per antibody molecule. Attempts to obtain higher drug-to-antibody ratios (“DAR”) often failed, particularly if both the drug and the linker were hydrophobic, due to aggregation of the conjugate. In many instances, DARs higher than 3-4 could be beneficial as a means of increasing potency. In instances where the payload compound is more hydrophobic in nature, it may be desirable to select linkers that are relatively hydrophilic as a means of reducing conjugate aggregation, especially in instances where DARs greater than 3-4 are desired. Thus, in some embodiments, a linker incorporates chemical moieties that reduce aggregation of the conjugates during storage and/or use. A linker may incorporate polar or hydrophilic groups such as charged groups or groups that become charged under physiological pH to reduce the aggregation of the conjugates. For example, a linker may incorporate charged groups such as salts or groups that deprotonate, e.g., carboxylates, or protonate, e.g., amines, at physiological pH.

In preferred embodiments, aggregation of conjugates during storage or use is less than about 40% as determined by size-exclusion chromatography (SEC). In particular embodiments, the aggregation of the conjugates during storage or use is less than about 35%, such as less than about 30%, about 25%, about 20%, about 15%, about 10%, about 5%, about 4%, or even less, as determined by size-exclusion chromatography (SEC).

Some exemplary linkers (L³) are described in the following paragraphs. In some embodiments for a compound or salt of Formulas (IA), (IB), (IC), (ID), (IE), (IF), and Table 16 wherein attachment of the linker is to a nitrogen of the compound and conjugation is to a cysteine residue of an antibody or targeting moiety, -L³ is represented by the formulas set forth in Table 9 below:

TABLE 9

| | |
|---|---|
| [chemical structure] | L44 |
| [chemical structure] | L45 |
| [chemical structure] | L46 |
| [chemical structure] | L47 |
| [chemical structure] | L48 |
| [chemical structure] | L49 |
| [chemical structure] | L50 |
| [chemical structure] | L51 |
| [chemical structure] | L52 |
| [chemical structure] | L53 |
| [chemical structure] | L54 |

wherein

[chemical structure]

represents attachment to a nitrogen of a compound or salt of any one of Formulas (IA), (IB), (IC), (ID), (IE), (IF), and Table 16; L⁴ represents the C-terminus of the peptide and L⁵ is selected from a bond, alkylene and heteroalkylene, wherein L⁵ is optionally substituted with one or more groups independently selected from R³⁰, and R³⁰ is independently selected at each occurrence from halogen, —OH, —CN, —O-alkyl, —SH, ═O, ═S, —NH₂, —NO₂; and C₁-C₁₀alkyl, C₂-C₁₀alkenyl, and C₂-C₁₀alkynyl, each of which is independently optionally substituted at each occurrence with one or more substituents selected from halogen, —OH, —CN, —O-alkyl, —SH, ═O, ═S, —NH₂, and —NO₂; and RX represents a reactive moiety. The reactive moiety may be selected, for example, from an electrophile, e.g., an α,β-unsaturated carbonyl, such as a maleimide, and a leaving group. For example, -L³ can be represented by the formulas set forth in Table 10 below:

TABLE 10

| | |
|---|---|
| [chemical structure] | L55 |
| [chemical structure] | L56 |
| [chemical structure] | L57 |
| [chemical structure] | L58 |
| [chemical structure] | L59 |
| [chemical structure] | L60 |
| [chemical structure] | L61 |
| [chemical structure] | L62 |
| [chemical structure] | L63 |
| [chemical structure] | L64 |
| [chemical structure] | L65 |
| [chemical structure] | L66 |
| [chemical structure] | L67 |
| [chemical structure] | L68 |
| [chemical structure] | L69 |

wherein

[chemical structure]

represents attachment to a nitrogen of a compound or salt of any one of Formulas (IA), (IB), (IC), (ID), (IE), (IF), and Table 16 and L⁴ represents the C-terminus of the peptide and L⁵ is selected from a bond, alkylene and heteroalkylene, wherein L⁵ is optionally substituted with one or more groups independently selected from R³⁰, and R³⁰ is independently selected at each occurrence from halogen, —OH, —CN, —O-alkyl, —SH, ═O, ═S, —NH₂, —NO₂; and C₁-C₁₀alkyl, C₂-C₁₀alkenyl, and C₂-C₁₀alkynyl, each of which is independently optionally substituted at each occurrence with one or more substituents selected from halogen, —OH, —CN, —O-alkyl, —SH, ═O, ═S, —NH₂, and —NO₂.

When conjugated to the cysteine residue of the antibody or targeting moiety, such linkers can be, for example, represented by the Formulas set forth in Table 11 below:

TABLE 11

| Cmpd | |
|---|---|
| Name | Structural Formula |
| L70 | [chemical structure] |
| L71 | [chemical structure] |
| L72 | [chemical structure] |
| L73 | [chemical structure] |
| L74 | [chemical structure] |
| L75 | [chemical structure] |
| L76 | [chemical structure] |
| L77 | [chemical structure] |
| L78 | [chemical structure] |
| L79 | [chemical structure] |
| L80 | [chemical structure] |
| L81 | [chemical structure] |

wherein RX* is a bond, a succinimide moiety, or a hydrolyzed succinimide moiety bound to a cysteine residue of the antibody construct, wherein

[chemical structure]

on RX* represents the point of attachment to such residue; L⁴ when present represents the C-terminus of the peptide and L⁵ is selected from a bond, alkylene and heteroalkylene, wherein L⁵ is optionally substituted with one or more groups independently selected from R³⁰; and R³⁰ when present is independently selected at each occurrence from halogen, —OH, —CN, —O-alkyl, —SH, ═O, ═S, —NH₂, —NO₂; and C₁-C₁₀alkyl, C₂-C₁₀alkenyl, and C₂-C₁₀alkynyl, each of which is independently optionally substituted at each occurrence with one or more substituents selected from halogen, —OH, —CN, —O-alkyl, —SH, ═O, ═S, —NH₂, and —NO₂. A particularly preferred peptide is val-ala or val-cit.

In some embodiments for a compound or salt of Formulas (IA), (IB), (IC), (ID), (IE), (IF), and Table 16 wherein attachment of the linker is to a nitrogen of a compound or salt of any one of Formulas (IA), (IB), (IC), (ID), (IE), (IF), and Table 16 and conjugation is to a lysine residue of an antibody or other targeting moiety, -L³ is represented by the formulas set forth in Table 12 below:

TABLE 12

| Cmpd | |
|---|---|
| Name | Structural Formula |
| L82 | [chemical structure] |
| L83 | [chemical structure] |

wherein

[chemical structure]

represents attachment to a nitrogen of a compound or salt of any one of Formulas (IA), (IB), (IC), and Table 16 and RX represents a reactive moiety. The reactive moiety may be selected from, for example, a leaving group. For example, -L³ can be represented by the formulas set forth in Table 13 below:

TABLE 13

| Cmpd | |
|---|---|
| Name | Structural Formula |
| L84 | [chemical structure] |
| L85 | [chemical structure] |

When conjugated to the lysine residue of an antibody or other targeting moiety, such linkers, can, for example, be represented by the Formulas set forth in Table 14 below wherein RX* is a bond to a nitrogen of the lysine residue of the antibody construct or targeting moiety, wherein

[chemical structure]

on RX* represents the point of attachment to such residue:

TABLE 14

[chemical structure]

[chemical structure]

As noted,

[chemical structure]

represents attachment to a nitrogen of a compound or salt of any one of Formulas (IA), (IB), ^{an}d Table 16. In exemplary embodiments, the linkers described herein, including those in the preceding paragraphs, are attached to a compound of the present invention through the nitrogen of the secondary acyclic amine depicted in the structure of formula (I), (IA), (IB), (IC), (ID), or (IE)

In exemplary embodiments, the linkers described herein, including those in the preceding paragraphs, are attached to a compound of the present invention at a nitrogen atom. For example, in some aspects, a compound of the invention covalently bound to the linker -L³ is represented by the following formulas:

[chemical structure]

or a pharmaceutically acceptable salt thereof.

In other aspects, a compound of the invention is covalently bound to the linker -L³ via the R⁶ substituent. For example, the compound covalently bound to a linker -L³ can be represented by the following formulas:

[chemical structure]

or a pharmaceutically acceptable salt thereof.

Such compound-linkers include, for examples, compound linkers of the following formula:

[chemical structure]

pharmaceutically acceptable salt thereof.

Exemplary compound-linkers of the present invention are as shown below in Table 15 wherein L³ represents the linker:

TABLE 15

| | |
|---|---|
| [chemical structure] | LP-1 |
| [chemical structure] | LP-14 |
| [chemical structure] | LP-2 |
| [chemical structure] | LP-3 |
| [chemical structure] | LP-4 |
| [chemical structure] | LP-5 |
| [chemical structure] | LP-10 |
| [chemical structure] | LP-9 |
| [chemical structure] | LP-8 |
| [chemical structure] | LP-7 |
| [chemical structure] | LP-6 |
| [chemical structure] | LP-15 |
| [chemical structure] | LP-16 |
| [chemical structure] | LP-17 |
| [chemical structure] | LP-18 |
| [chemical structure] | LP-24 |
| [chemical structure] | LP-23 |
| [chemical structure] | LP-22 |
| [chemical structure] | LP-21 |
| [chemical structure] | LP-20 |
| [chemical structure] | LP-19 |
| [chemical structure] | LP-25 |
| [chemical structure] | LP-26 |
| [chemical structure] | LP-27 |
| [chemical structure] | LP-28 |
| [chemical structure] | LP-32 |
| [chemical structure] | LP-36 |
| [chemical structure] | LP-35 |
| [chemical structure] | LP-34 |
| [chemical structure] | LP-33 |
| [chemical structure] | LP-37 |
| [chemical structure] | LP-35 |
| [chemical structure] | LP-63 |
| [chemical structure] | LP-64 |
| [chemical structure] | LP-69 |
| [chemical structure] | LP-71 |
| [chemical structure] | LP-73 |
| [chemical structure] | LP-72 |
| [chemical structure] | LP-70 |
| [chemical structure] | LP-68 |
| [chemical structure] | LP-67 |
| [chemical structure] | LP-66 |
| [chemical structure] | LP-65 |
| [chemical structure] | LP-77 |
| [chemical structure] | LP-78 |
| [chemical structure] | LP-79 |
| [chemical structure] | LP-80 |
| [chemical structure] | LP-84 |
| [chemical structure] | LP-86 |
| [chemical structure] | LP-87 |
| [chemical structure] | LP-85 |
| [chemical structure] | LP-83 |
| [chemical structure] | LP-82 |
| [chemical structure] | LP-81 |
| [chemical structure] | LP-49 |
| [chemical structure] | LP-50 |
| [chemical structure] | LP-51 |
| [chemical structure] | LP-52 |
| [chemical structure] | LP-56 |
| [chemical structure] | LP-58 |
| [chemical structure] | LP-61 |
| [chemical structure] | LP-59 |
| [chemical structure] | LP-56 |
| [chemical structure] | LP-55 |
| [chemical structure] | LP-54 |
| [chemical structure] | LP-53 |
| [chemical structure] | LP-60 |
| [chemical structure] | LP-88 |
| [chemical structure] | LP-91 |
| [chemical structure] | LP-89 |
| [chemical structure] | LP-90 |
| [chemical structure] | LP-38 |
| [chemical structure] | LP-37 |
| [chemical structure] | LP-39 |
| [chemical structure] | LP-40 |
| [chemical structure] | LP-41 |
| [chemical structure] | LP-42 |
| [chemical structure] | LP-43 |
| [chemical structure] | LP-44 |
| [chemical structure] | LP-45 |
| [chemical structure] | LP-46 |
| [chemical structure] | LP-47 |
| [chemical structure] | LP-48 |
| [chemical structure] | LP-13 |
| [chemical structure] | LP-12 |
| [chemical structure] | LP-11 |
| [chemical structure] | LP-29 |
| [chemical structure] | LP-30 |
| [chemical structure] | LP-31 |

As is known by skilled artisans, the linker selected for a particular conjugate may be influenced by a variety of factors, including but not limited to, the site of attachment to the antibody construct (e.g., lys, cys or other amino acid residues), structural constraints of the drug pharmacophore and the lipophilicity of the drug. The specific linker selected for a conjugate should seek to balance these different factors for the specific antibody construct/drug combination.

The properties of the linker, or linker-compound, may also impact aggregation of the conjugate under conditions of use and/or storage. Typically, conjugates reported in the literature contain no more than 3-4 drug molecules per antibody molecule. Attempts to obtain higher drug-to-antibody ratios (“DAR”) often failed, particularly if both the drug and the linker were hydrophobic, due to aggregation of the conjugate. In many instances, DARs higher than 3-4 could be beneficial as a means of increasing potency. In instances where the payload compound is more hydrophobic in nature, it may be desirable to select linkers that are relatively hydrophilic as a means of reducing conjugate aggregation, especially in instances where DARs greater than 3-4 are desired. Thus, in certain embodiments, the linker incorporates chemical moieties that reduce aggregation of the conjugates during storage and/or use. A linker may incorporate polar or hydrophilic groups such as charged groups or groups that become charged under physiological pH to reduce the aggregation of the conjugates. For example, a linker may incorporate charged groups such as salts or groups that deprotonate, e.g., carboxylates, or protonate, e.g., amines, at physiological pH.

In particular embodiments, the aggregation of the conjugates during storage or use is less than about 40% as determined by size-exclusion chromatography (SEC). In particular embodiments, the aggregation of the conjugates during storage or use is less than 35%, such as less than about 30%, such as less than about 25%, such as less than about 20%, such as less than about 15%, such as less than about 10%, such as less than about 5%, such as less than about 4%, or even less, as determined by size-exclusion chromatography (SEC).

Exemplary Linker-Compounds of the present invention include those set forth in Tables 15, 16, and 17, and salts thereof (including pharmaceutically acceptable salts thereof.

### Conjugates of PROTACS

In certain embodiments, a conjugate of a compound described herein can be designed to increase ubiquitin-mediated target protein destruction via the ubiquitin pathway. The process of attaching ubiquitin molecules to a protein target typically involves 3 enzymes and steps: 1) an E1 enzyme that can activate ubiquitin, 2) an E2 enzyme that can transfer activated ubiquitin, and 3) a multi-subunit E3 enzyme ligase that can receive the activated ubiquitin and catalyze a ubiquitin attachment to the target protein.

In some embodiments, a conjugate includes a proteolysis targeting module (PTM; also referred to as a proteolysis-targeting chimera or PROTAC). A PTM can comprise a small molecule that can bind to an E3 ubiquitin ligase subunit and a target binding moiety (a compound described herein) that binds a protein target. The E3 ubiquitin ligase binding small molecule is attached, directly or by a spacer (S), to the target binding moiety.

### Pharmaceutical Formulations

The compositions and methods described herein may be considered useful as pharmaceutical compositions for administration to a subject in need thereof. Pharmaceutical compositions may comprise at least the compositions described herein and one or more pharmaceutically acceptable carriers, diluents, excipients, stabilizers, dispersing agents, suspending agents, and/or thickening agents. The composition may comprise the conjugate having an antibody construct and a compound of the present invention. The composition may comprise the conjugate having an antibody construct and a compound of the present invention. The composition may comprise the conjugate having an antibody construct, a target binding domain, and a compound of the present invention. The composition may comprise any conjugate described herein. In some embodiments, the antibody construct is an anti-LRRC15 antibody. A conjugate may comprise an anti-LRRC15 antibody and a compound of the present invention. In some embodiments, the antibody construct is an anti-ASGR1 antibody. A conjugate may comprise an anti-ASGR1 antibody and a compound of the present invention. A pharmaceutical composition can comprise at least the compounds, salts or conjugates described herein and one or more of buffers, antibiotics, steroids, carbohydrates, drugs (e.g., chemotherapy drugs), radiation, polypeptides, chelators, adjuvants and/or preservatives.

Pharmaceutical compositions may be formulated using one or more physiologically-acceptable carriers comprising excipients and auxiliaries. Formulation may be modified depending upon the route of administration chosen. Pharmaceutical compositions comprising a compound, salt or conjugate may be manufactured, for example, by lyophilizing the compound, salt or conjugate, mixing, dissolving, emulsifying, encapsulating or entrapping the conjugate. The pharmaceutical compositions may also include the compounds, salts or conjugates in a free-base form or pharmaceutically-acceptable salt form.

Methods for formulation of the conjugates may include formulating any of the compounds, salts or conjugates with one or more inert, pharmaceutically-acceptable excipients or carriers to form a solid, semi-solid, or liquid composition. Solid compositions may include, for example, powders, tablets, dispersible granules and capsules, and in some aspects, the solid compositions further contain nontoxic, auxiliary substances, for example wetting or emulsifying agents, pH buffering agents, and other pharmaceutically-acceptable additives. Alternatively, the compounds, salts or conjugates may be lyophilized or in powder form for re-constitution with a suitable vehicle, e.g., sterile pyrogen-free water, before use.

Pharmaceutical compositions of the conjugates may comprise at least one active ingredient (e.g., a compound, salt or conjugate and other agents). The active ingredients may be entrapped in microcapsules prepared, for example, by coacervation techniques or by interfacial polymerization (e.g., hydroxymethylcellulose or gelatin microcapsules and poly-(methylmethacylate) microcapsules, respectively), in colloidal drug-delivery systems (e.g., liposomes, albumin microspheres, microemulsions, nano-particles and nanocapsules) or in macroemulsions.

Pharmaceutical compositions as often further may comprise more than one active compound (e.g., a compound, salt or conjugate and other agents) as necessary for the particular indication being treated. The active compounds may have complementary activities that do not adversely affect each other. For example, the composition may comprise a chemotherapeutic agent, cytotoxic agent, cytokine, growth-inhibitory agent, anti-hormonal agent, anti-angiogenic agent, and/or cardioprotectant. Such molecules may be present in combination in amounts that are effective for the purpose intended.

The compositions and formulations may be sterilized. Sterilization may be accomplished by filtration through sterile filtration.

The compositions may be formulated for administration as an injection. Non-limiting examples of formulations for injection may include a sterile suspension, solution or emulsion in oily or aqueous vehicles. Suitable oily vehicles may include, but are not limited to, lipophilic solvents or vehicles such as fatty oils or synthetic fatty acid esters, or liposomes. Aqueous injection suspensions may contain substances which increase the viscosity of the suspension. The suspension may also contain suitable stabilizers. Injections may be formulated for bolus injection or continuous infusion. Alternatively, the compositions may be lyophilized or in powder form for reconstitution with a suitable vehicle, e.g., sterile pyrogen-free water, before use.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrastemal injection and infusion.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (a) sugars, such as lactose, glucose and sucrose; (b) starches, such as corn starch and potato starch; (c) cellulose, and its derivatives, such as sodium carboxymethyl cellulose, ethyl cellulose and cellulose acetate; (d) powdered tragacanth; (e) malt; (f) gelatin; (g) talc; (h) excipients, such as cocoa butter and suppository waxes; (i) oils, such as peanut oil, cottonseed oil, safflower oil, sesame oil, olive oil, corn oil and soybean oil; (j) glycols, such as propylene glycol; (k) polyols, such as glycerin, sorbitol, mannitol and polyethylene glycol; (l) esters, such as ethyl oleate and ethyl laurate; (m) agar; (n) buffering agents, such as magnesium hydroxide and aluminum hydroxide; (o) alginic acid; (p) pyrogen-free water; (q) isotonic saline; (r) Ringer's solution; (s) ethyl alcohol; (t) phosphate buffer solutions; and (u) other non-toxic compatible substances employed in pharmaceutical formulations.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

For parenteral administration, the compounds, salts or conjugates may be formulated in a unit dosage injectable form (e.g., solution, suspension, emulsion) in association with a pharmaceutically acceptable parenteral vehicle. Such vehicles may be inherently non-toxic, and non-therapeutic. Vehicles may be water, saline, Ringer's solution, dextrose solution, and 5% human serum albumin. Non-aqueous vehicles such as fixed oils and ethyl oleate may also be used. Liposomes may be used as carriers. The vehicle may contain minor amounts of additives such as substances that enhance isotonicity and chemical stability (e.g., buffers and preservatives).

Sustained-release preparations may be also be prepared. Examples of sustained-release preparations may include semipermeable matrices of solid hydrophobic polymers that may contain the compound, salt or conjugate, and these matrices may be in the form of shaped articles (e.g., films or microcapsules). Examples of sustained-release matrices may include polyesters, hydrogels (e.g., poly(2-hydroxyethyl-methacrylate), or poly(vinyl alcohol)), polylactides, copolymers of L-glutamic acid and γ ethyl-L-glutamate, non-degradable ethylene-vinyl acetate, degradable lactic acid-glycolic acid copolymers such as the LUPRON DEPO™ (i.e., injectable microspheres composed of lactic acid-glycolic acid copolymer and leuprolide acetate), and poly-D-(−)-3-hydroxybutyric acid.

Pharmaceutical formulations may be prepared for storage by mixing a compound, salt or conjugate with a pharmaceutically acceptable carrier, excipient, and/or a stabilizer. This formulation may be a lyophilized formulation or an aqueous solution. Acceptable carriers, excipients, and/or stabilizers may be nontoxic to recipients at the dosages and concentrations used. Acceptable carriers, excipients, and/or stabilizers may include buffers such as phosphate, citrate, and other organic acids; antioxidants including ascorbic acid and methionine; preservatives, polypeptides; proteins, such as serum albumin or gelatin; hydrophilic polymers; amino acids; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes; and/or non-ionic surfactants or polyethylene glycol.

Pharmaceutical formulations of the conjugates may have an average drug-antibody construct ratio (“DAR”) selected from about 1 to about 20 or from about 1 to about 10, wherein the drug is a compound or salt of any one of Formulas (IA), (IB), (IC), (ID), (IE), (IF), and Table 16. In certain embodiments, the average DAR of the formulation is from about 2 to about 8, such as from about 3 to about 8, such as from about 3 to about 7, such as about 3 to about 5 or such as about 2. In certain embodiments, a pharmaceutical formulation has an average DAR of about 3, about 3.5, about 4, about 4.5 or about 5.

### Therapeutic Applications

The compositions, conjugates and methods of the present disclosure can be useful for a plurality of different subjects including, but are not limited to, a mammal, human, non-human mammal, a domesticated animal (e.g., laboratory animals, household pets, or livestock), non-domesticated animal (e.g., wildlife), dog, cat, rodent, mouse, hamster, cow, bird, chicken, fish, pig, horse, goat, sheep, rabbit, and any combination thereof.

The compositions, conjugates and methods can be useful as a therapeutic, for example, a treatment that can be administered to a subject in need thereof. A therapeutic effect of the present disclosure can be obtained in a subject by reduction, suppression, remission, or eradication of a disease state, including, but not limited to, a symptom thereof. A therapeutic effect in a subject having a disease or condition, or pre-disposed to have or is beginning to have the disease or condition, can be obtained by a reduction, a suppression, a prevention, a remission, or an eradication of the condition or disease, or pre-condition or pre-disease state.

In practicing the methods described herein, therapeutically-effective amounts of the compositions, and conjugates can be administered to a subject in need thereof, often for treating and/or preventing a condition or progression thereof. A pharmaceutical composition can affect the physiology of the subject, such as the immune system, an inflammatory response, or other physiologic affect. A therapeutically-effective amount can vary depending on the severity of the disease, the age and relative health of the subject, the potency of the compounds used, and other factors.

Treat and/or treating refer to any indicia of success in the treatment or amelioration of the disease or condition. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. Treat can be used herein to refer to a method that results in some level of treatment or amelioration of the disease or condition, and can contemplate a range of results directed to that end, including but not restricted to prevention of the condition entirely.

Prevent, preventing and the like refer to the prevention of the disease or condition, e.g., tumor formation, in the patient. For example, if an individual at risk of developing a tumor or other form of cancer is treated with the methods of the present disclosure and does not later develop the tumor or other form of cancer, then the disease has been prevented, at least over a period of time, in that individual. Preventing can also refer to preventing re-occurrence of a disease or condition in a patient that has previously been treated for the disease or condition, e.g., by preventing relapse.

A therapeutically effective amount (also referred to as an effective amount) can be the amount of a composition (e.g., conjugate or compound) or an active component thereof sufficient to provide a beneficial effect or to otherwise reduce a detrimental non-beneficial event to the individual to whom the composition is administered. A therapeutically effective dose can be a dose that produces one or more desired or desirable (e.g., beneficial) effects for which it is administered, such administration occurring one or more times over a given period of time. An exact dose can depend on the purpose of the treatment, and can be ascertainable by one skilled in the art using known techniques and the teachings provided herein.

The conjugates that can be used in therapy can be formulated and dosages established in a fashion consistent with good medical practice taking into account the disease or condition to be treated, the condition of the individual patient, the site of delivery of the composition, the method of administration and other factors known to practitioners. The compositions can be prepared according to the description of preparation described herein.

Pharmaceutical compositions can be used in the methods described herein and can be administered to a subject in need thereof using a technique known to one of ordinary skill in the art which can be suitable as a therapy for the disease or condition affecting the subject. One of ordinary skill in the art would understand that the amount, duration and frequency of administration of a pharmaceutical composition to a subject in need thereof depends on several factors including, for example but not limited to, the health of the subject, the specific disease or condition of the patient, the grade or level of a specific disease or condition of the patient, the additional treatments the subject is receiving or has received, and the like. The methods and compositions can be for administration to a subject in need thereof.

Often, administration of the compositions can include routes of administration, non-limiting examples of administration routes include intravenous, intraarterial, subcutaneous, subdural, intramuscular, intracranial, intrasternal, intratumoral, or intraperitoneally. Additionally, a pharmaceutical composition can be administered to a subject by additional routes of administration, for example, by inhalation, oral, dermal, intranasal, or intrathecal administration.

Compositions and conjugates of the present disclosure can be administered to a subject in need thereof in a first administration, and in one or more additional administrations. The one or more additional administrations can be administered to the subject in need thereof minutes, hours, days, weeks or months following the first administration. Any one of the additional administrations can be administered to the subject in need thereof less than 21 days, or less than 14 days, less than 10 days, less than 7 days, less than 4 days or less than 1 day after the first administration. The one or more administrations can occur more than once per day, more than once per week or more than once per month. The administrations can be weekly, biweekly (every two weeks), every three weeks, monthly or bimonthly.

The compositions, conjugates and methods provided herein may be useful for the treatment of a plurality of diseases, conditions, preventing a disease or a condition in a subject or other therapeutic applications for subjects in need thereof. Often the compositions, conjugates and methods provided herein may be useful for treatment of hyperplastic conditions, including but not limited to, neoplasms, cancers, tumors and the like. The compositions, conjugates and methods provided herein may be useful in specifically targeting TGFβ1, TGFβR1, TGFβR2, or combinations thereof. The compositions and methods provided herein may be useful in inhibiting TGFβ1, TGFβR1, TGFβR2, or combinations thereof. In one embodiment, the compounds of the present disclosure activate or enhance an immune response. In another embodiment, the conjugates of the present disclosure activate or enhance an immune response.

A condition, such as a cancer, may be associated with expression of a molecule on the cancer cells. Often, the molecule expressed by the cancer cells may comprise an extracellular portion capable of recognition by the antibody construct of the conjugate. A molecule expressed by the cancer cells may be a tumor antigen. An antibody construct of the conjugate may recognize a tumor antigen.

In certain embodiments, the antigen binding domain specifically binds to an antigen that is at least 80% identical to an antigen on a T cell, a B cell, a stellate cell, an endothelial cell, a tumor cell, an APC, a fibroblast cell, a fibrocyte cell, or a cell associated with the pathogenesis of fibrosis. In certain embodiments, the antigen binding domain specifically binds to an antigen that is at least 80% identical to an antigen on a T cell, an APC, and/or a B cell. In certain embodiments, the antigen binding domain may specifically bind to an antigen that is at least 80% identical to an antigen selected from the group consisting of CLTA4, PD-1, OX40, LAG-3, GITR, GARP, CD25, CD27, PD-L1, TNFR2, ICOS, 41BB, CD70, CD73, CD38, or VTCN1. In certain embodiments, the antigen binding domain specifically binds to an antigen that is at least 80% identical to an antigen on a stellate cell, an endothelial cell, a fibroblast cell, a fibrocyte cell, or a cell associated with the pathogenesis of fibrosis or cancer. In certain embodiments, the antigen binding domain may specifically bind to an antigen that is at least 80% identical to an antigen selected from the group consisting of PDGFRβ, integrin αvβ1, integrin αvβ3, integrin αvβ6, integrin αvβ8, Endosialin, FAP, ADAM12, LRRC15, MMP14, PDPN, CDH11 and F2RL2, In certain embodiments, the antigen binding domain may specifically bind to an antigen that is at least 80% identical to an antigen selected from the group consisting of FAP, ADAM12, LRRC15, MMP14, PDPN, CDH11 and F2RL2, In certain embodiments, the antigen binding domain specifically binds to an antigen that is at least 80% identical to an antigen on a tumor cell, a tumor antigen. In certain embodiments, the antigen binding domain specifically binds to an antigen that is at least 80% identical to an antigen selected from the group consisting of MUC16, UPK1B, VTCN1, TMPRSS3, TMEM238, Clorf186, TMPRSS4, CLDN6, CLDN8, STRA6, MSLN or CD73.

In certain embodiments, the antigen binding domain specifically binds to an antigen on a T cell, a B cell, a stellate cell, an endothelial cell, a tumor cell, an APC, a fibroblast cell, a fibrocyte cell, or a cell associated with the pathogenesis of fibrosis. In certain embodiments, the antigen binding domain specifically binds to an antigen on a T cell, an APC, and/or a B cell. In certain embodiments, the antigen binding domain may specifically bind to an antigen selected from the group consisting of CLTA4, PD-1, OX40, LAG-3, GITR, GARP, CD25, CD27, PD-L1, TNFR2, ICOS, 41BB, CD70, CD73, CD38 or VTCN1. In certain embodiments, the antigen binding domain specifically binds to an antigen on a stellate cell, an endothelial cell, a fibroblast cell, a fibrocyte cell, or a cell associated with the pathogenesis of fibrosis or cancer. In certain embodiments, the antigen binding domain may specifically bind to an antigen selected from the group consisting of, PDGFRβ, integrin αvβ1, integrin αvβ3, integrin αvβ6, integrin αvβ8, Endosialin, FAP, ADAM12, LRRC15, MMP14, PDPN, CDH11 and F2RL2. In certain embodiments, the antigen binding domain may specifically bind to an antigen selected from the group consisting of FAP, ADAM12, LRRC15, MMP14, PDPN, CDH11 and F2RL2. In certain embodiments, the antigen binding domain specifically binds to an antigen on a tumor cell, a tumor antigen. In certain embodiments, the antigen binding domain specifically binds to an antigen selected from the group consisting of MUC16, UPK1B, VTCN1, TMPRSS3, TMEM238, Clorf186, TMPRSS4, CLDN6, CLDN8, STRA6, MSLN or CD73.

Additionally, such antigens may be derived from the following specific conditions and/or families of conditions, including but not limited to, cancers such as brain cancers, skin cancers, lymphomas, sarcomas, lung cancer, liver cancer, leukemias, uterine cancer, breast cancer, ovarian cancer, cervical cancer, bladder cancer, kidney cancer, hemangiosarcomas, bone cancers, blood cancers, testicular cancer, prostate cancer, stomach cancer, intestinal cancers, pancreatic cancer, and other types of cancers as well as pre-cancerous conditions such as hyperplasia or the like.

Non-limiting examples of cancers may include Acute lymphoblastic leukemia (ALL); Acute myeloid leukemia; Adrenocortical carcinoma; Astrocytoma, childhood cerebellar or cerebral; Basal-cell carcinoma; Bladder cancer; Bone tumor, osteosarcoma/malignant fibrous histiocytoma; Brain cancer; Brain tumors, such as, cerebellar astrocytoma, malignant glioma, ependymoma, medulloblastoma, visual pathway and hypothalamic glioma; Brainstem glioma; Breast cancer; Bronchial adenomas/carcinoids; Burkitt's lymphoma; Cerebellar astrocytoma; Cervical cancer; Cholangiocarcinoma; Chondrosarcoma; Chronic lymphocytic leukemia; Chronic myelogenous leukemia; Chronic myeloproliferative disorders; Colon cancer; Cutaneous T-cell lymphoma; Endometrial cancer; Ependymoma; Esophageal cancer; Eye cancers, such as, intraocular melanoma and retinoblastoma; Gallbladder cancer; Glioma; Hairy cell leukemia; Head and neck cancer; Heart cancer; Hepatocellular (liver) cancer; Hodgkin lymphoma; Hypopharyngeal cancer; Islet cell carcinoma (endocrine pancreas); Kaposi sarcoma; Kidney cancer (renal cell cancer); Laryngeal cancer; Leukaemia, such as, acute lymphoblastic, acute myeloid, chronic lymphocytic, chronic myelogenous and, hairy cell; Lip and oral cavity cancer; Liposarcoma; Lung cancer, such as, non-small cell and small cell; Lymphoma, such as, AIDS-related, Burkitt; Lymphoma, cutaneous T-Cell, Hodgkin and Non-Hodgkin, Macroglobulinemia, Malignant fibrous histiocytoma of bone/osteosarcoma; Melanoma; Merkel cell cancer; Mesothelioma; Multiple myeloma/plasma cell neoplasm; Mycosis fungoides; Myelodysplastic syndromes; Myelodysplastic/myeloproliferative diseases; Myeloproliferative disorders, chronic; Nasal cavity and paranasal sinus cancer; Nasopharyngeal carcinoma; Neuroblastoma; Oligodendroglioma; Oropharyngeal cancer; Osteosarcoma/malignant fibrous histiocytoma of bone; Ovarian cancer; Pancreatic cancer; Parathyroid cancer; Pharyngeal cancer; Pheochromocytoma; Pituitary adenoma; Plasma cell neoplasia; Pleuropulmonary blastoma; Prostate cancer; Rectal cancer; Renal cell carcinoma (kidney cancer); Renal pelvis and ureter, transitional cell cancer; Rhabdomyosarcoma; Salivary gland cancer; Sarcoma, Ewing family of tumors; Sarcoma, Kaposi; Sarcoma, soft tissue; Sarcoma, uterine; Sezary syndrome; Skin cancer (non-melanoma); Skin carcinoma; Small intestine cancer; Soft tissue sarcoma; Squamous cell carcinoma; Squamous neck cancer with occult primary, metastatic; Stomach cancer; Testicular cancer; Throat cancer; Thymoma and thymic carcinoma; Thymoma; Thyroid cancer; Thyroid cancer, childhood; Uterine cancer; Vaginal cancer; Waldenstrom macroglobulinemia; Wilms tumor and any combination thereof.

Non-limiting examples of fibrosis or fibrotic diseases include adhesive capsulitis, arterial stiffness, arthrofibrosis, atrial fibrosis, cirrhosis, Crohn's disease, collagenous fibroma, cystic fibrosis, Desmoid-type fibromatosis, Dupuytren's contracture, elastofibroma, endomyocardial fibrosis, fibroma of tendon sheath, glial scar, idiopathic pulmonary fibrosis, keloid, mediastinal fibrosis, myelofibrosis, nuchal fibroma, nephrogenic systemic fibrosis, old myocardial infarction, Peyronie's disease, pulmonary fibrosis, progressive massive fibrosis, nonalcoholic steatohepatitis (otherwise known as NASH), radiation-induced lung injury, retroperitoneal fibrosis, scar, scleroderma/systemic sclerosis.

The invention provides any therapeutic compound or conjugate disclosed herein for use in a method of treatment of the human or animal body by therapy. Therapy may be by any mechanism disclosed herein, such as by stimulation of the immune system. The invention provides any therapeutic compound or conjugate disclosed herein for use in stimulation of the immune system, vaccination or immunotherapy, including for example enhancing an immune response. The invention further provides any therapeutic compound or conjugate disclosed herein for prevention or treatment of any condition disclosed herein, for example cancer, autoimmune disease, inflammation, sepsis, allergy, asthma, graft rejection, graft-versus-host disease, immunodeficiency or infectious disease (typically caused by an infectious pathogen).

The invention also provides any therapeutic compound or conjugate disclosed herein for obtaining any clinical outcome disclosed herein for any condition disclosed herein, such as reducing tumour cells in vivo. The invention also provides use of any therapeutic compound or conjugate disclosed herein in the manufacture of a medicament for preventing or treating any condition disclosed herein.

### Examples

### List of Abbreviations

As used above, and throughout the description of the invention, the following abbreviations, unless otherwise indicated, shall be understood to have the following meanings:
ACN or MeCN acetonitrile
Bn benzyl
BOC or Boc tert-butyl carbamate
CDI 1,1′-carbonyl diimidazole
Cy cyclohexyl
DCE dichloroethane (ClCH₂CH₂C₁)
DCM dichloromethane (CH₂Cl₂)
DIPEA or DIEA diisopropylethylamine
DMAP 4-(N,N-dimethylamino)pyridine
DMF dimethylformamide
DMA N,N-dimethylacetamide
DMSO dimethylsulfoxide
equiv equivalent(s)
Et ethyl
EtOH ethanol
EtOAc ethyl acetate
h hour(s)
HATU 1-[bis(dimethylamino)methylene]-1H-1,2,3-triazolo[4,5-b]pyridinium 3-oxid hexafluorophosphate
HFIP 1,1,1,3,3,3-hexafluoropropan-2-ol
HPLC high performance liquid chromatography
LAH lithium aluminum hydride
LCMS liquid chromatography-mass spectrometry
mc-Val-Cit-PAB-PNP [4-[[(2S)-5-(carbamoylamino)-2-[[(2S)-2-[6-(2,5-dioxopyrrol-1-yl)hexanoylamino]-3-methylbutanoyl]amino]pentanoyl]amino]phenyl]methyl(4-nitrophenyl) carbonate
Me methyl
MeOH methanol
MS mass spectroscopy
NMM N-methylmorpholine
NMR nuclear magnetic resonance
PdCl₂(dppf) [1,1′-Bis(diphenylphosphino)ferrocene]dichloropalladium(II)
Pd(OH)₂ palladium hydroxide
PMB para-methoxybenzyl
rt room temperature
TEA triethylamine
TFA trifluoroacetic acid
THF tetrahydrofuran
TLC thin layer chromatography

### General Synthetic Schemes and Examples

The following synthetic schemes are provided for purposes of illustration, not limitation. The following examples illustrate the various methods of making compounds described herein. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

### General Synthetic Schemes and Examples

The following synthetic schemes are provided for purposes of illustration, not limitation. The following examples illustrate the various methods of making compounds described herein. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

### General Scheme I for the Preparation of Exemplary ALK5 Inhibitors

[chemical structure]

[chemical structure]

### Example 1

### Synthesis of 1-(5-([1,2,4]triazolo[1,5-a]pyridin-6-yl)-4-(6-methylpyridin-2-yl)-1H-imidazol-2-yl)-N-benzylmethanamine (Compound 1)

[chemical structure]

### Step A. Preparation of Int 1a

[chemical structure]

To a stirred solution of 1-([1,2,4]triazolo[1,5-a]pyridin-6-yl)-2-(6-methylpyridin-2-yl) ethane-1,2-dione (CAS 356560-84-4; 2.7 g, 10.1 mmol) in a 2:1 mixture of tert-butyl methyl ether and methanol (25 mL) were added 60% 2,2-dimethoxyacetaldehyde in H₂O (3.5 mL, 20 mmol) and NH₄OAc (1.95 g, 25.2 mmol). The mixture was stirred at room temperature for 5 h before the solvent was removed. The pH of the reaction mixture was adjusted to 8 with saturated aqueous NaHCO₃ solution and extracted with CH₂Cl₂ (2×10 mL). The combined organic extracts were washed with brine (10 mL) and dried over anhydrous Na₂SO₄, filtered, and evaporated. The residue was purified on silica gel (ISCO gold, 40 g; 0% to 20% CH₂C₁₋₂/MeOH over 15 minutes) to give the desired imidazole product which dissolved in 1 N HCl (20 mL) and heated at 70° C. for 4 h. The reaction mixture was allowed to cool to 0° C. and then it was neutralized with saturated aqueous NaHCO₃ solution. The precipitate was collected and washed with water (20 mL) and ether (40 mL) to give the desired product Int 1a as a yellow-brown solid. ¹H NMR (DMSO-d⁶) δ 10.0 (dd, J=1.6, 0.8 Hz, 1H), 9.56 (s, 1H), 8.42 (s, 1H), 8.27 (dd, J=9.2, 1.6 Hz, 1H), 7.82 (br d, J=0.8 Hz, 1H), 7.72 (dd, J=9.2, 0.8 Hz, 1H), 7.65 (t, J=7.8 Hz, 1H), 7.05 (d, J=7.6 Hz, 1H), 2.46 (s, 3H). LCMS (M+H)=305.1.

### Step C. Preparation of Compound 1

[chemical structure]

To a solution of 4-(6-methyl-2-pyridyl)-5-([1,2,4]triazolo[1,5-a]pyridin-6-yl)-1H-imidazole-2-carbaldehyde (200 mg, 0.66 mmol) in dichloroethane (30 mL) was added acetic acid (79 mg, 1.3 mmol, 75 uL) and phenylmethanamine (106 mg, 0.99 mmol). The mixture was stirred at 60° C. for 2 h then cooled to 0° C. Methanol (20 mL) and THF (10 mL) were added followed by NaBH₃CN (165 mg, 2.63 mmol, 4.0 eq) and then the reaction mixture was allowed to warm to 15° C. and stirred for an additional 3 h at which time LCMS showed the reaction to be complete. The reaction mixture was quenched by addition of 0.10 mL of water at 0° C. then concentrated under reduced pressure to give a residue that was purified by silica gel chromatography (0→10% MeOH in DCM) to afford 240 mg of 1-(5-([1,2,4]triazolo[1,5-a]pyridin-6-yl)-4-(6-methylpyridin-2-yl)-1H-imidazol-2-yl)-N-benzylmethanamine as a off-white solid. ¹H NMR (DMSO-d₆, 400 MHz) δ 9.54 (s, 1H), 8.55 (s, 1H), 7.99-7.96 (m, 1H), 7.92-7.88 (m, 1H), 7.79 (t, J=7.8 Hz, 1H), 7.58-7.53 (m, 3H), 7.49-7.41 (m, 3H), 7.26 (d, J=7.7 Hz, 1H), 4.37 (d, J=12.8 Hz, 4H), 2.52 (s, 3H). LCMS M/z 396.1 [M+H]⁺

The compounds in Table 16 were prepared in a manner similar to that described for compound 1 using the appropriately substituted amine.

TABLE 16

| Compound | | |
|---|---|---|
| Number | Structure | ¹H NMR |
| 1 | [chemical structure] | ¹H NMR (DMSO-d6, 400 MHz) δ 9.54 (s, 1H), 8.55 (s, 1H), 7.99-7.96 (m, 1H), 7.92-7.88 (m, 1H), 7.79 (t, J = 7.8 Hz, 1H), 7.58-7.53 (m, 3H), 7.49-7.41 (m, 3H), 7.26 (d, J = 7.7 Hz, 1H), 4.37 (d, J = 12.8 Hz, 4H), 2.52 (s, 3H) m/z 396.1 [M + H]⁺ |
| 2 | [chemical structure] | ¹H NMR (DMSO-d₆, 400 MHz) δ 9.67 (s, 1H), 9.50 (s, 1H), 8.55 (s, 1H), 8.00-7.87 (m, 2H), 7.75 (t, J = 7.6 Hz, 1H), 7.68 (s, 1H), 7.55-7.48 (m, 4H), 7.22 (d, J = 8.0 Hz, 1H), 4.39 (s, 2H), 4.33 (s, 2H), 2.48 (s, 3H) m/z = 430.1 [M + H]⁺. |
| 3 | [chemical structure] | ¹H NMR (DMSO-d₆, 400 MHz) δ 9.50 (s, 1H), 8.56 (s, 1H), 7.97-7.87 (m, 1H), 7.79 (t, J = 8.0 Hz, 1H), 7.59-7.51 (m, 5H), 7.27 (d, J = 7.6 Hz, 1H), 4.38-7.33 (m, 4H), 2.52 (s, 3H) m/z = 430.3 [M + H]⁺ |
| 4 | [chemical structure] | ¹H NMR (MeOD, 400 MHz) δ 9.23-9.21 (m, 1H), 8.51 (s, 1H), 7.96 (t, J = 7.8 Hz, 1H), 7.88 (t, J = 1.6 Hz, 2H), 7.78 (d, J = 2.2 Hz, 1H), 7.67-7.63 (m, 1H), 7.57 (d, J = 8.0 Hz, 1H), 7.51-7.46 (m, 2H), 4.48 (d, J = 4.6 Hz, 4H), 2.71 (s, 3H) m/z = 464.1 [M + H]⁺ |
| 5 | [chemical structure] | ¹H NMR (MeOD, 400 MHz) δ 9.23 (t, J = 1.4 Hz, 1H), 8.52 (s, 1H), 7.97 (t, J = 7.8 Hz, 1H), 7.89 (dd, J = 1.4, 2.2 Hz, 2H), 7.73 (d, J = 2.4 Hz, 1H), 7.60-7.53 (m, 2H), 7.51-7.46 (m, 2H), 4.63 (s, 2H), 4.55 (s, 2H), 2.71 (s, 3H) m/z = 464.1[M + H]⁺ |
| 6 | [chemical structure] | ¹H NMR (CDCl₃, 400 MHz) δ 8.93 (s, 1H), 8.44 (s, 1H), 7.98-7.86 (m, 2H), 7.70-7.61 (m, 1H), 7.45-7.39 (m, 4H), 7.38-7.33 (m, 1H), 4.73 (s, 2H), 4.55 (s, 2H), 2.89 (s, 3H) m/z = 464.1 [M + H]⁺ |
| 7 | [chemical structure] | ¹H NMR (MeOD, 400 MHz) δ 9.15 (s, 1H), 8.42 (s, 1H), 7.87 (dd, J = 8.0 Hz, J = 4.0 Hz 1H), 7.75 (d, J = 8.4 Hz, 1H), 7.63 (t, J = 8.0 Hz, 1H), 7.34 (d, J = 7.6 Hz, 1H), 7.23-7.15 (m, 2H), 6.95-6.92 (m, 2H), 6.78 (dd, J = 12.0 Hz, J = 4.0 Hz 1H), 3.94 (s, 2H), 3.82 (s, 2H), 3.77 (s, 3H), 2.53 (s, 3H) m/z = 426.2 [M + H]⁺ |
| 8 | [chemical structure] | ¹H NMR (DMSO-d6, 400 MHz) δ 9.49 (s, 1H), 8.56 (s, 1H), 7.97-7.89 (m, 2H), 7.81 (t, J = 7.6 Hz, 1H), 7.53 (d, J = 8.0 Hz, 1H), 7.48 (d, J = 8.8 Hz, 2H), 7.28 (d, J = 7.6 Hz, 1H), 7.02 (d, J = 6.8 Hz 2H), 4.30 (d, J = 2.0 Hz, 4H), 3.77 (s, 3H), 2.53 (s, 3H) m/z = 426.0 [M + H]⁺ |
| 9 | [chemical structure] | ¹H NMR (MeOD, 400 MHz) δ 9.18 (s, 1H), 8.54 (s, 1H), 8.11 (t, J = 8.0 Hz, 1H), 7.95-7.81 (m, 2H), 7.62 (dd, J = 14.6, 8.0 Hz, 2H), 7.02 (s, 3H), 4.45 (d, J = 10.8 Hz, 4H), 3.87 (s, 3H), 3.75 (s, 3H), 2.79 (s, 3H) m/z = 456.1 [M + H]⁺ |
| 10 | [chemical structure] | ¹H NMR (MeOD, 400 MHz) δ 9.17 (s, 1H), 8.43 (s, 1H), 7.88 (dd, J = 1.6, 9.4 Hz, 1H), 7.78-7.74 (m, 1H), 7.67 (t, J = 7.8 Hz, 1H), 7.50 (dd, J = 1.6, 7.2 Hz, 1H), 7.39-7.35 (m, 2H), 7.31-7.22 (m, 2H), 7.18 (d, J = 7.8 Hz, 1H), 3.98 (d, J = 4.0 Hz, 4H), 2.51 (s, 3H) m/z = 430.2 [M + H]⁺ |
| 11 | [chemical structure] | ¹H NMR (MeOD, 400 MHz) δ 9.15 (s, 1H), 8.54 (s, 1H), 8.15 (t, J = 8.0 Hz, 1H), 7.95-7.79 (m, 2H), 7.65 (d, J = 7.6 Hz, J = 4.4 Hz 2H), 7.58 (d, J = 6.8 Hz, 2H), 7.53-7.43 (m, 3H), 4.70 (q, J = 6.4 Hz, 1H), 4.42-4.15 (m, 2H), 2.83 (s, 3H), 1.81 (d, J = 6.8 Hz, 3H) m/z = 410.1 [M + H]⁺ |
| 12 | [chemical structure] | ¹H NMR (MeOD, 400 MHz) δ 9.16 (s, 1H), 8.53 (s, 1H), 8.08 (t, J = 7.8 Hz, 1H), 7.91-7.88 (m, 1H), 7.85-7.81 (m, 1H), 7.58-7.54 (m, 2H), 7.50- 7.46 (m, 3H), 4.70 (q, J = 6.8 Hz, 1H), 4.37-4.31 (m, 1H), 4.23-4.18 (m, 1H), 2.78 (s, 3H), 1.80 (d, J = 6.8 Hz, 3H) m/z = 410.1 [M + H]⁺ |
| 13 | [chemical structure] | ¹H NMR (MeOD, 400 MHz) δ 9.13 (s, 1H), 8.40 (s, 1H), 7.89-7.58 (m, 3H), 7.47-7.09 (m, 12H), 4.93 (s, 1H), 3.91 (s, 2H), 2.49 (s, 3H) m/z = 472.3 [M + H]⁺ |
| 14 | [chemical structure] | ¹H NMR (MeOD, 400 MHz) δ 9.15 (s, 1H), 8.42 (s, 1H), 7.91-7.62 (m, 3H), 7.41-7.14 (m, 7H), 3.94-3.77 (m, 3H), 3.71-3.58 (m, 2H), 2.51 (s, 3H) m/z = 426.2 [M + H]⁺ |
| 15 | [chemical structure] | ¹H NMR (MeOD, 400 MHz) δ 9.17 (br s, 1H), 8.43 (s, 1H), 7.91-7.62 (m, 3H), 7.51-7.26 (m, 6H), 7.18 (d, J = 7.6 Hz, 1H), 4.63-4.48 (m, 1H), 3.92 (d, J = 2.2 Hz, 2H), 3.67 (s, 3H), 2.51 (s, 3H) m/z = 454.2 [M + H]⁺ |
| 16 | [chemical structure] | H NMR (MeOD, 400 MHz) δ 9.20 (s, 1H), 8.53 (s, 1H), 7.96-7.83 (m, 3H), 7.58-7.44 (m, 7H), 5.34 (s, 1H), 4.39-4.26 (m, 2H), 3.80 (s, 3H), 2.69 (s, 3H) m/z found 454.1 [M + H]⁺. |
| 17 | [chemical structure] | ¹H NMR (MeOD, 400 MHz) δ 9.18 (s, 1H), 8.54 (s, 1H), 8.15 (t, J = 8.0 Hz, 1H), 7.95-7.90 (m, 1H), 7.87-7.83 (m, 1H), 7.66 (dd, J = 8.0, 11.2 Hz, 2H), 7.50-7.40 (m, 2H), 7.10 (d, J = 8.4 Hz, 1H), 7.02 (dt, J = 0.8, 7.5 Hz, 1H), 4.48 (d, J = 3.6 Hz, 4H), 3.95-3.89 (m, 3H), 2.82 (s, 3H) m/z = 426.6 [M + H]⁺. |
| 18 | [chemical structure] | ¹H NMR (MeOD, 400 MHz) δ 9.19 (s, 1H), 8.54 (s, 1H), 8.17 (t, J = 8.0 Hz, 1H), 7.94-7.83 (m, 2H), 7.70-7.63 (m, 3H), 7.55-7.49 (m, 1H), 7.32- 7.23 (m, 2H), 4.56 (s, 2H), 4.59 (s, 2H), 2.84 (s, 3H) m/z = 414.2 [M + H]⁺ |
| 19 | [chemical structure] | ¹H NMR (MeOD, 400 MHz) δ 9.18 (s, 1H), 8.42 (s, 1H), 7.88 (dd, J = 1.6, 9.2 Hz, 1H), 7.82-7.75 (m, 2H), 7.70-7.65 (m, 2H), 7.62 (t, J = 7.6 Hz, 1H), 7.46-7.41 (m, 1H), 7.38 (d, J = 7.6 Hz, 1H), 7.18 (d, J = 7.6 Hz, 1H), 4.04 (s, 2H), 4.00 (s, 2H), 2.51 (s, 3H) m/z = 430.1 [M + H]⁺. |
| 20 | [chemical structure] | ¹H NMR (MeOD, 400 MHz) δ 9.16 (s, 1H), 8.49 (s, 1H), 8.15 (d, J = 8.0 Hz, 1H), 7.91 (t, J = 7.6 Hz, 1H), 7.87-7.81 (m, 3H), 7.78-7.74 (m, 1H), 7.72-7.67 (m, 1H), 7.52 (d, J = 8.0 Hz, 1H), 7.43 (d, J = 7.6 Hz, 1H), 5.29 (s, 2H), 5.06 (s, 2H), 2.65 (s, 3H) m/z = 421.2 [M + H]⁺. |
| 21 | [chemical structure] | ¹H NMR (MeOD, 400 MHz) δ 9.18 (s, 1H), 8.43 (s, 1H), 7.89 (dd, J = 1.6, 9.2 Hz, 1H), 7.79-7.76 (m, 1H), 7.67 (t, J = 7.8 Hz, 1H), 7.39-7.31 (m, 2H), 7.19 (d, J = 7.8 Hz, 1H), 7.16-7.12 (m, 3H), 4.00 (s, 2H), 3.85 (s, 2H), 2.52 (s, 3H), 2.35 (s, 3H) m/z = 410.2 [M + H]⁺ |
| 22 | [chemical structure] | ¹H NMR (MeOD, 400 MHz) δ 9.18 (s, 1H), 8.42 (s, 1H), 7.89 (d, J = 9 .2 Hz, 1H), 7.79-7.75 (m, 1H), 7.66 (t, J = 7.6 Hz, 1H), 7.40-7.33 (m, 2H), 7.20-7.16 (m, 3H), 7.16-7.12 (m, 1H), 4.00 (s, 2H), 3.87 (s, 2H), 2.70 (q, J = 7.6 Hz, 2H), 2.51 (s, 3H), 1.18 (t, J = 7.6 Hz, 3H) m/z = 424.2 [M + H]⁺. |
| 23 | [chemical structure] | ¹H NMR (MeOD, 400 MHz) δ 9.53 (s, 1H), 8.55 (s, 1H), 8.00-7.89 (m, 2H), 7.78 (t, J = 7.8 Hz, 1H), 7.56-7.50 (m, 2H), 7.40 (d, J = 3.8 Hz, 2H), 7.31-7.23 (m, 2H), 4.47 (s, 2H), 4.50 (s, 2H), 3.31 (td, J = 6.6, 13.6 Hz, 1H), 2.52 (s, 3H), 1.18 (d, J = 6.8 Hz, 6H) m/z 438.2 [M + H]⁺ |
| 24 | [chemical structure] | ¹H NMR (MeOD, 400 MHz) δ 9.11 (s, 1H), 8.42 (s, 1H), 7.86-7.72 (m, 2H), 7.65 (t, J = 7.8 Hz, 1H), 7.56 (d, J = 7.4 Hz, 1H), 7.41-7.23 (m, 7H), 7.22-7.13 (m, 3H), 3.81 (d, J = 17.6 Hz, 4H), 2.51 (s, 3H) m/z = 472.2 [M + H]⁺ |
| 25 | [chemical structure] | ¹H NMR (MeOD, 400 MHz) δ 9.15 (s, 1H), 8.42 (s, 1H), 7.87 (d, J = 8.4 Hz, 1H), 7.77 (d, J = 9.2 Hz, 1H), 7.65 (t, J = 7.8 Hz, 1H), 7.35 (dd, J = 3.4, 7.2 Hz, 2H), 7.28-7.12 (m, 3H), 7.04 (t, J = 8.0 Hz 1H), 3.94 (d, J = 6.8 Hz, 4H), 2.65 (s, 6H), 2.52 (s, 3H) m/z = 439.2 [M + H]⁺ |
| 26 | [chemical structure] | ¹H NMR (MeOD, 400 MHz) δ 9.50 (s, 1H), 8.56 (s, 1H), 7.97-7.89 (m, 2H), 7.83-7.76 (m, 2H), 7.61-7.44 (m, 5H), 7.28 (d, J = 7.8 Hz, 1H), 4.45 (s, 4H), 4.52 (s, 4H), 2.53 (s, 3H) m/z = 480.2 [M + H]⁺ |
| 27 | [chemical structure] | ¹H NMR (DMSO-d6, 400 MHz) δ 9.49 (s, 1H), 8.56 (s, 1H), 7.97-7.92 (m, 1H), 7.74 (t, J = 7.6 Hz, 1H), 7.66 (s, 1H), 7.52-7.47 (m, 4H), 7.25 (d, J = 7.6 Hz, 1H), 4.53 (s, 2H), 4.47 (s, 2H), 3.92 (q, J = 10.4 Hz, 2H), 2.51 (s, 3H) m/z = 478.2 [M + H]⁺. |
| 28 | [chemical structure] | ¹H NMR (MeOD, 400 MHz) δ 9.21 (s, 1H), 9.52 (s, 1H), 8.03 (t, J = 8.0 Hz, 1H), 7.89 (s, 2H), 7.63-7.61 (m, 2H), 7.60-7.52 (m, 4H), 5.41 (q, J = 7.6 Hz, 1H), 4.57-4.47 (m, 4H), 2.74 (s, 3H) m/z = 494.2 [M + H]⁺. |
| 29 | [chemical structure] | ¹H NMR (MeOD, 400 MHz) δ 9.15 (s, 1H), 8.52 (s, 1H), 7.93 (t, J = 7.8 Hz, 1H), 7.87-7.83 (m, 2H), 7.78-7.74 (m, 1H), 7.60-7.49 (m, 3H), 7.45 (d, J = 7.8 Hz, 1), 7.39-7.34 (m, 1H),7.29-7.24 (m, 1H), 7.21-7.17 (m, 2H), 7.15-7.11 (m, 1H), 4.43-4.26 (m, 4H), 3.96-3.85 (m, 2H), 2.69 (s, 3H), 1.35 (s, 9H) m/z = 601.3 [M + H]⁺ |
| 30 | [chemical structure] | ¹H NMR (DMSO-d₆, 400 MHz) δ 9.61-9.39 (m, 2H), 8.55 (s, 1H), 7.91-7.71 (m, 4H), 7.54-7.48 (m, 2H), 7.37-7.12 (m, 7H), 4.39 (s, 2H), 4.24 (s, 2H), 4.11 (s, 2H), 2.42 (m, 3H), 1.36 (s, 9H) m/z = 601.3 [M + H]⁺ |
| 31 | [chemical structure] | ¹H NMR (DMSO-d₆, 400 MHz) δ 9.41 (s, 1H), 8.60-8.51 (m, 1H), 7.93-7.83 (m, 2H), 7.78- 7.71 (m, 2H), 7.54-7.46 (m, 2H), 7.45-7.34 (m, 2H), 7.34-7.26 (m, 3H), 7.26-7.20 (m, 3H), 4.40 (s, 2H), 4.25 (s, 2H), 4.04 (d, J = 6.0 Hz, 2H), 1.37 (s, 9H) m/z = 601.3 [M + H]⁺. |
| 32 | [chemical structure] | ¹H NMR (MeOD, 400 MHz) δ 9.25 (s, 1H), 8.72 (s, 1H), 8.30-8.23 (m, 1H), 8.01 (d, J = 8.8 Hz, 1H), 7.96-7.86 (m, 2H), 7.76 (d, J = 7.2 Hz, 2H), 7.66-7.51 (m, 3H), 7.44-7.28 (m, 4H), 4.49-4.38 (m, 3H), 4.22 (d, J = 13.8 Hz, 1H), 4.02 (d, J = 13.8 Hz, 1H), 3.84 (d, J = 14.0 Hz, 1H), 2.89 (s, 3H) m/z = 501.2 [M + H]⁺ |
| 33 | [chemical structure] | ¹H NMR (DMSO-d6, 400 MHz) δ 10.08 (s, 1H), 9.51 (s, 1H), 8.58 (s, 1H), 8.45 (s, 2H), 7.92-7.81 (m, 3H), 7.57-7.49 (m, 3H), 7.39-7.29 (m, 4H), 4.39 (s, 2H), 4.31 (s, 2H), 4.10-4.05 (m, 2H), 2.54 (s, 3H) m/z = 501.3 [M + H]⁺ |
| 34 | [chemical structure] | ¹H NMR (MeOD, 400 MHz) δ 9.20 (s, 1H), 8.52 (s, 1H), 7.92-7.83 (m, 3H), 7.78-7.74 (m, 1H), 7.62-7.53 (m, 2H), 7.52-7.35 (m, 7H), 4.51 (s, 2H), 4.32 (s, 2H), 3.97 (s, 2H), 2.66 (s, 3H) m/z = 501.3 [M + H]⁺. |
| 35 | [chemical structure] | ¹H NMR (DMSO, 400 MHz) δ 9.39 (s, 1H), 8.53 (s, 1H), 7.89-7.71 (m, 4H), 7.49 (dtd, J = 1.2, 7.2, 18.0 Hz, 2H), 7.40 (d, J = 8.0 Hz, 1H), 7.36- 7.27 (m, 3H), 7.24 (d, J = 7.2 Hz, 1H), 7.14-7.08 (m, 2H), 4.36 (s, 2H), 4.23 (s, 2H), 2.50 (s, 3H) m/z = 490.1 [M + H]⁺ |
| 36 | [chemical structure] | ¹H NMR (DMSO, 400 MHz) δ 9.41 (s, 1H), 8.52 (s, 1H), 7.84 (dq, J = 1.2, 9.2 Hz, 2H), 7.76-7.71 (m, 2H), 7.56-7.29 (m, 6H), 7.22 (d, J = 7.2 Hz, 1H), 7.10 (s, 1H), 4.38 (s, 2H), 4.23 (s, 2H), 2.50 (s, 3H) m/z = 508.2 [M + H]⁺ |
| 37 | [chemical structure] | ¹H NMR (MeOD, 400 MHz): δ 9.13 (s, 1H), 8.52 (s, 1H), 8.01 (s, 1H), 7.89-7.86 (m, 1H), 7.82-7.79 (m, 1H), 7.73-7.70 (m, 1H),7.55 (br d, J = 7.6 Hz, 4H), 7.37-7.27 (m, 2H), 7.10-7.04 (m, 2H), 6.92- 6.86 (m, 1H), 4.52 (s, 2H), 4.32 (s, 2H), 2.72 (s, 3H) m/z = 490.2 [M + H]⁺ |
| 38 | [chemical structure] | ¹H NMR (DMSO-d6, 400 MHz) δ 12.42 (s, 1H), 9.69-9.46 (m, 1H), 8.50 (s, 1H), 7.93 (d, J = 9.4 Hz, 1H), 7.83 (s, 1H), 7.70 (d, J = 6.8 Hz, 2H), 7.43 (t, J = 7.6 Hz, 1H), 7.36-7.31 (m, 4H), 7.24- 7.14 (m, 4H), 3.70 (s, 2H), 3.63 (s, 2H), 2.51 (s, 3H) m/z = 490.3 [M + H]⁺ |
| 39 | [chemical structure] | ¹H NMR (DMSO-d6, 400 MHz) δ 12.47 (s, 1H), 9.61-9.48 (m, 1H), 8.49 (s, 1H), 7.95 (dd, J = 1.6, 9.4 Hz, 1H), 7.82 (s, 1H), 7.70-7.63 (m, 2H), 7.42 (t, J = 6.8 Hz, 1H), 7.35 (t, J = 7.0 Hz, 1H), 7.28- 7.25 (m, 1H), 7.19 (d, J = 6.8 Hz, 4H), 3.78 (s, 2H), 3.71 (s, 2H), 2.49 (s, 3H) m/z = 508.3 [M + H]⁺ |
| 40 | [chemical structure] | ¹H NMR (MeOD, 400 MHz) δ 9.15 (s, 1H), 8.54 (d, J = 4.4 Hz, 1H), 8.44 (s, 1H), 7.88-7.76 (m, 3H), 7.67 (t, J = 7.8 Hz, 1H), 7.58 (dd, J = 7.8, 11.0 Hz, 2H), 7.46-7.40 (m, 4H), 7.35 (d, J = 7.8 Hz, 1H), 7.26-7.18 (m, 2H), 3.97 (s, 2H), 3.82 (s, 2H), 2.53 (s, 3H) m/z = 473.3 [M + H]⁺ |
| 41 | [chemical structure] | ¹H NMR (MeOD, 400 MHz) δ 9.16 (s, 1H), 8.53 (d, J = 1.6 Hz, 1H), 8.43 (s, 1H), 8.39 (dd, J = 1.6, 5.2 Hz, 1H), 7.90-7.81 (m, 2H), 7.80-7.75 (m, 1H), 7.70-7.59 (m, 2H), 7.44 (t, J = 7.5 Hz, 1H), 7.40-7.31 (m, 3H), 7.25 (dd, J = 1.2, 7.5 Hz, 1H), 7.19 (d, J = 7.6 Hz, 1H), 3.87 (s, 2H), 3.76 (s, 2H), 2.52 (s, 3H) m/z = 473.2 [M + H]⁺. |
| 42 | [chemical structure] | ¹H NMR (MeOD, 400 MHz) δ 9.12 (s, 1H), 8.42 (s, 3H), 7.89-7.71 (m, 2H), 7.70-7.59 (m, 2H), 7.43 (s, 3H), 7.34 (dd, J = 7.6, 16.4 Hz, 2H), 7.24 (d, J = 7.2 Hz, 1H), 7.16 (d, J = 7.2 Hz, 1H), 3.88 (s, 2H), 3.76 (s, 2H), 2.51 (s, 3H) m/z = 473.2 [M + H]⁺. |
| 43 | [chemical structure] | ¹H NMR (MeOD, 400 MHz) δ 9.26 (s, 1H), 8.51 (s, 1H), 7.98-7.90 (m, 2H), 7.89-7.85 (m, 1H), 7.58 (d, J = 8.0 Hz, 1H), 7.50 (d, J = 7.6 Hz, 1H), 7.45 (d, J = 7.6 Hz, 1H), 7.43-7.38 (m, 2H), 7.34- 7.23 (m, 1H), 4.53 (s, 4H), 2.69 (s, 3H), 1.72 (t, J = 13.6 Hz, 4H), 1.59-1.38 (m, 4H), 1.21 (s, 2H), 1.30-1.12 (m, 1H) m/z = 478.3 [M + H]⁺ |
| 44 | [chemical structure] | ¹H NMR (MeOD, 400 MHz) δ 9.21 (s, 1H), 8.44 (s, 1H), 7.91 (dd, J = 1.8, 9.2 Hz, 1H), 7.79 (d, J = 9.4 Hz, 1H), 7.67 (t, J = 7.8 Hz, 1H), 7.39 (d, J = 8.0 Hz, 1H), 7.31 (dd, J = 7.2, 14.6 Hz, 2H), 7.25- 7.17 (m, 2H), 7.16-7.10 (m, 1H), 4.00 (s, 2H), 3.89 (s, 2H), 3.18 (d, J = 8.4 Hz, 1H), 2.53 (s, 3H), 1.96-2.02 (m, 2H), 1.74-1.80 (m, 2H), 1.54- 1.62 (m, 4H) m/z = 464.3 [M + H]+ |
| 45 | [chemical structure] | ¹H NMR (MeOD, 400 MHz) δ 9.11 (s, 1H), 8.43 (s, 1H), 7.85 (d, J = 9.2 Hz, 1H), 7.78 (d, J = 9.2 Hz, 1H), 7.69 (t, J = 7.6 Hz, 1H), 7.35 (d, J = 7.6 Hz, 1H), 7.20 (d, J = 7.6 Hz, 1H), 7.14-7.10 (m, 1H), 7.02-7.01 (m, 2H), 3.99 (s, 2H), 3.84 (s, 2H), 2.50 (s, 3H), 2.23 (s, 6H) m/z = 424.1 [M + H]⁺. |
| 46 | [chemical structure] | ¹H NMR (MeOD, 400 MHz) δ 9.17 (s, 1H), 8.42 (s, 1H), 7.89 (d, J = 1.6 Hz, 1H), 7.87 (d, J = 1.6 Hz, 1H), 7.78-7.66 (m, 1H), 7.35 (d, J = 7.6 Hz, 1H), 7.17 (d, J = 7.6 Hz, 1H), 7.13-7.09 (m, 1H), 6.92 (d, J = 7.6 Hz, 1H), 6.81 (d, J = 8.4 Hz, 1H), 3.98 (s, 2H), 3.84 (s, 2H), 3.78 (s, 3H), 2.51 (s, 3H), 2.19 (s, 3H) m/z = 440.2 [M + H]⁺. |
| 47 | [chemical structure] | ¹H NMR (CDCl3, 400 MHz): δ 9.18 (s, 1H), 8.44 (s, 1H), 7.94-7.85 (m, 1H), 7.84-7.74 (m, 1H), 7.73-7.63 (m, 1H), 7.44-7.07 (m, 6H), 4.00 (s, 2H), 3.90 (s, 2H), 2.53 (s, 3H), 2.41 (s, 3H) m/z = 444.0 [M + H]+ |
| 48 | [chemical structure] | ¹H NMR (MeOD, 400 MHz): δ 9.15 (s, 1H), 8.46 (s, 1H), 7.89-7.84 (m, 1H), 7.82-7.78 (m, 1H), 7.69 (t, J = 7.6 Hz, 1H), 7.42-7.25 (m, 5H), 7.23- 7.18 (m, 3H), 7.17-7.12 (m, 2H), 3.81 (s, 2H), 3.57 (s, 2H), 2.55 (s, 3H), 1.99 (s, 3H) m/z = 486.0 [M + H]⁺ |
| 49 | [chemical structure] | ¹H NMR (MeOD, 400 MHz) δ 9.16 (s, 1H), 8.42 (s, 1H), 7.87 (dd, J = 1.6, 9.6 Hz, 1H), 7.65 (t, J = 7.6 Hz, 1H), 7.42-7.26 (m, 5H), 7.23-7.13 (m, 4H), 7.07 (d, J = 7.6 Hz, 1H), 7.81-6.59 (m, 1H), 4.03 (s, 2H), 3.92 (s, 2H), 2.50 (s, 3H), 2.21 (s, 3H) m/z = 486.3 [M + H]⁺. |
| 50 | [chemical structure] | ¹H NMR (MeOD, 400 MHz) δ 9.21 (s, 1H), 8.53 (s, 1H), 8.03 (t, J = 8.0 Hz, 1H), 7.89 (s, 2H), 7.61 (d, J = 8.0 Hz, 1H), 7.54 (d, J = 7.6 Hz, 1H), 7.41- 7.28 (m, 4H), 4.46 (s, 2H), 4.43 (s, 2H), 2.74 (s, 3H), 2.39 (s, 3H) m/z = 410.1 [M + H]⁺. |
| 51 | [chemical structure] | ¹H NMR (MeOD, 400 MHz): δ 9.18 (d, J = 1.6 Hz, 1H), 8.53-8.50 (m, 1H), 8.03-7.97 (m, 1H), 7.89-7.80 (m, 4H), 7.75-7.70 (m, 1H), 7.65-7.60 (m, 2H), 7.59-7.49 (m, 5H), 7.45-7.38 (m, 2H), 7.37-7.32 (m, 1H), 4.55 (s, 2H), 4.51 (s, 2H), 2.72 (s, 3H) m/z = 472.2 [M + H]⁺ |
| 52 | [chemical structure] | ¹H NMR (MeOD, 400 MHz): δ 9.16 (s, 1H), 8.42 (s, 1H), 7.90-7.83 (m, 1H), 7.79-7.73 (m, 1H), 7.70-7.62 (m, 1H), 7.39-7.33 (m, 1H), 7.30-7.12 3.94 (s, 2H), 2.93 (s, 2H), 2.83 (s, 2H), 2.51 (s, 3H) m/z = 410.1 [M + H]⁺ |
| 53 | [chemical structure] | ¹H NMR (MeOD, 400 MHz) δ 9.09 (s, 1H), 8.43 (s, 1H), 7.84-7.80 (m, 1H), 7.76-7.73 (m, 1H), 7.65 (t, J = 7.8 Hz, 1H), 7.50 (d, J = 7.8 Hz, 1H), 7.32 (d, J = 7.8 Hz, 1H), 7.27-7.12 (m, 6H), 7.02-6.98 (m, 1H), 6.89 (d, J = 7.8 Hz, 2H), 6.84 (d, J = 8.2 Hz, 1H), 3.96 (s, 2H), 3.87 (s, 2H), 2.52 (s, 3H) m/z = 488.3 [M + H]⁺ |
| 54 | [chemical structure] | ¹H NMR (MeOD, 400 MHz) δ 9.11 (s, 1H), 8.43 (s, 1H), 8.24 (d, J = 2.6 Hz, 1H), 8.19 (dd, J = 1.4, 4.6 Hz, 1H), 7.84-7.81 (m, 1H), 7.77-7.72 (m, 1H), 7.65 (t, J = 7.8 Hz, 1H), 7.56 (d, J = 6.1 Hz, 1H), 7.36-7.27 (m, 4H), 7.25-7.17 (m, 2H), 6.92 (d, J = 8.2 Hz, 1H), 3.96 (s, 2H), 3.88 (s, 2H), 2.52 (s, 3H) m/z = 489.3 [M + H]⁺ |
| 55 | [chemical structure] | ¹H NMR (MeOD, 400 MHz) δ 9.13 (s, 1H), 8.43 (s, 1H), 7.89-7.84 (m, 3H), 7.79-7.76 (m, 1H), 7.72-7.65 (m, 2H), 7.56 (t, J = 7.2 Hz, 1H), 7.48 (t, J = 6.8 Hz, 1H), 7.39 (d, J = 7.4 Hz, 2H), 7.19 (d, J = 7.8 Hz, 1H), 6.43 (d, J = 7.4 Hz, 2H), 3.90 (s, 2H), 3.71 (s, 2H), 2.53 (s, 3H) m/z = 489.3 [M + H]⁺ |
| 56 | [chemical structure] | ¹H NMR (MeOD, 400 MHz): δ 9.10 (s, 1H), 8.36 (s, 1H), 7.83-7.76 (m, 1H), 7.73-7.66 (m, 1H), 7.59 (t, J = 7.6 Hz, 1H), 7.32-7.19 (m, 4H), 7.15- 7.04 (m, 2H), 7.03-6.95 (m, 2H), 6.92-6.84 (m, 2H), 6.82-6.75 (m, 1H), 3.88 (s, 2H), 3.78 (s, 2H), 2.45 (s, 3H) m/z = 488.3 [M + H]⁺ |
| 57 | [chemical structure] | ¹H NMR (MeOD, 400 MHz): δ 9.11-9.08 (m, 1H), 8.36 (s, 1H), 8.19 (s, 2H), 7.82-7.78 (m, 2H), 7.71-7.67 (m, 1H), 7.62-7.56 (m, 1H), 7.35-7.26 (m, 6H), 7.17-7.14 (m, 1H), 7.13-7.09 (m, 1H), 7.08-7.05 (m, 1H), 6.89-6.85 (m, 1H), 3.89 (s, 2H), 3.81 (s, 2H), 2.44 (s, 3H) m/z = 489.3 [M + H]⁺ |
| 58 | [chemical structure] | ¹H NMR (MeOD, 400 MHz): δ 9.19 (s, 1H), 8.43 (s, 1H), 8.40-8.32 (m, 2H), 7.88 (dd, J = 9.2, 1.6 Hz, 1H), 7.81-7.74 (m, 1H), 7.68 (t, J = 7.6 Hz, 1H), 7.50-7.42 (m, 1H), 7.40-7.32 (m, 2H), 7.27- 7.16 (m, 2H), 7.05 (dd, J = 8.0, 1.6 Hz, 1H), 6.97- 6.90 (m, 2H), 4.01 (s, 2H), 3.95 (s, 2H), 2.53 (s, 3H) m/z = 489.3 [M + H]⁺ |
| 59 | [chemical structure] | ¹H NMR (MeOD, 400 MHz) δ 9.17 (s, 1H), 8.43 (s, 1H), 7.88 (d, J = 9.2 Hz, 1H), 7.77 (d, J = 9.2 Hz, 1H), 7.67 (t, J = 7.6 Hz, 1H), 7.37 (d, J = 8.0 Hz, 1H), 7.30-7.26 (m, 1H), 7.24-7.17 (m, 2H), 7.10-7.01 (m, 1H), 3.97 (s, 2H), 2.95-2.92 (m, 4H), 2.52 (s, 3H) m/z = 428.2 [M + H]⁺. |
| 60 | [chemical structure] | ¹H NMR (MeOD, 400 MHz): δ 9.16 (s, 1H), 8.41 (s, 1H), 7.89-7.84 (m, 1H), 7.78-7.73 (m, 1H), 7.65 (t, J = 8.0 Hz, 1H), 7.35 (d, J = 8.0 Hz, 1H), 7.27-7.14 (m, 2H), 6.95-6.88 (m, 2H), 3.96 (s, 2H), 2.97-2.84 (m, 4H), 2.50 (s, 3H) m/z = 446.2 [M + H]⁺ |
| 61 | [chemical structure] | ¹H NMR (MeOD, 400 MHz) δ 9.17 (s, 1H), 8.42 (s, 1H), 7.88 (dd, J = 1.6, 9.2 Hz, 1H), 7.77 (d, J = 9.2 Hz, 1H), 7.66 (t, J = 7.6 Hz, 1H), 7.36 (d, J = 7.6 Hz, 1H), 7.31-7.24 (m, 1H), 7.18 (d, J = 7.6 Hz, 1H), 7.04 (d, J = 7.6 Hz, 1H), 6.98 (d, J = 10.0 Hz, 1H), 6.90 (dt, J = 2.4, 8.4 Hz, 1H), 3.96 (s, 2H), 3.01-2.90 (m, 2H), 2.89-2.81 (m, 2H), 2.52 (s, 3H) m/z = 428.3 [M + H]⁺ |
| 62 | [chemical structure] | ¹H NMR (MeOD, 400 MHz) δ 9.17 (s, 1H), 8.43 (s, 1H), 7.88 (dd, J = 1.6, 9.2 Hz, 1H), 7.77 (d, J = 9.2 Hz, 1H), 7.67 (t, J = 7.6 Hz, 1H), 7.36 (d, J = 7.6 Hz, 1H), 7.27-7.16 (m, 3H), 6.99 (t, J = 8.8 Hz, 2H), 3.96 (s, 2H), 2.96-2.89 (m, 2H), 2.87- 2.80 (m, 2H), 2.52 (s, 3H) m/z = 427.19 [M + H]⁺. |
| 63 | [chemical structure] | ¹H NMR (MeOD, 400 MHz) δ 9.18 (s, 1H), 8.43 (s, 1H), 7.88 (d, J = 9.2 Hz, 1H), 7.77 (d, J = 9.2 Hz, 1H), 7.65 (d, J = 8.0 Hz, 1H), 7.37 (d, J = 8.0 Hz, 1H), 7.33-7.27 (m, 1H), 7.18 (d, J = 7.6 Hz, 1H), 6.88-6.86 (m, 2H), 3.96 (s, 2H), 2.92-2.85 (m, 4H), 2.52 (s, 3H) m/z = 446.2 [M + H]⁺. |
| 64 | [chemical structure] | ¹H NMR (MeOD, 400 MHz) δ 9.19 (s, 1H), 8.43 (s, 1H), 7.89 (dd, J = 1.2, 9.2 Hz, 1H), 7.77 (d, J = 9.2 Hz, 1H), 7.67 (t, J = 7.6 Hz, 1H), 7.38 (d, J = 8.0 Hz, 1H), 7.27-7.10 (m, 6H), 4.07 (s, 2H), 2.93-2.79 (m, 2H), 2.70 (t, J = 7.6 Hz, 2H), 2.52 (s, 3H), 1.92-1.98 (m, 2H) m/z = 424.3 [M + H]⁺. |
| 65 | [chemical structure] | ¹H NMR (MeOD, 400 MHz) δ 9.15 (s, 1H), 8.42 (s, 1H), 7.90-7.83 (m, 1H), 7.79-7.72 (m, 1H), 7.65 (t, J = 7.6 Hz, 1H), 7.35 (d, J = 7.6 Hz, 1H), 7.27-7.14 (m, 3H), 6.96-6.84 (m, 3H), 4.10 (t, J = Hz, 2H), 4.03 (s, 2H), 3.09 (t, J = 5.2 Hz, 2H), 2.52 (s, 3H) m/z = 426.3 [M + H]⁺. |
| 66 | [chemical structure] | ¹H NMR (MeOD, 400 MHz) δ .16 (s, 1H), 8.43 (s, 1H), 7.89-7.84 (m, 1H), 7.79-7.74 (m, 1H), 7.67 (t, J = 7.6 Hz, 1H), 7.36 (d, J = 8.0 Hz, 1H), 7.19 (d, J = 7.6 Hz, 1H), 7.08 (t, J = 8.0 Hz, 2H), 6.66 (d, J = 7.6 Hz, 2H), 6.61 (t, J = 7.2 Hz, 1H), 4.11 (s, 2H), 3.36-3.32 (m, 2H), 3.06 (t, J = 6.0 Hz, 2H), 2.53 (s, 3H) m/z = 425.2 [M + H]⁺. |
| 67 | [chemical structure] | ¹H NMR (MeOD, 400 MHz) δ 9.16 (s, 1H), 8.43 (s, 1H), 7.85 (d, J = 9.2 Hz, 1H), 7.76 (d, J = 9.2 Hz, 1H), 7.67 (t, J = 7.6 Hz, 1H), 7.35 (d, J = 7.6 Hz, 1H), 7.19 (d, J = 7.6 Hz, 1H), 7.14 (t, J = 8.0 Hz, 2H), 6.77 (d, J = 8.0 Hz, 2H), 6.62 (t, J = 7.2 Hz, 1H), 3.97 (s, 2H), 3.48 (t, J = 6.8 Hz, 2H), 2.93 (s, 3H), 2.88 (t, J = 6.8 Hz, 2H), 2.52 (s, 3H) m/z = 439.3 [M + H]⁺. |
| 68 | [chemical structure] | ¹H NMR (MeOD, 400 MHz) δ 9.17 (dd, J = 1.6, 4.4 Hz, 1H), 8.93 (d, J = 8.4 Hz, 1H), 8.79 (d, J = 9.2 Hz, 1H), 8.58-8.51 (m, 2H), 8.41 (d, J = 7.6 Hz, 1H), 8.07 (dd, J = 4.4, 8.8 Hz, 1H), 7.90 (d, J = 7.6 Hz, 1H), 7.83-7.76 (m, 1H), 7.55 (J = 1.6, 7.2 Hz, 2H), 7.36 (dd, J = 1.6, 7.2 Hz, 2H), 7.31- 7.21 (m, 4H), 7.16-7.08 (m, 1H), 4.64 (s, 2H), 4.42 (s, 2H), 3.17 (s, 3H) m/z = 483.3 [M + H]⁺. |
| 69 | [chemical structure] | ¹H NMR (MeOD, 400 MHz): δ 9.25 (d, J = 5.6 Hz, 1H), 8.37-8.32 (m, 1H), 8.27-8.17 (m, 2H), 8.09 (d, J = 5.6 Hz, 1H), 7.97-7.80 (m, 3H), 7.61- 7.51 (m, 3H), 7.43-7.27 (m, 6H), 7.21-7.14 (m, 1H), 4.62 (s, 2H), 4.43 (s, 2H), 2.67 (s, 3H) m/z = 482.0 [M + H]⁺ |
| 70 | [chemical structure] | ¹H NMR (MeOD, 400 MHz): δ 8.79 (d, J = 7.2 Hz, 2H), 8.29-8.23 (m, 3H), 7.82-7.73 (m, 3H), 7.56-7.47 (m, 2H), 7.37-7.27 (m, 5H), 7.23-7.17 (m, 1H), 4.55 (s, 2H), 4.34 (s, 2H), 2.78 (s, 3H) m/z = 432.0 [M + H]⁺ |
| 71 | [chemical structure] | ¹H NMR (MeOD, 400 MHz) δ 8.29 (t, J = 8.0 Hz, 1H), 7.79-7.77 (m, 1H), 7.70 (t, J = 8.0 Hz, 2H), 7.59-7.53 (m, 2H), 7.79-7.37 (m, 1H), 7.30-7.29 (m, 4H), 7.19-7.18 (m, 1H), 7.03-7.00 (m, 3H), 6.11 (s, 2H), 4.54 (s, 2H), 4.31 (s, 2H), 2.87 (s, 3H) m/z = 475.3 [M + H]⁺. |
| 72 | [chemical structure] | ¹H NMR (MeOD, 400 MHz) δ 8.99 (s, 2H), 8.30- 8.25 (m, 3H), 7.95 (d, J = 8.4 Hz, 1H), 7.79 (t, J = 7.2 Hz, 2H), 7.69 (d, J = 8.4 Hz, 1H), 7.58-7.49 (m, 3H), 7.38 (d, J = 6.4 Hz, 2H), 7.31-7.30 (m, 4H), 7.20-7.13 (m, 1H), 4.57 (s, 2H), 4.37 (s, 2H), 2.87 (s, 3H) m/z = 483.3 [M + H]⁺. |
| 73 | [chemical structure] | ¹H NMR (MeOD, 400 MHz) δ 9.18 (dd, J = 1.4, 4.6 Hz, 1H), 8.93 (d, J = 8.8 Hz, 1H), 8.81 (d, J = 9.0 Hz, 1H), 8.60-8.52 (m, 2H), 8.08 (dd, J = 4.4, 8.8 Hz, 1H), 7.90 (d, J = 8.4 Hz, 1H), 7.62 (dd, J = 1.8, 7.6 Hz, 2H), 7.53-7.44 (m, 4H), 4.62 (s, 2H), 4.55 (s, 2H), 3.16 (s, 3H) m/z = 407.3 [M + H]⁺ |
| 74 | [chemical structure] | ¹H NMR (MeOD, 400 MHz): δ 9.23 (d, J = 5.6 Hz, 1H), 8.32 (d, J = 8.8 Hz, 2H), 8.22-8.14 (m, 2H), 7.96 (t, J = 8.0 Hz, 1H), 7.91-7.85 (m, 1H), 7.66-7.60 (m, 2H), 7.59-7.54 (m, 1H), 7.52-7.42 (m, 3H), 7.35 (d, J = 8.4 Hz, 1H), 4.58 (s, 2H), 4.57 (s, 2H), 2.71 (s, 3H) m/z = 406.0 [M + H]⁺ |
| 75 | [chemical structure] | ¹H NMR (MeOD, 400 MHz) δ 8.95 (d, J = 4.8 Hz, 1H), 8.46 (d, J = 7.2 Hz, 2H), 8.42-8.33 (m, 2H), 7.78 (ddd, J = 1.2, 5.6, 7.2 Hz, 1H), 7.74 (d, J = 7.6 Hz, 1H), 7.60 (dd, J = 2.0, 7.6 Hz, 2H), 7.53- 7.46 (m, 3H), 4.57 (s, 2H), 4.52 (s, 2H), 2.98 (s, 3H) m/z = 356.2 [M + H]⁺. |
| 76 | [chemical structure] | ¹H NMR (MeOD, 400 MHz) δ 8.23 (t, J = 8.0 Hz, 1H), 7.70-7.66 (m, 2H), 7.60-7.59 (m, 2H), 7.52- 7.48 (m, 3H), 7.07-6.99 (m, 3H), 6.08 (s, 2H), 4.50 (s, 2H), 4.46 (s, 2H), 2.86 (s, 3H) m/z = 399.2 [M + H]⁺. |
| 77 | [chemical structure] | ¹H NMR (MeOD, 400 MHz) δ 9.00 (s, 2H), 8.35 (d, J = 1.6 Hz, 1H), 8.28-8.24 (d, J = 1.6 Hz, 1H), 8.04 (dd, J = 2.0, 8.8 Hz, 1H), 7.75 (dd, J = 8.0, 10.8 Hz, 2H), 7.65-7.63 (m, 2H), 7.50-7.48 (m, 3H), 4.56 (s, 2H), 4.54 (s, 2H), 2.89 (s, 3H) m/z = 407.3 [M + H]⁺. |
| 78 | [chemical structure] | ¹H NMR (MeOD, 400 MHz) δ 9.17 (dd, J = 1.6, 4.4 Hz, 1H), 8.91 (d, J = 8.4 Hz, 1H), 8.82 (d, J = 9.2 Hz, 1H), 8.71-8.49 (m, 3H), 8.06 (dd, J = 4.4, 8.8 Hz, 1H), 7.91 (d, J = 8.4 Hz, 1H), 7.46-7.36 (m, 1H), 7.08 (t, J = 8.0 Hz, 2H), 4.70 (s, 2H), 3.66-3.56 (m, 2H), 3.32-3.27 (m, 2H), 3.19 (s, 3H) m/z = 457.2 [M + H]⁺. |
| 79 | [chemical structure] | ¹H NMR (MeOD, 400 MHz) δ 9.16 (d, J = 5.6 Hz, 1H), 8.25 (d, J = 8.4 Hz, 2H), 8.14-8.07 (m, 2H), 7.94-7.86 (m, 1H), 7.85-7.79 (m, 1H), 7.55-7.46 (m, 1H), 7.32-7.25 (m, 2H), 6.94 (t, J = 8.0 Hz, 2H), 4.55 (s, 2H), 3.53-3.42 (m, 2H), 3.30-3.35 (m, 2H), 2.65 (s, 3H) m/z = 456.3 [M + H]⁺. |
| 80 | [chemical structure] | ¹H NMR (MeOD, 400 MHz) δ 8.87 (d, J = 6.4 Hz, 2H), 8.46 (t, J = 8.0 Hz, 1H), 8.29 (d, J = 6.4 Hz, 2H), 8.02 (d, J = 8.0 Hz, 1H), 7.94 (d, J = 8.0 Hz, 1H), 7.43-7.32 (m, 1H), 7.11-6.96 (m, 2H), 4.63 (s, 2H), 3.57-3.50 (m, 2H), 3.30-3.22 (m, 2H), 2.91 (s, 3H) m/z = 406.3 [M + H]⁺. |
| 81 | [chemical structure] | ¹H NMR (MeOD, 400 MHz) δ 8.23 (t, J = 8.0 Hz, 1H), 7.68 (t, J = 9.2 Hz, 2H), 7.42-7.36 (m, 1H), 7.08-7.01 (m, 5H), 6.08 (s, 2H), 4.56 (s, 2H), 3.52-3.48 (m, 2H), 3.27-3.23 (m, 2H), 2.86 (s, 3H) m/z = 449.2 [M + H]⁺. |
| 82 | [chemical structure] | ¹H NMR (MeOD, 400 MHz) δ 8.95 (s, 2H), 8.33 (s, 1H), 8.19 (d, J = 8.8 Hz, 1H), 8.04-8.00 (m, 2H), 7.57 (dd, J = 8.0, 16.4 Hz, 2H), 7.40-7.34 (m, 1H), 7.06-7.02 (m, 2H), 4.54 (s, 2H), 3.54- 3.50 (m, 2H), 3.26-3.22 (m, 2H), 2.75 (s, 3H) m/z = 457.2 [M + H]⁺. |
| 83 | [chemical structure] | ¹H NMR (MeOD, 400 MHz) δ 8.90 (d, J = 4.4 Hz, 1H), 8.42 (s, 1H), 8.33 (d, J = 9.2 Hz, 1H), 8.21 (d, J = 8.8 Hz, 1H), 7.85-7.76 (m, 3H), 7.35-7.14 (m, 2H), 7.15 (d, J = 2.6 Hz, 3H), 4.05 (s, 2H), 3.86 (s, 2H), 2.56 (s, 3H), 2.36 (s, 3H) m/z = 421.2 [M + H]⁺ |
| 84 | [chemical structure] | ¹H NMR (MeOD, 400 MHz) δ 8.91-8.90 (m, 1H), 8.43 (d, J = 8.8 Hz, 1H), 8.32 (d, J = 8.8 Hz, 1H), 8.20 (d, J = 8.8 Hz, 1H), 7.84-7.74 (m, 3H), 7.27 (d, J = 7.0 Hz, 1H), 7.19-7.12 (m, 2H), 6.90-6.82 (m, 2H), 4.01 (s, 2H), 3.77 (s, 3H), 2.95-2.85 (m, 4H), 2.56 (s, 3H) m/z = 451.2 [M + H]⁺ |
| 85 | [chemical structure] | ¹NMR (MeOD, 400 MHz) δ 8.91-8.89 (m, 1H), 8.42 (d, J = 7.8 Hz, 1H), 8.32 (d, J = 9.6 Hz, 1H), 8.18 (d, J = 9.2 Hz, 1H), 7.80-7.73 (m, 3H), 7.27 (d, J = 6.6 Hz, 1H), 7.12 (dd, J = 7.6, 8.8 Hz, 2H), 6.78 (d, J = 7.8 Hz, 2H), 6.61 (t, J = 7.2 Hz, 1H), 4.02 (s, 2H), 3.49 (t, J = 7.0 Hz, 2H), 2.92 (s, 3H), 2.89 (t, J = 6.8 Hz, 2H), 2.55 (s, 3H) m/z = 450.2 [M + H]⁺ |
| 86 | [chemical structure] | ¹H NMR (MeOD, 400 MHz): δ 8.89 (q, J = 1.6 Hz, 2H), 8.30 (d, J = 1.6 Hz, 1H), 8.12-8.01 (m, 2H), 7.67 (t, J = 7.6 Hz, 1H), 7.40-7.32 (m, 2H), 7.22 (d, J = 8.0 Hz, 1H), 7.13-7.19 (m, 3H), 4.04 (s, 2H), 3.88 (s, 2H), 2.54 (s, 3H), 2.38 (s, 3H) m/z = 421.2 [M + H]⁺ |
| 87 | [chemical structure] | ¹H NMR (MeOD, 400 MHz): δ 8.91-8.86 (m, 2H), 8.30 (d, J = 1.6 Hz, 1H), 8.12-8.01 (m, 2H), 7.66 (t, J = 7.6 Hz, 1H), 7.37 (d, J = 8.0 Hz, 1H), 7.24- 7.14 (m, 3H), 6.94-6.84 (m, 2H), 3.99 (s, 2H), 3.80 (s, 3H), 2.86-2.96 (m, 4H), 2.54 (s, 3H) m/z = 451.3 [M + H]⁺ |
| 88 | [chemical structure] | ¹H NMR (MeOD, 400 MHz): δ 8.87-8.83 (m, 2H), 8.26 (d, J = 1.6 Hz, 1H), 8.08-8.03 (m, 1H), 8.01- 7.96 (m, 1H), 7.62 (t, J = 8.0 Hz, 1H), 7.32 (d, J = 7.6 Hz, 1H), 7.19 (d, J = 7.6 Hz, 1H), 7.15-7.08 (m, 2H), 6.77 (d, J = 8.0 Hz, 2H), 6.61 (t, J = 7.2 Hz, 1H), 3.98 (s, 2H), 3.48 (t, J = 6.8 Hz, 2H), 2.92 (s, 3H), 2.88 (t, J = 6.8 Hz, 2H), 2.51 (s, 3H) m/z = 450.3 [M + H]⁺ |
| 89 | [chemical structure] | ¹H NMR (MeOD, 400 MHz): δ 8.83 (dd, J = 4.0, 1.6 Hz, 1H), 8.33 (d, J = 8.4 Hz, 1H), 8.13 (d, J = 1.6 Hz, 1H), 8.00 (d, J = 8.8 Hz, 1H), 7.88 (dd, J = 9.2, 2.0 Hz, 1H), 7.62-7.52 (m, 2H), 7.28 (d, J = 8.0 Hz, 1H), 7.26-7.19 (m, 1H), 7.16 (d, J = 8.0 Hz, 1H), 6.96-6.89 (m, 2H), 3.98 (s, 2H), 2.98- 2.86 (m, 5H), 2.51 (s, 3H) m/z = 456.2 [M + H]⁺ |
| 90 | [chemical structure] | ¹H NMR (MeOD, 400 MHz): δ 8.87 (dd, J = 4.4, 1.6 Hz, 1H), 8.37 (d, J = 8.8 Hz, 1H), 8.17 (d, J = 1.6 Hz, 1H), 8.03 (d, J = 8.8 Hz, 1H), 7.94-7.89 (m, 1H), 7.66-7.55 (m, 2H), 7.43-7.39 (m, 2H), 7.39-7.25 (m, 5H), 7.20 (d, J = 7.6 Hz, 1H), 3.99 (s, 2H), 3.88 (s, 2H), 2.55 (s, 3H) m/z = 406.3 [M + H]⁺ |
| 91 | [chemical structure] | ¹H NMR (MeOD, 400 MHz): δ 8.89-8.84 (m, 1H), 8.37 (d, J = 7.6 Hz, 1H), 8.12 (d, J = 1.6 Hz, 1H), 8.06-8.00 (m, 1H), 7.91-7.85 (m, 1H), 7.66-7.55 (m, 3H), 7.43-7.26 (m, 8H), 7.25-7.17 (m, 3H), 3.89 (s, 2H), 3.83 (s, 2H), 2.55 (s, 3H) m/z = 482.3 [M + H]⁺ |

### Example 2: Synthesis of Linker-Modified Payloads

### Synthesis 4-((S)-2-((S)-2-(6-(2,5-dioxo-2,5-dihydro-1H-pyrrol-1-yl)hexanamido)-3-methylbutanamido)-5-ureidopentanamido)benzyl ((5-([1,2,4]triazolo[1,5-a]pyridin-6-yl)-4-(6-methylpyridin-2-yl)-1H-imidazol-2-yl)methyl(3-chlorobenzyl)carbamate, trifluoroacetate salt (Compound 2.1)

[chemical structure]

### Step A. Preparation of Compound 2.1

[chemical structure]

To a solution containing 21.9 mg (0.051 mmol) of 2 in 2.5 mL of DMF was added 33 mg (0.045 mmol) of mc-VC-PAB-PNP (Broad Pharm; CAS 159857-81-5) and 32 uL (0.18 mmol) of DIPEA. The reaction mixture was stirred at 40° C. for 15 then purified without work-up by HPLC (0% to 70% MeCN w/0.1% TFA/H₂O x/0.1% TFA). The product peak was collected and lyophilized to afford the TFA salt of Compound 2.1.

LCMS (M+H)=1028.2.

The compound-linkers in Table 17 were prepared in a manner similar to that described for compound 2.1 using the appropriate compound as starting material.

TABLE 17

| | | Mass |
|---|---|---|
| | | Spec. |
| Compound | | LCMS |
| Number | Structure | [M + H]⁺ |
| 2.1 | [chemical structure] | 1028.2 |
| 3.1 | [chemical structure] | 1028.2 |
| 4.1 | [chemical structure] | 1062.4 |
| 5.1 | [chemical structure] | 1062.4 |
| 7.1 | [chemical structure] | 1024.6 |
| 8.1 | [chemical structure] | 1024.6 |
| 9.1 | [chemical structure] | 1054.6 |
| 10.1 | [chemical structure] | 1028.6 |
| 17.1 | [chemical structure] | 1024.6 |
| 18.1 | [chemical structure] | 1012.6 |
| 21.1 | [chemical structure] | 1008.6 |
| 24.1 | [chemical structure] | 1070.6 |
| 25.1 | [chemical structure] | LCMS (M + H]⁺: 1037.5 |
| 22.1 | [chemical structure] | 1022.6 |
| 24.1 | [chemical structure] | 1089.3 |
| 26.1 | [chemical structure] | 1212.6 |
| 32.1 | [chemical structure] | 1233.6 |
| 33.1 | [chemical structure] | 1233.6 |
| 34.1 | [chemical structure] | 1233.6 |
| 38.1 | [chemical structure] | 1088.3 |
| 39.1 | [chemical structure] | 1106.2 |
| 40.1 | [chemical structure] | 1071.2 |
| 41.1 | [chemical structure] | 1071.2 |
| 42.1 | [chemical structure] | 1071.2 |
| 43.1 | [chemical structure] | 1076.3 |
| 44.1 | [chemical structure] | 1062.3 |
| 45.1 | [chemical structure] | 1022.2 |
| 46.1 | [chemical structure] | 1038.2 |
| 47.1 | [chemical structure] | 1042.6 |
| 48.1 | [chemical structure] | 1084.2 |
| 49.1 | [chemical structure] | 1084.2 |
| 50.1 | [chemical structure] | 1008.2 |
| 51.1 | [chemical structure] | 1070.2 |
| 52.1 | [chemical structure] | 1008.2 |
| 53.1 | [chemical structure] | 1086.2 |
| 54.1 | [chemical structure] | 1087.2 |
| 56.1 | [chemical structure] | 1086.2 |
| 57.1 | [chemical structure] | 1087.2 |
| 58.1 | [chemical structure] | 1087.2 |
| 59.1 | [chemical structure] | 1026.1 |
| 60.1 | [chemical structure] | 1055.2 |
| 61.1 | [chemical structure] | 1026.1 |
| 62.1 | [chemical structure] | 1026.1 |
| 63.1 | [chemical structure] | 1044.1 |
| 64.1 | [chemical structure] | 1022.2 |
| 65.1 | [chemical structure] | 1024.2 |
| 66.1 | [chemical structure] | 1023.2 |
| 74.1 | [chemical structure] | 1004.2 |
| 76.1 | [chemical structure] | 997.2 |
| 77.1 | [chemical structure] | 1005.1 |
| 79.1 | [chemical structure] | 1054.1 |
| 80.1 | [chemical structure] | 1004.1 |
| 81.1 | [chemical structure] | 1047.2 |
| 82.1 | [chemical structure] | 1055.2 |

### Example 3

### Generation of Antibodv-TGFb Inhibitor Conjugates Through Partial Reduction of Native Disulfide Bonds of Non-Engineered Antibodies

### Reduction of Native Disulfide Bonds of Non-Engineered Antibodies

The mAb (3-8 mg/mL in PBS) was exchanged into HEPES (100 mM, pH 7.0, 1 mM DTP A) via molecular weight cut-off centrifugal filtration (Millipore, 30 kDa). The resultant mAb solution was transferred to a tared 50 mL conical tube. The mAb concentration was determined to be 3-8 mg/mL by A₂₈₀. To the mAb solution was added TCEP (2.0-4.0 equivalents, 1 mM stock) at room temperature and the resultant mixture was incubated at 37° C. for 30-90 minutes, with gentle shaking. Upon being cooled to room temperature, a stir bar was added to the reaction tube. With stirring, a linker-payload (5-10 equivalents, 10 mM DMSO) was added dropwise. The resultant reaction mixture was allowed to stir at ambient temperature for 30-60 minutes, at which point N-ethyl maleimide (3.0 equivalents, 100 mM DMA) was added. After an additional 15 minutes of stirring, N-acetylcysteine (6.0-11.0 equivalents, 50 mM HEPES) was added. The crude ADC was then exchanged into PBS and purified by preparative SEC (e.g. HiLoad 26/600, Superdex 200 μg) using PBS as the mobile phase. The pure fractions were concentrated via molecular weight cut-off centrifugal filtration (Millipore, 30 kDa), sterile filtered, and transferred to 15 mL conical tubes. Drug-antibody construct ratios (molar ratios) were determined by methods described herein.

### Example 4

### General Procedure for the Determination of the Drug-Antibody-Ratios

### Hydrophobic Interaction Chromatography

10 μL of a 6 mg/mL solution of a conjugate is injected into an HPLC system set-up with a TOSOH TSKgel Butyl-NPR™ hydrophobic interaction chromatography (HIC) column (2.5 μM particle size, 4.6 mm×35 mm) attached. Then, over the course of 18 minutes, a method is run in which the mobile phase gradient is run from 100% mobile phase A to 100% mobile phase B over the course of 12 minutes, followed by a six-minute re-equilibration at 100% mobile phase A. The flow rate is 0.8 mL/min and the detector is set at 280 nM. Mobile phase A is 1.5 M ammonium sulfate, 25 mM sodium phosphate (pH 7). Mobile phase B is 25% isopropanol in 25 mM sodium phosphate (pH 7). Post-run, the chromatogram is integrated and the molar ratio is determined by summing the weighted peak area.

### Example 5

### TGFβ Reporter Assay

### Materials and General Procedures

TGFβ/SMAD Signaling Pathway SBE reporter cell line was obtained from BPS Bioscience. Cells were passed, expanded, and stored in liquid nitrogen as per the supplier's instructions with the exception that growth media is changed to DMEM-C with Geneticin (DMEM supplemented with 10% fetal bovine serum, 1×NEAA, 1 mM Pyruvate, 2 mM glutamine, 50 μg/mL penicillin, 50 U/mL streptomycin and 400 μg/mL of Geneticin). The assay media was MEM supplemented with 0.5% fetal bovine serum, 1×NEAA, 1 mM Pyruvate, 50 μg/mL penicillin and 50 U/mL streptomycin.

Compounds of Formula (I) were assayed to measure their activity as ALK5 inhibitors.

Enzyme Inhibition Assay

ALK5 enzyme inhibition assays were performed by Reaction Biology Corp (Malvern, Pa.). 1 mg/mL of peptide substrate (casein) and 10 uM ATP were prepared in a mixture of fresh reaction buffer. The kinase was delivered into the substrate solution which was gently mixed. Compounds in 100% DMSO were added to the kinase reaction mixture by Acoustic technology (Echo550; nanoliter range) and the mixture was incubated for 20 min at room temperature. ³³P-ATP (Specific activity 10 uCi/uL) was added into the reaction mixture to initiate the reaction and the reaction mixture was incubated for 2 hours at room temperature. Radioactivity was detected by filter-binding method and kinase activity data were expressed as the percent remaining kinase activity in test samples compared to vehicle (dimethyl sulfoxide) reactions. IC₅₀ values and curve fits were obtained using Prism (GraphPad Software). Compounds having an IC₅₀ value between 0.1 nM and 50 nM are denoted as ++++, 50 nM and 100 nM as +++, 100 nM and 1000 nM as ++, and 1000 nM to 10,000 nM as + in Table 18 below.

TABLE 18
INHIBITION OF ALK5 BY COMPOUNDS
DESCRIBED HEREIN.

| Compound | IC₅₀ | Compound | IC₅₀ |
|---|---|---|---|
| 1 | ++ | 48 | ++++ |
| 2 | ++ | 49 | ++ |
| 3 | + | 50 | ++ |
| 4 | ++ | 51 | ++ |
| 5 | +++ | 52 | +++ |
| 6 | ++++ | 53 | ++++ |
| 7 | ++ | 54 | +++ |
| 8 | + | 55 | NT |
| 9 | ++ | 56 | NT |
| 10 | ++++ | 57 | NT |
| 11 | ++ | 58 | NT |
| 12 | + | 59 | ++++ |
| 13 | +++ | 60 | ++++ |
| 14 | +++ | 61 | ++++ |
| 15 | +++ | 62 | ++ |
| 16 | ++++ | 63 | ++ |
| 17 | +++ | 64 | ++ |
| 18 | ++++ | 65 | ++ |
| 19 | ++++ | 66 | ++ |
| 20 | + | 67 | +++ |
| 21 | ++++ | 68 | ++++ |
| 22 | ++++ | 69 | ++ |
| 23 | +++ | 70 | NT |
| 24 | ++++ | 71 | ++++ |
| 25 | ++ | 72 | NT |
| 26 | ++++ | 73 | NT |
| 27 | +++ | 74 | NT |
| 28 | ++++ | 75 | NT |
| 29 | ++ | 76 | NT |
| 30 | ++ | 77 | NT |
| 31 | ++ | 78 | NT |
| 32 | ++ | 79 | NT |
| 33 | ++ | 80 | NT |
| 34 | ++ | 81 | ++++ |
| 35 | ++++ | 82 | ++++ |
| 36 | +++ | 83 | +++ |
| 37 | +++ | 84 | ++ |
| 38 | ++++ | 85 | +++ |
| 39 | ++++ | 86 | ++++ |
| 40 | ++ | 87 | ++++ |
| 41 | +++ | 88 | ++++ |
| 42 | ++ | 89 | ++++ |
| 43 | ++ | 90 | +++ |
| 44 | ++ | 91 | ++ |
| 45 | +++ | | |
| 46 | +++ | | |
| 47 | ++++ | | |

TGFb Reporter Assay

TGFb/SMAD Signaling Pathway SBE reporter cell line was obtained from BPS Bioscience. Cells were passed/expanded/stored in liquid nitrogen per supplier's instruction with the exception that growth media was changed to DMEM-C with Geneticin (DMEM supplemented with 10% fetal bovine serum, IX NEAA, 1 mM Pyruvate, 2 mM glutamine, 50 μg/mL penicillin, 50 U/mL streptomycin and 400 ug/mL of Geneticin). The assay media was MEM supplemented with 0.5% fetal bovine serum, IX NEAA, 1 mM Pyruvate, 50 μg/mL penicillin and 50 U/mL streptomycin.

Reporter cells were harvested from the tissue culture flasks by incubation in small quantity of Versene at room temperature for three to five minutes after the media in the flask was removed and cells rinsed with PBS. Cells were counted and diluted in the assay media at 0.8×10⁶ cells/mL then 50 uL/well were added to 96-well assay plate. Test samples (at desired concentrations diluted in assay media) were added to assay plate containing the 50 uL/well of cells (or media only), 50 uL per well, and incubated for 5-6 hours at 37° C. in a 5% CO₂ humidified incubator. After that time, 15 uL of TGFb diluted to 12.5 ng/mL in the assay media was added to the plate. Controls included TGFb titration (from 25 to 0 ng/mL) without inhibitors, and media only (without cells, inhibitor or TGFb). Plates were incubated at 37° C. in a % CO₂ humidified incubator for 18 h. Luciferase substrate solution was subsequently added at 75 uL per well, incubated in dark with shaking at room temperature for 10 min, and luminescence was measured using a luminometer. Compounds having an EC₅₀ value between 0.1 nM and 10 nM are denoted as ++++, 10 nM and 100 nM as +++, 100 nM and 1000 nM as ++, and 1000 nM to 10,000 nM as + in Table 19 below.

TABLE 19
TGFB REPORTER ACTIVITY BY COMPOUNDS
DESCRIBED HEREIN.

| Compound | EC₅₀ | Compound | EC₅₀ |
|---|---|---|---|
| 1 | ++++ | 48 | +++ |
| 2 | +++ | 49 | +++ |
| 3 | +++ | 50 | +++ |
| 4 | ++ | 51 | ++ |
| 5 | +++ | 52 | ++++ |
| 6 | ++++ | 53 | ++++ |
| 7 | ++++ | 54 | ++++ |
| 8 | ++ | 55 | + |
| 9 | +++ | 56 | +++ |
| 10 | ++++ | 57 | +++ |
| 11 | +++ | 58 | +++ |
| 12 | ++ | 59 | ++++ |
| 13 | ++++ | 60 | ++++ |
| 14 | +++ | 61 | ++++ |
| 15 | ++ | 62 | ++++ |
| 16 | ++ | 63 | ++++ |
| 17 | +++ | 64 | ++++ |
| 18 | ++++ | 65 | ++++ |
| 19 | ++++ | 66 | ++++ |
| 20 | ++ | 67 | ++++ |
| 21 | ++++ | 68 | +++ |
| 22 | ++++ | 69 | ++ |
| 23 | ++++ | 70 | NT |
| 24 | ++++ | 71 | ++ |
| 25 | ++++ | 72 | +++ |
| 26 | ++++ | 73 | ++ |
| 27 | ++++ | 74 | ++ |
| 28 | ++++ | 75 | ++ |
| 29 | ++ | 76 | +++ |
| 30 | ++ | 77 | ++++ |
| 31 | ++ | 78 | ++++ |
| 32 | ++ | 79 | ++ |
| 33 | ++ | 80 | ++ |
| 34 | ++ | 81 | +++ |
| 35 | ++++ | 82 | ++++ |
| 36 | +++ | 83 | ++++ |
| 37 | +++ | 84 | ++++ |
| 38 | ++++ | 85 | ++++ |
| 39 | +++ | 86 | ++++ |
| 40 | +++ | 87 | ++++ |
| 41 | ++++ | 88 | ++++ |
| 42 | +++ | 89 | NT |
| 43 | ++ | 90 | ++++ |
| 44 | +++ | 91 | +++ |
| 45 | ++++ | | |
| 46 | ++++ | | |
| 47 | ++++ | | |

### Example 6

### Conjugates

The linker payloads in Table 17 were covalently attached to an anti-LRRC15 antibody. The LRRC15 antibody is the murine M25 antibody or a humanized variant thereof (see International Application No. WO 2017/095805, incorporated herein by reference in its entirety and for all purposes). Conjugation to the linker-payload is via the interchain disulfides. The antibodies have either a wild-type Fc region or domain or a null Fc region or domain. The Fcnull mutations for human IgG1 are L234A, L236A, G237A, and K322A and the Fcnull mutations for murine IgG2a are L234A, L236A, G237A, K322A, and P329G; numbering by EU index.

The resultant antibody drug conjugates were tested via a cell reporter assay. HEK293 SMAD2p luciferase reporter cells transfected to stably express full length human LRRC15 were seeded in 96 well plates at 40,000 cells/well in an assay media of MEM+0.5% FBS, 1% NEAA, 1% NaPyr & 1% Pen/Strep. Conjugates and controls were added to wells in a dose titration ranging from 500 nM to 0.3 nM. After 24 hours of culture at 37° C. in a 5% CO₂ environment human TGFβ1 was added (PeproTech Inc.) to a final concentration of 1.6 ng/ml followed by an additional 18 hour of culture. Luciferase Steady Glo reagent (Promega Corporation) was added as recommended by manufacturer. After incubating 10 minutes with shaking, SMAD2p activity was determined by measuring luminescence with an Envision Plate Reader (Perkin-Elmer Inc.) and an absolute IC₅₀ was determined using Prism Software v8.01 (GraphPad Inc.).

The potency of the antibody drug conjugates track proportionally with the activity observed for the small molecule activity within the small molecule cell-based reporter assay. For examples in which the observed activity of a small molecule is low when assessed by the small molecule cell-based reporter assay and high by measure within the small molecule cell-free enzymatic inhibition assay, it is believed, without being bound by theory, that this can most often be attributed to the molecule possessing low cell permeability. In these cases, the rank order of potency of the applicable antibody drug conjugate tracks more closely with the observed activity within the small molecule cell-free enzymatic inhibition assay.

### Example 7

### Effect of Anti-ASGR1-ALK5 Inhibitor Conjugates on TGFβ Production

Anti-ASGR1 antibodies conjugated to an ALK5 inhibitor were tested via a cell reporter assay. Briefly, HEK293 SBE-LUC reporter cells transfected to stably express full length human ASGR1 were seeded in 96 well plates at 40,000 cells/well in an assay media of minimum essential media containing 0.5% fetal bovine serum, 1% nonessential amino acids, 1% sodium pyruvate and 1% Pen/Strep. Anti-ASGR1-ALK5 inhibitor conjugates and controls were added to wells in a dose titration ranging from 5 μM to 0.064 nM to HEK293 SBE-LUC and ASGR1-HEK293 SBE-LUC cells. After incubating for 6 hours at 37° C. in a 5% CO₂ environment, human TGFβ1 (PeproTech Inc.) was added to a final concentration of 1.6 ng/ml followed by an additional 18 hour incubation under the same conditions. Luciferase Steady-Glo® reagent (Promega Corporation) was added at 70 μl/well and incubated with shaking for 10 minutes. Luciferase activity was determined by measuring luminescence with an EnVision® Plate Reader (Perkin-Elmer Inc.). Data were fit with a four-parameter non-linear regression to calculate IC₅₀ values using Prism Software v7.04 (GraphPad Inc.).

Table 20 shows that each of the anti-ASGR1-ALK5 inhibitor conjugates could efficiently inhibit TGFβ1-mediated luciferase expression as compared to an unconjugated anti-ASGR antibody (mAb-A) alone. Furthermore, the ASGR1-ALK5 inhibitor conjugates were more potent than the ALK5 inhibitor (Compound 60) alone.

TABLE 20
ASGR1-ALK5 INHIBITOR CONJUGATE INHIBITION
OF TGFβ1-MEDIATED LUCIFERASE EXPRESSION

| Test Article† | IC₅₀ (nM) |
|---|---|
| Compound 60 | 13.4 |
| ASGR1-mAb-A | —* |
| ASGR1-mAb-A-LP Cmpd 60.1 | 7.2 |
| ASGR1-mAb-B-LP Cmpd 60.1 | 9.1 |
| chG2D-LP Cmpd 60.1 | 4.3 |
| chJ4F-LP Cmpd 60.1 | 6.5 |
| ASGR1-mAb-C-LP Cmpd 60.1 | 13.9 |
| chL4L-LP Cmpd 60.1 | 8.5 |
| chH8K-LP Cmpd 60.1 | 3.8 |

—*No detectable activity
†Monoclonal antibodies (mAbs) ASGR1 mAb-A, ASGR1 mAb-B, and ASGR1 mAb-C correspond to anti-ASGR mAbs 4A2, 72G9 and 4F3, respectively (see, e.g., PCT Publication Nos. WO 2017/058944 and WO 2014/023709);
mAb chG2D is comprised of heavy chain (HC) SEQ ID NO: 399 and light chain (LC) SEQ ID NO: 400;
mAb chJ4F is comprised of HC SEQ ID NO: 401 and LC SEQ ID NO: 402;
mAb chL4L is comprised of HC SEQ ID NO: 403 and LC SEQ ID NO: 404;
and mAb chH8K is comprised of HC SEQ ID NO: 405 and LC SEQ ID NO: 406.

>chG2D Heavy Chain
(SEQ ID NO: 399)
EVQLQQSGPELVKPGASVKISCKASGYSFTGYYMHWVKQSHVKS
LEWIGRINPNNGATNYNQNFKDKASLTVDKSSSTAYMEFHSLTS
EDSAVYYCTSVNFYYWGQGTTLTVSSASTKGPSVFPLAPSSKST
SGGTAALGCLVKDYFPEPVTVSWNSGALTSGVHTFPAVLQSSGL
YSLSSVVTVPSSSLGTQTYICNVNHKPSNTKVDKKVEPKSCDKT
HTCPPCPAPELLGGPSVFLFPPKPKDTLMISRTPEVTCVVVDVS
HEDPEVKFNWYVDGVEVHNAKTKPREEQYNSTYRVVSVLTVLHQ
DWLNGKEYKCKVSNKALPAPIEKTISKAKGQPREPQVYTLPPSR
EEMTKNQVSLTCLVKGFYPSDIAVEWESNGQPENNYKTTPPVLD
SDGSFFLYSKLTVDKSRWQQGNVFSCSVMHEALHNHYTQKSLSL
SPGK
>chG2D Light Chain
(SEQ ID NO: 400)
DIKMTQSPSSMYASLGERVTITCKASQVINSYLSWFQQKPGKSP
ETLIYRANTLVDGVPSRFSGSGSGQDYSLTISSLEYEDLGIYYC
LQYAEFPYTFGGGTKLEIKRTVAAPSVFIFPPSDEQLKSGTASV
VCLLNNFYPREAKVQWKVDNALQSGNSQESVTEQDSKDSTYSLS
STLTLSKADYEKHKVYACEVTHQGLSSPVTKSFNRGEC
>chJ4F Heavy Chain
(SEQ ID NO: 401)
QVQLQQSGDDLVKPGASVKLSCKASGYTFTSYWINWIKQRPGQG
LEWIGRIVPGSGSTYYNEMFKDKATLTVDTSSSTAYIQLSSLSS
EDSAVYFCARKPNFDVWGAGTTVTVSSASTKGPSVFPLAPSSKS
TSGGTAALGCLVKDYFPEPVTVSWNSGALTSGVHTFPAVLQSSG
LYSLSSVVTVPSSSLGTQTYICNVNHKPSNTKVDKKVEPKSCDK
THTCPPCPAPELLGGPSVFLFPPKPKDTLMISRTPEVTCVVVDV
SHEDPEVKFNWYVDGVEVHNAKTKPREEQYNSTYRVVSVLTVLH
QDWLNGKEYKCKVSNKALPAPIEKTISKAKGQPREPQVYTLPPS
REEMTKNQVSLTCLVKGFYPSDIAVEWESNGQPENNYKTTPPVL
DSDGSFFLYSKLTVDKSRWQQGNVFSCSVMHEALHNHYTQKSLS
LSPGK
>chJ4F Light Chain
(SEQ ID NO: 402)
DIVMTQSQKFMSTSVGDRVSVTCKASQNVGTNVAWYQQKPGQSP
KAVIYSASYRFSGVPDRLTGSGSGTDFTLTITNVQSEDLAEYFC
QQYNSYPLTFGAGTKLELKRTVAAPSVFIFPPSDEQLKSGTASV
VCLLNNFYPREAKVQWKVDNALQSGNSQESVTEQDSKDSTYSLS
STLTLSKADYEKHKVYACEVTHQGLSSPVTKSFNRGEC
>chL4L Heavy Chain
(SEQ ID NO: 403)
DVQLQESGPGLVKPSQSLSLTCTVTGYSITSDYAWNWIRQFPGN
KLEWMGYISYSGSTRYNPSLKSRISITRDTSKNQFFLQLNSVTT
EDTATYYCARRYRYDEGYGMDYWGQGTSVTVSSASTKGPSVFPL
APSSKSTSGGTAALGCLVKDYFPEPVTVSWNSGALTSGVHTFPA
VLQSSGLYSLSSVVTVPSSSLGTQTYICNVNHKPSNTKVDKKVE
PKSCDKTHTCPPCPAPELLGGPSVELEPPKPKDTLMISRTPEVT
CVVVDVSHEDPEVKENWYVDGVEVHNAKTKPREEQYNSTYRVVS
VLTVLHQDWLNGKEYKCKVSNKALPAPIEKTISKAKGQPREPQV
YTLPPSREEMTKNQVSLTCLVKGEYPSDIAVEWESNGQPENNYK
TTPPVLDSDGSFFLYSKLTVDKSRWQQGNVFSCSVMHEALHNHY
TQKSLSLSPGK
>chL4L Light Chain
(SEQ ID NO: 404)
DIQMTQSPASLSVFVGEIVTITCRASENIYSNLAWYQQKQGKS
PQLLVYAATNLADGVPSRFTGSGSGTQFSLKINSLQSEDFGNY
YCQHFWGTPPWTFGGGTKLEIKRTVAAPSVFIFPPSDEQLKSG
TASVVCLLNNFYPREAKVQWKVDNALQSGNSQESVTEQDSKDS
TYSLSSTLTLSKADYEKHKVYACEVTHQGLSSPVTKSFNRGEC
>chH8K Heavy Chain
(SEQ ID NO: 405)
DVQLQESGPGLVKPSQSLSLTCTVTGFSITSDYAWNWIRQFPG
NQLEWMGYRSYRGSTSYNPSLKSRISITRDTSKNQFFLQLNSV
TTEDTATYYCARRGYYGSSSHWYFDVWGAGTTVTVSSASTKGP
SVFPLAPSSKSTSGGTAALGCLVKDYFPEPVTVSWNSGALTSG
VHTFPAVLQSSGLYSLSSVVTVPSSSLGTQTYICNVNHKPSNT
KVDKKVEPKSCDKTHTCPPCPAPELLGGPSVELEPPKPKDTLM
ISRTPEVTCVVVDVSHEDPEVKENWYVDGVEVHNAKTKPREEQ
YNSTYRVVSVLTVLHQDWLNGKEYKCKVSNKALPAPIEKTISK
AKGQPREPQVYTLPPSREEMTKNQVSLTCLVKGEYPSDIAVEW
ESNGQPENNYKTTPPVLDSDGSFFLYSKLTVDKSRWQQGNVFS
CSVMHEALHNHYTQKSLSLSPGK
>chH8K Light Chain
(SEQ ID NO: 406)
DIQMTQSPASLSASVGETVTITCRASENIYSYLAWYQQKQGKS
PQLLVYNAKTLAEGVPSRESGSGSGTQFSLKINSLQPEDEGSY
YCQHHYGTPLTEGSGTKLELKRTVAAPSVFIFPPSDEQLKSGT
ASVVCLLNNEYPREAKVQWKVDNALQSGNSQESVTEQDSKDST
YSLSSTLTLSKADYEKHKVYACEVTHQGLSSPVTKSFNRGEC

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A compound having the structure of Formula (I): [chemical structure]
or a pharmaceutically acceptable isomer, racemate, hydrate, solvate, tautomer, isotope, or salt thereof, wherein:
one of M¹ and M² is [chemical structure]
and
the other of M¹ and M² is [chemical structure]
R¹ and R² are, at each occurrence, independently hydrogen, halogen, —OR¹¹, —SR¹¹, —N(R¹¹)₂, —NO₂, —CN, phenyl, or —C₁-C₆ alkyl, wherein said —C₁-C₆ alkyl is optionally substituted with one or more substituents independently selected from halogen, —OR¹¹, —SR¹¹, —S(O)R¹⁰, —S(O)₂R¹¹, —S(O)₂N(R¹¹)₂—N(R¹¹)₂, —C(O)R¹⁰, —C(O)N(R¹¹)₂, —N(R¹¹)C(O)R¹⁰, —C(O)OR¹¹, —OC(O)R¹⁰, —NO₂, and —CN;
R³ is, at each occurrence, independently halogen, —C₁-C₃ alkyl, —C₁-C₃ haloalkyl, —OH, —NO₂, —CN, —O—C₁-C₃ alkyl, or —O—C₁-C₃ haloalkyl;
R⁴ is, at each occurrence, independently hydrogen or C₁-C₃ alkyl, or two R⁴ join together with atoms to which they are attached to form a 5- or 6-membered heterocycle optionally substituted with one or more substituents independently selected from halogen, —C₁-C₃ alkyl, —OH, —O—C₁-C₃ alkyl, and —O—C₁-C₃ haloalkyl;
R⁵ is hydrogen, halogen, —OR⁶¹, —SR⁶¹, —N(R⁶¹)₂, —NO₂, —CN, and —C₁-C₆ alkyl, wherein said —C₁-C₆ alkyl is optionally substituted with one or more substituents independently selected from halogen, —OR⁶¹, —SR⁶¹, —N(R⁶¹)₂, —NO₂, and —CN;
R⁶ is, at each occurrence, independently:
halogen, —OR²¹, —SR²¹, —N(R²¹)₂, —C(O)R²⁰, —C(O)N(R²¹)₂, —N(R²¹)C(O)R²⁰, —C(O)OR²¹, —OC(O)R²¹, —S(O)R²⁰, —S(O)₂R²¹, —S(O)₂N(R²¹)₂, —OC(O)OR²¹, —OC(O)N(R²¹)₂, —NR²¹C(═O)OR²¹, —N(R²¹)C(O)N(R²¹)_{2J}—NO₂, —CN;
C₁-C₁₀ alkyl, C₂-C₁₀ alkenyl, and C₂-C₁₀ alkynyl, each of which is optionally substituted with one or more substituents independently selected from halogen, —OR²¹, —SR²¹, —N(R²¹)₂, —C(O)R²⁰, —C(O)N(R²¹)₂, —N(R²¹)C(O)R²⁰, —C(O)OR²¹, —OC(O)R²¹, —S(O)R²⁰, —S(O)₂R²¹, —S(O)₂N(R²¹)₂, —OC(O)OR²¹, —OC(O)N(R²¹)₂, —NR²¹C(═O)OR²¹, —N(R²¹)C(O)N(R²¹)_{2J}—NO₂, ═O, ═S, ═N(R²¹), —CN, a C₃-C₁₀ carbocycle, and a 3- to 10-membered heterocycle wherein said C₃-C₁₀ carbocycle and said 3- to 10-membered heterocycle are optionally substituted with one or more R^{X}; and
a C₃-C₁₀ carbocycle and a 3- to 10-membered heterocycle, each of which is optionally substituted with one or more substituents independently selected from halogen, —OR²⁰, —OH, —SR²⁰, —SH, —N(R²¹)₂, —C(O)R²⁰, —C(O)N(R²¹)₂, —N(R²¹)C(O)R²⁰, —C(O)OR²¹, —OC(O)R²¹, —S(O)R²⁰, —S(O)₂R²¹, —S(O)₂N(R²¹)₂, —OC(O)OR²¹, —OC(O)N(R²¹)₂, —NR²¹C(═O)OR²¹, —N(R²¹)C(O)N(R²¹)_{2J}—NO₂, ═O, ═S, ═N(R²¹), —CN, —C₂-C₆ alkenyl, —C₂-C₆ alkynyl and C₁-C₆ alkyl wherein said C₁-C₆ alkyl is optionally substituted with one or more substituents independently selected from R^{Y};
R⁷ and R⁸ are independently selected from hydrogen, halogen, C₁-C₃ alkyl, —OH, —O—C₁-C₃ alkyl, and —O—C₁-C₃ haloalkyl, or R⁷ and R⁸ join together with the atoms to which they are attached to form a C₅-C₆ carbocycle or 5- or 6-membered heterocycle each of which is optionally substituted with one or more substituents independently selected from halogen, —OR³¹, —SR³¹, —N(R³¹)₂, —NO₂, —CN and —C₁-C₆ alkyl wherein said C₁-C₆ alkyl is optionally substituted with one or more substituents independently selected from halogen, —OR³¹, —SR³¹, —N(R³¹)₂, —NO₂, and —CN;
Y is selected from —O— and —N(R⁹)— and R⁹ is, at each occurrence, independently selected from:
hydrogen; and —C₁-C₆ alkyl optionally substituted with one or more substituents independently selected from halogen, —OR⁴¹, —SR⁴¹, —S(O)R⁴⁰, —S(O)₂R⁴¹, —S(O)₂N(R⁴¹)₂—N(R⁴¹)₂, —C(O)R⁴⁰, —C(O)N(R⁴¹)₂, —N(R⁴¹)C(O)R⁴⁰—C(O)OR⁴¹, —OC(O)R⁴⁰, —NO₂, and —CN;
each R¹⁰, R²⁰, and R⁴⁰ is independently selected at each occurrence from:
C₁-C₁₀ alkyl, —C₂-C₁₀ alkenyl, and —C₂-C₁₀ alkynyl, each of which is optionally substituted with one or more substituents independently selected from R^{Y}; and
a C₃-C₁₂ carbocycle and a 3- to 12-membered heterocycle, each of which is optionally substituted with one or more substituents independently selected from R^{X};
each R¹¹, R²¹, R³¹, R⁴¹, and R⁶¹ is independently selected at each occurrence from:
hydrogen;
C₁-C₁₀ alkyl, —C₂-C₁₀ alkenyl, and —C₂-C₁₀ alkynyl, each of which is optionally substituted with one or more substituents independently selected from R^{Y}; and
a C₃-C₁₂ carbocycle and a 3- to 12-membered heterocycle, each of which is optionally substituted with one or more substituents independently selected from R^{X},
or two R¹¹, R²¹, R³¹, R⁴¹, or R⁶¹ on the same N atom are taken together with the N atom to which they are attached to form a N-containing heterocycle optionally substituted with R^{X};
each R^{X} is independently selected at each occurrence from:
halogen, —OR⁵¹, —SR⁵¹, —N(R⁵¹)₂, —C(O)R⁵⁰, —C(O)N(R⁵¹)₂, —N(R⁵¹)C(O)R⁵⁰, —C(O)OR⁵¹, —OC(O)R⁵¹, —S(O)R⁵⁰, —S(O)₂R⁵¹, —S(O)₂N(R⁵¹)₂, —OC(O)OR⁵¹, —OC(O)N(R⁵¹)₂, —NR⁵¹C(═O)OR⁵¹, —N(R⁵¹)C(O)N(R⁵¹)_{2J}—NO₂, ═O, ═S, ═N(R⁵¹), —CN, —C₂-C₆ alkenyl, —C₂-C₆ alkynyl and C₁-C₆ alkyl wherein said C₁-C₆ alkyl is optionally substituted with one or more substituents independently selected from —OR⁵¹, —SR⁵¹, —N(R⁵¹)₂, —C(O)R⁵⁰, —C(O)N(R⁵¹)₂, —N(R⁵¹)C(O)R⁵⁰—C(O)OR⁵¹, —OC(O)R⁵¹, —S(O)R⁵⁰, —S(O)₂R⁵¹, —S(O)₂N(R⁵¹)₂, —OC(O)OR⁵¹, —OC(O)N(R⁵¹)₂, —NR⁵¹C(═O)OR⁵¹, —N(R⁵¹)C(O)N(R⁵¹)_{2J} and ═O;
each R^{Y} is independently selected at each occurrence from:
halogen, —OR⁵¹, —SR⁵¹, —N(R⁵¹)₂, —C(O)R⁵⁰, —C(O)N(R⁵¹)₂, —N(R⁵¹)C(O)R⁵⁰, —C(O)OR⁵¹, —OC(O)R⁵¹, —S(O)R⁵⁰, —S(O)₂R⁵¹, —S(O)₂N(R⁵¹)₂, —OC(O)OR⁵¹, —OC(O)N(R⁵¹)₂, —NR⁵¹C(═O)OR⁵¹, —N(R⁵¹)C(O)N(R⁵¹)_{2J}—NO₂, ═O, ═S, ═N(R⁵¹), and —CN;
each R⁵⁰ is independently selected at each occurrence from:
—C₁-C₁₀ alkyl, —C₂-C₁₀ alkenyl, and —C₂-C₁₀ alkynyl, each of which is optionally substituted with one or more substituents independently selected from halogen, —OH, —CN, —NO₂, —NH₂, ═O, ═S, —O—C₁-C₁₀ alkyl, C₃-C₁₂ carbocycle, and a 3- to 12-membered heterocycle; and
a C₃-C₁₂ carbocycle and a 3- to 12-membered heterocycle, each of which is optionally substituted with one or more substituents independently selected from halogen, —OH, —CN, —NO₂, —NH₂, ═O, ═S, —C₁-C₁₀ alkyl, —O—C₁-C₁₀ alkyl, and —C₁-C₁₀ haloalkyl;
each R⁵¹ is independently selected at each occurrence from:
hydrogen;
C₁-C₁₀ alkyl, —C₂-C₁₀ alkenyl, and —C₂-C₁₀ alkynyl, each of which is optionally substituted with one or more substituents independently selected from halogen, —OH, —CN, —NO₂, —NH₂, ═O, ═S, —O—C₁-C₁₀ alkyl, C₃-C₁₂ carbocycle, and a 3- to 12-membered heterocycle; and
a C₃-C₁₂ carbocycle and a 3- to 12-membered heterocycle, each of which is optionally substituted with one or more substituents independently selected from halogen, —OH, —CN, —NO₂, —NH₂, ═O, ═S, —C₁-C₁₀ alkyl, —O—C₁-C₁₀ alkyl, and —C₁-C₁₀ haloalkyl;
Z¹, Z², Z³, and Z⁴ are independently selected from N or C(H);
n is selected from 1, 2, and 3;
m is 0, 1, or 2;
s is selected from 0 and 1; and
w is selected from 0, 1, 2, 3, 4, and 5.

2-23. (canceled)

24. The compound of claim 1, or a pharmaceutically acceptable isomer, racemate, hydrate, solvate, tautomer, isotope, or salt thereof, wherein
one of M¹ and M² is [chemical structure]
and
the other of M¹ and M² is selected from:

[chemical structure]

25-60. (canceled)

61. The compound or salt of claim 1, or a pharmaceutically acceptable isomer, racemate, hydrate, solvate, tautomer, isotope, or salt thereof, wherein the compound is selected from any one of the compounds in Table 16.

62. A pharmaceutical composition, comprising a compound of claim 1, or a pharmaceutically acceptable isomer, racemate, hydrate, solvate, tautomer, isotope, or salt thereof, and a pharmaceutically acceptable excipient.

63. The compound of claim 1, or a pharmaceutically acceptable isomer, racemate, hydrate, solvate, tautomer, isotope, or salt thereof, wherein the compound is covalently bound to a linker, L³ to form a compound-linker.

64. (canceled)

65. The compound of claim 63, or a pharmaceutically acceptable isomer, racemate, hydrate, solvate, tautomer, isotope, or salt thereof, wherein L³ is a cleavable linker.

66-69. (canceled)

70. The compound of claim 63, or a pharmaceutically acceptable isomer, racemate, hydrate, solvate, tautomer, isotope, or salt thereof, wherein the compound-linker is selected from any one of the compound-linkers set forth in Table 15.

71. The compound of claim 63, or a pharmaceutically acceptable isomer, racemate, hydrate, solvate, tautomer, isotope, or salt thereof, wherein L³ is selected from any one of the linkers set forth in Table 3, Table 4, Table 5, Table 6, Table 7, Table 8, Table 9, Table 10, Table 11, Table 12, Table 13, or Table 14.

72. The compound of claim 63, or a pharmaceutically acceptable isomer, racemate, hydrate, solvate, tautomer, isotope, or salt thereof, wherein -L³ is represented by the formula: [chemical structure]
wherein:
L⁴ represents the C-terminus of the peptide and L⁵ is selected from a bond, alkylene and heteroalkylene, wherein L⁵ is optionally substituted with one or more groups independently selected from R³⁰, and RX is a reactive moiety; and
R³⁰ is independently selected at each occurrence from halogen, —OH, —CN, —O-alkyl, —SH, ═O, ═S, —NH₂, —NO₂; and C₁-C₁₀alkyl, C₂-C₁₀alkenyl, and C₂-C₁₀alkynyl, each of which is independently optionally substituted at each occurrence with one or more substituents selected from halogen, —OH, —CN, —O-alkyl, —SH, ═O, ═S, —NH₂, and —NO₂.

73. (canceled)

74. The compound of claim 72, or a pharmaceutically acceptable isomer, racemate, hydrate, solvate, tautomer, isotope, or salt thereof, wherein RX is a maleimide or an alpha-halo carbonyl.

75. The compound of claim 72, or a pharmaceutically acceptable isomer, racemate, hydrate, solvate, tautomer, isotope, or salt thereof, wherein the peptide of L³ comprises Val-Cit or Val-Ala.

76. The compound of claim 63, or a pharmaceutically acceptable isomer, racemate, hydrate, solvate, tautomer, isotope, or salt thereof, wherein the compound-linker is selected from any one of the compounds in Table 17 or a pharmaceutically acceptable salt of any one thereof.

77. The compound of claim 63, or a pharmaceutically acceptable isomer, racemate, hydrate, solvate, tautomer, isotope, or salt thereof, wherein L³ is further covalently bound to an antibody, an antibody construct, or a targeting moiety to form a conjugate.

78-82. (canceled)

83. A conjugate represented by the formula: [chemical structure]
wherein:
Antibody is an antibody, antibody construct, or a targeting moiety;
n is 1-20;
D is the compound or pharmaceutically acceptable salt of claim 1; and
L³ is a linker moiety.

84. The conjugate of claim 83, or a pharmaceutically acceptable isomer, racemate, hydrate, solvate, tautomer, isotope, or salt thereof, wherein -L³- is represented by any one of the linkers set forth in Table 5, Table 8, Table 11, or Table 14.

85. The conjugate of claim 84, or a pharmaceutically acceptable isomer, racemate, hydrate, solvate, tautomer, isotope, or salt thereof, wherein -L³- is represented by the formula: [chemical structure]
wherein:
L⁴ represents the C-terminus of the peptide and L⁵ is selected from a bond, alkylene and heteroalkylene, wherein L⁵ is optionally substituted with one or more groups independently selected from R³⁰; RX* is a bond, a succinimide moiety, or a hydrolyzed succinimide moiety bound to a residue of an antibody construct, wherein [chemical structure]
on RX* represents the point of attachment to the residue of the antibody construct; and
R³⁰ is independently selected at each occurrence from halogen, —OH, —CN, —O-alkyl, —SH, ═O, ═S, —NH₂, —NO₂; and C₁-C₁₀ alkyl, C₂-C₁₀ alkenyl, and C₂-C₁₀ alkynyl, each of which is independently optionally substituted at each occurrence with one or more substituents selected from halogen, —OH, —CN, —O-alkyl, —SH, ═O, ═S, —NH₂, and —NO₂.

86. The conjugate of claim 85, or a pharmaceutically acceptable isomer, racemate, hydrate, solvate, tautomer, isotope, or salt thereof, wherein RX* is a succinamide moiety, hydrolyzed succinamide moiety or a mixture thereof and is bound to a cysteine residue of an antibody construct.

87-88. (canceled)

89. The conjugate of claim 83, or a pharmaceutically acceptable isomer, racemate, hydrate, solvate, tautomer, isotope, or salt thereof,
(a) wherein the antibody, antibody construct, or targeting moiety comprises an antigen binding domain that specifically binds to a tumor antigen or an antigen associated with the pathogenesis of fibrosis; or
(b) wherein the antibody, antibody construct, or targeting moiety comprises an antigen binding domain that specifically binds to an antigen selected from the group consisting of CLTA4, PD-1, OX40, LAG-3, GITR, GARP, CD25, CD27, PD-L1, TNFR2, ICOS, 41BB, CD70, CD73, CD38, and VTCN1; or
(c) wherein the antibody, antibody construct, or targeting moiety comprises an antigen binding domain that specifically binds to an antigen selected from the group consisting of PDGFRβ, integrin αvβ1, integrin αvβ3, integrin αvβ6, integrin αvβ8, Endosialin, FAP, ADAM12, LRRC15, MMP14, PDPN, CDH11 and F2RL2; or
(d) wherein the antibody, antibody construct, or targeting moiety comprises an antigen binding domain that specifically binds to the LRRC15 antigen; or
(e) wherein the antibody, antibody construct, or targeting moiety comprises an antigen binding domain that specifically binds to an antigen on a hepatocyte; or
(f) wherein the antibody, antibody construct or targeting moiety is an antibody; or
(g) wherein the antibody construct comprises a wild-type Fc region or domain; or
(h) wherein the antibody construct comprises a null Fc region or domain.

90-94. (canceled)

95. The conjugate of claim 83, or a pharmaceutically acceptable isomer, racemate, hydrate, solvate, tautomer, isotope, or salt thereof, wherein the antigen is ASGR1 or ASGR2.

96. (canceled)

97. The conjugate of claim 83, or a pharmaceutically acceptable isomer, racemate, hydrate, solvate, tautomer, isotope, or salt thereof, wherein the antibody is a monoclonal antibody.

98-99. (canceled)

100. A pharmaceutical composition comprising a conjugate of claim 83, or a pharmaceutically acceptable isomer, racemate, hydrate, solvate, tautomer, isotope, or salt thereof, and a pharmaceutically acceptable excipient.

101. The pharmaceutical composition of claim 100, or a pharmaceutically acceptable isomer, racemate, hydrate, solvate, tautomer, isotope, or salt thereof, wherein the average Drug-to-Antibody Ratio (DAR) is 1-8, 3-5, or 1-3.

102-103. (canceled)

104. A method for the treatment of cancer, comprising administering an effective amount of the pharmaceutical composition of claim 100 to a subject in need thereof.

105-107. (canceled)

108. A method for the treatment of fibrosis, comprising administering an effective amount of the pharmaceutical composition of claim 100 to a subject in need thereof.

109-122. (canceled)


SEQUENCE LISTING (398 sequences)

SEQ ID NO: 1 - protein, 8 aa, Artificial sequence
  Ipilumumab HCDR1
GFTFSSYT

SEQ ID NO: 2 - protein, 8 aa, Artificial sequence
  Ipilumumab HCDR2
ISYDGNNK

SEQ ID NO: 3 - protein, 11 aa, Artificial sequence
  Ipilumumab HCDR3
ARTGWLGPFDY

SEQ ID NO: 4 - protein, 7 aa, Artificial sequence
  Ipilumumab LCDR1
QSVGSSY

SEQ ID NO: 5 - protein, 3 aa, Artificial sequence
  Ipilumumab LCDR2
SSY

SEQ ID NO: 6 - protein, 9 aa, Artificial sequence
  Ipilumumab LCDR3
QQYGSSPWT

SEQ ID NO: 7 - protein, 8 aa, Artificial sequence
  nivolumab HCDR1
GITFSNSG

SEQ ID NO: 8 - protein, 8 aa, Artificial sequence
  nivolumab HCDR2
IWYDGSKR

SEQ ID NO: 9 - protein, 6 aa, Artificial sequence
  nivolumab HCDR3
ATNDDY

SEQ ID NO: 10 - protein, 7 aa, Artificial sequence
  nivolumab LCDR1
QSVSSYL

SEQ ID NO: 11 - protein, 3 aa, Artificial sequence
  nivolumab LCDR2
DAS

SEQ ID NO: 12 - protein, 9 aa, Artificial sequence
  nivolumab LCDR3
QQSSNWPRT

SEQ ID NO: 13 - protein, 8 aa, Artificial sequence
  pembrolizumab HCDR1
GYTFTNYY

SEQ ID NO: 14 - protein, 8 aa, Artificial sequence
  pembrolizumab HCDR2
INPSNGGT

SEQ ID NO: 15 - protein, 13 aa, Artificial sequence
  pembrolizumab HCDR3
ARRDYRFDMGFDY

SEQ ID NO: 16 - protein, 10 aa, Artificial sequence
  pembrolizumab LCDR1
KGVSTSGYSY

SEQ ID NO: 17 - protein, 3 aa, Artificial sequence
  pembrolizumab LCDR2
LAS

SEQ ID NO: 18 - protein, 9 aa, Artificial sequence
  pembrolizumab LCDR3
QHSRDLPLT

SEQ ID NO: 19 - protein, 8 aa, Artificial sequence
  Vonlerolizumab HCDR1
GYTFTDSY

SEQ ID NO: 20 - protein, 8 aa, Artificial sequence
  Vonlerolizumab HCDR2
MYPDNGDS

SEQ ID NO: 21 - protein, 10 aa, Artificial sequence
  Vonlerolizumab HCDR3
VLAPRWYFSV

SEQ ID NO: 22 - protein, 6 aa, Artificial sequence
  Vonlerolizumab LCDR1
QDISNY

SEQ ID NO: 23 - protein, 3 aa, Artificial sequence
  Vonlerolizumab LCDR2
YTS

SEQ ID NO: 24 - protein, 9 aa, Artificial sequence
  Vonlerolizumab LCDR3
QQGHTLPPT

SEQ ID NO: 25 - protein, 8 aa, Artificial sequence
  Varlilumab HCDR1
GFTFSSYD

SEQ ID NO: 26 - protein, 8 aa, Artificial sequence
  Varlilumab HCDR2
IWYDGSNK

SEQ ID NO: 27 - protein, 12 aa, Artificial sequence
  Varlilumab HCDR3
ARGSGNWGFFDY

SEQ ID NO: 28 - protein, 6 aa, Artificial sequence
  Varlilumab LCDR1
QGISRW

SEQ ID NO: 29 - protein, 3 aa, Artificial sequence
  Varlilumab LCDR2
AAS

SEQ ID NO: 30 - protein, 9 aa, Artificial sequence
  Varlilumab LCDR3
QQYNTYPRT

SEQ ID NO: 31 - protein, 8 aa, Artificial sequence
  Daclizumab HCDR1
GYTFTSYR

SEQ ID NO: 32 - protein, 8 aa, Artificial sequence
  Daclizumab HCDR2
INPSTGYT

SEQ ID NO: 33 - protein, 9 aa, Artificial sequence
  Daclizumab HCDR3
ARGGGVFDY

SEQ ID NO: 34 - protein, 6 aa, Artificial sequence
  Daclizumab LCDR1
SSSISY

SEQ ID NO: 35 - protein, 3 aa, Artificial sequence
  Daclizumab LCDR2
TTS

SEQ ID NO: 36 - protein, 9 aa, Artificial sequence
  Daclizumab LCDR3
HQRSTYPLT

SEQ ID NO: 37 - protein, 5 aa, Artificial sequence
  Antibody to GITR HCDR1
SYGMH

SEQ ID NO: 38 - protein, 17 aa, Artificial sequence
  Antibody to GITR HCDR2
VIWYEGSNKYYADSVKG

SEQ ID NO: 39 - protein, 15 aa, Artificial sequence
  Antibody to GITR HCDR3
GGSMVRGDYYYGMDV

SEQ ID NO: 40 - protein, 11 aa, Artificial sequence
  Antibody to GITR LCDR1
RASQGISSALA

SEQ ID NO: 41 - protein, 7 aa, Artificial sequence
  Antibody to GITR LCDR2
DASSLES

SEQ ID NO: 42 - protein, 9 aa, Artificial sequence
  Antibody to GITR LCDR3
QQFNSYPYT

SEQ ID NO: 43 - protein, 5 aa, Artificial sequence
  Antibody to LAG-3 HCDR1
DYYWN

SEQ ID NO: 44 - protein, 16 aa, Artificial sequence
  Antibody to LAG-4 HCDR2
EINHRGSTNSNPSLKS

SEQ ID NO: 45 - protein, 12 aa, Artificial sequence
  Antibody to LAG-5 HCDR3
GYSDYEYNWFDP

SEQ ID NO: 46 - protein, 11 aa, Artificial sequence
  Antibody to LAG-6 LCDR1
RASQSISSYLA

SEQ ID NO: 47 - protein, 7 aa, Artificial sequence
  Antibody to LAG-7 LCDR2
DASNRAT

SEQ ID NO: 48 - protein, 9 aa, Artificial sequence
  Antibody to LAG-8 LCDR3
QQRSNWPLT

SEQ ID NO: 49 - protein, 5 aa, Artificial Sequence
  LPXTG recognition motif
  misc_feature (3)..(3): Xaa can be any naturally occurring amino acid
LPXTG

SEQ ID NO: 50 - protein, 8 aa, Artificial sequence
  Utomilumab HCDR1
GYSFSTYW

SEQ ID NO: 51 - protein, 8 aa, Artificial sequence
  Utomilumab HCDR2
IYPGDSYT

SEQ ID NO: 52 - protein, 9 aa, Artificial sequence
  Utomilumab HCDR3
ARGYGIFDY

SEQ ID NO: 53 - protein, 6 aa, Artificial sequence
  Utomilumab LCDR1
NIGDQY

SEQ ID NO: 54 - protein, 3 aa, Artificial sequence
  Utomilumab LCDR2
QDK

SEQ ID NO: 55 - protein, 11 aa, Artificial sequence
  Utomilumab LCDR3
ATYTGFGSLAV

SEQ ID NO: 56 - protein, 8 aa, Artificial sequence
  Antibody to TNFR2 variant 1 HCDR1
GYTFTDYN

SEQ ID NO: 57 - protein, 8 aa, Artificial sequence
  Antibody to TNFR2 variant 1 HCDR2
INPNYEST

SEQ ID NO: 58 - protein, 9 aa, Artificial sequence
  Antibody to TNFR2 variant 1 HCDR3
RDKGWYFDV

SEQ ID NO: 59 - protein, 5 aa, Artificial sequence
  Antibody to TNFR2 variant 1 LCDR1
SSVKN

SEQ ID NO: 60 - protein, 3 aa, Artificial sequence
  Antibody to TNFR2 variant 1 LCDR2
YTS

SEQ ID NO: 61 - protein, 9 aa, Artificial sequence
  Antibody to TNFR2 variant 1 LCDR3
QQFTSSPYT

SEQ ID NO: 62 - protein, 10 aa, Artificial sequence
  Antibody to TNFR2 variant 2 HCDR1
GFSLSTSGMG

SEQ ID NO: 63 - protein, 7 aa, Artificial sequence
  Antibody to TNFR2 variant 2 HCDR2
IWWDDDK

SEQ ID NO: 64 - protein, 11 aa, Artificial sequence
  Antibody to TNFR2 variant 2 HCDR3
ARLTGTRYFDY

SEQ ID NO: 65 - protein, 6 aa, Artificial sequence
  Antibody to TNFR2 variant 2 LCDR1
QDINKF

SEQ ID NO: 66 - protein, 3 aa, Artificial sequence
  Antibody to TNFR2 variant 2 LCDR2
YTS

SEQ ID NO: 67 - protein, 8 aa, Artificial sequence
  Antibody to TNFR2 variant 2 LCDR3
LQYGNLWT

SEQ ID NO: 68 - protein, 8 aa, Artificial sequence
  Antibody to TNFR2 variant 3 HCDR1
GYTFTDYS

SEQ ID NO: 69 - protein, 8 aa, Artificial sequence
  Antibody to TNFR2 variant 3 HCDR2
INTETGEP

SEQ ID NO: 70 - protein, 12 aa, Artificial sequence
  Antibody to TNFR2 variant 3 HCDR3
ATYYGSSYVPDY

SEQ ID NO: 71 - protein, 6 aa, Artificial sequence
  Antibody to TNFR2 variant 3 LCDR1
QNVGTA

SEQ ID NO: 72 - protein, 3 aa, Artificial sequence
  Antibody to TNFR2 variant 3 LCDR2
WTS

SEQ ID NO: 73 - protein, 8 aa, Artificial Sequence
  Antibody to TNFR2 variant 3 LCDR3
QYSDYPYT

SEQ ID NO: 74 - protein, 7 aa, Artificial sequence
  Antibody to TNFR2 variant 4 HCDR1
GYTFTDY

SEQ ID NO: 75 - protein, 8 aa, Artificial sequence
  Antibody to TNFR2 variant 4 HCDR2
WVDPEYGS

SEQ ID NO: 76 - protein, 12 aa, Artificial sequence
  Antibody to TNFR2 variant 4 HCDR3
ARDDGSYSPFDY

SEQ ID NO: 77 - protein, 6 aa, Artificial sequence
  Antibody to TNFR2 variant 4 LCDR1 (major)
QNINKY

SEQ ID NO: 78 - protein, 3 aa, Artificial sequence
  Antibody to TNFR2 variant 4 LCDR2 (major)
YTS

SEQ ID NO: 79 - protein, 8 aa, Artificial sequence
  Antibody to TNFR2 variant 4 LCDR3 (major)
LQYVNLLT

SEQ ID NO: 80 - protein, 6 aa, Artificial sequence
  Antibody to TNFR2 variant 4 LCDR1 (minor)
ENVVTY

SEQ ID NO: 81 - protein, 3 aa, Artificial sequence
  Antibody to TNFR2 variant 4 LCDR2 (minor)
GAS

SEQ ID NO: 82 - protein, 8 aa, Artificial sequence
  Antibody to TNFR2 variant 4 LCDR3 (minor)
QGYSYPYT

SEQ ID NO: 83 - protein, 118 aa, Artificial sequence
  Ipilumumab VH
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 84 - protein, 108 aa, Artificial sequence
  Ipilumumab VL
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 85 - protein, 113 aa, Artificial sequence [sequence omitted]

SEQ ID NO: 86 - protein, 107 aa, Artificial sequence [sequence omitted]

SEQ ID NO: 87 - protein, 120 aa, Artificial sequence [sequence omitted]

SEQ ID NO: 88 - protein, 111 aa, Artificial sequence [sequence omitted]

SEQ ID NO: 89 - protein, 118 aa, Artificial sequence [sequence omitted]

SEQ ID NO: 90 - protein, 107 aa, Artificial sequence [sequence omitted]

SEQ ID NO: 91 - protein, 121 aa, Artificial sequence
  Durvalumab VH
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 92 - protein, 108 aa, Artificial sequence
  Durvalumab VL
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 93 - protein, 123 aa, Artificial sequence
  MDX-1106 VH
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 94 - protein, 106 aa, Artificial sequence
  MDX-1106 VL
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 95 - protein, 117 aa, Artificial sequence
  Antibody to TNFR2 variant 1 VH
EVQLQQSGAELVKPGASVKISCKASGYTFTDYNMDWVKQSHGKSLEWIGDINPNYESTSYNQKFKGKATLTVDKSSSTAYMEVRSLTSEDTAVFYCARDKGWYFDVWGAGTTVTVSS

SEQ ID NO: 96 - protein, 106 aa, Artificial sequence
  Antibody to TNFR2 variant 1 VL
ENVLTQSPAIMSASLGEKVTMSCRASSSVKNMYWYQQKSDASPKLWIYYTSNLAPGVPARFSGSGSGNSYSLTISSMEGEDAATYYCQQFTSSPYTFGGGTKLELK

SEQ ID NO: 97 - protein, 119 aa, Artificial sequence
  Antibody to TNFR2 variant 2 VH
QVTLKESGPGILQPSQTLSLTCSFSGFSLSTSGMGVGWIRQPSGKGLEWLAHIWWDDDKFYNPSLKSQLTISKDTSRNQVFLKLTSVVTADTATYYCARLTGTRYFDYWGQGTTLTVSS

SEQ ID NO: 98 - protein, 106 aa, Artificial sequence
  Antibody to TNFR2 variant 2 VL
DVQMTQSPSSLSASLGGKVTITCKASQDINKFIAWYQHKPGKGPRLLIHYTSTLQPGIPSKFSGSGSGRDYSFSISNLEPEDIATYYCLQYGNLWTFGGGTKLEIT

SEQ ID NO: 99 - protein, 119 aa, Artificial sequence
  Antibody to TNFR2 variant 3 VH
QIQLVQSGPELKKPGETVKISCKASGYTFTDYSMHWVKQAPGKGLKWMGWINTETGEPTYADDFKGRFAFSSETSTSTAYLQINNLKNDDTTTYFCATYYGSSYVPDYWGQGTSLTVSS

SEQ ID NO: 100 - protein, 107 aa, Artificial sequence
  Antibody to TNFR2 variant 3 VL
DIVMTQSHKFMSTSVGDRVSITCKASQNVGTAVAWYQHKPGQSPKLLIYWTSSRHTGVPDRFTGSGSGTEFTLTISNVQSEDLADYFCHQYSDYPYTFGGGTKLEIK

SEQ ID NO: 101 - protein, 119 aa, Artificial sequence
  Antibody to TNFR2 variant 4 VH
EVQLQQSGPEVGRPGSSVKISCKASGYTFTDYIMHWVKQSPGQGLEWIGWVDPEYGSTDYAEKFKKKATLTADTSSNTAYIQLSSLTSEDTATYFCARDDGSYSPFDYWGQGVMVTVSS

SEQ ID NO: 102 - protein, 106 aa, Artificial sequence
  Antibody to TNFR2 variant 4 VL (major)
DIQMTQSPPSLSASLGDKVTITCQASQNINKYIAWYQQKPGKAPRLLIRYTSTLESGTPSRFSGSGSGRDYSFSISNVESEDIASYYCLQYVNLLTFGAGTKLEIK

SEQ ID NO: 103 - protein, 107 aa, Artificial sequence
  Antibody to TNFR2 variant 4 VL (minor)
NIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIK

SEQ ID NO: 104 - protein, 120 aa, Artificial sequence
  Antibody to TNFR2 variant 5 VH
EVQLVESGGGLVQPGGSLRLSCAASGFTFSDYAMSWVRQAPGKGLEWVAVISENGSDTYYADSVKGRFTISRDDSKNTLYLQMNSLRAEDTAVYYCARDRGGAVSYFDVWGQGTLVTVSS

SEQ ID NO: 105 - protein, 109 aa, Artificial sequence
  Antibody to TNFR2 variant 5 VL
DIQMTQSPSSLSASVGDRVTITCRASQDVSSYLAWYQQKPGKAPKLLIYAASSLESGVPSRFSGSGSGTDFTLTISSLQPEDFATYYCQQYNSLPYTFGQGTKVEIKRT

SEQ ID NO: 106 - protein, 117 aa, Artificial sequence
  Vonlerolizumab VH
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRWYFSVWGQGTLVTVSS

SEQ ID NO: 107 - protein, 107 aa, Artificial sequence
  Vonlerolizumab VL
DIQMTQSPSSLSASVGDRVTITCRASQDISNYLNWYQQKPGKAPKLLIYYTSRLRSGVPSRFSGSGSGTDFTLTISSLQPEDFATYYCQQGHTLPPTFGQGTKVEIK

SEQ ID NO: 108 - protein, 107 aa, Artificial sequence
  TRX518 VL
EIVMTQSPATLSVSPGERATLSCKASQNVGTNVAWYQQKPGQAPRLLIYSASYRYSGIPARFSGSGSGTEFTLTISSLQSEDFAVYYCQQYNTDPLTFGGGTKVEIK

SEQ ID NO: 109 - protein, 119 aa, Artificial sequence
  TRX518 VH
QVTLRESGPALVKPTQTLTLTCTFSGFSLSTSGMGVGWIRQPPGKALEWLAHIWWDDDKYYNPSLKSRLTISKDTSKNQVVLTMTNMDPVDTATYYCARTRRYFPFAYWGQGTLVTVSS

SEQ ID NO: 110 - protein, 119 aa, Artificial sequence
  TRX518 VH
QVTLRESGPALVKPTQTLTLTCTFSGFSLSTSGMGVGWIRQPPGKALEWLAHIWWDDDKYYQPSLKSRLTISKDTSKNQVVLTMTNMDPVDTATYYCARTRRYFPFAYWGQGTLVTVSS

SEQ ID NO: 111 - protein, 120 aa, Artificial sequence
  Antibody to LAG-3 VH
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSS

SEQ ID NO: 112 - protein, 107 aa, Artificial sequence
  Antibody to LAG-3 VL
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIK

SEQ ID NO: 113 - protein, 140 aa, Artificial sequence
  Antibody to GARP variant 1 VH
MAVLALLFCLVTFPSCILSQVQLKESGPGLVAPSQSLSITCTVSGFSLTGYGINWVRQPPGKGLEWLGMIWSDGSTDYNSVLTSRLRISKDNSNSQVFLKMNSLQVDDTARYYCARDRNYYDYDGAMDYWGQGTSVTVSS

SEQ ID NO: 114 - protein, 121 aa, Artificial sequence
  Antibody to GARP variant 1 VL
QVQLKESGPGLVAPSQSLSITCTVSGFSLTGYGINWVRQPPGKGLEWLGMIWSDGSTDYNSVLTSRLRISKDNSNSQVFLKMNSLQVDDTARYYCARDRNYYDYDGAMDYWGQGTSVTVSS

SEQ ID NO: 115 - protein, 127 aa, Artificial sequence
  Antibody to GARP variant 2 VH
MKFPSQLLLFLLFRITGIICDIQVTQSSSYLSVSLGDRVTITCKASDHIKNWLAWYQQKPGIAPRLLVSGATSLEAGVPSRFSGSGSGKNFTLSITSLQTEDVATYYCQQYWSTPWTFGGGTTLEIR

SEQ ID NO: 116 - protein, 107 aa, Artificial sequence
  Antibody to GARP variant 2 VL
DIQVTQSSSYLSVSLGDRVTITCKASDHIKNWLAWYQQKPGIAPRLLVSGATSLEAGVPSRFSGSGSGKNFTLSITSLQTEDVATYYCQQYWSTPWTFGGGTTLEIR

SEQ ID NO: 117 - protein, 126 aa, Artificial sequence
  Antibody to GARP variant 3 VH
EVQLVQPGAELRNSGASVKVSCKASGYRFTSYYIDWVRQAPGQGLEWMGRIDPEDGGTKYAQKFQGRVTFTADTSTSTAYVELSSLRSEDTAVYYCARNEWETVVVGDLMYEYEYWGQGTQVTVSS

SEQ ID NO: 118 - protein, 107 aa, Artificial sequence
  Antibody to GARP variant 3 VL
DIQMTQSPTSLSASLGDRVTITCQASQSISSYLAWYQQKPGQAPKLLIYGASRLQTGVPSRFSGSGSGTSFTLTISGLEAEDAGTYYCQQYDSLPVTFGQGTKVELK

SEQ ID NO: 119 - protein, 107 aa, Artificial sequence
  Antibody to GARP variant 3 VL
DIQMTQSPSSLSASLGDRVTITCQASQSIVSYLAWYQQKPGQAPKLLIYGASRLQTGVPSRFSGSGSGTSFTLTISGLEAEDAGTYYCQQYASAPVTFGQGTGVELK

SEQ ID NO: 120 - protein, 107 aa, Artificial sequence
  Antibody to GARP variant 3 VL
DIQMTQSPSSLSASLGDRVTITCQASQSISSYLAWYQQKPGQAPKLLIYGTSRLKTGVPSRFSGSGSGTSFTLTISGLEAEDAGTYYCQQYYSAPVTFGQGTKVELK

SEQ ID NO: 121 - protein, 107 aa, Artificial sequence
  Antibody to GARP variant 3 VL
DIQMTQSPSSLSPSLGDRVTITCQASQTISSFLAWYHQKPGQPPKLLIYRASIPQTGVPSRFSGSGSGTSFTLTIGGLEAEDAGTYYCQQYVSAPPTFGQGTKVELK

SEQ ID NO: 122 - protein, 107 aa, Artificial sequence
  Antibody to GARP variant 3 VL
DIQMTQSPSSLSASLGDRVTITCQASQSISSYLAWYQQKPGQAPNILIYGASRLKTGVPSRFSGSGSGTSFTLTISGLEAEDAGTYYCQQYASVPVTFGQGTKVELK

SEQ ID NO: 123 - protein, 121 aa, Artificial sequence [sequence omitted]

SEQ ID NO: 124 - protein, 109 aa, Artificial sequence [sequence omitted]

SEQ ID NO: 125 - protein, 114 aa, Artificial sequence
  Antibody to 4-1BB variant 2 VH
EVQLVESGGGLVQPGGSLRLSCAASGFTFSDYWMSWVRQAPGKGLEWVADIKNDGSYTNYAPSLTNRFTISRDNAKNSLYLQMNSLRAEDTAVYYCARELTGTWGQGTMVTVSS

SEQ ID NO: 126 - protein, 113 aa, Artificial sequence
  Antibody to 4-1BB variant 2 VL
DIVMTQSPDSLAVSLGERATINCKSSQSLLSSGNQKNYLAWYQQKPGQPPKLLIYYASTRQSGVPDRFSGSGSGTDFTLTISSLQAEDVAVYYCLQYDRYPFTFGQGTKLEIK

SEQ ID NO: 127 - protein, 116 aa, Artificial sequence
  Utomilumab VH
EVQLVQSGAEVKKPGESLRISCKGSGYSFSTYWISWVRQMPGKGLEWMGKIYPGDSYTNYSPSFQGQVTISADKSISTAYLQWSSLKASDTAMYYCARGYGIFDYWGQGTLVTVSS

SEQ ID NO: 128 - protein, 108 aa, Artificial sequence
  Utomilumab VL
SYELTQPPSVSVSPGQTASITCSGDNIGDQYAHWYQQKPGQSPVLVIYQDKNRPSGIPERFSGSNSGNTATLTISGTQAMDEADYYCATYTGFGSLAVFGGGTKLTVL

SEQ ID NO: 129 - protein, 107 aa, Artificial sequence
  Antibody to ICOS variant 1 VL
DIQMTQSPSSVSASVGDRVTITCRASQGISRLLAWYQQKPGKAPKLLIYVASSLQSGVPSRFSGSGSGTDFTLTISSLQPEDFATYYCQQANSFPWTFGQGTKVEIK

SEQ ID NO: 130 - protein, 125 aa, Artificial sequence
  Antibody to ICOS variant 1 VH
QVQLVQSGAEVKKPGASVKVSCKASGYTFTGYYMHWVRQAPGQGLEWMGWINPHSGGTNYAQKFQGRVTMTRDTSISTAYMELSRLRSDDTAVYYCARTYYYDSSGYYHDAFDIWGQGTMVTVSS

SEQ ID NO: 131 - protein, 117 aa, Artificial sequence
  Antibody to ICOS variant 2 VH
EVQLVESGGGLVQPGGSLRLSCAASGFTFSDYWMDWVRQAPGKGLVWVSNIDEDGSITEYSPFVKGRFTISRDNAKNTLYLQMNSLRAEDTAVYYCTRWGRFGFDSWGQGTLVTVSS

SEQ ID NO: 132 - protein, 111 aa, Artificial sequence
  Antibody to ICOS variant 2 VL
DIVMTQSPDSLAVSLGERATINCKSSQSLLSGSFNYLTWYQQKPGQPPKLLIFYASTRHTGVPDRFSGSGSGTDFTLTISSLQAEDVAVYYCHHHYNAPPTFGPGTKVDIK

SEQ ID NO: 133 - protein, 118 aa, Artificial sequence
  Vorsetuzumab VH
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSS

SEQ ID NO: 134 - protein, 111 aa, Artificial sequence
  Vorsetuzumab VL
DIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIK

SEQ ID NO: 135 - protein, 122 aa, Artificial sequence
  Rinucumab VH
QLQLQESGPGLVKPSETLSLTCTVSGGSITSSSYYWGWIRQPPGKGLEWIGSIYYRGSTNYNPSLKSRVTISVDSSKNQFYLKVSSVTAVDTAVYYCARQNGAARPSWFDPWGQGTLVTVSS

SEQ ID NO: 136 - protein, 108 aa, Artificial sequence
  Rinucumab VL
EIVLTQSPDTISLSPGERATLSCRASQSISSIYLAWYQQKPGQAPRLLIYGASSRVTGIPDRFSVSGSGTDFTLTISRLEPEDFAVYYCQHYGISPFTFGPGTKVDIR

SEQ ID NO: 137 - protein, 117 aa, Artificial sequence
  Oleclumab VH
EVQLLESGGGLVQPGGSLRLSCAASGFTFSSYAYSWVRQAPGKGLEWVSAISGSGGRTYYADSVKGRFTISRDNSKNTLYLQMNSLRAEDTAVYYCARLGYGRVDEWGRGTLVTVSS

SEQ ID NO: 138 - protein, 110 aa, Artificial sequence
  Oleclumab VL
QSVLTQPPSASGTPGQRVTISCSGSLSNIGRNPVNWYQQLPGTAPKLLIYLDNLRLSGVPDRFSGSKSGTSASLAISGLQSEDEADYYCATWDDSHPGWTFGGGTKLTVL

SEQ ID NO: 139 - protein, 107 aa, Artificial sequence
  Antibody to CD73 VL
DIQMTQSPSSLSASVGDRVTITCRASQGISSWLAWYQQKPEKAPKSLIYAASSLQSGVPSRFSGSGSGTDFTLTISSLQPEDFATYYCQQYNSYPLTFGGGTKVEIK

SEQ ID NO: 140 - protein, 121 aa, Artificial sequence
  Antibody to CD73 VH
QVQLVESGGGVVQPGRSLRLSCAASGFTFSNYGMHWVRQAPGKGLEWVAVILYDGSNKYYPDSVKGRFTISRDNSKNTLYLQMNSLRAEDTAVYYCARGGSSWYPDSFDIWGQGTMVTVSS

SEQ ID NO: 141 - protein, 122 aa, Artificial sequence [sequence omitted]

SEQ ID NO: 142 - protein, 107 aa, Artificial sequence [sequence omitted]

SEQ ID NO: 143 - protein, 117 aa, Artificial sequence
  Etaracizumab VH
QVQLVESGGGVVQPGRSLRLSCAASGFTFSSYDMSWVRQAPGKGLEWVAKVSSGGGSTYYLDTVQGRFTISRDNSKNTLYLQMNSLRAEDTAVYYCARHLHGSFASWGQGTTVTVSS

SEQ ID NO: 144 - protein, 107 aa, Artificial sequence
  Etaracizumab VL
EIVLTQSPATLSLSPGERATLSCQASQSISNFLHWYQQRPGQAPRLLIRYRSQSISGIPARFSGSGSGTDFTLTISSLEPEDFAVYYCQQSGSWPLTFGGGTKVEIK

SEQ ID NO: 145 - protein, 119 aa, Artificial sequence
  Intetumumab VH
QVQLVESGGGVVQPGRSRRLSCAASGFTFSRYTMHWVRQAPGKGLEWVAVISFDGSNKYYVDSVKGRFTISRDNSENTLYLQVNILRAEDTAVYYCAREARGSYAFDIWGQGTMVTVSS

SEQ ID NO: 146 - protein, 108 aa, Artificial sequence
  Intetumumab VL
EIVLTQSPATLSLSPGERATLSCRASQSVSSYLAWYQQKPGQAPRLLIYDASNRATGIPARFSGSGSGTDFTLTISSLEPEDFAVYYCQQRSNWPPFTFGPGTKVDIK

SEQ ID NO: 147 - protein, 116 aa, Artificial sequence
  Antibody to Integrin avB8 VH
EVQLVESGGGLVQPGGSLRLSCAVSGFVFSRYWMSWVRQAPGKGLEWIGEINPDSSTINYTSSLKDRFTISRDNAKNSLYLQMNSLRAEDTAVYYCASLITTEDYWGQGTTVTVSS

SEQ ID NO: 148 - protein, 108 aa, Artificial sequence
  Antibody to Integrin avB8 VL
EIVLTQSPSSLSLSPGERVTITCKASQDINSYLSWYQQKPGKAPKLLIYYANRLVDGVPARFSGSGSGQDYTLTISSLEPEDFAVYYCLQYDEFPYTFGGGTKLEIKR

SEQ ID NO: 149 - protein, 124 aa, Artificial sequence
  Ontuxizumab VH
QVQLQESGPGLVRPSQTLSLTCTASGYTFTDYVIHWVKQPPGRGLEWIGYINPYDDDTTYNQKFKGRVTMLVDTSSNTAYLRLSSVTAEDTAVYYCARRGNSYDGYFDYSMDYWGSGTPVTVSS

SEQ ID NO: 150 - protein, 108 aa, Artificial sequence
  Ontuxizumab VL
DIQMTQSPSSLSASVGDRVTITCRASQNVGTAVAWLQQTPGKAPKLLIYSASNRYTGVPSRFSGSGSGTDYTFTISSLQPEDIATYYCQQYTNYPMYTFGQGTKVQIK

SEQ ID NO: 151 - protein, 121 aa, Artificial sequence
  Antibody to FAP variant 1 VH
QVQLQESGPGLVKPSQTLSLTCAISGDSVSSNSVTWNWIRQSPSRGLEWLGRTYYRSKWYNDYAVSVKGRITINPDTSKNQFYLQLKSVTPEDAAVYYCARDSSILYGDYWGQGTLVTVSS

SEQ ID NO: 152 - protein, 120 aa, Artificial sequence
  Antibody to FAP variant 1 VH
QVQLQQSGPGLVKPSQTLSLTCAISGDSVSSNSVTWNWIRQSPSRGLEWLGRTYYRSKWYNDYAVSVKGRITINPDTSKNQFYLQLKSVTPEDAAVYYCARDSSILYGDYWGQGTLVTVS

SEQ ID NO: 153 - protein, 115 aa, Artificial sequence
  Antibody to FAP variant 1 VL
QAVLTQPSSLSASPGASASLTCTLPSGINVGTYRIFWFQQKPGSPPQYLLSYKSDSDNHQGSGVPSRFSGSKDASANAGILLISGLQSEDEADYYCMIWHSSAWVFGGGTKLTVL

SEQ ID NO: 154 - protein, 122 aa, Artificial sequence
  Antibody to FAP variant 2 VH
QVQLVQSGAEVKKPGASVKVSCKTSGYTFTDYYIHWVRQAPGQGLEWMGWINPNRGGTNYAQKFQGRVTMTRDTSIATAYMELSRLRSDDTAVYYCATASLKIAAVGTFDCWGQGTLVTVSS

SEQ ID NO: 155 - protein, 107 aa, Artificial sequence
  Antibody to FAP variant 2 VL
SYELTQPPSVSVSPGQTARITCSGDALSKQYAFWFQQKPGQAPILVIYQDTKRPSGIPGRFSGSSSGTTVTLTISGAQADDEADYYCQSADSSGTYVFGTGTKVTVL

SEQ ID NO: 156 - protein, 116 aa, Artificial sequence
  Antibody to FAP variant 3 VH
EVQLVETGGGVVQPGRSLRLSCAASGFSFSTHGMYWVRQPPGKGLEWVAVISYDGSDKKYADSVKGRFTISRDNSKNTVYLEMSSVRAEDTALYYCFCRRDAFDLWGQGTMVTVSS

SEQ ID NO: 157 - protein, 108 aa, Artificial sequence
  Antibody to FAP variant 3 VL
SYVLTQPPSVSVSPGQTARITCSGDALPKKYAYWYQQKSGQAPVLVIYEDTKRPSGIPERFSGSSSGTMATLTISGAQVEDEADYYCYSTDSSGNYWVFGGGTEVTVL

SEQ ID NO: 158 - protein, 124 aa, Artificial sequence
  Antibody to FAP variant 4 VH
EVQLVESGGGLVEPGGSLRLSCAASGFTFSDAWMNWVRQAPGKGLEWVGRIKTKSDGGTTDYAAPVRGRFSISRDDSKNTLFLEMNSLKTEDTAIYYCFITVIVVSSESPLDHWGQGTLVTVSS

SEQ ID NO: 159 - protein, 108 aa, Artificial sequence
  Antibody to FAP variant 4 VL
SYELTQPPSVSVSPGQTARITCSGDELPKQYAYWYQQKPGQAPVLVIYKDRQRPSGIPERFSGSSSGTTVTLTISGVQAEDEADYYCQSAYSINTYVIFGGGTKLTVL

SEQ ID NO: 160 - protein, 125 aa, Artificial sequence
  Antibody to FAP variant 5 VH
EVQLVESGGGLVKPGGSLRLSCAASGFTFSDYYMSWIRQAPGKGLEWISYISSGSSYTNYADSVKGRFTISRDNAKKSVYLEVNGLTVEDTAVYYCARVRYGDREMATIGGFDFWGQGTLVTVSS

SEQ ID NO: 161 - protein, 108 aa, Artificial sequence
  Antibody to FAP variant 5 VL
SYELTQPPSVSVSPGQTARITCSGDALPKQYAYWYQQSPGQAPVLVIYKDSERPSGIPERFSGSSSGTTVTLTISGVQAEDEADYYCQSADSGGTSRIFGGGTKLTVL

SEQ ID NO: 162 - protein, 124 aa, Artificial sequence
  Antibody to FAP variant 6 VH
QVQLQESGPGLVRSTETLSLTCLVSGDSINSHYWSWLRQSPGRGLEWIGYIYYTGPTNYNPSLKSRVSISLGTSKDQFSLKLSSVTAADTARYYCARNKVFWRGSDFYYYMDVWGKGTTVTVSS

SEQ ID NO: 163 - protein, 110 aa, Artificial sequence
  Antibody to FAP variant 6 VL
EIVLTQSPGTLSLSLGERATLSCRASQSLANNYLAWYQQKPGQAPRLLMYDASTRATGIPDRFSGSGSGTDFTLTISRLEPEDFAVYYCQQFVTSHHMYIFGQGTKVEIK

SEQ ID NO: 164 - protein, 129 aa, Artificial sequence
  Antibody to FAP variant 7 VH
HVQLQESGPGLVKPSETLSLTCTVSGGSISSNNYYWGWIRQTPGKGLEWIGSIYYSGSTNYNPSLKSRVTISVDTSKNQFSLKLSSVTAADTAVYYCARGARWQARPATRIDGVAFDIWGQGTMVTVSS

SEQ ID NO: 165 - protein, 129 aa, Artificial sequence
  Antibody to FAP variant 7 VH
QVQLQESGPGLVKPSETLSLTCTVSGGSISSNNYYWGWIRQTPGKGLEWIGSIYYSGSTNYNPSLKSRVTISVDTSKNQFSLKLSSVTAADTAVYYCARGARWQARPATRIDGVAFDIWGQGTMVTVSS

SEQ ID NO: 166 - protein, 121 aa, Artificial sequence
  Antibody to FAP variant 7 VH
EVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDWSRSGYYLPDYWGQGTLVTVSS

SEQ ID NO: 167 - protein, 107 aa, Artificial sequence
  Antibody to FAP variant 7 VL
ETTLTQSPGTLSLSPGERATLSCRASQTVTRNYLAWYQQKPGQAPRLLMYGASNRAAGVPDRFSGSGSGTDFTLTISRLEPEDFAVYYCQQFGSPYTFGQGTKVEIK

SEQ ID NO: 168 - protein, 112 aa, Artificial sequence
  Antibody to FAP variant 7 VL
DVVMTQSPLSLPVTLGQPASISCRSSQSLLHSNGYNYLDWYLQRPGQSPHLLIFLGSNRASGVPDRFSGSGSGTDFTLKISRVEAEDVGIYYCMQALQTPPTFGQGTKVEIK

SEQ ID NO: 169 - protein, 115 aa, Artificial sequence
  Antibody to ASGR1 hzJ4.11F V78A VH
QVQLVQSGAEVKKPGASVKVSCKASGYTFTSYWINWVRQAPGQGLEWMGRIVPGSGSTYYNEMFKDRVTMTRDTSTSTAYMELSSLRSEDTAVYYCARKPNFDVWGQGTTVTVSS

SEQ ID NO: 170 - protein, 115 aa, Artificial sequence
  Antibody to ASGR1 hzJ4.12F V78A, M80I, E81Q VH
QVQLVQSGAEVKKPGASVKVSCKASGYTFTSYWINWVRQAPGQGLEWMGRIVPGSGSTYYNEMFKDRVTMTRDTSTSTAYIQLSSLRSEDTAVYYCARKPNFDVWGQGTTVTVSS

SEQ ID NO: 171 - protein, 119 aa, Artificial sequence; identical to SEQ ID NO: 145
  Antibody to Integrin av variant 1 VH

SEQ ID NO: 172 - protein, 108 aa, Artificial sequence; identical to SEQ ID NO: 146
  Antibody to Integrin av variant 1 VL

SEQ ID NO: 173 - protein, 107 aa, Artificial sequence [sequence omitted]

SEQ ID NO: 174 - protein, 118 aa, Artificial sequence [sequence omitted]

SEQ ID NO: 175 - protein, 124 aa, Artificial sequence
  Antibody to Integrin avB6 variant 1 VH
QVQLQESGPGLVKPSQTLSLTCTVSGGSISSGVYYWTWIRQHPGNGLEWIGYIYYSGSTSYNPSLKSRVTISVDTSKKQFSLNLTSVTAADTAVYYCAREGPLRGDYYYGLDVWGQGTTVTVSS

SEQ ID NO: 176 - protein, 108 aa, Artificial sequence
  Antibody to Integrin avB6 variant 1 VL
EIVLTQSPGTLSLSPGERATLSCRAGQTISSRYLAWYQQKPGQAPRPLIYGASSRATGIPDRFSGSGSGTDFTLTISRLEPEDFAVYYCQQYGSSPRTFGQGTKVEIK

SEQ ID NO: 177 - protein, 128 aa, Artificial sequence
  Antibody to Integrin avB6 variant 2 VH
QVQLQESGPGLVKPSQTLSLTCTVSGGSISSGGYYWSWIRQHPGKGLEWIGYIYYSGSTYYNPSLKSRVTISVDTSKNQFSLKLSSVTAADTAMYYCARYRGPAAGRGDFYYFGMDVWGQGTTVTVSS

SEQ ID NO: 178 - protein, 113 aa, Artificial sequence
  Antibody to Integrin avB6 variant 2 VL
DIVMTQTPLSLSVTPGQPASIFCKSSQSLLNSDGKTYLCWYLQKPGQPPQLLIYEVSNRFSGVPDRFSGSGSGTDFTLKISRVEAEDVGVYYCMQGIQLPWAFFGQGTKVEIK

SEQ ID NO: 179 - protein, 125 aa, Artificial sequence
  Antibody to Integrin avB6 variant 3 VH
QVQLVESGGGVVQPGRSLRLSCAASGFTFSSYGMHWVRQAPGKGLEWVAVIWYGGSNKYYADSVKGRFTISRDNSKNTLYLQMNSLRAEDTAVYYCARDLAARRGDYYYYGMDVWGQGTTVTVSS

SEQ ID NO: 180 - protein, 107 aa, Artificial sequence
  Antibody to Integrin avB6 variant 3 VL
SSELTQDPVVSVALGQTVRITCQGDSLRSYYLSWYQQKPGQAPVLVIYGKNNRPSGIPDRFSGSNSGNTASLTITGAQAEDEADYYCNSRDSSGNHLFGGGTKLTVL

SEQ ID NO: 181 - protein, 125 aa, Artificial sequence
  Antibody to Integrin avB6 variant 4 VH
QVQLQESGPGLVKPSQTLSLTCTVSGGSISSGGYYWSWIRQHPGKGLEWIGYIYYSGRTYNNPSLKSRVTISVDTSKNQFSLKLSSVTAADTAVYYCARVATGRADYHFYAMDVWGQGTTVTVSS

SEQ ID NO: 182 - protein, 106 aa, Artificial sequence
  Antibody to Integrin avB6 variant 4 VL
SYELTQPSSVSVSPGQTARITCSGDVLAKKSARWFHQKPGQAPVLVIYKDSERPSGIPERFSGSSSGTTVTLTISGAQVEDEAAYYCYSAADNNLVFGGGTKLTVL

SEQ ID NO: 183 - protein, 119 aa, Artificial sequence
  Varlilumab VH
QVQLVESGGGVVQPGRSLRLSCAASGFTFSSYDMHWVRQAPGKGLEWVAVIWYDGSNKYYADSVKGRFTISRDNSKNTLYLQMNSLRAEDTAVYYCARGSGNWGFFDYWGQGTLVTVSS

SEQ ID NO: 184 - protein, 107 aa, Artificial sequence
  Varlilumab VL
DIQMTQSPSSLSASVGDRVTITCRASQGISRWLAWYQQKPEKAPKSLIYAASSLQSGVPSRFSGSGSGTDFTLTISSLQPEDFATYYCQQYNTYPRTFGQGTKVEIK

SEQ ID NO: 185 - protein, 116 aa, Artificial sequence
  Daclizumab VH
QVQLVQSGAEVKKPGSSVKVSCKASGYTFTSYRMHWVRQAPGQGLEWIGYINPSTGYTEYNQKFKDKATITADESTNTAYMELSSLRSEDTAVYYCARGGGVFDYWGQGTLVTVSS

SEQ ID NO: 186 - protein, 106 aa, Artificial sequence
  Daclizumab VL
DIQMTQSPSTLSASVGDRVTITCSASSSISYMHWYQQKPGKAPKLLIYTTSNLASGVPARFSGSGSGTEFTLTISSLQPDDFATYYCHQRSTYPLTFGQGTKVEVK

SEQ ID NO: 187 - protein, 124 aa, Artificial sequence
  Antibody to GITR VH
QVQLVESGGGVVQPGRSLRLSCAASGFTFSSYGMHWVRQAPGKGLEWVAVIWYEGSNKYYADSVKGRFTISRDNSKNTLYLQMNSLRAEDTAVYYCARGGSMVRGDYYYGMDVWGQGTTVTVSS

SEQ ID NO: 188 - protein, 107 aa, Artificial sequence
  Antibody to GITR VL
AIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIK

SEQ ID NO: 189 - protein, 120 aa, Artificial sequence
  Antibody huM25 to LRRC15 VH
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGNYRAWFGYWGQGTLVTVSS

SEQ ID NO: 190 - protein, 107 aa, Artificial sequence
  Antibody huM25 to LRRC15 VL
DIQMTQSPSSLSASVGDRVTITCRASQDISNYLNWYQQKPGGAVKFLIYYTSRLHSGVPSRFSGSGSGTDYTLTISSLQPEDFATYFCQQGEALPWTFGGGTKVEIK

SEQ ID NO: 191 - protein, 119 aa, Artificial sequence
  Antibody huAD208.4.1 to LRRC15 VH
EVQLVQSGAEVKKPGSSVKVSCKASGFTFTDYYIHWVKQAPGQGLEWIGLVYPYIGGTNYNQKFKGKATLTVDTSTTTAYMEMSSLRSEDTAVYYCARGDNKYDAMDYWGQGTTVTVSS

SEQ ID NO: 192 - protein, 110 aa, Artificial sequence
  Antibody huAD208.4.1 to LRRC15 VL
DIVLTQSPDSLAVSLGERATINCRASQSVSTSSYSYMHWYQQKPGQPPKLLIKYASSLESGVPDRFSGSGSGTDFTLTISSLQAEDVAVYYCEQSWEIRTFGGGTKVEIK

SEQ ID NO: 193 - protein, 121 aa, Artificial sequence
  Antibody huAD208.12.1 to LRRC15 VH
EVQLVQSGAEVKKPGSSVKVSCKASGYTFTNYWMHWVKQAPGQGLEWIGMIHPNSGSTKHNEKFRGKATLTVDESTTTAYMELSSLRSEDTAVYYCARSDFGNYRWYFDVWGQGTTVTVSS

SEQ ID NO: 194 - protein, 107 aa, Artificial sequence
  Antibody huAD208.12.1 to LRRC15 VL
EIVLTQSPATLSLSPGERATLSCRASQSSSNNLHWYQQKPGQAPRVLIKYVSQSISGIPARFSGSGSGTDFTLTISSLEPEDFAVYFCQQSNSWPFTFGQGTKLEIK

SEQ ID NO: 195 - protein, 119 aa, Artificial sequence
  Antibody huAD208.14.1 to LRRC15 VH
EVQLVQSGAEVKKPGSSVKVSCKASGFTFTDYYIHWVKQAPGQGLEWIGLVYPYIGGSSYNQQFKGKATLTVDTSTSTAYMELSSLRSEDTAVYYCARGDNNYDAMDYWGQGTTVTVSS

SEQ ID NO: 196 - protein, 110 aa, Artificial sequence
  Antibody huAD208.14.1 to LRRC15 VL
DIVLTQSPDSLAVSLGERATISCRASQSVSTSTYNYMHWYQQKPGQPPKLLVKYASNLESGVPDRFSGSGSGTDFTLTISSLQAEDVAVYYCHHTWEIRTFGGGTKVEIK

SEQ ID NO: 197 - protein, 120 aa, Artificial sequence
  Antibody hu139.10 to LRRC15 VH
EVQLVESGGGLVQPGGSLRLSCAVSGFSLTSYGVHWVRQATGKGLEWLGVIWAGGSTNYNSALMSRLTISKENAKSSVYLQMNSLRAGDTAMYYCATHMITEDYYGMDYWGQGTTVTVSS

SEQ ID NO: 198 - protein, 112 aa, Artificial sequence
  Antibody hu139.10 to LRRC15 VL
DIVMTQSPDSLAVSLGERATINCKSSQSLLNSRTRKNYLAWYQQKPGQSPKLLIYWASTRESGVPDRFSGSGSGTDFTLTISSLQAEDVAVYYCKQSYNLPTFGGGTKVEIK

SEQ ID NO: 199 - protein, 5 aa, Artificial sequence
  Antibody huAD208.4.1 to LRRC15 HCDR1
DYYIH

SEQ ID NO: 200 - protein, 17 aa, Artificial sequence
  Antibody huAD208.4.1 to LRRC15 HCDR2
LVYPYIGGTNYNQKFKG

SEQ ID NO: 201 - protein, 10 aa, Artificial sequence
  Antibody huAD208.4.1 to LRRC15 HCDR3
GDNKYDAMDY

SEQ ID NO: 202 - protein, 15 aa, Artificial sequence
  Antibody huAD208.4.1 to LRRC15 LCDR1
RASQSVSTSSYSYMH

SEQ ID NO: 203 - protein, 7 aa, Artificial sequence
  Antibody huAD208.4.1 to LRRC15 LCDR2
YASSLES

SEQ ID NO: 204 - protein, 8 aa, Artificial sequence
  Antibody huAD208.4.1 to LRRC15 LCDR3
EQSWEIRT

SEQ ID NO: 205 - protein, 5 aa, Artificial sequence
  Antibody huAD208.12.1 to LRRC15 HCDR1
NYWMH

SEQ ID NO: 206 - protein, 17 aa, Artificial sequence
  Antibody huAD208.12.1 to LRRC15 HCDR2
MIHPNSGSTKHNEKFRG

SEQ ID NO: 207 - protein, 12 aa, Artificial sequence
  Antibody huAD208.12.1 to LRRC15 HCDR3
SDFGNYRWYFDV

SEQ ID NO: 208 - protein, 11 aa, Artificial sequence
  Antibody huAD208.12.1 to LRRC15 LCDR1
RASQSSSNNLH

SEQ ID NO: 209 - protein, 7 aa, Artificial sequence
  Antibody huAD208.12.1 to LRRC15 LCDR2
YVSQSIS

SEQ ID NO: 210 - protein, 9 aa, Artificial sequence
  Antibody huAD208.12.1 to LRRC15 LCDR3
QQSNSWPFT

SEQ ID NO: 211 - protein, 5 aa, Artificial sequence
  Antibody huAD208.14.1 to LRRC15 HCDR1
DYYIH

SEQ ID NO: 212 - protein, 17 aa, Artificial sequence
  Antibody huAD208.14.1 to LRRC15 HCDR2
LVYPYIGGSSYNQQFKG

SEQ ID NO: 213 - protein, 10 aa, Artificial sequence
  Antibody huAD208.14.1 to LRRC15 HCDR3
GDNNYDAMDY

SEQ ID NO: 214 - protein, 15 aa, Artificial sequence
  Antibody huAD208.14.1 to LRRC15 LCDR1
RASQSVSTSTYNYMH

SEQ ID NO: 215 - protein, 7 aa, Artificial sequence
  Antibody huAD208.14.1 to LRRC15 LCDR2
YASNLES

SEQ ID NO: 216 - protein, 8 aa, Artificial sequence
  Antibody huAD208.14.1 to LRRC15 LCDR3
HHTWEIRT

SEQ ID NO: 217 - protein, 5 aa, Artificial sequence
  Antibody hu139.10 to LRRC15 HCDR1
SYGVH

SEQ ID NO: 218 - protein, 16 aa, Artificial sequence
  Antibody hu139.10 to LRRC15 HCDR2
VIWAGGSTNYNSALMS

SEQ ID NO: 219 - protein, 12 aa, Artificial sequence
  Antibody hu139.10 to LRRC15 HCDR3
HMITEDYYGMDY

SEQ ID NO: 220 - protein, 17 aa, Artificial sequence
  Antibody hu139.10 to LRRC15 LCDR1
KSSQSLLNSRTRKNYLA

SEQ ID NO: 221 - protein, 7 aa, Artificial sequence
  Antibody hu139.10 to LRRC15 LCDR2
WASTRES

SEQ ID NO: 222 - protein, 8 aa, Artificial sequence
  Antibody hu139.10 to LRRC15 LCDR3
KQSYNLPT

SEQ ID NO: 223 - protein, 5 aa, Artificial sequence
  Antibody muAD210.40.9 to LRRC15 HCDR1
NYWLG

SEQ ID NO: 224 - protein, 17 aa, Artificial sequence
  Antibody muAD210.40.9 to LRRC15 HCDR2
DIYPGGGNTYYNEKLKG

SEQ ID NO: 225 - protein, 11 aa, Artificial sequence
  Antibody muAD210.40.9 to LRRC15 HCDR3
WGDKKGNYFAY

SEQ ID NO: 226 - protein, 12 aa, Artificial sequence
  Antibody muAD210.40.9 to LRRC15 LCDR1
TASSSVYSSYLH

SEQ ID NO: 227 - protein, 7 aa, Artificial sequence
  Antibody muAD210.40.9 to LRRC15 LCDR2
STSNLAS

SEQ ID NO: 228 - protein, 8 aa, Artificial sequence
  Antibody muAD210.40.9 to LRRC15 LCDR3
HQYHRSPT

SEQ ID NO: 229 - protein, 5 aa, Artificial sequence
  Antibody muAD209.9.1 to LRRC15 HCDR1
NFGMN

SEQ ID NO: 230 - protein, 17 aa, Artificial sequence
  Antibody muAD209.9.1 to LRRC15 HCDR2
WINLYTGEPTFADDFKG

SEQ ID NO: 231 - protein, 13 aa, Artificial sequence
  Antibody muAD209.9.1 to LRRC15 HCDR3
KGETYYRYDGFAY

SEQ ID NO: 232 - protein, 16 aa, Artificial sequence
  Antibody muAD209.9.1 to LRRC15 LCDR1
RSSKSLLHSNGNTHLY

SEQ ID NO: 233 - protein, 7 aa, Artificial sequence
  Antibody muAD209.9.1 to LRRC15 LCDR2
RMSNLAS

SEQ ID NO: 234 - protein, 9 aa, Artificial sequence
  Antibody muAD209.9.1 to LRRC15 LCDR3
MQLLEYPYT

SEQ ID NO: 235 - protein, 5 aa, Artificial sequence
  Antibody mAb-C to ASGR1 HCDR1
SYAMS

SEQ ID NO: 236 - protein, 17 aa, Artificial sequence
  Antibody mAb-C to ASGR1 HCDR2
AISGSGGSTYYADSVKG

SEQ ID NO: 237 - protein, 11 aa, Artificial sequence
  Antibody mAb-C to ASGR1 HCDR3
DFSSRRWYLEY

SEQ ID NO: 238 - protein, 11 aa, Artificial sequence
  Antibody mAb-C to ASGR1 LCDR1
QGDSLRSYYAS

SEQ ID NO: 239 - protein, 7 aa, Artificial sequence
  Antibody mAb-C to ASGR1 LCDR2
GKNNRPS

SEQ ID NO: 240 - protein, 12 aa, Artificial sequence
  Antibody mAb-C to ASGR1 LCDR3
NSLERIGYLSYV

SEQ ID NO: 241 - protein, 5 aa, Artificial sequence
  Antibody G2D to ASGR1 HCDR1
GYYMH

SEQ ID NO: 242 - protein, 17 aa, Artificial sequence
  Antibody G2D to ASGR1 HCDR2
RINPNNGATNYNQNFKD

SEQ ID NO: 243 - protein, 17 aa, Artificial sequence
  Antibody G2D to ASGR1 HCDR2
RINPNQGATNYNQNFKD

SEQ ID NO: 244 - protein, 17 aa, Artificial sequence
  Antibody G2D to ASGR1 HCDR2
RINPNNAATNYNQNFKD

SEQ ID NO: 245 - protein, 5 aa, Artificial sequence
  Antibody G2D to ASGR1 HCDR3
VNFYY

SEQ ID NO: 246 - protein, 11 aa, Artificial sequence
  Antibody G2D to ASGR1 LCDR1
KASQVINSYLS

SEQ ID NO: 247 - protein, 7 aa, Artificial sequence
  Antibody G2D to ASGR1 LCDR2
RANTLVD

SEQ ID NO: 248 - protein, 7 aa, Artificial sequence
  Antibody G2D to ASGR1 LCDR2
RANTLVS

SEQ ID NO: 249 - protein, 7 aa, Artificial sequence
  Antibody G2D to ASGR1 LCDR2
RANTLVE

SEQ ID NO: 250 - protein, 9 aa, Artificial sequence
  Antibody G2D to ASGR1 LCDR3
LQYAEFPYT

SEQ ID NO: 251 - protein, 5 aa, Artificial sequence
  Antibody K2E to ASGR1 HCDR1
SYTMH

SEQ ID NO: 252 - protein, 17 aa, Artificial sequence
  Antibody K2E to ASGR1 HCDR2
YISPSSGYTEYNQKFKD

SEQ ID NO: 253 - protein, 4 aa, Artificial sequence
  Antibody K2E to ASGR1 HCDR3
KFDY

SEQ ID NO: 254 - protein, 11 aa, Artificial sequence
  Antibody K2E to ASGR1 LCDR1
KASQDINSYLS

SEQ ID NO: 255 - protein, 7 aa, Artificial sequence
  Antibody K2E to ASGR1 LCDR2
RANRLVD

SEQ ID NO: 256 - protein, 7 aa, Artificial sequence
  Antibody K2E to ASGR1 LCDR2
RANRLVE

SEQ ID NO: 257 - protein, 9 aa, Artificial sequence
  Antibody K2E to ASGR1 LCDR3
LQYDEFPFT

SEQ ID NO: 258 - protein, 5 aa, Artificial sequence
  Antibody J4F to ASGR1 HCDR1
SYWIN

SEQ ID NO: 259 - protein, 17 aa, Artificial sequence
  Antibody J4F to ASGR1 HCDR2
RIVPGSGSTYYNEMFKD

SEQ ID NO: 260 - protein, 6 aa, Artificial sequence
  Antibody J4F to ASGR1 HCDR3
KPNFDV

SEQ ID NO: 261 - protein, 11 aa, Artificial sequence
  Antibody J4F to ASGR1 LCDR1
KASQNVGTNVA

SEQ ID NO: 262 - protein, 7 aa, Artificial sequence
  Antibody J4F to ASGR1 LCDR2
SASYRFS

SEQ ID NO: 263 - protein, 9 aa, Artificial sequence
  Antibody J4F to ASGR1 LCDR3
QQYNSYPLT

SEQ ID NO: 264 - protein, 6 aa, Artificial sequence
  Antibody L4L to ASGR1 HCDR1
SDYAWN

SEQ ID NO: 265 - protein, 16 aa, Artificial sequence
  Antibody L4L to ASGR1 HCDR2
YISYSGSTRYNPSLKS

SEQ ID NO: 266 - protein, 12 aa, Artificial sequence
  Antibody L4L to ASGR1 HCDR3
RYRYDEGYGMDY

SEQ ID NO: 267 - protein, 11 aa, Artificial sequence
  Antibody L4L to ASGR1 LCDR1
RASENIYSNLA

SEQ ID NO: 268 - protein, 7 aa, Artificial sequence
  Antibody L4L to ASGR1 LCDR2
AATNLAD

SEQ ID NO: 269 - protein, 7 aa, Artificial sequence
  Antibody L4L to ASGR1 LCDR2
AATNLAS

SEQ ID NO: 270 - protein, 7 aa, Artificial sequence
  Antibody L4L to ASGR1 LCDR2
AATNLAE

SEQ ID NO: 271 - protein, 10 aa, Artificial sequence
  Antibody L4L to ASGR1 LCDR3
QHFWGTPPWT

SEQ ID NO: 272 - protein, 6 aa, Artificial sequence
  Antibody H8K to ASGR1 HCDR1
SDYAWN

SEQ ID NO: 273 - protein, 16 aa, Artificial sequence
  Antibody H8K to ASGR1 HCDR2
YRSYRGSTSYNPSLKS

SEQ ID NO: 274 - protein, 14 aa, Artificial sequence
  Antibody H8K to ASGR1 HCDR3
RGYYGSSSHWYFDV

SEQ ID NO: 275 - protein, 11 aa, Artificial sequence
  Antibody H8K to ASGR1 LCDR1
RASENIYSYLA

SEQ ID NO: 276 - protein, 7 aa, Artificial sequence
  Antibody H8K to ASGR1 LCDR2
NAKTLAE

SEQ ID NO: 277 - protein, 9 aa, Artificial sequence
  Antibody H8K to ASGR1 LCDR3
QHHYGTPLT

SEQ ID NO: 278 - protein, 330 aa, Artificial sequence
  Human IgG1 Heavy Chain Constant Region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 279 - protein, 330 aa, Artificial sequence
  Human IgG1null Heavy Chain Constant Region L117A L118A G120A K205A
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 280 - protein, 107 aa, Artificial sequence [sequence omitted]

SEQ ID NO: 281 - protein, 330 aa, Artificial sequence
  Mouse IgG2a Heavy Chain Constant Region
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 282 - protein, 107 aa, Artificial sequence
  Mouse Kappa Light Chain Constant Domain
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 283 - protein, 331 aa, Artificial sequence
  Rat IgG2b Heavy Chain Constant Region
AQTTAPSVYPLAPGCGDTTSSTVTLGCLVKGYFPEPVTVTWNSGALSSDVHTFPAVLQSGLYTLTSSVTSSTWPSQTVTCNVAHPASSTKVDKKVERRNGGIGHKCPTCPTCHKCPVPELLGGPSVFIFPPKPKDILLISQNAKVTCVVVDVSEEEPDVQFSWFVNNVEVHTAQTQPREEQYNSTFRVVSALPIQHQDWMSGKEFKCKVNNKALPSPIEKTISKPKGLVRKPQVYVMGPPTEQLTEQTVSLTCLTSGFLPNDIGVEWTSNGHIEKNYKNTEPVMDSDGSFFMYSKLNVERSRWDSRAPFVCSVVHEGLHNHHVEKSISRPP

SEQ ID NO: 284 - protein, 107 aa, Artificial sequence
  Rat kappa Light Chain Constant Domain
RADAAPTVSIFPPSTEQLATGGASVVCLMNNFYPRDISVKWKIDGTERRDGVLDSVTDQDSKDSTYSMSSTLSLTKADYESHNLYTCEVVHKTSSSPVVKSFNRNEC

SEQ ID NO: 285 - protein, 120 aa, Artificial sequence
  Antibody to ASGR1 VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDFSSRRWYLEYWGQGTLVTVSS

SEQ ID NO: 286 - protein, 109 aa, Artificial sequence
  Antibody to ASGR1 VL
SSELTQDPAVSVALGQTVRITCQGDSLRSYYASWYQQKPGQAPVLVIYGKNNRPSGIPDRFSGSSSGNTASLTITGAQAEDEADYYCNSLERIGYLSYVFGGGTKLTVL

SEQ ID NO: 287 - protein, 120 aa, Artificial sequence
  Antibody to mzASGR1 VH
EVKLVESGGGLVKPGGSLKLSCAASGFTFSSYAMSWVRQTPEKRLEWVSAISGSGGSTYYADSVKGRFTISRDNAKNTLYLQMSSLRSEDTAMYYCAKDFSSRRWYLEYWGQGTTLTVSS

SEQ ID NO: 288 - protein, 109 aa, Artificial sequence
  Antibody to mzASGR1 VL
QAVLTQEPALSVSLGQTVTLTCQGDSLRSYYASWYQQKPGQAPVLVIYGKNNRPSGVPARFSGSLIGDKAALTITGAQTEDEAIYYCNSLERIGYLSYVFGGGTKVTVL

SEQ ID NO: 289 - protein, 114 aa, Artificial sequence
  Antibody to ASGR1 G2D VH
EVQLQQSGPELVKPGASVKISCKASGYSFTGYYMHWVKQSHVKSLEWIGRINPNNGATNYNQNFKDKASLTVDKSSSTAYMEFHSLTSEDSAVYYCTSVNFYYWGQGTTLTVSS

SEQ ID NO: 290 - protein, 107 aa, Artificial sequence
  Antibody to ASGR1 G2D VL
DIKMTQSPSSMYASLGERVTITCKASQVINSYLSWFQQKPGKSPETLIYRANTLVDGVPSRFSGSGSGQDYSLTISSLEYEDLGIYYCLQYAEFPYTFGGGTKLEIK

SEQ ID NO: 291 - protein, 113 aa, Artificial sequence
  Antibody to ASGR1 K2E VH
QVQLQQSAAELARPGASVKMSCKASGYTFTSYTMHWVKQRPGQGLEWIGYISPSSGYTEYNQKFKDKTTLTADKSSSTAYMQLSSLTSEDSAVYYCARKFDYWGQGTTLTVSS

SEQ ID NO: 292 - protein, 107 aa, Artificial sequence
  Antibody to ASGR1 K2E VL
DIKMTQSPSSMYASLGERVTITCKASQDINSYLSWFQQKPGKSPKTLIYRANRLVDGVPSRFSGSGSGQDFSLTISSLEYEDMGIYYCLQYDEFPFTFGSGTKLEIK

SEQ ID NO: 293 - protein, 121 aa, Artificial sequence
  Antibody to ASGR1 L4L VH
DVQLQESGPGLVKPSQSLSLTCTVTGYSITSDYAWNWIRQFPGNKLEWMGYISYSGSTRYNPSLKSRISITRDTSKNQFFLQLNSVTTEDTATYYCARRYRYDEGYGMDYWGQGTSVTVSS

SEQ ID NO: 294 - protein, 108 aa, Artificial sequence
  Antibody to ASGR1 L4L VL
DIQMTQSPASLSVFVGEIVTITCRASENIYSNLAWYQQKQGKSPQLLVYAATNLADGVPSRFTGSGSGTQFSLKINSLQSEDFGNYYCQHFWGTPPWTFGGGTKLEIK

SEQ ID NO: 295 - protein, 123 aa, Artificial sequence
  Antibody to ASGR1 H8K VH
DVQLQESGPGLVKPSQSLSLTCTVTGFSITSDYAWNWIRQFPGNQLEWMGYRSYRGSTSYNPSLKSRISITRDTSKNQFFLQLNSVTTEDTATYYCARRGYYGSSSHWYFDVWGAGTTVTVSS

SEQ ID NO: 296 - protein, 107 aa, Artificial sequence
  Antibody to ASGR1 H8K VL
DIQMTQSPASLSASVGETVTITCRASENIYSYLAWYQQKQGKSPQLLVYNAKTLAEGVPSRFSGSGSGTQFSLKINSLQPEDFGSYYCQHHYGTPLTFGSGTKLELK

SEQ ID NO: 297 - protein, 115 aa, Artificial sequence
  Antibody to ASGR1 J4F VH
QVQLQQSGDDLVKPGASVKLSCKASGYTFTSYWINWIKQRPGQGLEWIGRIVPGSGSTYYNEMFKDKATLTVDTSSSTAYIQLSSLSSEDSAVYFCARKPNFDVWGAGTTVTVSS

SEQ ID NO: 298 - protein, 107 aa, Artificial sequence
  Antibody to ASGR1 J4F VL
DIVMTQSQKFMSTSVGDRVSVTCKASQNVGTNVAWYQQKPGQSPKAVIYSASYRFSGVPDRLTGSGSGTDFTLTITNVQSEDLAEYFCQQYNSYPLTFGAGTKLELK

SEQ ID NO: 299 - protein, 114 aa, Artificial sequence
  Antibody to ASGR1 hzG2D VH
QVQLVQSGAEVKKPGASVKVSCKASGYTFTGYYMHWVRQAPGQRLEWMGRINPNNGATNYNQNFKDRVTITRDTSASTAYMELSSLRSEDTAVYYCARVNFYYWGQGTTLTVSS

SEQ ID NO: 300 - protein, 107 aa, Artificial sequence
  Antibody to ASGR1 hzG2D VL
DIQMTQSPSSLSASVGDRVTITCKASQVINSYLSWFQQKPGKAPKSLIYRANTLVDGVPSRFSGSGSGTDFTLTISSLQPEDFATYYCLQYAEFPYTFGGGTKVEIK

SEQ ID NO: 301 - protein, 114 aa, Artificial sequence
  Antibody to ASGR1 hzG2.1D T28S VH
QVQLVQSGAEVKKPGASVKVSCKASGYSFTGYYMHWVRQAPGQRLEWMGRINPNNGATNYNQNFKDRVTITRDTSASTAYMELSSLRSEDTAVYYCARVNFYYWGQGTTLTVSS

SEQ ID NO: 302 - protein, 107 aa, Artificial sequence
  Antibody to ASGR1 hzG2.1D D56S VL
DIQMTQSPSSLSASVGDRVTITCKASQVINSYLSWFQQKPGKAPKSLIYRANTLVSGVPSRFSGSGSGTDFTLTISSLQPEDFATYYCLQYAEFPYTFGGGTKVEIK

SEQ ID NO: 303 - protein, 114 aa, Artificial sequence
  Antibody to ASGR1 hzG2.2D T28S, R66K, V67A VH
QVQLVQSGAEVKKPGASVKVSCKASGYSFTGYYMHWVRQAPGQRLEWMGRINPNNGATNYNQNFKDKATITRDTSASTAYMELSSLRSEDTAVYYCARVNFYYWGQGTTLTVSS

SEQ ID NO: 304 - protein, 107 aa, Artificial sequence
  Antibody to ASGR1 hzG2.2D D56E VL
DIQMTQSPSSLSASVGDRVTITCKASQVINSYLSWFQQKPGKAPKSLIYRANTLVEGVPSRFSGSGSGTDFTLTISSLQPEDFATYYCLQYAEFPYTFGGGTKVEIK

SEQ ID NO: 305 - protein, 114 aa, Artificial sequence
  Antibody to ASGR1 hzG2.3D T28S, R66K, V67A, A93T, R94S VH
QVQLVQSGAEVKKPGASVKVSCKASGYSFTGYYMHWVRQAPGQRLEWMGRINPNNGATNYNQNFKDKATITRDTSASTAYMELSSLRSEDTAVYYCTSVNFYYWGQGTTLTVSS

SEQ ID NO: 306 - protein, 114 aa, Artificial sequence
  Antibody to ASGR1 hzG2.4D T28S, R66K, V67A, T68S, I69L, R71V VH
QVQLVQSGAEVKKPGASVKVSCKASGYSFTGYYMHWVRQAPGQRLEWMGRINPNNGATNYNQNFKDKASLTVDTSASTAYMELSSLRSEDTAVYYCARVNFYYWGQGTTLTVSS

SEQ ID NO: 307 - protein, 114 aa, Artificial sequence
  Antibody to ASGR1 hzG2.5D T28S, R66K, V67A, T68S, I69L, R71V, A93T, R94S VH
QVQLVQSGAEVKKPGASVKVSCKASGYSFTGYYMHWVRQAPGQRLEWMGRINPNNGATNYNQNFKDKASLTVDTSASTAYMELSSLRSEDTAVYYCTSVNFYYWGQGTTLTVSS

SEQ ID NO: 308 - protein, 114 aa, Artificial sequence
  Antibody to ASGR1 hzG2.6D T28S, T68S, I69L, R71V VH
QVQLVQSGAEVKKPGASVKVSCKASGYSFTGYYMHWVRQAPGQRLEWMGRINPNNGATNYNQNFKDRVSLTVDTSASTAYMELSSLRSEDTAVYYCARVNFYYWGQGTTLTVSS

SEQ ID NO: 309 - protein, 114 aa, Artificial sequence
  Antibody to ASGR1 hzG2.7D T28S, T68S, I69L, R71V, A93T, R94S VH
QVQLVQSGAEVKKPGASVKVSCKASGYSFTGYYMHWVRQAPGQRLEWMGRINPNNGATNYNQNFKDRVSLTVDTSASTAYMELSSLRSEDTAVYYCTSVNFYYWGQGTTLTVSS

SEQ ID NO: 310 - protein, 114 aa, Artificial sequence
  Antibody to ASGR1 hzG2.7D T28S, T68S, I69L, R71V, A93T, R94S VH
QVQLVQSGAEVKKPGASVKVSCKASGYSFTGYYMHWVRQAPGQRLEWMGRINPNNGATNYNQNFKDRVTITRDTSASTAYMELSSLRSEDTAVYYCTSVNFYYWGQGTTLTVSS

SEQ ID NO: 311 - protein, 114 aa, Artificial sequence
  Antibody to ASGR1 hzG2.9D T28S, N54Q VH
QVQLVQSGAEVKKPGASVKVSCKASGYSFTGYYMHWVRQAPGQRLEWMGRINPNQGATNYNQNFKDRVTITRDTSASTAYMELSSLRSEDTAVYYCARVNFYYWGQGTTLTVSS

SEQ ID NO: 312 - protein, 114 aa, Artificial sequence
  Antibody to ASGR1 hzG2.10D T28S, N54Q, R66K, V67A VH
QVQLVQSGAEVKKPGASVKVSCKASGYSFTGYYMHWVRQAPGQRLEWMGRINPNQGATNYNQNFKDKATITRDTSASTAYMELSSLRSEDTAVYYCARVNFYYWGQGTTLTVSS

SEQ ID NO: 313 - protein, 114 aa, Artificial sequence
  Antibody to ASGR1 hzG2.11D T28S, N54Q, R66K, V67A, A93T, R94S VH
QVQLVQSGAEVKKPGASVKVSCKASGYSFTGYYMHWVRQAPGQRLEWMGRINPNQGATNYNQNFKDKATITRDTSASTAYMELSSLRSEDTAVYYCTSVNFYYWGQGTTLTVSS

SEQ ID NO: 314 - protein, 114 aa, Artificial sequence
  Antibody to ASGR1 hzG2.12D T28S, N54Q, R66K, V67A, T68S, I69L, R71V VH
QVQLVQSGAEVKKPGASVKVSCKASGYSFTGYYMHWVRQAPGQRLEWMGRINPNQGATNYNQNFKDKASLTVDTSASTAYMELSSLRSEDTAVYYCARVNFYYWGQGTTLTVSS

SEQ ID NO: 315 - protein, 114 aa, Artificial sequence
  Antibody to ASGR1 hzG2.13D T28S, N54Q, R66K, V67A, T68S, I69L, R71V, A93T, R94S VH
QVQLVQSGAEVKKPGASVKVSCKASGYSFTGYYMHWVRQAPGQRLEWMGRINPNQGATNYNQNFKDKASLTVDTSASTAYMELSSLRSEDTAVYYCTSVNFYYWGQGTTLTVSS

SEQ ID NO: 316 - protein, 114 aa, Artificial sequence
  Antibody to ASGR1 hzG2.14D T28S, N54Q, T68S, I69L, R71V VH
QVQLVQSGAEVKKPGASVKVSCKASGYSFTGYYMHWVRQAPGQRLEWMGRINPNQGATNYNQNFKDRVSLTVDTSASTAYMELSSLRSEDTAVYYCARVNFYYWGQGTTLTVSS

SEQ ID NO: 317 - protein, 114 aa, Artificial sequence
  Antibody to ASGR1 hzG2.15D T28S, N54Q, T68S, I69L, R71V, A93T, R94S VH
QVQLVQSGAEVKKPGASVKVSCKASGYSFTGYYMHWVRQAPGQRLEWMGRINPNQGATNYNQNFKDRVSLTVDTSASTAYMELSSLRSEDTAVYYCTSVNFYYWGQGTTLTVSS

SEQ ID NO: 318 - protein, 114 aa, Artificial sequence
  Antibody to ASGR1 hzG2.16D T28S, N54Q, A93T, R94S VH
QVQLVQSGAEVKKPGASVKVSCKASGYSFTGYYMHWVRQAPGQRLEWMGRINPNQGATNYNQNFKDRVTITRDTSASTAYMELSSLRSEDTAVYYCTSVNFYYWGQGTTLTVSS

SEQ ID NO: 319 - protein, 114 aa, Artificial sequence
  Antibody to ASGR1 hzG2.17D T28S, G55A VH
QVQLVQSGAEVKKPGASVKVSCKASGYSFTGYYMHWVRQAPGQRLEWMGRINPNNAATNYNQNFKDRVTITRDTSASTAYMELSSLRSEDTAVYYCARVNFYYWGQGTTLTVSS

SEQ ID NO: 320 - protein, 114 aa, Artificial sequence
  Antibody to ASGR1 hzG2.18D T28S, G55A, R66K, V67A VH
QVQLVQSGAEVKKPGASVKVSCKASGYSFTGYYMHWVRQAPGQRLEWMGRINPNNAATNYNQNFKDKATITRDTSASTAYMELSSLRSEDTAVYYCARVNFYYWGQGTTLTVSS

SEQ ID NO: 321 - protein, 114 aa, Artificial sequence
  Antibody to ASGR1 hzG2.19D T28S, G55A, R66K, V67A, A93T, R94S VH
QVQLVQSGAEVKKPGASVKVSCKASGYSFTGYYMHWVRQAPGQRLEWMGRINPNNAATNYNQNFKDKATITRDTSASTAYMELSSLRSEDTAVYYCTSVNFYYWGQGTTLTVSS

SEQ ID NO: 322 - protein, 114 aa, Artificial sequence
  Antibody to ASGR1 hzG2.20D T28S, G55A, R66K, V67A, T68S, I69L, R71V VH
QVQLVQSGAEVKKPGASVKVSCKASGYSFTGYYMHWVRQAPGQRLEWMGRINPNNAATNYNQNFKDKASLTVDTSASTAYMELSSLRSEDTAVYYCARVNFYYWGQGTTLTVSS

SEQ ID NO: 323 - protein, 114 aa, Artificial sequence
  Antibody to ASGR1 hzG2.21D T28S, G55A, R66K, V67A, T68S, I69L, R71V, A93T, R94S VH
QVQLVQSGAEVKKPGASVKVSCKASGYSFTGYYMHWVRQAPGQRLEWMGRINPNNAATNYNQNFKDKASLTVDTSASTAYMELSSLRSEDTAVYYCTSVNFYYWGQGTTLTVSS

SEQ ID NO: 324 - protein, 114 aa, Artificial sequence
  Antibody to ASGR1 hzG2.22D T28S, G55A, T68S, I69L, R71V VH
QVQLVQSGAEVKKPGASVKVSCKASGYSFTGYYMHWVRQAPGQRLEWMGRINPNNAATNYNQNFKDRVSLTVDTSASTAYMELSSLRSEDTAVYYCARVNFYYWGQGTTLTVSS

SEQ ID NO: 325 - protein, 114 aa, Artificial sequence
  Antibody to ASGR1 hzG2.23D T28S, G55A, T68S, I69L, R71V, A93T, R94S VH
QVQLVQSGAEVKKPGASVKVSCKASGYSFTGYYMHWVRQAPGQRLEWMGRINPNNAATNYNQNFKDRVSLTVDTSASTAYMELSSLRSEDTAVYYCTSVNFYYWGQGTTLTVSS

SEQ ID NO: 326 - protein, 114 aa, Artificial sequence
  Antibody to ASGR1 hzG2.24D T28S, G55A, A93T, R94S VH
QVQLVQSGAEVKKPGASVKVSCKASGYSFTGYYMHWVRQAPGQRLEWMGRINPNNAATNYNQNFKDRVTITRDTSASTAYMELSSLRSEDTAVYYCTSVNFYYWGQGTTLTVSS

SEQ ID NO: 327 - protein, 114 aa, Artificial sequence
  Antibody to ASGR1 hzG2.25D N54Q, R66K, V67A VH
QVQLVQSGAEVKKPGASVKVSCKASGYTFTGYYMHWVRQAPGQRLEWMGRINPNQGATNYNQNFKDKATITRDTSASTAYMELSSLRSEDTAVYYCARVNFYYWGQGTTLTVSS

SEQ ID NO: 328 - protein, 114 aa, Artificial sequence
  Antibody to ASGR1 hzG2.26D N54Q, R66K, V67A, A93T, R94S VH
QVQLVQSGAEVKKPGASVKVSCKASGYTFTGYYMHWVRQAPGQRLEWMGRINPNQGATNYNQNFKDKATITRDTSASTAYMELSSLRSEDTAVYYCTSVNFYYWGQGTTLTVSS

SEQ ID NO: 329 - protein, 114 aa, Artificial sequence
  Antibody to ASGR1 hzG2.27D N54Q, R66K, V67A, T68S, I69L, R71V VH
QVQLVQSGAEVKKPGASVKVSCKASGYTFTGYYMHWVRQAPGQRLEWMGRINPNQGATNYNQNFKDKASLTVDTSASTAYMELSSLRSEDTAVYYCARVNFYYWGQGTTLTVSS

SEQ ID NO: 330 - protein, 114 aa, Artificial sequence
  Antibody to ASGR1 hzG2.28D N54Q, R66K, V67A, T68S, I69L, R71V, A93T, R94S VH
QVQLVQSGAEVKKPGASVKVSCKASGYTFTGYYMHWVRQAPGQRLEWMGRINPNQGATNYNQNFKDKASLTVDTSASTAYMELSSLRSEDTAVYYCTSVNFYYWGQGTTLTVSS

SEQ ID NO: 331 - protein, 114 aa, Artificial sequence
  Antibody to ASGR1 hzG2.29D N54Q, T68S, I69L, R71V VH
QVQLVQSGAEVKKPGASVKVSCKASGYTFTGYYMHWVRQAPGQRLEWMGRINPNQGATNYNQNFKDRVSLTVDTSASTAYMELSSLRSEDTAVYYCARVNFYYWGQGTTLTVSS

SEQ ID NO: 332 - protein, 114 aa, Artificial sequence
  Antibody to ASGR1 hzG2.30D N54Q, T68S, I69L, R71V, A93T, R94S VH
QVQLVQSGAEVKKPGASVKVSCKASGYTFTGYYMHWVRQAPGQRLEWMGRINPNQGATNYNQNFKDRVSLTVDTSASTAYMELSSLRSEDTAVYYCTSVNFYYWGQGTTLTVSS

SEQ ID NO: 333 - protein, 114 aa, Artificial sequence
  Antibody to ASGR1 hzG2.31D N54Q, A93T, R94S VH
QVQLVQSGAEVKKPGASVKVSCKASGYTFTGYYMHWVRQAPGQRLEWMGRINPNQGATNYNQNFKDRVTITRDTSASTAYMELSSLRSEDTAVYYCTSVNFYYWGQGTTLTVSS

SEQ ID NO: 334 - protein, 114 aa, Artificial sequence
  Antibody to ASGR1 hzG2.32D G55A, R66K, V67A VH
QVQLVQSGAEVKKPGASVKVSCKASGYTFTGYYMHWVRQAPGQRLEWMGRINPNNAATNYNQNFKDKATITRDTSASTAYMELSSLRSEDTAVYYCARVNFYYWGQGTTLTVSS

SEQ ID NO: 335 - protein, 114 aa, Artificial sequence
  Antibody to ASGR1 hzG2.33D G55A, R66K, V67A, A93T, R94S VH
QVQLVQSGAEVKKPGASVKVSCKASGYTFTGYYMHWVRQAPGQRLEWMGRINPNNAATNYNQNFKDKATITRDTSASTAYMELSSLRSEDTAVYYCTSVNFYYWGQGTTLTVSS

SEQ ID NO: 336 - protein, 114 aa, Artificial sequence
  Antibody to ASGR1 hzG2.34D G55A, R66K, V67A, T68S, I69L, R71V VH
QVQLVQSGAEVKKPGASVKVSCKASGYTFTGYYMHWVRQAPGQRLEWMGRINPNNAATNYNQNFKDKASLTVDTSASTAYMELSSLRSEDTAVYYCARVNFYYWGQGTTLTVSS

SEQ ID NO: 337 - protein, 114 aa, Artificial sequence
  Antibody to ASGR1 hzG2.35D G55A, R66K, V67A, T68S, I69L, R71V, A93T, R94S VH
QVQLVQSGAEVKKPGASVKVSCKASGYTFTGYYMHWVRQAPGQRLEWMGRINPNNAATNYNQNFKDKASLTVDTSASTAYMELSSLRSEDTAVYYCTSVNFYYWGQGTTLTVSS

SEQ ID NO: 338 - protein, 114 aa, Artificial sequence
  Antibody to ASGR1 hzG2.36D G55A, T68S, I69L, R71V VH
QVQLVQSGAEVKKPGASVKVSCKASGYTFTGYYMHWVRQAPGQRLEWMGRINPNNAATNYNQNFKDRVSLTVDTSASTAYMELSSLRSEDTAVYYCARVNFYYWGQGTTLTVSS

SEQ ID NO: 339 - protein, 114 aa, Artificial sequence
  Antibody to ASGR1 hzG2.37D G55A, T68S, I69L, R71V, A93T, R94S VH
QVQLVQSGAEVKKPGASVKVSCKASGYTFTGYYMHWVRQAPGQRLEWMGRINPNNAATNYNQNFKDRVSLTVDTSASTAYMELSSLRSEDTAVYYCTSVNFYYWGQGTTLTVSS

SEQ ID NO: 340 - protein, 114 aa, Artificial sequence
  Antibody to ASGR1 hzG2.38D G55A, A93T, R94S VH
QVQLVQSGAEVKKPGASVKVSCKASGYTFTGYYMHWVRQAPGQRLEWMGRINPNNAATNYNQNFKDRVTITRDTSASTAYMELSSLRSEDTAVYYCTSVNFYYWGQGTTLTVSS

SEQ ID NO: 341 - protein, 114 aa, Artificial sequence
  Antibody to ASGR1 hzG2.39D R66K, V67A VH
QVQLVQSGAEVKKPGASVKVSCKASGYTFTGYYMHWVRQAPGQRLEWMGRINPNNGATNYNQNFKDKATITRDTSASTAYMELSSLRSEDTAVYYCARVNFYYWGQGTTLTVSS

SEQ ID NO: 342 - protein, 114 aa, Artificial sequence
  Antibody to ASGR1 hzG2.40D R66K, V67A, A93T, R94S VH
QVQLVQSGAEVKKPGASVKVSCKASGYTFTGYYMHWVRQAPGQRLEWMGRINPNNGATNYNQNFKDKATITRDTSASTAYMELSSLRSEDTAVYYCTSVNFYYWGQGTTLTVSS

SEQ ID NO: 343 - protein, 114 aa, Artificial sequence
  Antibody to ASGR1 hzG2.41D R66K, V67A, T68S, I69L, R71V VH
QVQLVQSGAEVKKPGASVKVSCKASGYTFTGYYMHWVRQAPGQRLEWMGRINPNNGATNYNQNFKDKASLTVDTSASTAYMELSSLRSEDTAVYYCARVNFYYWGQGTTLTVSS

SEQ ID NO: 344 - protein, 114 aa, Artificial sequence
  Antibody to ASGR1 hzG2.42D R66K, V67A, T68S, I69L, R71V, A93T, R94S VH
QVQLVQSGAEVKKPGASVKVSCKASGYTFTGYYMHWVRQAPGQRLEWMGRINPNNGATNYNQNFKDKASLTVDTSASTAYMELSSLRSEDTAVYYCTSVNFYYWGQGTTLTVSS

SEQ ID NO: 345 - protein, 114 aa, Artificial sequence
  Antibody to ASGR1 hzG2.43D T68S, I69L, R71V VH
QVQLVQSGAEVKKPGASVKVSCKASGYTFTGYYMHWVRQAPGQRLEWMGRINPNNGATNYNQNFKDRVSLTVDTSASTAYMELSSLRSEDTAVYYCARVNFYYWGQGTTLTVSS

SEQ ID NO: 346 - protein, 114 aa, Artificial sequence
  Antibody to ASGR1 hzG2.44D T68S, I69L, R71V, A93T, R94S VH
QVQLVQSGAEVKKPGASVKVSCKASGYTFTGYYMHWVRQAPGQRLEWMGRINPNNGATNYNQNFKDRVSLTVDTSASTAYMELSSLRSEDTAVYYCTSVNFYYWGQGTTLTVSS

SEQ ID NO: 347 - protein, 114 aa, Artificial sequence
  Antibody to ASGR1 hzG2.45D A93T, R94S VH
QVQLVQSGAEVKKPGASVKVSCKASGYTFTGYYMHWVRQAPGQRLEWMGRINPNNGATNYNQNFKDRVTITRDTSASTAYMELSSLRSEDTAVYYCTSVNFYYWGQGTTLTVSS

SEQ ID NO: 348 - protein, 113 aa, Artificial sequence
  Antibody to ASGR1 hzK2E VH
QVQLVQSGAEVKKPGASVKVSCKASGYTFTSYTMHWVRQAPGQGLEWMGYISPSSGYTEYNQKFKDRVTMTRDTSTSTVYMELSSLRSEDTAVYYCARKFDYWGQGTTVTVSS

SEQ ID NO: 349 - protein, 107 aa, Artificial sequence
  Antibody to ASGR1 hzK2E VL
DIQMTQSPSSLSASVGDRVTITCKASQDINSYLSWFQQKPGKAPKSLIYRANRLVDGVPSRFSGSGSGTDFTLTISSLQPEDFATYYCLQYDEFPFTFGQGTKLEIK

SEQ ID NO: 350 - protein, 113 aa, Artificial sequence
  Antibody to ASGR1 hzK2.1E R66K, V67T VH
QVQLVQSGAEVKKPGASVKVSCKASGYTFTSYTMHWVRQAPGQGLEWMGYISPSSGYTEYNQKFKDKTTMTRDTSTSTVYMELSSLRSEDTAVYYCARKFDYWGQGTTVTVSS

SEQ ID NO: 351 - protein, 107 aa, Artificial sequence
  Antibody to ASGR1 hzK2.1E S46T VL
DIQMTQSPSSLSASVGDRVTITCKASQDINSYLSWFQQKPGKAPKTLIYRANRLVDGVPSRFSGSGSGTDFTLTISSLQPEDFATYYCLQYDEFPFTFGQGTKLEIK

SEQ ID NO: 352 - protein, 113 aa, Artificial sequence
  Antibody to ASGR1 hzK2.2E R66K, V67T, M69L, R71A VH
QVQLVQSGAEVKKPGASVKVSCKASGYTFTSYTMHWVRQAPGQGLEWMGYISPSSGYTEYNQKFKDKTTLTADTSTSTVYMELSSLRSEDTAVYYCARKFDYWGQGTTVTVSS

SEQ ID NO: 353 - protein, 107 aa, Artificial sequence
  Antibody to ASGR1 hzK2.2E D56E VL
DIQMTQSPSSLSASVGDRVTITCKASQDINSYLSWFQQKPGKAPKSLIYRANRLVEGVPSRFSGSGSGTDFTLTISSLQPEDFATYYCLQYDEFPFTFGQGTKLEIK

SEQ ID NO: 354 - protein, 113 aa, Artificial sequence
  Antibody to ASGR1 hzK2.3E R66K, V67T, M69L, R71A, T73K VH
QVQLVQSGAEVKKPGASVKVSCKASGYTFTSYTMHWVRQAPGQGLEWMGYISPSSGYTEYNQKFKDKTTLTADKSTSTVYMELSSLRSEDTAVYYCARKFDYWGQGTTVTVSS

SEQ ID NO: 355 - protein, 107 aa, Artificial sequence
  Antibody to ASGR1 hzK2.3E S46T D56E VL
DIQMTQSPSSLSASVGDRVTITCKASQDINSYLSWFQQKPGKAPKTLIYRANRLVEGVPSRFSGSGSGTDFTLTISSLQPEDFATYYCLQYDEFPFTFGQGTKLEIK

SEQ ID NO: 356 - protein, 113 aa, Artificial sequence
  Antibody to ASGR1 hzK2.4E M69L, R71A VH
QVQLVQSGAEVKKPGASVKVSCKASGYTFTSYTMHWVRQAPGQGLEWMGYISPSSGYTEYNQKFKDRVTLTADTSTSTVYMELSSLRSEDTAVYYCARKFDYWGQGTTVTVSS

SEQ ID NO: 357 - protein, 121 aa, Artificial sequence
  Antibody to ASGR1 hzL4L VH
QVQLQESGPGLVKPSQTLSLTCTVSGGSISSDYAWNWIRQHPGKGLEWIGYISYSGSTRYNPSLKSRVTISVDTSKNQFSLKLSSVTAADTAVYYCARRYRYDEGYGMDYWGQGTTVTVSS

SEQ ID NO: 358 - protein, 108 aa, Artificial sequence
  Antibody to ASGR1 hzL4L VL
DIQMTQSPSSLSASVGDRVTITCRASENIYSNLAWYQQKPGKAPKLLIYAATNLADGVPSRFSGSGSGTDFTLTISSLQPEDFATYYCQHFWGTPPWTFGGGTKVEIK

SEQ ID NO: 359 - protein, 121 aa, Artificial sequence
  Antibody to ASGR1 hzL4.1L S30T, V67I, T68S, S70T, V71R VH
QVQLQESGPGLVKPSQTLSLTCTVSGGSITSDYAWNWIRQHPGKGLEWIGYISYSGSTRYNPSLKSRISITRDTSKNQFSLKLSSVTAADTAVYYCARRYRYDEGYGMDYWGQGTTVTVSS

SEQ ID NO: 360 - protein, 108 aa, Artificial sequence
  Antibody to ASGR1 hzL4.1L D56S VL
DIQMTQSPSSLSASVGDRVTITCRASENIYSNLAWYQQKPGKAPKLLIYAATNLASGVPSRFSGSGSGTDFTLTISSLQPEDFATYYCQHFWGTPPWTFGGGTKVEIK

SEQ ID NO: 361 - protein, 121 aa, Artificial sequence
  Antibody to ASGR1 hzL4.2L G27Y, S30T, V67I, T68S, S70T, V71R VH
QVQLQESGPGLVKPSQTLSLTCTVSGYSITSDYAWNWIRQHPGKGLEWIGYISYSGSTRYNPSLKSRISITRDTSKNQFSLKLSSVTAADTAVYYCARRYRYDEGYGMDYWGQGTTVTVSS

SEQ ID NO: 362 - protein, 108 aa, Artificial sequence
  Antibody to ASGR1 hzL4.2L D56E VL
DIQMTQSPSSLSASVGDRVTITCRASENIYSNLAWYQQKPGKAPKLLIYAATNLAEGVPSRFSGSGSGTDFTLTISSLQPEDFATYYCQHFWGTPPWTFGGGTKVEIK

SEQ ID NO: 363 - protein, 121 aa, Artificial sequence
  Antibody to ASGR1 hzL4.3L G27Y, S30T VH
QVQLQESGPGLVKPSQTLSLTCTVSGYSITSDYAWNWIRQHPGKGLEWIGYISYSGSTRYNPSLKSRVTISVDTSKNQFSLKLSSVTAADTAVYYCARRYRYDEGYGMDYWGQGTTVTVSS

SEQ ID NO: 364 - protein, 108 aa, Artificial sequence
  Antibody to ASGR1 hzL4.3L G57A VL
DIQMTQSPSSLSASVGDRVTITCRASENIYSNLAWYQQKPGKAPKLLIYAATNLADAVPSRFSGSGSGTDFTLTISSLQPEDFATYYCQHFWGTPPWTFGGGTKVEIK

SEQ ID NO: 365 - protein, 121 aa, Artificial sequence
  Antibody to ASGR1 hzL4.4L G27Y, V67I, T68S, S70T, V71R VH
QVQLQESGPGLVKPSQTLSLTCTVSGYSISSDYAWNWIRQHPGKGLEWIGYISYSGSTRYNPSLKSRISITRDTSKNQFSLKLSSVTAADTAVYYCARRYRYDEGYGMDYWGQGTTVTVSS

SEQ ID NO: 366 - protein, 121 aa, Artificial sequence
  Antibody to ASGR1 hzL4.5L G27Y, S30T, S70T, V71R VH
QVQLQESGPGLVKPSQTLSLTCTVSGYSITSDYAWNWIRQHPGKGLEWIGYISYSGSTRYNPSLKSRVTITRDTSKNQFSLKLSSVTAADTAVYYCARRYRYDEGYGMDYWGQGTTVTVSS

SEQ ID NO: 367 - protein, 121 aa, Artificial sequence
  Antibody to ASGR1 hzL4.6L G27Y, S70T, V71R VH
QVQLQESGPGLVKPSQTLSLTCTVSGYSISSDYAWNWIRQHPGKGLEWIGYISYSGSTRYNPSLKSRVTITRDTSKNQFSLKLSSVTAADTAVYYCARRYRYDEGYGMDYWGQGTTVTVSS

SEQ ID NO: 368 - protein, 121 aa, Artificial sequence
  Antibody to ASGR1 hzL4.7L G27Y, S30T, V67I, T68S VH
QVQLQESGPGLVKPSQTLSLTCTVSGYSITSDYAWNWIRQHPGKGLEWIGYISYSGSTRYNPSLKSRISISVDTSKNQFSLKLSSVTAADTAVYYCARRYRYDEGYGMDYWGQGTTVTVSS

SEQ ID NO: 369 - protein, 121 aa, Artificial sequence
  Antibody to ASGR1 hzL4.8L G27Y, V67I, T68S VH
QVQLQESGPGLVKPSQTLSLTCTVSGYSISSDYAWNWIRQHPGKGLEWIGYISYSGSTRYNPSLKSRISISVDTSKNQFSLKLSSVTAADTAVYYCARRYRYDEGYGMDYWGQGTTVTVSS

SEQ ID NO: 370 - protein, 123 aa, Artificial sequence
  Antibody to ASGR1 hzH8K VH
QVQLQESGPGLVKPSQTLSLTCTVSGGSISSDYAWNWIRQHPGKGLEWIGYRSYRGSTSYNPSLKSRVTISVDTSKNQFSLKLSSVTAADTAVYYCARRGYYGSSSHWYFDVWGQGTTVTVSS

SEQ ID NO: 371 - protein, 107 aa, Artificial sequence
  Antibody to ASGR1 hzH8K VL
DIQMTQSPSSLSASVGDRVTITCRASENIYSYLAWYQQKPGKAPKLLIYNAKTLAEGVPSRFSGSGSGTDFTLTISSLQPEDFATYYCQHHYGTPLTFGGGTKVEIK

SEQ ID NO: 372 - protein, 123 aa, Artificial sequence
  Antibody to ASGR1 hzH8.1K S30T VH
QVQLQESGPGLVKPSQTLSLTCTVSGGSITSDYAWNWIRQHPGKGLEWIGYRSYRGSTSYNPSLKSRVTISVDTSKNQFSLKLSSVTAADTAVYYCARRGYYGSSSHWYFDVWGQGTTVTVSS

SEQ ID NO: 373 - protein, 123 aa, Artificial sequence
  Antibody to ASGR1 hzH8.2K G27F, S30T VH
QVQLQESGPGLVKPSQTLSLTCTVSGFSITSDYAWNWIRQHPGKGLEWIGYRSYRGSTSYNPSLKSRVTISVDTSKNQFSLKLSSVTAADTAVYYCARRGYYGSSSHWYFDVWGQGTTVTVSS

SEQ ID NO: 374 - protein, 123 aa, Artificial sequence
  Antibody to ASGR1 hzH8.3K V67I, T68S, S70T, V71R VH
QVQLQESGPGLVKPSQTLSLTCTVSGGSISSDYAWNWIRQHPGKGLEWIGYRSYRGSTSYNPSLKSRISITRDTSKNQFSLKLSSVTAADTAVYYCARRGYYGSSSHWYFDVWGQGTTVTVSS

SEQ ID NO: 375 - protein, 123 aa, Artificial sequence
  Antibody to ASGR1 hzH8.4K S30T, V67I, T68S, S70T, V71R VH
QVQLQESGPGLVKPSQTLSLTCTVSGGSITSDYAWNWIRQHPGKGLEWIGYRSYRGSTSYNPSLKSRISITRDTSKNQFSLKLSSVTAADTAVYYCARRGYYGSSSHWYFDVWGQGTTVTVSS

SEQ ID NO: 376 - protein, 123 aa, Artificial sequence
  Antibody to ASGR1 hzH8.5K G27F, S30T, V67I, T68S, S70T, V71R VH
QVQLQESGPGLVKPSQTLSLTCTVSGFSITSDYAWNWIRQHPGKGLEWIGYRSYRGSTSYNPSLKSRISITRDTSKNQFSLKLSSVTAADTAVYYCARRGYYGSSSHWYFDVWGQGTTVTVSS

SEQ ID NO: 377 - protein, 123 aa, Artificial sequence
  Antibody to ASGR1 hzH8.6K S30T, V67I, T68S VH
QVQLQESGPGLVKPSQTLSLTCTVSGGSITSDYAWNWIRQHPGKGLEWIGYRSYRGSTSYNPSLKSRISISVDTSKNQFSLKLSSVTAADTAVYYCARRGYYGSSSHWYFDVWGQGTTVTVSS

SEQ ID NO: 378 - protein, 123 aa, Artificial sequence
  Antibody to ASGR1 hzH8.7K S30T, S70T, V71R VH
QVQLQESGPGLVKPSQTLSLTCTVSGGSITSDYAWNWIRQHPGKGLEWIGYRSYRGSTSYNPSLKSRVTITRDTSKNQFSLKLSSVTAADTAVYYCARRGYYGSSSHWYFDVWGQGTTVTVSS

SEQ ID NO: 379 - protein, 123 aa, Artificial sequence
  Antibody to ASGR1 hzH8.8K V67I, T68S VH
QVQLQESGPGLVKPSQTLSLTCTVSGGSISSDYAWNWIRQHPGKGLEWIGYRSYRGSTSYNPSLKSRISISVDTSKNQFSLKLSSVTAADTAVYYCARRGYYGSSSHWYFDVWGQGTTVTVSS

SEQ ID NO: 380 - protein, 123 aa, Artificial sequence
  Antibody to ASGR1 hzH8.9K S70T, V71R VH
QVQLQESGPGLVKPSQTLSLTCTVSGGSISSDYAWNWIRQHPGKGLEWIGYRSYRGSTSYNPSLKSRVTITRDTSKNQFSLKLSSVTAADTAVYYCARRGYYGSSSHWYFDVWGQGTTVTVSS

SEQ ID NO: 381 - protein, 115 aa, Artificial sequence
  Antibody to ASGR1 hzJ4F VH
QVQLVQSGAEVKKPGASVKVSCKASGYTFTSYWINWVRQAPGQGLEWMGRIVPGSGSTYYNEMFKDRVTMTRDTSTSTVYMELSSLRSEDTAVYYCARKPNFDVWGQGTTVTVSS

SEQ ID NO: 382 - protein, 107 aa, Artificial sequence
  Antibody to ASGR1 hzJ4F VL
DIQMTQSPSSLSASVGDRVTITCKASQNVGTNVAWYQQKPGKAPKLLIYSASYRFSGVPSRFSGSGSGTDFTLTISSLQPEDFATYYCQQYNSYPLTFGGGTKVEIK

SEQ ID NO: 383 - protein, 115 aa, Artificial sequence
  Antibody to ASGR1 hzJ4.1F V37I VH
QVQLVQSGAEVKKPGASVKVSCKASGYTFTSYWINWIRQAPGQGLEWMGRIVPGSGSTYYNEMFKDRVTMTRDTSTSTVYMELSSLRSEDTAVYYCARKPNFDVWGQGTTVTVSS

SEQ ID NO: 384 - protein, 107 aa, Artificial sequence
  Antibody to ASGR1 hzJ4.1F Q3V VL
DIVMTQSPSSLSASVGDRVTITCKASQNVGTNVAWYQQKPGKAPKLLIYSASYRFSGVPSRFSGSGSGTDFTLTISSLQPEDFATYYCQQYNSYPLTFGGGTKVEIK

SEQ ID NO: 385 - protein, 115 aa, Artificial sequence
  Antibody to ASGR1 hzJ4.2F V37I, R38K, M48I VH
QVQLVQSGAEVKKPGASVKVSCKASGYTFTSYWINWIKQAPGQGLEWIGRIVPGSGSTYYNEMFKDRVTMTRDTSTSTVYMELSSLRSEDTAVYYCARKPNFDVWGQGTTVTVSS

SEQ ID NO: 386 - protein, 107 aa, Artificial sequence
  Antibody to ASGR1 hzJ4.2F Q3V L46A VL
DIVMTQSPSSLSASVGDRVTITCKASQNVGTNVAWYQQKPGKAPKALIYSASYRFSGVPSRFSGSGSGTDFTLTISSLQPEDFATYYCQQYNSYPLTFGGGTKVEIK

SEQ ID NO: 387 - protein, 115 aa, Artificial sequence
  Antibody to ASGR1 hzJ4.3F V37I, R66K, V67A VH
QVQLVQSGAEVKKPGASVKVSCKASGYTFTSYWINWIRQAPGQGLEWMGRIVPGSGSTYYNEMFKDKATMTRDTSTSTVYMELSSLRSEDTAVYYCARKPNFDVWGQGTTVTVSS

SEQ ID NO: 388 - protein, 107 aa, Artificial sequence
  Antibody to ASGR1 hzJ4.3F L46A VL
DIQMTQSPSSLSASVGDRVTITCKASQNVGTNVAWYQQKPGKAPKALIYSASYRFSGVPSRFSGSGSGTDFTLTISSLQPEDFATYYCQQYNSYPLTFGGGTKVEIK

SEQ ID NO: 389 - protein, 115 aa, Artificial sequence
  Antibody to ASGR1 hzJ4.4F V37I, R38K, M48I, R66K, V67A VH
QVQLVQSGAEVKKPGASVKVSCKASGYTFTSYWINWIKQAPGQGLEWIGRIVPGSGSTYYNEMFKDKATMTRDTSTSTVYMELSSLRSEDTAVYYCARKPNFDVWGQGTTVTVSS

SEQ ID NO: 390 - protein, 107 aa, Artificial sequence
  Antibody to ASGR1 hzJ4.4F L46A, L47V VL
DIQMTQSPSSLSASVGDRVTITCKASQNVGTNVAWYQQKPGKAPKAVIYSASYRFSGVPSRFSGSGSGTDFTLTISSLQPEDFATYYCQQYNSYPLTFGGGTKVEIK

SEQ ID NO: 391 - protein, 115 aa, Artificial sequence
  Antibody to ASGR1 hzJ4.5F R66K, V67A VH
QVQLVQSGAEVKKPGASVKVSCKASGYTFTSYWINWVRQAPGQGLEWMGRIVPGSGSTYYNEMFKDKATMTRDTSTSTVYMELSSLRSEDTAVYYCARKPNFDVWGQGTTVTVSS

SEQ ID NO: 392 - protein, 107 aa, Artificial sequence
  Antibody to ASGR1 hzJ4.5F L46A, L47V, F62L VL
DIQMTQSPSSLSASVGDRVTITCKASQNVGTNVAWYQQKPGKAPKAVIYSASYRFSGVPSRLSGSGSGTDFTLTISSLQPEDFATYYCQQYNSYPLTFGGGTKVEIK

SEQ ID NO: 393 - protein, 115 aa, Artificial sequence
  Antibody to ASGR1 hzJ4.6F R66K, V67A, V78A VH
QVQLVQSGAEVKKPGASVKVSCKASGYTFTSYWINWVRQAPGQGLEWMGRIVPGSGSTYYNEMFKDKATMTRDTSTSTAYMELSSLRSEDTAVYYCARKPNFDVWGQGTTVTVSS

SEQ ID NO: 394 - protein, 107 aa, Artificial sequence
  Antibody to ASGR1 hzJ4.6F L46A, L47V, S60D, F62L, S63T VL
DIQMTQSPSSLSASVGDRVTITCKASQNVGTNVAWYQQKPGKAPKAVIYSASYRFSGVPDRLTGSGSGTDFTLTISSLQPEDFATYYCQQYNSYPLTFGGGTKVEIK

SEQ ID NO: 395 - protein, 115 aa, Artificial sequence
  Antibody to ASGR1 hzJ4.7F R66K, V67A, V78A, M80I, E81Q VH
QVQLVQSGAEVKKPGASVKVSCKASGYTFTSYWINWVRQAPGQGLEWMGRIVPGSGSTYYNEMFKDKATMTRDTSTSTAYIQLSSLRSEDTAVYYCARKPNFDVWGQGTTVTVSS

SEQ ID NO: 396 - protein, 115 aa, Artificial sequence
  Antibody to ASGR1 hzJ4.8F R66K, V67A, M69L, R71V VH
QVQLVQSGAEVKKPGASVKVSCKASGYTFTSYWINWVRQAPGQGLEWMGRIVPGSGSTYYNEMFKDKATLTVDTSTSTVYMELSSLRSEDTAVYYCARKPNFDVWGQGTTVTVSS

SEQ ID NO: 397 - protein, 115 aa, Artificial sequence
  Antibody to ASGR1 hzJ4.9F M69L, R71V VH
QVQLVQSGAEVKKPGASVKVSCKASGYTFTSYWINWVRQAPGQGLEWMGRIVPGSGSTYYNEMFKDRVTLTVDTSTSTVYMELSSLRSEDTAVYYCARKPNFDVWGQGTTVTVSS

SEQ ID NO: 398 - protein, 115 aa, Artificial sequence
  Antibody to ASGR1 hzJ4.10F R71V VH
QVQLVQSGAEVKKPGASVKVSCKASGYTFTSYWINWVRQAPGQGLEWMGRIVPGSGSTYYNEMFKDRVTMTVDTSTSTVYMELSSLRSEDTAVYYCARKPNFDVWGQGTTVTVSS